SEQUENCE LISTING (184 sequences)

SEQ ID NO: 1 - protein, 1273 aa, SARS-Cov2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 200 aa, artificial sequence
  Synthetic SARS-CoV-2 Spike receptor-binding domain (RBD)
NITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGS

SEQ ID NO: 3 - protein, 123 aa, Artificial sequence
  synthetic polypeptide (Cv2.1169 VH)
QVQLVQSGPEVKKPGTSVKVSCKASGFTFTTSAVQWVRQARGQRLEWIGWIVVGSGNTNYAQKFQERVTITRDMSTTTAYMELSSLRSEDTAVYYCAAPYCSGGTCLDGFDIWGQGTMVTVSS

SEQ ID NO: 4 - protein, 108 aa, Artificial Sequence
  synthetic polypeptide (Cv2.1169 VL)
EIVLTQSPGTLSLSPGERATLSCRASQSVSRSYLAWYQQKPGQAPRLLIYSASSRATGIPDRFSGSGSGTDFTLTISRLEPEDFAVYYCQQYGSSPWTFGQGTKVEIK

SEQ ID NO: 5 - protein, 117 aa, Artificial sequence
  synthetic polypeptide (Cv2.1353 VH)
EVQLVESGGGLVQPGGSLRLSCAASGVTVSSNYMNWVRQAPGKGLEWVSVIYSGGSTFYADSVKGRFIISRDNSKNTLYLQMNSLKTEDTAVYYCARDLGPMGFDIWGQGTMVTVSS

SEQ ID NO: 6 - protein, 108 aa, Artificial Sequence
  synthetic polypeptide (Cv2.1353 VL)
DIQMTQSPSFLSASVGDRVTITCRASQGISSYLAWYQQKPGKAPKLLIYAASTLQSGVPSRFSGSGSGTEFTLTISSLQPEDFATYYCQQLNSDPPVTFGPGTKVDIK

SEQ ID NO: 7 - protein, 117 aa, Artificial Sequence
  synthetic polypeptide (Cv2.3194 VH)
EVQLVESGGGLIQPGGSLRLSCAASGITVTSNYMSWVRQAPGKGLEWVSVIYPGGSTFYADSVKGRFTISRDNSKNTLYLQMNSLRAEDTAVYYCARDLVVYGMDVWGQGTTVTVSS

SEQ ID NO: 8 - protein, 104 aa, Artificial Sequence
  synthetic polypeptide (Cv2.3194 VL)
EIVLTQSPGTLSLSPGERATLSCRASQSVSSSYLAWYQQKPGQAPRLLIYGASSRATGIPGRFSGSGSGTDFTLTISRLEPEDFAIYYCQQGVTFGGGTKVEIK

SEQ ID NO: 9 - protein, 117 aa, Artificial sequence
  synthetic polypeptide (Cv2.3235 VH)
EVQLVESGGGLIQPGGSLRLSCAASGFTVSRNYMSWVRQAPGKGLEWVSVIYSGGSTFYADSVKGRFTISRDNSKNTLYLQMNSLRAEDTAVYYCARDGDYYGMDVWGQGTTVTVSS

SEQ ID NO: 10 - protein, 108 aa, Artificial sequence
  synthetic polypeptide (Cv2.3235 VL)
DIQMTQSPSFLSASVGDRVTITCRASQGISSFLAWYQQKPGKAPKLLIYGASTLQSGVTSRFSGSGSGTEFTLTISSLQPEDFATYYCQRLDSYPPITFGQGTRLEIK

SEQ ID NO: 11 - protein, 117 aa, Artificial sequence
  synthetic polypeptide (Cv2.5213 VH)
EVQLVESGGGLIQPGGSLRLSCAASGFTVSSNYMSWVRQAPGKGLEWVSLIYSGGSTYYADSVKGRFTISRDNSKNTLYLQMNSLRAEDTAVYYCARDLNYYGMDVWGQGATVTVSS

SEQ ID NO: 12 - protein, 108 aa, Artificial sequence
  synthetic polypeptide (Cv2.5213 VL)
DIQMTQSPSSLSASVGDRVTITCRASQGISSYLAWYQQKPGKAPKLLIYAASTLQSGVPSRFSGSGSGTDFTLTISSLQPEDFATYYCQQLDSYSPFTFGPGTKVDIK

SEQ ID NO: 13 - protein, 452 aa, Artificial sequence
  synthetic polypeptide (Cv2.1169 full length heavy chain)
QVQLVQSGPEVKKPGTSVKVSCKASGFTFTTSAVQWVRQARGQRLEWIGWIVVGSGNTNYAQKFQERVTITRDMSTTTAYMELSSLRSEDTAVYYCAAPYCSGGTCLDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 215 aa, Artificial sequence
  synthetic polypeptide (Cv2.1169 full length light chain)
EIVLTQSPGTLSLSPGERATLSCRASQSVSRSYLAWYQQKPGQAPRLLIYSASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 446 aa, Artificial sequence
  synthetic polypeptide (Cv2.1353 full length heavy chain)
EVQLVESGGGLVQPGGSLRLSCAASGVTVSSNYMNWVRQAPGKGLEWVSVIYSGGSTFYADSVKGRFIISRDNSKNTLYLQMNSLKTEDTAVYYCARDLGPMGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 215 aa, Artificial sequence
  synthetic polypeptide (Cv2.1353 full length light chain)
DIQMTQSPSFLSASVGDRVTITCRASQGISSYLAWYQQKPGKAPKLLIYAASTLQSGVPSRFSGSGSGTEFTLTISSLQPEDFATYYCQQLNSDPPVTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 446 aa, Artificial sequence
  synthetic polypeptide (Cv2.3194 full length heavy chain)
EVQLVESGGGLIQPGGSLRLSCAASGITVTSNYMSWVRQAPGKGLEWVSVIYPGGSTFYADSVKGRFTISRDNSKNTLYLQMNSLRAEDTAVYYCARDLVVYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 211 aa, Artificial sequence
  synthetic polypeptide (Cv2.3194 full length light chain)
EIVLTQSPGTLSLSPGERATLSCRASQSVSSSYLAWYQQKPGQAPRLLIYGASSRATGIPGRFSGSGSGTDFTLTISRLEPEDFAIYYCQQGVTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 446 aa, Artificial sequence
  synthetic polypeptide (Cv2.3235 full length heavy chain)
EVQLVESGGGLIQPGGSLRLSCAASGFTVSRNYMSWVRQAPGKGLEWVSVIYSGGSTFYADSVKGRFTISRDNSKNTLYLQMNSLRAEDTAVYYCARDGDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 215 aa, Artificial sequence
  synthetic polypeptide (Cv2.3235 full length light chain)
DIQMTQSPSFLSASVGDRVTITCRASQGISSFLAWYQQKPGKAPKLLIYGASTLQSGVTSRFSGSGSGTEFTLTISSLQPEDFATYYCQRLDSYPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 446 aa, Artificial sequence
  synthetic polypeptide (Cv2.5213 full length heavy chain)
EVQLVESGGGLIQPGGSLRLSCAASGFTVSSNYMSWVRQAPGKGLEWVSLIYSGGSTYYADSVKGRFTISRDNSKNTLYLQMNSLRAEDTAVYYCARDLNYYGMDVWGQGATVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 215 aa, Artificial sequence
  synthetic polypeptide (Cv2.5213 full length light chain)
DIQMTQSPSSLSASVGDRVTITCRASQGISSYLAWYQQKPGKAPKLLIYAASTLQSGVPSRFSGSGSGTDFTLTISSLQPEDFATYYCQQLDSYSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 5 aa, Artificial sequence
  synthetic peptide (Cv2.1169 CDR-H1)
TSAVQ

SEQ ID NO: 24 - protein, 17 aa, Artificial sequence
  synthetic peptide (Cv2.1169 CDR-H2)
WIVVGSGNTNYAQKFQE

SEQ ID NO: 25 - protein, 14 aa, Artificial sequence
  synthetic peptide (Cv2.1169 CDR-H3)
PYCSGGTCLDGFDI

SEQ ID NO: 26 - protein, 12 aa, Artificial sequence
  synthetic peptide (Cv2.1169 CDR-L1)
RASQSVSRSYLA

SEQ ID NO: 27 - protein, 7 aa, Artificial sequence
  synthetic peptide (Cv2.1169-CDR-L2)
SASSRAT

SEQ ID NO: 28 - protein, 9 aa, Artificial sequence
  synthetic peptide (Cv2.1169 CDR-L3)
QQYGSSPWT

SEQ ID NO: 29 - protein, 5 aa, Artificial sequence
  synthetic peptide (Cv2.1353 CDR-H1)
SNYMN

SEQ ID NO: 30 - protein, 16 aa, Artificial sequence
  synthetic peptide (Cv2.1353 CDR-H2)
VIYSGGSTFYADSVKG

SEQ ID NO: 31 - protein, 9 aa, Artificial sequence
  synthetic peptide (Cv2.1353 CDR-H3)
DLGPMGFDI

SEQ ID NO: 32 - protein, 11 aa, Artificial sequence
  synthetic peptide (Cv2.1353 CDR-L1)
RASQGISSYLA

SEQ ID NO: 33 - protein, 7 aa, Artificial sequence
  synthetic peptide (Cv2.1353-CDR-L2)
AASTLQS

SEQ ID NO: 34 - protein, 10 aa, Artificial sequence
  synthetic peptide (Cv2.1353 CDR-L3)
QQLNSDPPVT

SEQ ID NO: 35 - protein, 5 aa, Artificial sequence
  synthetic peptide (Cv2.3194 CDR-H1)
SNYMS

SEQ ID NO: 36 - protein, 16 aa, Artificial sequence
  synthetic peptide (Cv2.3194 CDR-H2)
VIYPGGSTFYADSVKG

SEQ ID NO: 37 - protein, 9 aa, Artificial sequence
  synthetic peptide (Cv2.3194 CDR-H3)
DLVVYGMDV

SEQ ID NO: 38 - protein, 12 aa, Artificial sequence
  synthetic peptide (Cv2.3194 CDR-L1)
RASQSVSSSYLA

SEQ ID NO: 39 - protein, 7 aa, Artificial sequence
  synthetic peptide (Cv2.3194-CDR-L2)
GASSRAT

SEQ ID NO: 40 - protein, 5 aa, Artificial sequence
  synthetic peptide (Cv2.3194 CDR-L3)
QQGVT

SEQ ID NO: 41 - protein, 5 aa, Artificial sequence
  synthetic peptide (Cv2.3235 CDR-H1)
RNYMS

SEQ ID NO: 42 - protein, 16 aa, Artificial sequence
  synthetic peptide (Cv2.3235 CDR-H2)
VIYSGGSTFYADSVKG

SEQ ID NO: 43 - protein, 9 aa, Artificial sequence
  synthetic peptide (Cv2.3235 CDR-H3)
DGDYYGMDV

SEQ ID NO: 44 - protein, 11 aa, Artificial sequence
  synthetic peptide (Cv2.3235 CDR-L1)
RASQGISSFLA

SEQ ID NO: 45 - protein, 7 aa, Artificial sequence
  synthetic peptide (Cv2.3235-CDR-L2)
GASTLQS

SEQ ID NO: 46 - protein, 10 aa, Artificial sequence
  synthetic peptide (Cv2.3235 CDR-L3)
QRLDSYPPIT

SEQ ID NO: 47 - protein, 5 aa, Artificial sequence
  synthetic peptide (Cv2.5213 CDR-H1)
SNYMS

SEQ ID NO: 48 - protein, 16 aa, Artificial sequence
  synthetic peptide (Cv2.5213 CDR-H2)
LIYSGGSTYYADSVKG

SEQ ID NO: 49 - protein, 9 aa, Artificial sequence
  synthetic peptide (Cv2.5213 CDR-H3)
DLNYYGMDV

SEQ ID NO: 50 - protein, 11 aa, Artificial sequence
  synthetic peptide (Cv2.5213 CDR-L1)
RASQGISSYLA

SEQ ID NO: 51 - protein, 7 aa, Artificial sequence
  synthetic peptide (Cv2.5213-CDR-L2)
AASTLQS

SEQ ID NO: 52 - protein, 10 aa, Artificial sequence
  synthetic peptide (Cv2.5213 CDR-L3)
QQLDSYSPFT

SEQ ID NO: 53 - protein, 30 aa, Artificial sequence
  synthetic peptide (Cv2.1169 H-FW1)
QVQLVQSGPEVKKPGTSVKVSCKASGFTFT

SEQ ID NO: 54 - protein, 14 aa, Artificial sequence
  synthetic peptide (Cv2.1169 H-FW2)
WVRQARGQRLEWIG

SEQ ID NO: 55 - protein, 32 aa, Artificial sequence
  synthetic peptide (Cv2.1169 H-FW3)
RVTITRDMSTTTAYMELSSLRSEDTAVYYCAA

SEQ ID NO: 56 - protein, 11 aa, Artificial sequence
  synthetic peptide (Cv2.1169 H-FW4)
WGQGTMVTVSS

SEQ ID NO: 57 - protein, 23 aa, Artificial sequence
  synthetic peptide (Cv2.1169 L-FW1)
EIVLTQSPGTLSLSPGERATLSC

SEQ ID NO: 58 - protein, 15 aa, Artificial sequence
  synthetic peptide (Cv2.1169 L-FW2)
WYQQKPGQAPRLLIY

SEQ ID NO: 59 - protein, 32 aa, Artificial sequence
  synthetic peptide (Cv2.1169 L-FW3)
GIPDRFSGSGSGTDFTLTISRLEPEDFAVYYC

SEQ ID NO: 60 - protein, 10 aa, Artificial sequence
  synthetic peptide (Cv2.1169 L-FW4)
FGQGTKVEIK

SEQ ID NO: 61 - protein, 30 aa, Artificial sequence
  synthetic peptide (Cv2.1353 H-FW1)
EVQLVESGGGLVQPGGSLRLSCAASGVTVS

SEQ ID NO: 62 - protein, 14 aa, Artificial sequence
  synthetic peptide (Cv2.1353 H-FW2)
WVRQAPGKGLEWVS

SEQ ID NO: 63 - protein, 32 aa, Artificial sequence
  synthetic peptide (Cv2.1353 H-FW3)
RFIISRDNSKNTLYLQMNSLKTEDTAVYYCAR

SEQ ID NO: 64 - protein, 11 aa, Artificial sequence
  synthetic peptide (Cv2.1353 H-FW4)
WGQGTMVTVSS

SEQ ID NO: 65 - protein, 23 aa, Artificial sequence
  synthetic peptide (Cv2.1353 L-FW1)
DIQMTQSPSFLSASVGDRVTITC

SEQ ID NO: 66 - protein, 15 aa, Artificial sequence
  synthetic peptide (Cv2.1353 L-FW2)
WYQQKPGKAPKLLIY

SEQ ID NO: 67 - protein, 32 aa, Artificial sequence
  synthetic peptide (Cv2.1353 L-FW3)
GVPSRFSGSGSGTEFTLTISSLQPEDFATYYC

SEQ ID NO: 68 - protein, 10 aa, artificial sequence
  synthetic peptide (Cv2.1353 L-FW4)
FGPGTKVDIK

SEQ ID NO: 69 - protein, 30 aa, Artificial sequence
  synthetic peptide (Cv2.3194 H-FW1)
EVQLVESGGGLIQPGGSLRLSCAASGITVT

SEQ ID NO: 70 - protein, 14 aa, Artificial sequence
  synthetic peptide (Cv2.3194 H-FW2)
WVRQAPGKGLEWVS

SEQ ID NO: 71 - protein, 32 aa, Artificial sequence
  synthetic peptide (Cv2.3194 H-FW3)
RFTISRDNSKNTLYLQMNSLRAEDTAVYYCAR

SEQ ID NO: 72 - protein, 11 aa, Artificial sequence
  synthetic peptide (Cv2.3194 H-FW4)
WGQGTTVTVSS

SEQ ID NO: 73 - protein, 23 aa, Artificial sequence
  synthetic peptide (Cv2.3194 L-FW1)
EIVLTQSPGTLSLSPGERATLSC

SEQ ID NO: 74 - protein, 15 aa, Artificial sequence
  synthetic peptide (Cv2.3194 L-FW2)
WYQQKPGQAPRLLIY

SEQ ID NO: 75 - protein, 32 aa, Artificial sequence
  synthetic peptide (Cv2.3194 L-FW3)
GIPGRFSGSGSGTDFTLTISRLEPEDFAIYYC

SEQ ID NO: 76 - protein, 10 aa, Artificial sequence
  synthetic peptide (Cv2.3194 L-FW4)
FGGGTKVEIK

SEQ ID NO: 77 - protein, 30 aa, Artificial sequence
  synthetic peptide (Cv2.3235 H-FW1)
EVQLVESGGGLIQPGGSLRLSCAASGFTVS

SEQ ID NO: 78 - protein, 14 aa, Artificial sequence
  synthetic peptide (Cv2.3235 H-FW2)
WVRQAPGKGLEWVS

SEQ ID NO: 79 - protein, 32 aa, Artificial sequence; identical to SEQ ID NO: 71
  synthetic peptide (Cv2.3235 H-FW3)

SEQ ID NO: 80 - protein, 11 aa, Artificial sequence
  synthetic peptide (Cv2.3235 H-FW4)
WGQGTTVTVSS

SEQ ID NO: 81 - protein, 23 aa, Artificial sequence
  synthetic peptide (Cv2.3235 L-FW1)
DIQMTQSPSFLSASVGDRVTITC

SEQ ID NO: 82 - protein, 15 aa, Artificial sequence
  synthetic peptide (Cv2.3235 L-FW2)
WYQQKPGKAPKLLIY

SEQ ID NO: 83 - protein, 32 aa, Artificial sequence
  synthetic peptide (Cv2.3235 L-FW3)
GVTSRFSGSGSGTEFTLTISSLQPEDFATYYC

SEQ ID NO: 84 - protein, 10 aa, Artificial sequence
  synthetic peptide (Cv2.3235 L-FW4)
FGQGTRLEIK

SEQ ID NO: 85 - protein, 30 aa, Artificial sequence; identical to SEQ ID NO: 77
  synthetic peptide (Cv2.5213 H-FW1)

SEQ ID NO: 86 - protein, 14 aa, Artificial sequence
  synthetic peptide (Cv2.5213 H-FW2)
WVRQAPGKGLEWVS

SEQ ID NO: 87 - protein, 32 aa, Artificial sequence; identical to SEQ ID NO: 71
  synthetic peptide (Cv2.5213 H-FW3)

SEQ ID NO: 88 - protein, 11 aa, Artificial sequence
  synthetic peptide (Cv2.5213 H-FW4)
WGQGATVTVSS

SEQ ID NO: 89 - protein, 23 aa, Artificial sequence
  synthetic peptide (Cv2.5213 L-FW1)
DIQMTQSPSSLSASVGDRVTITC

SEQ ID NO: 90 - protein, 15 aa, Artificial sequence
  synthetic peptide (Cv2.5213 L-FW2)
WYQQKPGKAPKLLIY

SEQ ID NO: 91 - protein, 32 aa, Artificial sequence
  synthetic peptide (Cv2.5213 L-FW3)
GVPSRFSGSGSGTDFTLTISSLQPEDFATYYC

SEQ ID NO: 92 - protein, 10 aa, Artificial sequence
  synthetic peptide (Cv2.5213 L-FW4)
FGPGTKVDIK

SEQ ID NO: 93 - DNA, 369 bp, Artificial sequence
  synthetic polynucleotide (Cv2.1169 VH)
caggtgcagctggtgcagtctgggcctgaggtgaagaagcctgggacctcagtgaaggtctcctgcaaggcttctggattcacctttactacctctgctgtgcagtgggtgcgacaggctcgtggacaacgccttgagtggataggctggatcgtcgttggcagtggtaacacaaactacgcacagaagttccaggaaagagtcaccattaccagggacatgtccacaaccacagcctacatggagctgagcagcctgagatccgaggacacggccgtgtattactgtgcggcgccatattgtagtggtgggacctgcttagatggttttgatatctggggccaagggacaatggtcaccgtctcttca

SEQ ID NO: 94 - DNA, 324 bp, Artificial sequence
  synthetic polynucleotide (Cv2.1169 VL)
gaaattgtgttgacgcagtctccaggcaccctgtctttgtctccgggggaaagagccaccctctcctgcagggccagtcagagtgttagccgcagctacttagcctggtaccagcagaaacctggccaggctcccaggctcctcatctatagtgcatccagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggagatcaaa

SEQ ID NO: 95 - DNA, 351 bp, Artificial sequence
  synthetic polynucleotide (Cv2.1353 VH)
gaggtgcagctggtggagtctgggggaggcttggtccagcctggggggtccctgagactctcctgtgcagcctctggagtcaccgtcagtagcaactacatgaactgggtccgccaggctccagggaaggggctggagtgggtctcagttatttatagcggtggtagcacattctacgcagactccgtgaagggccgattcatcatctccagagacaattccaagaacacgctgtatcttcaaatgaacagcctgaaaactgaggacacggctgtgtattactgtgcgagagatttagggcccatgggttttgatatctggggccaggggacaatggtcaccgtctcttca

SEQ ID NO: 96 - DNA, 324 bp, Artificial sequence
  synthetic polynucleotide (Cv2.1353 VL)
gacatccagatgacccagtctccatccttcctgtctgcatctgtaggagacagagtcaccatcacttgccgggccagtcagggcattagcagttatttagcctggtatcagcaaaaaccagggaaagccccaaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagtgatcctccggtcactttcggccctgggaccaaagtggatatcaaa

SEQ ID NO: 97 - DNA, 351 bp, Artificial sequence
  synthetic polynucleotide (Cv2.3194 VH)
gaggtgcagctggtggagtctggaggaggcttgatccagcctggggggtccctgagactctcctgtgcagcctctgggatcaccgtcactagcaactacatgagctgggtccgccaggctccagggaaggggctggagtgggtctcagttatttatcccggtggtagcacattctacgcagactccgtgaagggccgattcaccatctccagagacaattccaagaacacgctgtatcttcaaatgaacagcctgagagccgaggacacggccgtgtattactgtgcgagagatcttgtagtatacggtatggacgtctggggccaagggaccacggtcaccgtctcctca

SEQ ID NO: 98 - DNA, 312 bp, Artificial sequence
  synthetic polynucleotide (Cv2.3194 VL)
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccaggcaggttcagtggcagtgggtctgggacagacttcactctcaccatcagcagactggagcctgaagattttgcaatctattactgtcaacaaggggtcactttcggcggagggaccaaggtggagatcaaa

SEQ ID NO: 99 - DNA, 351 bp, artificial sequence
  synthetic polynucleotide (Cv2.3235_VH)
gaggtgcagctggtggagtctggaggaggcttgatccagcctggggggtccctgagactctcctgtgcagcctctgggttcaccgtcagtaggaactacatgagctgggtccgccaggctccagggaaggggctggagtgggtctcagttatttatagcggtggtagcacattctacgcagactccgtgaagggccgattcaccatctccagagacaactccaagaacacgctgtatcttcaaatgaacagcctgagagccgaggacacggccgtgtattactgtgcgagagatggggactactacggtatggacgtctggggccaagggaccacggtcaccgtctcctca

SEQ ID NO: 100 - DNA, 324 bp, Artificial sequence
  synthetic polynucleotide (Cv2.3235 VL)
gacatccagatgacccagtctccatccttcctgtctgcatctgtaggagacagagtcaccatcacttgccgggccagtcagggcattagcagttttttagcctggtatcagcaaaaaccagggaaagcccctaagctcctgatctatggtgcatccactttgcaaagtggggtcacatcaaggttcagcggcagtggatctgggacagaattcactctcacaatcagcagcctgcagcctgaagattttgcaacttattactgtcaacggcttgatagttaccctccgatcaccttcggccaagggacacgactggagattaaa

SEQ ID NO: 101 - DNA, 351 bp, Artificial sequence
  synthetic polynucleotide (Cv2.5213 VH)
gaggtgcagctggtggagtctggaggaggcttgatccagcctggggggtccctgagactctcctgtgcagcctctgggttcaccgtcagtagcaactacatgagctgggtccgccaggctccagggaaggggctggagtgggtctcacttatctatagcggtggtagcacatactacgcagactccgtgaagggccgattcaccatctccagagacaattccaagaacacgctgtatcttcaaatgaacagcctgagagccgaggacacggccgtgtattactgtgcgagggacctgaactactacggtatggacgtctggggccaaggggccacggtcaccgtctcctca

SEQ ID NO: 102 - DNA, 324 bp, Artificial sequence
  synthetic polynucleotide (Cv2.5213 VL)
gacatccagatgacccagtctccatcctccctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagatttcactctcaccatcagcagcctgcagcctgaagattttgcaacttattactgtcaacagcttgatagttactctccattcactttcggccctgggaccaaagtggatatcaaa

SEQ ID NO: 103 - protein, 644 aa, artificial sequence
  synthetic polypeptide (ACE2 ectodomain)
METDTLLLWVLLLWVPGSTGSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSGLVPRGSHHHHHHHHSAWSHPQFEK

SEQ ID NO: 104 - protein, 457 aa, artificial sequence
  Synthetic polypeptide (SARS-CoV-2 Nucleocapsid protein (N))
MGWSCIILFLVATATGVHSQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGSHHHHHHHHGSGLNDIFEAQKIEWHE

SEQ ID NO: 105 - protein, 22 aa, artificial sequence
  synthetic peptide (SARS-CoV-2 Fusion peptide (FP))
KRSFIEDLLFNKVTLADAGFIK

SEQ ID NO: 106 - protein, 1276 aa, artificial sequence
  synthetic polypeptide (SARS-CoV-2 Spike ectodomain (tri-S))
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGGSHHHHHHHHSAWSHPQFEKGTGGLNDIFEAQKIEWHE

SEQ ID NO: 107 - protein, 696 aa, artificial sequence
  synthetic polypeptide (SARS-CoV-2 S1 domain)
MGWSCIILFLVATATGVHSVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSHHHHHHHH

SEQ ID NO: 108 - protein, 263 aa, artificial sequence
  synthetic polypeptide (SARS-CoV-2 RBD domain)
METDTLLLWVLLLWVPGSTGNITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSGLVPRGSHHHHHHHHSAWSHPQFEKGTGGLNDIFEAQKIEWHE

SEQ ID NO: 109 - protein, 263 aa, artificial sequence
  synthetic polypeptide (SARS-CoV-2 variant B.1.1.7 RBD domain)
METDTLLLWVLLLWVPGSTGNITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKGSGLVPRGSHHHHHHHHSAWSHPQFEKGTGGLNDIFEAQKIEWHE

SEQ ID NO: 110 - protein, 263 aa, artificial sequence
  synthetic polypeptide (SARS-CoV-2 variant B.1.351 RBD domain)
METDTLLLWVLLLWVPGSTGNITNLCPFGEVFNATRFASVYAWNRKRISNCVADYSFLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKGSGLVPRGSHHHHHHHHSAWSHPQFEKGTGGLNDIFEAQKIEWHE

SEQ ID NO: 111 - protein, 263 aa, artificial sequence
  synthetic polypeptide (SARS-CoV-2 variant P.1 RBD domain)
METDTLLLWVLLLWVPGSTGNITNLCPFGEVFNATRFASVYAWNRKRISNCVADYSFLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKGSGLVPRGSHHHHHHHHSAWSHPQFEKGTGGLNDIFEAQKIEWHE

SEQ ID NO: 112 - protein, 322 aa, artificial sequence
  synthetic polypeptide (SARS-CoV-2 S1 NTD domain)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSGSGLVPRGSHHHHHHHH

SEQ ID NO: 113 - protein, 238 aa, artificial sequence
  synthetic polypeptide (SARS-CoV-2 S1 connecting domain (CD))
MGWSCIILFLVATATGVHSTVEKGIYQTSNFRVQPTESIVRFPGGGSGGKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGGSGLVPRGSHHHHHHHH

SEQ ID NO: 114 - protein, 582 aa, artificial sequence
  synthetic polypeptide (SARS-CoV-2 S2 domain)
MGWSCIILFLVATATGVHSSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGGSHHHHHHHH

SEQ ID NO: 115 - protein, 1322 aa, artificial sequence
  synthetic polypeptide (CoV-OC43 Spike ectodomain)
MPMGSLQPLATLYLLGMLVASVLAVIGDLKCTSDNINDKDTGPPPISTDTVDVTNGLGTYYVLDRVYLNTTLFLNGYYPTSGSTYRNMALKGSVLLSRLWFKPPFLSDFINGIFAKVKNTKVIKDRVMYSEFPAITIGSTFVNTSYSVVVQPRTINSTQDGDNKLQGLLEVSVCQYNMCEYPQTICHPNLGNHRKELWHLDTGVVSCLYKRNFTYDVNADYLYFHFYQEGGTFYAYFTDTGVVTKFLFNVYLGMALSHYYVMPLTCNSKLTLEYWVTPLTSRQYLLAFNQDGIIFNAVDCMSDFMSEIKCKTQSIAPPTGVYELNGYTVQPIADVYRRKPNLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPRPAGVLTNHDVVYAQHCFKAPKNFCPCKLNGSCVGSGPGKNNGIGTCPAGTNYLTCDNLCTPDPITFTGTYKCPQTKSLVGIGEHCSGLAVKSDYCGGNSCTCRPQAFLGWSADSCLQGDKCNIFANFILHDVNSGLTCSTDLQKANTDIILGVCVNYDLYGILGQGIFVEVNATYYNSWQNLLYDSNGNLYGFRDYITNRTFMIRSCYSGRVSAAFHANSSEPALLFRNIKCNYVFNNSLTRQLQPINYFDSYLGCVVNAYNSTAISVQTCDLTVGSGYCVDYSKNGGSGGAITTGYRFTNFEPFTVNSVNDSLEPVGGLYEIQIPSEFTIGNMVEFIQTSSPKVTIDCAAFVCGDYAACKSQLVEYGSFCDNINAILTEVNELLDTTQLQVANSLMNGVTLSTKLKDGVNFNVDDINFSPVLGCLGSECSKASSRSAIEDLLFDKVKLSDVGFVEAYNNCTGGAEIRDLICVQSYKGIKVLPPLLSENQFSGYTLAATSASLFPPWTAAAGVPFYLNVQYRINGLGVTMDVLSQNQKLIANAFNNALYAIQEGFDATNSALVKIQAVVNANAEALNNLLQQLSNRFGAISASLQEILSRLDALEAEAQIDRLINGRLTALNAYVSQQLSDSTLVKFSAAQAMEKVNECVKSQSSRINFCGNGNHIISLVQNAPYGLYFIHFSYVPTKYVTARVSPGLCIAGDRGIAPKSGYFVNVNNTWMYTGSGYYYPEPITENNVVVMSTCAVNYTKAPYVMLNTSIPNLPDFKEELDQWFKNQTSVAPDLSLDYINVTFLDLLIKRMKQIEDKIEEIESKQKKIENEIARIKKIKLVPRGSLEWSHPQFEK

SEQ ID NO: 116 - protein, 1328 aa, artificial sequence
  synthetic polypeptide (CoV-HKU1 Spike ectodomain)
METDTLLLWVLLLWVPGSTGVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVKPVATVYRRIPNLPDCDIDNWLNNVSVPSPLNWERRIFSNCNFNLSTLLRLVHVDSFSCNNLDKSKIFGSCFNSITVDKFAIPNRRRDDLQLGSSGFLQSSNYKIDISSSSCQLYYSLPLVNVTINNFNPSSWNRRYGFGSFNLSSYDVVYSDHCFSVNSDFCPCADPSVVNSCAKSKPPSAICPAGTKYRHCDLDTTLYVKNWCRCSCLPDPISTYSPNTCPQKKVVVGIGEHCPGLGINEEKCGTQLNHSSCFCSPDAFLGWSFDSCISNNRCNIFSNFIFNGINSGTTCSNDLLYSNTEISTGVCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSGGSGSGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSLLEDLLFNKVKLSDVGFVEAYNNCTGGSEIRDLLCVQSFNGIKVLPPILSETQISGYTTAATVAAMFPPWSAAAGVPFSLNVQYRINGLGVTMDVLNKNQKLIANAFNKALLSIQNGFTATNSALAKIQSVVNANAQALNSLLQQLFNKFGAISSSLQEILSRLDPPEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNGSGYIPEAPRDGQAYVRKDGEWVLLSTFLGSLVPRGSHHHHHHHH

SEQ ID NO: 117 - protein, 1356 aa, artificial sequence
  synthetic polypeptide (CoV-MERS Spike ectodomain)
METDTLLLWVLLLWVPGSTGVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPASVGSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDPPEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKGSGYIPEAPRDGQAYVRKDGEWVLLSTFLGSLVPRGSHHHHHHHHGLNDIFEAQKIEWHE

SEQ ID NO: 118 - protein, 1245 aa, artificial sequence
  synthetic polypeptide (SARS-CoV-1 Spike ectodomain)
METDTLLLWVLLLWVPGSTGSDLDRCTTFDDVQAPNYTQHTSSMRGVYYPDEIFRSDTLYLTQDLFLPFYSNVTGFHTINHTFDNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDTWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKGSGYIPEAPRDGQAYVRKDGEWVLLSTFLGSLVPRGSHHHHHHHH

SEQ ID NO: 119 - protein, 1165 aa, artificial sequence
  synthetic polypeptide (CoV-229E Spike ectodomain)
METDTLLLWVLLLWVPGSTGAGCQTTNGLNTSYSVCNGCVGYSENVFAVESGGYIPSDFAFNNWFLLTNTSSVVDGVVRSFQPLLLNCLWSVSGLRFTTGFVYFNGTGRGDCKGFSSDVLSDVIRYNLNFEENLRRGTILFKTSYGVVVFYCTNNTLVSGDAHIPFGTVLGNFYCFVNTTIGTETTSAFVGALPKTVREFVISRTGHFYINGYRYFTLGNVEAVNFNVTTAETTDFFTVALASYADVLVNVSQTSIANIIYCNSVINRLRCDQLSFYVPDGFYSTSPIQSVELPVSIVSLPVYHKHMFIVLYVDFKPQSGGGKCFNCYPAGVNITLANFNETKGPLCVDTSHFTTKYVAVYANVGRWSASINTGNCPFSFGKVNNFVKFGSVCFSLKDIPGGCAMPIVANWAYSKYYTIGTLYVSWSDGDGITGVPQPVEGVSSFMNVTLDKCTKYNIYDVSGVGVIRVSNDTFLNGITYTSTSGNLLGFKDVTKGTIYSITPCNPPDQLVVYQQAVVGAMLSENFTSYGFSNVVELPKFFYASNGTYNCTDAVLTYSSFGVCADGSIIAVQPRNVSYDSVSAIVTANLSIPSNWTISVQVEYLQITSTPIVVDCSTYVCNGNVRCVELLKQYTSACKTIEDALRNSARLESADVSEMLTFDKKAFTLANVSSFGDYNLSSVIPSLPTSGSRVAGRSAIEDILFSKIVTSGLGTVDADYKNCTKGLSIADLACAQYYNGIMVLPGVADAERMAMYTGSLIGGIALGGLTSAVSIPFSLAIQARLNYVALQTDVLQENQKILAASFNKAMTNIVDAFTGVNDAITQTSQALQTVATALNKIQDVVNQQGNSLNHLTSQLRQNFQAISSSIQAIYDRLDPPQADQQVDRLITGRLAALNVFVSHTLTKYTEVRASRQLAQQKVNECVKSQSKRYGFCGNGTHIFSIVNAAPEGLVFLHTVLLPTQYKDVEAWSGLCVDGTNGYVLRQPNLALYKEGNYYRITSRIMFEPRIPTMADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETYIKSGGYIPEAPRDGQAYVRKDGEWVLLSTFLGSLVPRGSHHHHHHHH

SEQ ID NO: 120 - protein, 1345 aa, artificial sequence
  synthetic polypeptide (CoV-NL63 Spike ectodomain)
METDTLLLWVLLLWVPGSTGFFTCNSNANLSMLQLGVPDNSSTIVTGLLPTHWFCANQSTSVYSANGFFYIDVGNHRSAFALHTGYYDANQYYIYVTNEIGLNASVTLKICKFSRNTTFDFLSNASSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVVDVMRYNLNFSANSLDNLKSGVIVFKTLQYDVLFYCSNSSSGVLDTTIPFGPSSQPYYCFINSTINTTHVSTFVGILPPTVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIRSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDSPPADQQVDRLITGRLAALNAFVSQVLNKYTEVRGSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLKLLNRFENLIKRMKQIEDKIEEIESKQKKIENEIARIKKIKGSLVPRGSHHHHHHHH

SEQ ID NO: 121 - protein, 8 aa, artificial sequence
  synthetic peptide (StrepTag)
WSHPQFEK

SEQ ID NO: 122 - protein, 263 aa, Artificial Sequence
  Synthetic polypeptide (SARS-CoV-2 kappa variant B.1.617.1/3 RBD domain)
METDTLLLWVLLLWVPGSTGNITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKGSGLVPRGSHHHHHHHHSAWSHPQFEKGTGGLNDIFEAQKIEWHE

SEQ ID NO: 123 - protein, 263 aa, Artificial Sequence
  Synthetic polypeptide (SARS-CoV-2 delta variant B.1.617.2/3 RBD domain)
METDTLLLWVLLLWVPGSTGNITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKGSGLVPRGSHHHHHHHHSAWSHPQFEKGTGGLNDIFEAQKIEWHE

SEQ ID NO: 124 - protein, 263 aa, Artificial Sequence
  Synthetic polypeptide (SARS-CoV-2 delta plus variant)
METDTLLLWVLLLWVPGSTGNITNLCPFGEVFNATRFASVYAWNRKRISNCVADYSFLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKGSGLVPRGSHHHHHHHHSAWSHPQFEKGTGGLNDIFEAQKIEWHE

SEQ ID NO: 125 - protein, 263 aa, Artificial Sequence
  Synthetic polypeptide (SARS-CoV-2 omicron variant B.1.1.529 / RBD domain)
METDTLLLWVLLLWVPGSTGNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKGSGLVPRGSHHHHHHHHSAWSHPQFEKGTGGLNDIFEAQKIEWHE

SEQ ID NO: 126 - protein, 1273 aa, Artificial Sequence
  Synthetic polypeptide (SARS-CoV-2 omicron variant B.1.1.529 / Spike ectodomain)
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSAGSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGGSHHHHHHHHSAWSHPQFEKGTGGLNDIFEAQKIEWHE

SEQ ID NO: 127 - protein, 20 aa, Artificial Sequence
  Synthetic Endoneous Leader Sequence
METDTLLLWVLLLWVPGSTG

SEQ ID NO: 128 - protein, 6 aa, Artificial Sequence
  Synthetic Thrombin Cleavage Site
LVPRGS

SEQ ID NO: 129 - protein, 8 aa, Artificial Sequence
  Synthetic Poly His-Tag
HHHHHHHH

SEQ ID NO: 130 - protein, 26 aa, Artificial Sequence
  Synthetic Foldon Trimerization motif
YIPEAPRDGQAYVRKDGEWVLLSTFL

SEQ ID NO: 131 - protein, 15 aa, Artificial Sequence
  Synthetic Avi-Tag
GLNDIFEAQKIEWHE

SEQ ID NO: 132 - protein, 330 aa, Artificial Sequence [sequence omitted]

SEQ ID NO: 133 - protein, 453 aa, Artificial Sequence
  Synthetic Cv2.1169 Full length Heavy Chain Prime
QVQLVQSGPEVKKPGTSVKVSCKASGFTFTTSAVQWVRQARGQRLEWIGWIVVGSGNTNYAQKFQERVTITRDMSTTTAYMELSSLRSEDTAVYYCAAPYCSGGTCLDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 447 aa, Artificial Sequence
  Synthetic Cv2.1353 Full length Heavy Chain Prime
EVQLVESGGGLVQPGGSLRLSCAASGVTVSSNYMNWVRQAPGKGLEWVSVIYSGGSTFYADSVKGRFIISRDNSKNTLYLQMNSLKTEDTAVYYCARDLGPMGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 447 aa, Artificial Sequence
  Synthetic Cv2.3194 Full length Heavy Chain Prime
EVQLVESGGGLIQPGGSLRLSCAASGITVTSNYMSWVRQAPGKGLEWVSVIYPGGSTFYADSVKGRFTISRDNSKNTLYLQMNSLRAEDTAVYYCARDLVVYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 447 aa, Artificial Sequence
  Synthetic Cv2.3235 Full length Heavy Chain Prime
EVQLVESGGGLIQPGGSLRLSCAASGFTVSRNYMSWVRQAPGKGLEWVSVIYSGGSTFYADSVKGRFTISRDNSKNTLYLQMNSLRAEDTAVYYCARDGDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 447 aa, Artificial Sequence
  Synthetic Cv2.5213 Full length Heavy Chain Prime
EVQLVESGGGLIQPGGSLRLSCAASGFTVSSNYMSWVRQAPGKGLEWVSLIYSGGSTYYADSVKGRFTISRDNSKNTLYLQMNSLRAEDTAVYYCARDLNYYGMDVWGQGATVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 5 aa, Artificial Sequence
  Synthetic Cv2.5179 HCDR1
SSAVQ

SEQ ID NO: 139 - protein, 17 aa, Artificial Sequence
  Synthetic Cv2.5179 HCDR2
WIVVGSGNTNYAQKFQE

SEQ ID NO: 140 - protein, 14 aa, Artificial Sequence
  Synthetic Cv2.5179 HCDR3
PHCSSTSCYDAFDI

SEQ ID NO: 141 - protein, 12 aa, Artificial Sequence
  Synthetic Cv2.5179 LCDR1
RASQSVSSSYLA

SEQ ID NO: 142 - protein, 7 aa, Artificial Sequence
  Synthetic Cv2.5179 LCDR2
GASSRAT

SEQ ID NO: 143 - protein, 9 aa, Artificial Sequence
  Synthetic Cv2.5179 LCDR3
QQYGRSPWT

SEQ ID NO: 144 - protein, 30 aa, Artificial Sequence; identical to SEQ ID NO: 53
  Synthetic Cv2.5179 H-FR1

SEQ ID NO: 145 - protein, 14 aa, Artificial Sequence
  Synthetic Cv2.5179 H-FR2
WVRQARGQRLEWIG

SEQ ID NO: 146 - protein, 32 aa, Artificial Sequence
  Synthetic Cv2.5179 H-FR3
RVTITRDMSTSTAYMELSSLRSEDTAVYYCAA

SEQ ID NO: 147 - protein, 11 aa, Artificial Sequence
  Synthetic Cv2.5179 H-FR4
WGQGTMVTVSS

SEQ ID NO: 148 - protein, 23 aa, Artificial Sequence
  Synthetic Cv2.5179 L-FR1
EIVLTQSPGTLSLSPGERATLSC

SEQ ID NO: 149 - protein, 15 aa, Artificial Sequence
  Synthetic Cv2.5179 L-FR2
WYQQKPGQAPRLLIY

SEQ ID NO: 150 - protein, 32 aa, Artificial Sequence
  Synthetic Cv2.5179 L-FR3
GIPDRFSGSGSGTDFTLTISRLEAEDFAVYYC

SEQ ID NO: 151 - protein, 10 aa, Artificial Sequence
  Synthetic Cv2.5179 L-FR4
FGQGTKLEIK

SEQ ID NO: 152 - protein, 123 aa, Artificial Sequence
  Synthetic Cv2.5179 VH
QVQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPHCSSTSCYDAFDIWGQGTMVTVSS

SEQ ID NO: 153 - protein, 108 aa, Artificial Sequence
  Synthetic Cv2.5179 VL
EIVLTQSPGTLSLSPGERATLSCRASQSVSSSYLAWYQQKPGQAPRLLIYGASSRATGIPDRFSGSGSGTDFTLTISRLEAEDFAVYYCQQYGRSPWTFGQGTKLEIK

SEQ ID NO: 154 - protein, 453 aa, Artificial Sequence
  Synthetic Cv2.5179 Heavy chain
QVQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPHCSSTSCYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 215 aa, Artificial Sequence
  Synthetic Cv2.5179 Light chain
EIVLTQSPGTLSLSPGERATLSCRASQSVSSSYLAWYQQKPGQAPRLLIYGASSRATGIPDRFSGSGSGTDFTLTISRLEAEDFAVYYCQQYGRSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - DNA, 369 bp, Artificial Sequence
  Synthetic Nucleic acid coding for Cv2.5179 Heavy chain
caggtgcagctggtgcagtctgggcctgaggtgaagaagcctgggacctcagtgaaggtctcctgcaaggcttctggattcacctttactagctctgctgtgcagtgggtgcgacaggcccgtggacaacgccttgagtggataggatggatcgtcgttggcagtggtaacacaaactacgcacagaagttccaagaaagagtcaccattaccagggacatgtccacaagcacagcctacatggagctgagcagcctgagatccgaggacacggccgtgtattactgtgcggcaccacattgtagtagtaccagttgctatgacgcttttgatatctggggccaagggacaatggtcaccgtctcttca

SEQ ID NO: 157 - protein, 324 aa, Artificial Sequence
  Synthetic Nucleic acid coding for Cv2.5179 Light chain
GAAATTGTGTTGACGCAGTCTCCAGGCACCCTGTCTTTGTCTCCAGGGGAAAGGGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGGCTGAAGATTTTGCAGTGTATTACTGTCAGCAGTATGGTAGGTCACCGTGGACGTTCGGCCAAGGGACCAAGCTGGAGATCAAA

SEQ ID NO: 158 - protein, 123 aa, Artificial Sequence
  Synthetic Cv2.5156 VH
QVQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAANYCSSTSCSDAFDIWGQGTMVTVSS

SEQ ID NO: 159 - protein, 111 aa, Artificial Sequence
  Synthetic Cv2.5156 VL
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGWVFGGGTKLTVL

SEQ ID NO: 160 - protein, 120 aa, Artificial Sequence
  Synthetic Cv2.1174 VH
EVQLVESGPEVKKPGTSVKVSCKASGFTFTRSAMQWVRQARGQRLEWIGWIVVGSGNTNYAQKFQERVTITRDMSTSTAYMELSSLRSEDTAVYYCAARQDYYYYGMDVWGQGTTVTVSS

SEQ ID NO: 161 - protein, 108 aa, Artificial Sequence
  Synthetic Cv2.1174 VL
DIQMTQSPSSLSASVGDRVTITCRASQGMSNSLDWDQQKPGKAPKLLLYAASRLKSGVPSRFSGSGSGTDYTLTISSLQPEDFATYYCQQYYSDPPKTFGQGTKVEIK

SEQ ID NO: 162 - protein, 128 aa, Artificial Sequence
  Synthetic Cv2.1388 VH
QVQLVQSGPEVKKPGTSVKVSCKASGFTFTSSAVQWVRQARGQRLEWIGWIVVGSGDTNYAQKFQERVTITRDMSTSTAYMELSSLRSEDTAVYYCATDRQAGSRTYYFPGEGWFDPWGQGTLVTVSS

SEQ ID NO: 163 - protein, 111 aa, Artificial Sequence
  Synthetic Cv2.1388 VL
QSVLTQPPSVSAAPGQKVTISCSGSTTNIGNNYVSWYQRLPGTAPKLLIYDNNKRPSGIPDRFSGSKSGTSATLGITGLQTGDEADYYCGTWDSSLSGVVFRRRDQADRPR

SEQ ID NO: 164 - protein, 123 aa, Artificial Sequence
  Synthetic Cv2.3132 VH
EVQLVESGGGLVKPGGSLRLSCAASGFTFSDYYMSWIRQAPGKGLEWVSYISSSGSVQYYADSVKGRFTISRDNAENSLYLQMNSLRAEDTAVYYCARYYYDSSGYWKRLDYWGQGTLVTVSS

SEQ ID NO: 165 - protein, 107 aa, Artificial Sequence
  Synthetic Cv2.3132 VL
DIQMTQSPSSLSASVGDRVTITCRASQSISRYLNWYQQKPGKAPKLLIYAASSLQSGVPSRFSGSGSGTDFTLTISSLQPEDFATYHCQQSYSTPLTFGGGTKVEIK

SEQ ID NO: 166 - protein, 120 aa, Artificial Sequence
  Synthetic Casirivimab VH
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSS

SEQ ID NO: 167 - protein, 107 aa, Artificial Sequence
  Synthetic Casirivimab VL
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIK

SEQ ID NO: 168 - protein, 119 aa, Artificial Sequence
  Synthetic Etesevimab VH
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSS

SEQ ID NO: 169 - protein, 109 aa, Artificial Sequence
  Synthetic Etesevimab VL
DIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIK

SEQ ID NO: 170 - protein, 128 aa, Artificial Sequence
  Synthetic Regdanvimab VH
QITLKESGPTLVKPTQTLTLTCSFSGFSLSTSGVGVGWIRQPPGKALEWLALIDWDDNKYHTTSLKTRLTISKDTSKNQVVLTMTNMDPVDTATYYCARIPGFLRYRNRYYYYGMDVWGQGTTVTVSS

SEQ ID NO: 171 - protein, 110 aa, Artificial Sequence
  Synthetic Regdanvimab VL
ELVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGVFGGGTELTVL

SEQ ID NO: 172 - protein, 123 aa, Artificial Sequence
  Synthetic Tixagevimab VH
QMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSS

SEQ ID NO: 173 - protein, 109 aa, Artificial Sequence
  Synthetic Tixagevimab VL
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIK

SEQ ID NO: 174 - protein, 124 aa, Artificial Sequence
  Synthetic Adintrevimab VH
EVQLVESGGGLVKPGGSLRLSCAASGFTFSSYYMNWVRQAPGKGLEWVSSISEDGYSTYYPDSLKGRFTISRDSAKNSLYLQMNSLRADDTAVYYCARDFSGHTAWAGTGFEYWGQGTLVTVSS

SEQ ID NO: 175 - protein, 112 aa, Artificial Sequence
  Synthetic Adintrevimab VL
QSVLTQPPSVSGAPGQRITISCTGSSSNIGAGYDVHWYQQLPGTAPKLLIYGSSSRNSGVPDRFSGSKSGTSASLAITGLQAEDEADYYCQSYDSSLSVLYTFGTGTKVTVL

SEQ ID NO: 176 - protein, 125 aa, Artificial Sequence
  Synthetic Bamlanivimab VH
QVQLVQSGAEVKKPGSSVKVSCKASGGTFSNYAISWVRQAPGQGLEWMGRIIPILGIANYAQKFQGRVTITADKSTSTAYMELSSLRSEDTAVYYCARGYYEARHYYYYYAMDVWGQGTAVTVSS

SEQ ID NO: 177 - protein, 107 aa, Artificial Sequence
  Synthetic Bamlanivimab VL
DIQMTQSPSSLSASVGDRVTITCRASQSISSYLSWYQQKPGKAPKLLIYAASSLQSGVPSRFSGSGSGTDFTLTITSLQPEDFATYYCQQSYSTPRTFGQGTKVEIK

SEQ ID NO: 178 - protein, 131 aa, Artificial Sequence
  Synthetic Cilgavimab VH
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSS

SEQ ID NO: 179 - protein, 112 aa, Artificial Sequence
  Synthetic Cilgavimab VL
DIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIK

SEQ ID NO: 180 - protein, 127 aa, Artificial Sequence
  Synthetic Sotrovimab VH
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSS

SEQ ID NO: 181 - protein, 107 aa, Artificial Sequence [sequence omitted]

SEQ ID NO: 182 - protein, 120 aa, Artificial Sequence
  Synthetic Imdevimab VH
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSS

SEQ ID NO: 183 - protein, 110 aa, Artificial Sequence
  Synthetic Imdevimab VL
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVL

SEQ ID NO: 184 - protein, 263 aa, Artificial Sequence
  Synthetic polypeptide (Omicron subvariant BA.2)
METDTLLLWVLLLWVPGSTGNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKGSGLVPRGSHHHHHHHHSAWSHPQFEKGTGGLNDIFEAQKIEWHE

(19) United States
(12) Patent Application Publication
(10) Pub. No.: US 2024/0336672 A1
(43) Pub. Date: Oct. 10, 2024

(54) HUMAN NEUTRALIZING MONOCLONAL ANTIBODIES AGAINST SARS-COV-2 AND USES THEREOF

(71) Applicant: INSTITUT PASTEUR, Paris Cedex 15 (FR)

(72) Inventors: Hugo MOUQUET, Paris (FR); Cyril PLANCHAIS, Paris (FR); Olivier SCHWARTZ, Paris (FR); Ignacio FERNANDEZ, Paris (FR); Timothée BRUEL, Paris (FR); Xavier MONTAGUTELLI, Paris (FR); Hervé BOURHY, Paris (FR); Guilherme DIAS DE MELO, Paris (FR); Félix REY, Paris (FR)

(21) Appl. No.: 18/288,115

(22) PCT Filed: Apr. 1, 2022
(86) PCT No.: PCT/EP2022/058777

§ 371 Date: Oct. 24, 2023

(30) Foreign Application Priority Data
Apr. 26, 2021 (WO) PCT/IB2021/000314
Dec. 22, 2021 (EP) 21306908.1
May 25, 2022 (WO) PCT/IB2022/000108

(51) Int. Cl. C07K 16/10; A61K 39/00; A61P 31/14; G01N 33/569

(52) U.S. Cl. CPC C07K 16/1003; A61P 31/14; G01N 33/56983; A61K 2039/505; C07K 2317/21; C07K 2317/24; C07K 2317/33; C07K 2317/565; C07K 2317/76; C07K 2317/92; C07K 2317/94

(57) ABSTRACT

The invention relates to antibodies against Severe Acute Respiratory Syndrome-related Coronavirus 2 (SARS-CoV-2), in particular human neutralizing monoclonal antibodies against Severe Acute Respiratory Syndrome-related Coronavirus 2 (SARS-CoV-2) and their use for the diagnosis, monitoring, prevention, and treatment of SARS-CoV-2 infection and associated disease (COV-ID-19).

### FIELD OF THE INVENTION

The invention relates to antibodies against Severe Acute Respiratory Syndrome-related Coronavirus 2 (SARS-CoV-2), in particular human neutralizing monoclonal antibodies against Severe Acute Respiratory Syndrome-related Coronavirus 2 (SARS-CoV-2) and their use for the diagnosis, monitoring, prevention, and treatment of SARS-CoV-2 infection and associated disease (COVID-19).

### BACKGROUND OF THE INVENTION

The pandemic caused by emerging Severe Acute Respiratory Syndrome Coronavirus 2 (SARS-CoV-2), which causes Coronavirus Disease-2019 (COVID-19) and accounts to date for nearly 470 million infection cases and 6 million deaths worldwide (https://www.who.int/), presents a serious global public health emergency in urgent need for prophylactic and therapeutic interventions.

Coronaviruses are enveloped, positive-sense, single-stranded RNA viruses that infect humans and mammals. Coronaviruses genomes encode non-structural polyprotein and structural proteins, including the homotrimeric spike (S) glycoprotein, envelope (E), membrane (M) and nucleocapsid (N) proteins. Several coronaviruses are pathogenic to human, leading to varying degrees of symptoms severity (Cui et al., Nat Rev Microbiol. 2019 March; 17(3):181-92). The betacoronavirus genus (Beta-CoV or β-CoV) which is divided in 4 lineages or groups (A, B, C, D) comprises the highly human-pathogenic coronaviruses in group B/C. Beta-CoV group B/C includes the severe acute respiratory syndrome coronavirus (SARS-CoV or SARS-CoV-1) that emerged in China in 2002, the Middle East respiratory syndrome coronavirus (MERS-CoV), first detected in Saudi Arabia in 2012, and the new coronavirus named SARS-CoV-2 that causes COVID-19, isolated in China in 2019 (SARS-CoV-2 isolate Wuhan-Hu-1), in association with cases of severe acute respiratory syndrome (Peiris et al., Nat Med., 2004 December; 10(12 Suppl):S88-97; Zaki et al., N Engl J Med., 2012 Nov. 8; 367(19):1814-20; Lee et al., BMC Infect Dis. 2017 Jul. 14; 17(1):498; Zhu N et al., N Engl J Med., 2020 Jan. 24). In contrast, Beta-CoV group A includes HCoV-OC43 and HCoV-HKU1 which can cause the common cold.

Antibodies developing in response to SARS-CoV-2 infection and vaccination are essential for long-term protection against COVID-19. Human neutralizing SARS-CoV-2 antibodies appear to play a key role in the control of COVID-19 infection and represent promising immunotherapeutic tools for treating SARS-CoV-2 infected humans with mild-to-moderate disease. Decoding antibody responses in COVID-19 is fundamental in understanding the basic mechanisms of humoral immunity to the SARS-CoV-2 Spike protein (SARS-CoV-2-S), the target of neutralizing antibodies, but also to develop effective vaccine and monoclonal antibody-based immunotherapy strategies.

The Spike glycoprotein has key roles in the viral cycle, as it is involved in receptor recognition, virus attachment and entry, and is thus a crucial determinant of host tropism and transmission capacity. SARS-CoV-2 cellular entry depends on binding between the viral Spike protein receptor-binding domain (RBD) and the angiotensin converting enzyme 2 (ACE2) target receptor. Binding with ACE2 triggers a cascade of cell membrane fusion events for viral entry. Each S protomer consists of two subunits that are cleaved by proteases: a globular S1 domain and the N-terminal region, and the membrane-proximal S2 and transmembrane domains. Determinants of host range and cellular tropisms are found in the RBD within the S1 domain, while mediators of membrane fusion have been identified within the S2 domain. Anti-SARS-CoV-2 antibody neutralizing potency is determined by competition with ACE2 receptor for RBD binding.

Antibodies rapidly develop in response to SARS-CoV-2 infection, including neutralizing antibodies recognizing distinct S protein regions. The RBD is the primary target of neutralizing antibodies including potent neutralizers, but the NTD and S2 stem region also contain neutralizing epitopes. SARS-CoV-2 neutralizing IgA antibodies are detected as early as a week after onset of symptoms, contribute to seroneutralization and can be as potent as IgGs. Neutralizing antibodies are the main correlate of protection for COVID-19 vaccines. Still, SARS-CoV-2 spike-specific antibodies, including non-neutralizers, can exert antiviral Fc-dependent effector functions important for in vivo protection i.e., antibody-dependent cellular cytotoxicity (ADCC), and phagocytosis (ADCP).

Since mid-2020, emerging variants of SARS-CoV-2 with increased transmissibility and/or reduced sensitivity to neutralizing antibodies due to predicted mutations in the Spike protein, in particular its receptor-binding region (RBD), were reported in several countries and are currently spreading worldwide. The first variant reported was in UK (lineage B.1.1.7; notable mutations N501Y, 69-70del, P681H); then in South Africa (SA) (lineage B.1.351; notable mutations N501Y, E484K, K417N) and Brazil (BR) (lineage P.1; notable mutations N501Y, E484K, K417T). Some monoclonal and serum-derived antibodies are reported to be from 10 to 60 time less effective in neutralizing virus bearing the E484K mutation (SARS-CoV-2 variants SA and BR). Some vaccines might see their efficacy reduced against these variants.

Since then, new emerging variants of SARS-CoV-2 are spreading worldwide, including lineages of Variants of Concerns (VOCs) such as the ones cited below, or VOCs comprising the same mutations in the spike proteins responsible for increased affinity to hACE2 and potential immune escape:
Alpha (B.1.1.7) N501Y; A570D; P681H; T716I; S982A; D1118H
Beta (B.1.351) D80A; D215G; K417N; E484K; N501Y; A701V
Gamma (P.1) L18F; T20 N; P26S; D138Y; R190S; K417T; E484K; N501Y; H655Y; T1027I
Delta (B.1.617.2) T19R; L452R; T478K; P681R; D950N
Omicron (BA.1 sublineage) (B. 1.1.529) A67V, H69-, V70-, T95I, G142-, V143-, Y144-, Y145D, N211-, L2121, G339D, S371L, S373P, S375F, K417N, N440K, G446S, S477N, T478K, E484A, Q493R, G496S, Q498R, N501Y, T547K, D614G, H655Y, N679K, P681H, N764K, D796Y, N856K, Q954H, N969K, L981F
Omicron (BA.2 sublineage) G339D; F367V; S371F; S373P; S375F; T376A; D405N; R408S; K417N; N440K; S477N; T478K; E484A; Q493R; Q498R; N501Y; Y505H

To develop prophylactic and therapeutic approaches specific to SARS-CoV-2, there is a need for neutralizing antibodies against SARS-CoV-2, in particular human neutralizing antibodies against SARS-CoV-2 including human antibodies capable of neutralizing SARS-CoV-2 variants, especially the above VOCs and variants of such VOCs and VOCs having a combination of the above mutations. The challenge is to provide monoclonal antibodies, alone or in combination, which retain efficient neutralizing properties to confer protection to individuals at risk of developing a SARS from present and future VOCs. While it cannot be expected that a universal SARS-CoV2 Spike monoclonal antibody (mAb) would be such that it would keep sufficient efficacy against most VOCs and future VOCs, it is a goal to provide mAbs that are consistent in neutralizing the majority of these VOCs and VOCs comprising different combinations of these mutations; so that it remains useful overtime to prevent or treat SARS.

Immunotherapies based on SARS-CoV-2 neutralizing antibodies have been rapidly explored, and this led to the clinical use of several mAbs alone or in bi-therapies. Highly potent human SARS-CoV-2 neutralizing antibodies isolated so far, including those tested or used in clinics, all target the RBD and can prevent or protect animals from infection in preclinical models. However, viral variants with spike mutations conferring resistance to antibody neutralization emerged during the pandemics and annihilated some of these therapies. The search for broadly neutralizing mAbs is pursued. Novel antibodies active against all VOCs, including the currently prevalent omicron lineage, have been described.

This challenge of providing mAbs that are consistent in neutralizing the majority of these VOCs and VOCs comprising different combinations of these mutations is enormous considering the results presented herein which shows the loss of potency of most of the currently FDA/EMEA approved therapeutic mAbs. The present invention fulfills this need and provides specific mAbs with retained potency across majors VOCs displaying such mutations.

The most potent, Cv2.1169 IgA and Cv2.3194 IgG, were fully active against VOCs Alpha, Beta, Gamma, and Delta, and still strongly blocked Omicron BA.1 and BA.2 infection in vitro. J-chain dimerization of Cv2.1169 IgA greatly improved its neutralization potency against BA.1 and BA.2. Cv2.1169 showed therapeutic efficacy in mouse and hamster SARS-CoV-2 infection models.

### SUMMARY OF THE INVENTION

The invention provides antibodies against Severe Acute Respiratory Syndrome-related Coronavirus 2 (SARS-CoV-2) and fragments thereof, including antigen-binding fragments thereof, in particular human neutralizing antibodies against Severe Acute Respiratory Syndrome-related Coronavirus 2 (SARS-CoV-2), nucleic acids, vectors encoding the antibodies, compositions, reagents, medical devices, and kits comprising the antibodies, nucleic acids, vectors according to the present disclosure.

The invention encompasses methods of making and using, as well as uses of the antibodies, nucleic acids, vectors, according to the present disclosure, in particular for the detection, diagnosis, monitoring, prevention and treatment of SARS-CoV-2 infection and associated disease (COVID-19).

The disclosure relates to a human neutralizing monoclonal antibody against Severe Acute Respiratory Syndrome-related Coronavirus 2 (SARS-CoV-2), or an antigen-binding fragment thereof, which specifically binds to the viral Spike protein receptor-binding domain (RBD) and is a competitive inhibitor of binding to the RBD of at least one of the following reference antibodies:
the reference human antibody Cv2.1169 comprising (i) a heavy chain variable region comprising the amino acid sequence of SEQ ID NO: 3 and (ii) a light chain variable region comprising the amino acid sequence of SEQ ID NO: 4;
the reference human antibody Cv2.3194 comprising (i) a heavy chain variable region comprising the amino acid sequence of SEQ ID NO: 7 and (ii) a light chain variable region comprising the amino acid sequence of SEQ ID NO: 8;
the reference human antibody Cv2.1353 comprising (i) a heavy chain variable region comprising the amino acid sequence of SEQ ID NO: 5 and (ii) a light chain variable region comprising the amino acid sequence of SEQ ID NO: 6;
the reference human antibody Cv2.5213 comprising (i) a heavy chain variable region comprising the amino acid sequence of SEQ ID NO: 11 and (ii) a light chain variable region comprising the amino acid sequence of SEQ ID NO: 12; and
the reference human antibody Cv2.3235 comprising (i) a heavy chain variable region comprising the amino acid sequence of SEQ ID NO: 9 and (ii) a light chain variable region comprising the amino acid sequence of SEQ ID NO: 10.

The disclosure also relates to a human neutralizing monoclonal antibody against Severe Acute Respiratory Syndrome-related Coronavirus 2 (SARS-CoV-2), or an antigen-binding fragment thereof, which specifically binds to the viral Spike protein receptor-binding domain (RBD) and is a competitive inhibitor of binding to the RBD of the following reference antibody: reference human antibody Cv2.5179 comprising (i) a heavy chain variable region comprising the amino acid sequence of SEQ ID NO: 152 and (ii) a light chain variable region comprising the amino acid sequence of SEQ ID NO: 153.

Thus the disclosure also relates to a human neutralizing monoclonal antibody against Severe Acute Respiratory Syndrome-related Coronavirus 2 (SARS-CoV-2), or antigen-binding fragment thereof, which specifically binds to the viral Spike protein receptor-binding domain (RBD) and is a competitive inhibitor of binding to the RBD of at least one of the following reference antibodies:
the reference human antibody Cv2.3194 comprising (i) a heavy chain variable region comprising the amino acid sequence of SEQ ID NO: 7 and a light chain variable region comprising the amino acid sequence of SEQ ID NO: 8;
the reference human antibody Cv2.5179 comprising (i) a heavy chain variable region comprising the amino acid sequence of SEQ ID NO: 152 and (ii) a light chain variable region comprising the amino acid sequence of SEQ ID NO: 153
the reference human antibody Cv2.1169 comprising (i) a heavy chain variable region comprising the amino acid sequence of SEQ ID NO: 3 and (ii) a light chain variable region comprising the amino acid sequence of SEQ ID NO: 4;
the reference human antibody Cv2.1353 comprising (i) a heavy chain variable region comprising the amino acid sequence of SEQ ID NO: 5 and (ii) a light chain variable region comprising the amino acid sequence of SEQ ID NO: 6;
the reference human antibody Cv2.5213 comprising (i) a heavy chain variable region comprising the amino acid sequence of SEQ ID NO: 11 and (ii) a light chain variable region comprising the amino acid sequence of SEQ ID NO: 12; and
the reference human antibody Cv2.3235 comprising (i) a heavy chain variable region comprising the amino acid sequence of SEQ ID NO: 9 and a light chain variable region comprising the amino acid sequence of SEQ ID NO: 10;
   said human neutralizing antibody, or antigen-binding fragment thereof, comprising:
a) a heavy chain variable domain comprising: a heavy chain CDR1 of SEQ ID NO: 35, a heavy chain CDR2 of SEQ ID NO: 36 and a heavy chain CDR3 of SEQ ID NO: 37, and a light chain variable domain comprising: a light chain CDR1 of SEQ ID NO: 38, a light chain CDR2 of SEQ ID NO: 39, and a light chain CDR3 of SEQ ID NO: 40;
b) a heavy chain variable domain comprising: a heavy chain CDR1 of SEQ ID NO: 23, a heavy chain CDR2 of SEQ ID NO: 24, and a heavy chain CDR3 of SEQ ID NO: 25, and a light chain variable domain comprising: a light chain CDR1 of SEQ ID NO: 26, a light chain CDR2 of SEQ ID NO: 27 and a light chain CDR3 of SEQ ID NO: 28;
c) a heavy chain variable domain comprising: a heavy chain CDR1 of SEQ ID NO: 23, a heavy chain CDR2 of SEQ ID NO: 24, and a heavy chain CDR3 of SEQ ID NO: 25, and a light chain variable domain comprising: a light chain CDR1 of SEQ ID NO: 26, a light chain CDR2 of SEQ ID NO: 27 and a light chain CDR3 of SEQ ID NO: 28;
d) a heavy chain variable domain comprising: a heavy chain CDR1 of SEQ ID NO: 29, a heavy chain CDR2 of SEQ ID NO: 30 and a heavy chain CDR3 of SEQ ID NO: 31, and a light chain variable domain comprising: a light chain CDR1 of SEQ ID NO: 32, a light chain CDR2 of SEQ ID NO: 33, and a light chain CDR3 of SEQ ID NO: 34;
e) a heavy chain variable domain comprising: a heavy chain CDR1 of SEQ ID NO: 41, a heavy chain CDR2 of SEQ ID NO: 42 and a heavy chain CDR3 of SEQ ID NO: 43, and a light chain variable domain comprising: a light chain CDR1 of SEQ ID NO: 44, a light chain CDR2 of SEQ ID NO: 45, and a light chain CDR3 of SEQ ID NO: 46;
f) a heavy chain variable domain comprising: a heavy chain CDR1 of SEQ ID NO: 47, a heavy chain CDR2 of SEQ ID NO: 48 and a heavy chain CDR3 of SEQ ID NO: 49 and a light chain variable domain comprising: a light chain CDR1 of SEQ ID NO: 50, a light chain CDR2 of SEQ ID NO: 51, and a light chain CDR3 of SEQ ID NO: 52;
g) a heavy chain variable domain comprising heavy chain CDR1, CDR2 and CDR3 which differ from SEQ ID NO: 23 to 25, or 138 to 140, respectively by up to 2 amino acid mutations in the sequence of 1, 2 or 3 CDRs; and a light chain variable domain comprising: light chain CDR1, CDR2, and CDR3 which differ from SEQ ID NO: 26 to 28, or 141 to 143, respectively by up to 2 amino acid mutations in the sequence of 1, 2 or 3 CDRs.

Thus the disclosure also relates to an isolated antibody directed against the viral Spike protein receptor binding-domain (RBD) of SARS-CoV-2, or antigen-binding fragment thereof, characterized in that it comprises:
a heavy chain variable domain selected from:
a heavy chain variable domain comprising at least one of, preferably all three of: a heavy chain CDR1 of SEQ ID NO: 138, a heavy chain CDR2 of SEQ ID NO: 139 and a heavy chain CDR3 of SEQ ID NO: 140, or a variant with one or two conservative substitution(s); or
a heavy chain variable domain comprising at least one of, preferably all three of: a heavy chain CDR1 of SEQ ID NO: 23, a heavy chain CDR2 of SEQ ID NO: 24 and a heavy chain CDR3 of SEQ ID NO: 25, or a variant with one or two conservative substitution(s); or
a heavy chain variable domain comprising at least one of, preferably all three of: a heavy chain CDR1 of SEQ ID NO: 29, a heavy chain CDR2 of SEQ ID NO: 30 and a heavy chain CDR3 of SEQ ID NO: 31, or a variant with one or two conservative substitution(s); or
a heavy chain variable domain comprising at least one, preferably all three of: a heavy chain CDR1 of SEQ ID NO: 35, a heavy chain CDR2 of SEQ ID NO: 36 and a heavy chain CDR3 of SEQ ID NO: 37, or a variant with one or two conservative substitution(s); or
a heavy chain variable domain comprising at least one of, preferably all three of: a heavy chain CDR1 of SEQ ID NO: 41, a heavy chain CDR2 of SEQ ID NO: 42 and a heavy chain CDR3 of SEQ ID NO: 43, or a variant with one or two conservative substitution(s); or
a heavy chain variable domain comprising at least one of, preferably all three of: a heavy chain CDR1 of SEQ ID NO: 47, a heavy chain CDR2 of SEQ ID NO: 48 and a heavy chain CDR3 of SEQ ID NO: 49, or a variant with one or two conservative substitution(s);
and
a light chain variable domain selected from:
a light chain variable domain comprising at least one of, preferably all three of: a light chain CDR1 of SEQ ID NO: 141, a light chain CDR2 of SEQ ID NO: 142 and a light chain CDR3 of SEQ ID NO: 143, or a variant with one or two conservative substitution(s); or
a light chain variable domain comprising at least one of, preferably all three of: a light chain CDR1 of SEQ ID NO: 26, a light chain CDR2 of SEQ ID NO: 27 and a light chain CDR3 of SEQ ID NO: 28, or a variant with one or two conservative substitution(s); or
a light chain variable domain comprising at least one of, preferably all three of: a light chain CDR1 of SEQ ID NO: 32, a light chain CDR2 of SEQ ID NO: 33 and a light chain CDR3 of SEQ ID NO: 34, or a variant with one or two conservative substitution(s); or
a light chain variable domain comprising at least one, preferably all three of: a light chain CDR1 of SEQ ID NO: 38, a light chain CDR2 of SEQ ID NO: 39 and a light chain CDR3 of SEQ ID NO: 40, or a variant with one or two conservative substitution(s); or
a light chain variable domain comprising at least one of, preferably all three of: a light chain CDR1 of SEQ ID NO: 44, a light chain CDR2 of SEQ ID NO: 45 and a light chain CDR3 of SEQ ID NO: 46, or a variant with one or two conservative substitution(s); or
a light chain variable domain comprising at least one of, preferably all three of: a light chain CDR1 of SEQ ID NO: 50, a light chain CDR2 of SEQ ID NO: 51 and a light chain CDR3 of SEQ ID NO: 52, or a variant with one or two conservative substitution(s).

In some particular embodiments, the antibody or antigen-binding fragment according to the present disclosure competes for binding with the reference human antibody Cv2.5179, Cv2.1169 or Cv2.3194; for example Cv2.1169 or Cv.3194; for example Cv2.1169 or Cv2.5179; preferably the reference human antibody Cv2.1169.

In some particular embodiments, the antibody or antigen-binding fragment according to the present disclosure comprises: a heavy chain variable domain comprising a heavy chain CDR1 of SEQ ID NO: 23, a heavy chain CDR2 of SEQ ID NO: 24 and a heavy chain CDR3 of SEQ ID NO: 25 or a variant thereof comprising up to 6 (1, 2, 3, 4, 5 or 6) amino acid mutations in the sequence of 1, 2 or 3 CDRs; preferably conservative amino acid substitutions.

In some preferred embodiments, the antibody or antigen-binding fragment according to the present disclosure comprises:
a) a heavy chain variable domain comprising: a heavy chain CDR1 of SEQ ID NO: 23, a heavy chain CDR2 of SEQ ID NO: 24, and a heavy chain CDR3 of SEQ ID NO: 25, and a light chain variable domain comprising: a light chain CDR1 of SEQ ID NO: 26, a light chain CDR2 of SEQ ID NO: 27 and a light chain CDR3 of SEQ ID NO: 28;
b) a heavy chain variable domain comprising: a heavy chain CDR1 of SEQ ID NO: 29, a heavy chain CDR2 of SEQ ID NO: 30 and a heavy chain CDR3 of SEQ ID NO: 31, and a light chain variable domain comprising: a light chain CDR1 of SEQ ID NO: 32, a light chain CDR2 of SEQ ID NO: 33, and a light chain CDR3 of SEQ ID NO: 34;
c) a heavy chain variable domain comprising: a heavy chain CDR1 of SEQ ID NO: 35, a heavy chain CDR2 of SEQ ID NO: 36 and a heavy chain CDR3 of SEQ ID NO: 37, and a light chain variable domain comprising: a light chain CDR1 of SEQ ID NO: 38, a light chain CDR2 of SEQ ID NO: 39, and a light chain CDR3 of SEQ ID NO: 40;
d) a heavy chain variable domain comprising: a heavy chain CDR1 of SEQ ID NO: 41, a heavy chain CDR2 of SEQ ID NO: 42 and a heavy chain CDR3 of SEQ ID NO: 43, and a light chain variable domain comprising: a light chain CDR1 of SEQ ID NO: 44, a light chain CDR2 of SEQ ID NO: 45, and a light chain CDR3 of SEQ ID NO: 46;
e) a heavy chain variable domain comprising: a heavy chain CDR1 of SEQ ID NO: 47, a heavy chain CDR2 of SEQ ID NO: 48 and a heavy chain CDR3 of SEQ ID NO: 49 and a light chain variable domain comprising: a light chain CDR1 of SEQ ID NO: 50, a light chain CDR2 of SEQ ID NO: 51, and a light chain CDR3 of SEQ ID NO: 52; or
f) a heavy chain variable domain comprising heavy chain CDR1, CDR2 and CDR3 which differ from SEQ ID NO: 23 to 25, 29 to 31, 35 to 37, 41 to 43 or 47 to 49, respectively by up to 6 (1, 2, 3, 4, 5 or 6) amino acid mutations in the sequence of 1, 2 or 3 CDRs; and a light chain variable domain comprising: light chain CDR1, CDR2, and CDR3 which differ from SEQ ID NO: 26 to 28, 32 to 34, 38 to 40, 44 to 46 or 50 to 52, respectively by up to 6 (1, 2, 3, 4, 5 or 6) amino acid mutations in the sequence of 1, 2 or 3 CDRs; preferably conservative amino acid substitutions.

In some more preferred embodiments, the antibody or antigen-binding fragment according to the present disclosure comprises: a heavy chain variable domain comprising: a heavy chain CDR1 of SEQ ID NO: 23, a heavy chain CDR2 of SEQ ID NO: 24, and a heavy chain CDR3 of SEQ ID NO: 25, and a light chain variable domain comprising: a light chain CDR1 of SEQ ID NO: 26, a light chain CDR2 of SEQ ID NO: 27 and a light chain CDR3 of SEQ ID NO: 28.

In some other more preferred embodiments, the antibody or antigen-binding fragment according to the present disclosure comprises: a heavy chain CDR1 of SEQ ID NO: 35, a heavy chain CDR2 of SEQ ID NO: 36, and a heavy chain CDR3 of SEQ ID NO: 37, and a light chain variable domain comprising: a light chain CDR1 of SEQ ID NO: 38, a light chain CDR2 of SEQ ID NO: 39 and a light chain CDR3 of SEQ ID NO: 40.

In some preferred embodiments, the antibody according to the present disclosure comprises:
a) a heavy chain variable domain comprising an amino acid sequence having at least 90% identity with SEQ ID NO: 3 and a light chain variable domain comprising an amino acid sequence having at least 90% identity with SEQ ID NO: 4,
b) a heavy chain variable domain comprising an amino acid sequence having at least 90% identity with SEQ ID NO: 5 and a light chain variable domain comprising an amino acid sequence having at least 90% identity with SEQ ID NO: 6,
c) a heavy chain variable domain comprising an amino acid sequence having at least 90% identity with SEQ ID NO: 7 and a light chain variable domain comprising an amino acid sequence having at least 90% identity with SEQ ID NO: 8,
d) a heavy chain variable domain comprising an amino acid sequence having at least 90% identity with SEQ ID NO: 9 and a light chain variable domain comprising an amino acid sequence having at least 90% identity with SEQ ID NO: 10, or
e) a heavy chain variable domain comprising an amino acid sequence having at least 90% identity with SEQ ID NO: 11 and a light chain variable domain comprising an amino acid sequence having at least 90% identity with SEQ ID NO: 12.

In some embodiments, the antibody or antigen-binding fragment according to the present disclosure does not comprise:
a) a heavy chain variable domain consisting of SEQ ID NO: 3 and a light chain variable domain consisting of SEQ ID NO: 4,
b) a heavy chain variable domain consisting of SEQ ID NO: 5 and a light chain variable domain consisting of SEQ ID NO: 6,
c) a heavy chain variable domain consisting of SEQ ID NO: 7 and a light chain variable domain consisting of SEQ ID NO: 8,
d) a heavy chain variable domain consisting of SEQ ID NO: 9 and a light chain variable domain consisting of SEQ ID NO: 10, or
e) a heavy chain variable domain consisting of SEQ ID NO: 11 and a light chain variable domain consisting of SEQ ID NO: 12.

In some preferred embodiments, the antibody according to the present disclosure comprises a heavy chain variable domain of SEQ ID NO: 3 and a light chain variable region of SEQ ID NO: 4.

In some embodiments, the heavy chain variable domains of the antibody according to the present disclosure are associated with a heavy chain constant region having at least 90% sequence identity with SEQ ID NO: 132.

In some embodiments, the heavy chain variable domains of the antibody or antigen-binding fragment according to the present disclosure are associated with IgG or IgA constant region. In some particular embodiments, the antibody comprising IgA constant region further comprises a J chain and/or a secretory component. In some particular embodiments, the constant region comprises mutation(s) and/or modifications that silence antibody effector functions and/or increase antibody half-life in vivo.

In some preferred embodiments, the antibody or antigen-binding fragment according to the present disclosure is associated with IgG1 constant region. Preferably, the antibody comprises a heavy chain amino acid sequence having at least 90% identity with any one of SEQ ID NO: 13, 15, 17, 19 and 21, preferably SEQ ID NO: 13. More preferably, the antibody comprises:
a) a heavy chain comprising a sequence having at least 90% identity with amino acid sequence of SEQ ID NO: 13 and a light chain comprising a sequence having at least 90% identity with amino acid sequence of SEQ ID NO: 14;
b) a heavy chain comprising a sequence having at least 90% identity with amino acid sequence of SEQ ID NO: 15 and a light chain comprising a sequence having at least 90% identity with amino acid sequence of SEQ ID NO: 16;
c) a heavy chain comprising a sequence having at least 90% identity with amino acid sequence of SEQ ID NO: 17 and a light chain comprising a sequence having at least 90% identity with amino acid sequence of SEQ ID NO: 18;
d) a heavy chain comprising a sequence having at least 90% identity with amino acid sequence of SEQ ID NO: 19 and a light chain comprising a sequence having at least 90% identity with amino acid sequence of SEQ ID NO: 20; or
e) a heavy chain comprising a sequence having at least 90% identity with amino acid sequence of SEQ ID NO: 21 and a light chain comprising a sequence having at least 90% identity with amino acid sequence of SEQ ID NO: 22.

In some other preferred embodiments, the antibody or antigen-binding fragment comprises a heavy chain amino acid sequence having at least 90% identity with any one of SEQ ID NO: 133, 134, 135, 136 and 137, preferably SEQ ID NO: 133. More preferably, the antibody comprises:
a) a heavy chain comprising a sequence having at least 90% identity with amino acid sequence of SEQ ID NO: 133 and a light chain comprising a sequence having at least 90% identity with amino acid sequence of SEQ ID NO: 14;
b) a heavy chain comprising a sequence having at least 90% identity with amino acid sequence of SEQ ID NO: 134 and a light chain comprising a sequence having at least 90% identity with amino acid sequence of SEQ ID NO: 16;
c) a heavy chain comprising a sequence having at least 90% identity with amino acid sequence of SEQ ID NO: 135 and a light chain comprising a sequence having at least 90% identity with amino acid sequence of SEQ ID NO: 18;
d) a heavy chain comprising a sequence having at least 90% identity with amino acid sequence of SEQ ID NO: 136 and a light chain comprising a sequence having at least 90% identity with amino acid sequence of SEQ ID NO: 20; or
e) a heavy chain comprising a sequence having at least 90% identity with amino acid sequence of SEQ ID NO: 137 and a light chain comprising a sequence having at least 90% identity with amino acid sequence of SEQ ID NO: 22.

In some more preferred embodiments, the antibody or antigen-binding fragment according to the present disclosure comprises a heavy chain amino acid sequence of SEQ ID NO: 13 and a light chain amino acid sequence of SEQ ID NO: 14.

In some other more preferred embodiments, the antibody or antigen-binding fragment according to the present disclosure comprises a heavy chain amino acid sequence of SEQ ID NO: 133 and a light chain amino acid sequence of SEQ ID NO: 14.

In some particular embodiments, the antibody or antigen-binding fragment according to the present disclosure is a recombinant human monoclonal antibody; preferably of IgG1 or IgA isotype; wherein the IgA may be monomeric, polymeric or secretory IgA; preferably the IgA is polymeric or secretory IgA.

In some particular embodiments, the antibody or antigen-binding fragment according to the present disclosure binds to recombinant SARS-CoV-2 S-trimer of SEQ ID NO: 106 with a KD selected from 10 nM or less, 1 nM or less, 500 pM or less, 400 pM or less, and 300 pM or less.

In some particular embodiments, the antibody or antigen-binding fragment according to the present disclosure binds to recombinant SARS-CoV-2 S1 protein of SEQ ID NO: 107 with a KD selected from 25 nM or less, 10 nM or less, 5 nM or less, and 1 nM or less.

In some particular embodiments, the antibody or antigen-binding fragment according to the present disclosure binds to recombinant SARS-CoV-2 RBD protein of any one of SEQ ID NO: 108 to 111 and 122 to 125 with a KD selected from 25 nM or less, 10 nM or less, 1 nM or less, 500 pM or less, 400 pM or less, 300 pM or less, and 100 pM or less.

In some particular embodiments, the antibody or antigen-binding fragment according to the present disclosure binds to at least one recombinant SARS-CoV-2 S protein selected from: a tri-Si protein of SEQ ID NO: 106, a S1 protein of SEQ ID NO: 107 and a RBD protein of SEQ ID NO: 108 to 111 and 122 to 125 with a binding affinity which is higher than that of recombinant angiotensin-converting enzyme 2 (ACE2) ectodomain protein of SEQ ID NO: 103; preferably at least 5, 10, 25, 50, 100, 250, 500 or 1000 folds higher; preferably wherein the binding affinity of the antibody for the RBD protein is at least 10, 25, 50, 100, 250, 500 or 1000 folds higher compared to that of the ACE2 ectodomain protein.

In some particular embodiments, the antibody or antigen-binding fragment according to the present disclosure competitively inhibits the binding of recombinant SARS-CoV-2 Spike protein of SEQ ID NO: 106 or 126 to recombinant ACE2 ectodomain protein of SEQ ID NO: 103 with an EC₅₀ selected from 1 μg/mL or less, 0.5 μg/mL or less, 0.4 μg/mL or less, 0.3 μg/mL or less, 0.2 μg/mL or less, and 0.1 μg/mL or less.

In some particular embodiments, the antibody or antigen-binding fragment according to the present disclosure blocks at least 70%, 80% or 90% of binding of recombinant SARS-CoV-2 Spike of SEQ ID NO: 106 or 126 or RBD proteins of SEQ ID NO: 108 to 111 and 122 to 125 to recombinant ACE2 ectodomain protein.

In some particular embodiments, the antibody or antigen-binding fragment according to the present disclosure blocks at least 70%, 80% or 90% of binding of recombinant SARS-CoV-2 Spike of SEQ ID NO: 106 or 126 or RBD proteins of SEQ ID NO: 108 to 111 and 122 to 125 and 184 to recombinant ACE2 ectodomain protein

In some particular embodiments of the antibody or antigen-binding fragment according to the present disclosure, the recombinant SARS-CoV-2 S-trimer, S1, and/or RBD protein is chosen from isolate Wuhan-Hu-1, B.1.1.7 lineage, P.1 lineage, B.1.351 lineage, B.1.617 lineage, B.1.1.529 lineage (including the “Omicron variant”), or any sublineage, variant of Concern (VOC), or Variant of Interest (VOI) thereof; for example any Omicron variant, lineage or sublineage such as BA.1, BA.1.1 or BA.2.

In some particular embodiments, the B.1.617 lineage may include any one of the sublineages selected from B.1.617.1, B.1.617.2 and B.1.617.3.

In some particular embodiments, the antibody or antigen-binding fragment according to the present disclosure neutralizes at least one SARS-CoV-2 variant chosen from the lineages B.1.1.7, P.1 and B.1.351 and B.1.617 and B.1.1.529; in particular chosen from the lineages B.1.1.7, P.1 and B.1.351 and B.1.617.2 and B.1.617.2.1 and B.1.617.1.3.

In some particular embodiments, the antibody or antigen-binding fragment according to the present disclosure neutralizes at least one SARS-CoV-2 variant chosen from the lineages B.1.1.7, P.1 and B.1.351 and B.1.617 and B.1.1.529 and BA.2; in particular chosen from the lineages B.1.1.7, P.1 and B.1.351 and B.1.617.2 and B.1.617.2.1 and B.1.617.1.3 and BA.2.

In some particular embodiments, the antibody or antigen-binding fragment according to the present disclosure neutralizes at least one SARS-CoV-2 variant, lineage or sublineage; for example chosen from the lineages of sublineages of the Omicron variant; for example the BA.2 variant sublineage.

In some particular embodiments, the antibody or antigen-binding fragment according to the present disclosure neutralizes SARS-CoV-2 with a half maximal effective concentration (EC₅₀) selected from 20 ng/mL or less, 15 ng/mL or less, 10 ng/mL or less, 5 ng/mL or less, and 1 ng/mL or less.

In some particular embodiments, the antibody or antigen-binding fragment according to the present disclosure neutralizes at least one SARS-CoV-2 chosen from: SARS-CoV-2 isolate Wuhan-Hu-1, SARS-CoV-2 variant D614G, and a SARS-CoV-2 variant comprising mutation(s) in the RBD domain.

According to preferred embodiments, the mutation(s) in the RBD domain are selected from one or more of N501Y, E484K, K417N and K417T substitutions. According to other preferred embodiments, the mutation(s) in the RBD domain are selected from one or more of N501Y, E484K, E484Q, K417N, K417T, L452R, T478K substitutions.

According to other preferred embodiments, the mutation(s) in the RBD domain are selected from one or more of K417N, K417T, N440K, L452R, G446S, S477N, T478K, E484A, E484K, E484Q, Q493R, G496S, Q498R and N501Y substitutions.

According to other preferred embodiments, the mutation(s) in the RBD domain are selected from one or more of K417N, N440K, G446S, S477N, T478K, E484A, E484K Q493R, G496S, Q498R and N501Y substitutions

Preferably the SARS-CoV-2 variant is chosen from the B.1.1.7, P.1 lineages and B.1.351 lineages and B.1.617 lineages and B.1.1.529 lineages.

In some particular embodiments, the antibody or antigen-binding fragment according to the present disclosure does not cross-react with at least one human coronavirus selected from SARS-CoV-1, MERS-CoV, NL63-CoV, OC43-CoV, HKU1-CoV and 229E-CoV; preferably the antibody does not cross-react with all of said human coronaviruses.

In some particular embodiments, the antibody or antigen-binding fragment according to the present disclosure has normal levels of Antibody-dependent-cellular-cytotoxicity (ADCC) as compared to a control antibody; in particular the antibody has normal affinity to a CD16 (FcγRI) receptor as compared to a control antibody.

In some particular embodiments, the antibody or antigen-binding fragment according to the present disclosure has modulated (i.e. low or decreased) levels of Antibody-dependent-cellular-cytotoxicity (ADCC) as compared to a control (positive or endogenous) antibody; in particular the antibody has modulated (i.e. low or decreased) affinity to a CD16 (FcγRI) receptor as compared to a control antibody.

In some particular embodiments, the antibody or antigen-binding fragment according to the present disclosure has low levels of Antibody-dependent-cellular-cytotoxicity (ADCC) as compared to a control antibody.

In some particular embodiments, the antibody or antigen-binding fragment according to the present disclosure has normal levels of Antibody-dependent-cellular-phagocytosis (ADCP) as compared to a control antibody; in particular the antibody has normal affinity to a CD32A (FcγRIIA) receptor as compared to a control antibody.

In some particular embodiments, the antibody or antigen-binding fragment according to the present disclosure has modulated (i.e. normal or improved) levels of Antibody-dependent-cellular-phagocytosis (ADCP) as compared to a control (positive or endogenous) antibody; in particular the antibody has modulated (i.e. normal or improved) affinity to a CD32A (FcγRIIA) receptor as compared to a control antibody.

In some particular embodiments, the antibody or antigen-binding fragment according to the present disclosure has improved levels of Antibody-dependent-cellular-phagocytosis (ADCP) as compared to a control antibody.

When determining the ADCP or ADCC activity of a given antibody, the control (negative) antibody may be selected from mGO53 as the isotype control.

In some particular embodiments, the antibody or antigen-binding fragment according to the present disclosure has no predicted reactivity to human proteins, no self-reactivity as compared to a control antibody, and/or no polyreactivity as compared to a control antibody.

In some particular embodiments, the antibody or antigen-binding fragment according to the present disclosure is produced in a eukaryotic recombinant system.

In some particular embodiments, the antibody or antigen-binding fragment according to the present disclosure is produced in a prokaryotic recombinant system.

In some particular embodiments, the antibody or antigen-binding fragment according to the present disclosure comprises a non-native human glycosylation pattern and/or a non-human glycosylation pattern.

In some particular embodiments, the antibody or antigen-binding fragment according to the present disclosure is produced recombinantly and comprises a non-native human glycosylation pattern and/or a non-human glycosylation pattern.

In some particular embodiments, the antibody or antigen-binding fragment according to the present disclosure is produced recombinantly and comprises a non-human glycosylation pattern.

Another aspect of the disclosure relates to an isolated antibody directed against the viral Spike protein receptor binding-domain (RBD) of SARS-CoV-2, or antigen-binding fragment thereof, characterized in that it comprises:
a heavy chain variable domain selected from:
a heavy chain variable domain comprising at least one of, preferably all three of: a heavy chain CDR1 of SEQ ID NO: 23, a heavy chain CDR2 of SEQ ID NO: 24 and a heavy chain CDR3 of SEQ ID NO: 25, or a variant with one or two conservative substitution(s); or
a heavy chain variable domain comprising at least one of, preferably all three of: a heavy chain CDR1 of SEQ ID NO: 29, a heavy chain CDR2 of SEQ ID NO: 30 and a heavy chain CDR3 of SEQ ID NO: 31, or a variant with one or two conservative substitution(s); or
a heavy chain variable domain comprising at least one of, preferably all three of: a heavy chain CDR1 of SEQ ID NO: 35, a heavy chain CDR2 of SEQ ID NO: 36 and a heavy chain CDR3 of SEQ ID NO: 37, or a variant with one or two conservative substitution(s); or
a heavy chain variable domain comprising at least one of, preferably all three of: a heavy chain CDR1 of SEQ ID NO: 41, a heavy chain CDR2 of SEQ ID NO: 42 and a heavy chain CDR3 of SEQ ID NO: 43, or a variant with one or two conservative substitution(s); or
a heavy chain variable domain comprising at least one of, preferably all three of: a heavy chain CDR1 of SEQ ID NO: 47, a heavy chain CDR2 of SEQ ID NO: 48 and a heavy chain CDR3 of SEQ ID NO: 49, or a variant with one or two conservative substitution(s);
and
a light chain variable domain selected from:
a light chain variable domain comprising at least one of, preferably all three of: a light chain CDR1 of SEQ ID NO: 26, a light chain CDR2 of SEQ ID NO: 27 and a light chain CDR3 of SEQ ID NO: 28, or a variant with one or two conservative substitution(s); or
a light chain variable domain comprising at least one of, preferably all three of: a light chain CDR1 of SEQ ID NO: 32, a light chain CDR2 of SEQ ID NO: 33 and a light chain CDR3 of SEQ ID NO: 34, or a variant with one or two conservative substitution(s); or
a light chain variable domain comprising at least one of, preferably all three of: a light chain CDR1 of SEQ ID NO: 38, a light chain CDR2 of SEQ ID NO: 39 and a light chain CDR3 of SEQ ID NO: 40, or a variant with one or two conservative substitution(s); or
a light chain variable domain comprising at least one of, preferably all three of: a light chain CDR1 of SEQ ID NO: 44, a light chain CDR2 of SEQ ID NO: 45 and a light chain CDR3 of SEQ ID NO: 46, or a variant with one or two conservative substitution(s); or
a light chain variable domain comprising at least one of, preferably all three of: a light chain CDR1 of SEQ ID NO: 50, a light chain CDR2 of SEQ ID NO: 51 and a light chain CDR3 of SEQ ID NO: 52, or a variant with one or two conservative substitution(s).

In some embodiments, the antibody or antigen-binding fragment comprises a heavy and/or light chain variable domain comprising the sequences as disclosed above; preferably associated with IgG or IgA constant region as disclosed above; for example associated with a heavy chain constant region having at least 90% sequence identity with SEQ ID NO: 132. In some particular embodiments, the antibody or antigen-binding fragment comprises a heavy and/or light chain comprising the sequences as disclosed above.

In some embodiments, the antibody according to the present disclosure or antigen-binding fragment thereof, further comprises a detectable label.

According to one of its main embodiments, the invention relates to an isolated antibody directed against the viral Spike protein receptor binding-domain (RBD) of SARS-CoV-2, or antigen-binding fragment thereof, characterized in that it comprises:
a heavy chain variable domain comprising all three of: a heavy chain CDR1 of SEQ ID NO: 23, a heavy chain CDR2 of SEQ ID NO: 24 and a heavy chain CDR3 of SEQ ID NO: 25, or variants thereof comprising up to 1 amino acid mutation in the sequence of 1, 2 or 3 CDRs; and a light chain variable domain comprising all three of: a light chain CDR1 of SEQ ID NO: 26, a light chain CDR2 of SEQ ID NO: 27 and a light chain CDR3 of SEQ ID NO: 28, or variants thereof comprising up to 1 amino acid mutation in the sequence of 1, 2 or 3 CDRs; or
a heavy chain variable domain comprising all three of: a heavy chain CDR1 of SEQ ID NO: 35, a heavy chain CDR2 of SEQ ID NO: 36 and a heavy chain CDR3 of SEQ ID NO: 37; and a light chain variable domain comprising all three of: a light chain CDR1 of SEQ ID NO: 38, a light chain CDR2 of SEQ ID NO: 39 and a light chain CDR3 of SEQ ID NO: 40.

According to a second main embodiment, the invention relates to a human neutralizing monoclonal antibody against Severe Acute Respiratory Syndrome-related Coronavirus 2 (SARS-CoV-2), or antigen-binding fragment thereof, which specifically binds to the viral Spike protein receptor-binding domain (RBD) and is a competitive inhibitor of binding to the RBD of at least one of the following reference antibodies:
the reference human antibody Cv2.1169 comprising (i) a heavy chain variable region comprising the amino acid sequence of SEQ ID NO: 3 and (ii) a light chain variable region comprising the amino acid sequence of SEQ ID NO: 4; and
the reference human antibody Cv2.3194 comprising (i) a heavy chain variable region comprising the amino acid sequence of SEQ ID NO: 7 and a light chain variable region comprising the amino acid sequence of SEQ ID NO: 8;
   said human neutralizing antibody, or antigen-binding fragment thereof, comprising:
a) a heavy chain variable domain comprising: a heavy chain CDR1 of SEQ ID NO: 23, a heavy chain CDR2 of SEQ ID NO: 24, and a heavy chain CDR3 of SEQ ID NO: 25, and a light chain variable domain comprising: a light chain CDR1 of SEQ ID NO: 26, a light chain CDR2 of SEQ ID NO: 27 and a light chain CDR3 of SEQ ID NO: 2; or
b) a heavy chain variable domain comprising: a heavy chain CDR1 of SEQ ID NO: 35, a heavy chain CDR2 of SEQ ID NO: 36 and a heavy chain CDR3 of SEQ ID NO: 37, and a light chain variable domain comprising: a light chain CDR1 of SEQ ID NO: 38, a light chain CDR2 of SEQ ID NO: 39, and a light chain CDR3 of SEQ ID NO: 40.

Another aspect of the invention relates to an isolated nucleic acid encoding an antibody or antigen-binding fragment according to the present disclosure; preferably comprising at least a nucleic acid sequence encoding the heavy and/or light chain of said antibody according to the present disclosure.

In some particular embodiments, the isolated nucleic acid according to the present disclosure is mRNA, preferably modified mRNA.

In some particular embodiments, the isolated nucleic acid according to the present disclosure is DNA.

Another aspect of the invention relates to an expression vector for the recombinant production of an antibody according to the present disclosure in a host cell, comprising at least one nucleic acid encoding said antibody according to the present disclosure.

In some particular embodiments, the expression vector according to the present disclosure comprises a pair of nucleic acid sequences selected from: a sequence having at least 90% identify with SEQ ID NO: 93 and a sequence having at least 90% identify with SEQ ID NO: 94; a sequence having at least 90% identify with SEQ ID NO: 95 and a sequence having at least 90% identify with SEQ ID NO: 96; a sequence having at least 90% identify with SEQ ID NO: 97 and a sequence having at least 90% identify with SEQ ID NO: 98; a sequence having at least 90% identify with SEQ ID NO: 99 and a sequence having at least 90% identify with SEQ ID NO: 100; and a sequence having at least 90% identify with SEQ ID NO: 101 and a sequence having at least 90% identify with SEQ ID NO: 102.

In some particular embodiments, the expression vector according to according to the present disclosure is contained in a bacteria strain chosen from Cv2.1169_pIgH and Cv2.1169_pIgL deposited under the terms of the Budapest Treaty at the Collection Nationale de Cultures de Microorganismes (CNCM) at the Institut Pasteur, 25 rue du Docteur Roux, 75724 Paris, FR, on Jan. 28, 2021 under the number I-5651 and I-5652, respectively.

In some particular embodiments, the expression vector according to the present disclosure is contained in a bacteria strain selected from the group consisting of: Cv2.1353_IgH, Cv2.1353_IgL, Cv2.3194_IgH, Cv2.3194_IgL, Cv2.3235_IgH, Cv2.3235_IgL, Cv2.5213_IgH, Cv2.5213_IgL deposited under the terms of the Budapest Treaty at the Collection Nationale de Cultures de Microorganismes (CNCM) at the Institut Pasteur, 25 rue du Docteur Roux, 75724 Paris, FR, on Apr. 2, 2021 under the number I-5668, I-5669, I-5670, I-5671, I-5672, I-5673, I-5674, and I-5675, respectively.

In some particular embodiments, the expression vector according to the present disclosure is contained in a bacteria strain selected from the group consisting of Cv2.5179_IgH and Cv2.5179_IgL registered under the terms of the Budapest Treaty at the Collection Nationale de Cultures de Microorganismes (CNCM) at the Institut Pasteur, 25 rue du Docteur Roux, 75724 Paris, FR, on Nov. 15, 2021 under the number CNCM I-5775 and CNCM I-5776 respectively.

Another aspect relates to a host cell comprising an expression vector according to the present disclosure or a nucleic acid according to the present disclosure.

In some particular embodiments, the host cell according to the present disclosure is an antibody producing cell-line stably transformed with the expression vector.

In some particular embodiments, the host cell according to the present disclosure is a eukaryotic cell; preferably chosen from yeast, insect and mammalian cells.

Another aspect relates to a method of production of the antibody according to the present disclosure, comprising: (i) culturing the host cell of the present disclosure for expression of said antibody by the host cell; and optionally (ii) recovering the antibody; and (iii) purifying said antibody.

Another aspect relates to a pharmaceutical composition comprising the antibody, antigen-binding fragment thereof, nucleic acid or vector according to the present disclosure, and at least one of a pharmaceutically acceptable carrier, an adjuvant, and a preservative.

Another particular aspect of the disclosure relates to a pharmaceutical composition comprising:
(i) an antibody, or antigen-binding fragment thereof, nucleic acid or vector; and
(ii) an antibody selected from the group of at least one of the following reference antibodies: Adintrevimab, Cilgavimab, Sotrovimab, Imdevimab;
Another aspect of the disclosure relates to a kit comprising:
(i) an antibody, or antigen-binding fragment thereof, nucleic acid or vector; and
(ii) an antibody selected from the group of at least one of the following reference antibodies: Adintrevimab, Cilgavimab, Sotrovimab, Imdevimab.

In some particular embodiments, the nucleic acid is mRNA, in particular modified mRNA; preferably formulated in a vesicle or particle, in particular a lipid nanoparticle (LNP).

In some particular embodiments, the pharmaceutical composition according to the present disclosure is for parenteral injection, infusion, local delivery, inhalation, or sustained delivery.

Another aspect relates to the antibody according to the present disclosure, antigen-binding fragment thereof, nucleic acid, vector, or pharmaceutical composition according to the present disclosure, for use as a medicament.

Another aspect relates to the pharmaceutical composition according to the present disclosure, for use in the prevention or treatment of SARS-CoV-2 infection and associated COVID-19 disease.

Another aspect relates to the use of a pharmaceutical composition according to the present disclosure for the manufacture of a medicament for the prevention or treatment of SARS-CoV-2 infection and associated COVID-19 disease.

Another aspect relates to a method for the detection of a SARS-CoV-2 in a sample comprising: contacting said sample with an antibody according to the present disclosure or antigen-binding fragment thereof, and detecting the antigen-antibody complexes formed, thereby detecting the presence, absence or level of SARS-CoV-2 in the sample.

In some particular embodiments of the method according to the present disclosure, the sample is a biological sample from a subject suspected to be contaminated with SARS-CoV-2 and the method is for the diagnosis of SARS-CoV-2 infection and associated COVID-19 disease.

In some particular embodiments of the method according to the present disclosure, the sample is a biological sample from a COVID-19 patient before or during treatment of COVID-19 disease and the method is for the monitoring of treatment of COVID-19 disease.

Another aspect relates to a kit for the detection or diagnosis of SARS-CoV-2 infection or contamination, or the monitoring of treatment of COVID-19 disease, comprising at least an antibody according to the present disclosure or antigen-binding fragment thereof, preferably further including a detectable label.

Another aspect relates to a method of reducing the risk of developing SARS-CoV-2-associated COVID-19 disease in a subject, comprising administering an effective amount of an antibody, an antigen-binding fragment thereof, a nucleic acid or vector, or a pharmaceutical composition according to the present disclosure, to the subject.

In some particular embodiments of the method, the risk of hospitalization is reduced.

In some particular embodiments of the method, the risk of death is reduced.

Another aspect relates to a method of treating SARS-CoV-2-associated COVID-19 disease in a subject, comprising administering an effective amount of an antibody, an antigen-binding fragment thereof, a nucleic acid or vector, or a pharmaceutical composition according to the present disclosure, to the subject.

In some particular embodiments of the method, the likelihood of developing severe disease is reduced by the treatment.

In some particular embodiments of the method, the likelihood of hospitalization is reduced by the treatment.

In some particular embodiments of the method, the subject is hospitalized.

Another aspect relates to a method of treating SARS-CoV-2-associated COVID-19 disease in a subject, comprising administering an effective amount of a combination of at least two antibodies, or antigen-binding fragments thereof, according to the present disclosure.

Another aspect relates to a method of treating SARS-CoV-2-associated COVID-19 disease in a subject, comprising administering an effective amount of a combination of an antibody, or antigen-binding fragment thereof, according to the present disclosure with an antibody selected from Adintrevimab, Cilgavimab, Sotrovimab and Imdevimab.

Another aspect relates to a method according to the present disclosure, wherein the subject is at risk of developing a SARS, more particularly a subject with concurrent underlying conditions such as obesity, diabetes, cancer, under immunosuppressive therapy, primary immune deficiency or unresponsive to vaccines.

Another aspect relates to a method comprising administering an effective amount of an antibody, or antigen-binding fragment, or nucleic acid or vector, or pharmaceutical composition according to the present disclosure, for preventing and/or reducing the likelihood of occurrence of a Coronaviridae infection; in particular a SARS-CoV-2 infection.

Another aspect relates to a method comprising administering an effective amount of an antibody, or antigen-binding fragment, or nucleic acid or vector, or pharmaceutical composition according to the present disclosure, for preventing and/or reducing the likelihood of occurrence of a complication of a Coronaviridae infection, in particular of a respiratory, nervous, gastrointestinal or cardiovascular complication of a Coronaviridae infection; in particular of a SARS-CoV-2 infection.

Another particular aspect relates to a method comprising administering an effective amount of an antibody, or antigen-binding fragment, or nucleic acid or vector, or pharmaceutical composition according to the present disclosure, for preventing and/or reducing the likelihood of occurrence of a severe acute respiratory complication of a Coronaviridae infection; in particular of a SARS-CoV-2 infection.

Another aspect relates to a method comprising administering an effective amount of an antibody, or antigen-binding fragment, or nucleic acid or vector, or pharmaceutical composition according to the present disclosure, for preventing and/or reducing the likelihood of occurrence of a Coronaviridae infection in an individual, said individual being characterized in that
the individual has not been administered a vaccine against the said Coronaviridae infection; or
the individual is not responding to the said vaccine; or
the individual's level of antibodies directed against the Coronaviridae infection is at or below a protecting threshold level.

Another aspect relates to a method comprising administering an effective amount of an antibody, or antigen-binding fragment, or nucleic acid or vector, or pharmaceutical composition according to the present disclosure, for improving an immune response against a Coronaviridae virus; in particular of a SARS-CoV-2 infection.

Another particular aspect relates to a method comprising administering an effective amount of an antibody, or antigen-binding fragment, or nucleic acid or vector, or pharmaceutical composition according to the present disclosure, for improving an immune response against a viral Spike protein receptor-binding domain (RBD) of a Coronaviridae virus; or a fragment thereof.

Another aspect relates to a method comprising administering an effective amount of an antibody, or antigen-binding fragment, or nucleic acid or vector, or pharmaceutical composition according to the present disclosure, in combination with a vaccine against a Coronaviridae infection, in particular of a SARS-CoV-2 infection.

Another aspect relates to a method comprising administering an effective amount of an antibody, or antigen-binding fragment, or nucleic acid or vector, or pharmaceutical composition according to the present disclosure, in combination with a second antibody which specifically neutralizes the Severe Acute Respiratory Syndrome-related Coronavirus 2 (SARS-CoV-2), said second antibody being not a competitive inhibitor of binding to the RBD with the first antibody.

Another aspect relates to a method comprising administering an effective amount of an antibody, or antigen-binding fragment, or nucleic acid or vector, or pharmaceutical composition according to the present disclosure, in combination with a second antibody which specifically binds to a viral Spike protein receptor-binding domain (RBD) of a Severe Acute Respiratory Syndrome-related Coronavirus 2 (SARS-CoV-2), said second antibody being a class 2 or class 3 anti-SARS-CoV2 Spike protein antibody.

Another aspect relates to a method comprising administering an effective amount of an antibody, or antigen-binding fragment, or nucleic acid or vector, or pharmaceutical composition according to the present disclosure, in combination with a second antibody selected from the group of at least one of the following reference antibodies: Adintrevimab, Cilgavimab, Imdevimab, and Sotrovimab.

Another aspect relates to an antibody, or antigen-binding fragment, or nucleic acid or vector, or pharmaceutical composition according to the present disclosure, in combination with a vaccine against a Coronaviridae infection, in particular of a SARS-CoV-2 infection; for use as a medicament.

Another aspect relates to a use of an antibody, or antigen-binding fragment, or nucleic acid or vector, or pharmaceutical composition according to the present disclosure, in combination with a vaccine against a Coronaviridae infection, in particular of a SARS-CoV-2 infection, for the preparation of a medicament.

Another aspect relates to an antibody, or antigen-binding fragment, or nucleic acid or vector, or pharmaceutical composition according to the present disclosure, in combination with a second antibody which specifically neutralizes the Severe Acute Respiratory Syndrome-related Coronavirus 2 (SARS-CoV-2), said second antibody being not a competitive inhibitor of binding to the RBD with the first antibody; for use as a medicament.

Another aspect relates to a use of an antibody, or antigen-binding fragment, or nucleic acid or vector, or pharmaceutical composition according to the present disclosure, in combination with a second antibody which specifically neutralizes the Severe Acute Respiratory Syndrome-related Coronavirus 2 (SARS-CoV-2), said second antibody being not a competitive inhibitor of binding to the RBD with the first antibody; for the preparation of a medicament.

Another aspect relates to an antibody, or antigen-binding fragment, or nucleic acid or vector, or pharmaceutical composition according to the present disclosure, in combination with a second antibody which specifically binds to a viral Spike protein receptor-binding domain (RBD) of a Severe Acute Respiratory Syndrome-related Coronavirus 2 (SARS-CoV-2), said second antibody being a class 2 or class 3 anti-SARS-CoV2 Spike protein antibody; for use as a medicament.

Another aspect relates to a use of an antibody, or antigen-binding fragment, or nucleic acid or vector, or pharmaceutical composition according to the present disclosure, in combination with a second antibody which specifically binds to a viral Spike protein receptor-binding domain (RBD) of a Severe Acute Respiratory Syndrome-related Coronavirus 2 (SARS-CoV-2), said second antibody being a class 2 or class 3 anti-SARS-CoV2 Spike protein antibody; for the preparation of a medicament.

Another aspect relates to an antibody, or antigen-binding fragment, or nucleic acid or vector, or pharmaceutical composition according to the present disclosure, in combination with a second antibody selected from the group of at least one of the following reference antibodies: Adintrevimab, Cilgavimab, Imdevimab, and Sotrovimab; for use as a medicament, in particular for the above-mentioned indications.

Another aspect relates to a use of an antibody, or antigen-binding fragment, or nucleic acid or vector, or pharmaceutical composition according to the present disclosure, in combination with a second antibody selected from the group of at least one of the following reference antibodies: Adintrevimab, Cilgavimab, Imdevimab, and Sotrovimab; for the preparation of a medicament.

The uses, methods, compositions and kits according to the present disclosure may be advantageously suitable for humans and non-human patients such as non-human mammals.

Another aspect relates to a medical device, comprising the pharmaceutical composition according to the present disclosure; preferably in a form suitable for administration by injection or inhalation.

### DETAILED DESCRIPTION OF THE INVENTION

The disclosure provides antibodies, including antigen-binding fragments thereof, against SARS-CoV-2 Spike protein, in particular recombinant human monoclonal antibodies against SARS-CoV-2 Spike protein having the following properties:
they bind specifically to SARS-CoV-2 Spike protein receptor-binding domain (RBD) with a binding affinity which is higher than that of angiotensin-converting enzyme 2 (ACE2) ectodomain; results of antibody and ACE2-ectodomain protein binding assay to SARS-CoV-2 Spike (S trimer or tri-S), S1 sub-unit, Spike-RBD (S-RBD) proteins are shown in FIG. 3A;
they block the binding to ACE2 ectodomain protein of Spike and S-RBD proteins from SARS-CoV-2 and viral variants (B.1.1.7, B.1.351, P.1, B.1.617 and B.1.1.529); (FIGS. 3C, 3F, 3I, and 3L);
they neutralize SARS-CoV-2 virus, including Wuhan-Hu-1 isolate and variants thereof (D614G, B.1.1.7, B.1.351, P.1), in particular variants harboring the E484K immune escape mutation (P.1, B.1.351); FIGS. 3D, 3G, and 3I;
they compete with each other for binding to SARS-CoV-2 Spike and S-RBD proteins (FIG. 3B) indicating that they may bind to the same or related (spatially proximal) epitope on SARS-CoV-2 Spike receptor-binding domain; and
they do not cross-react with other coronavirus including human pathogenic betacoronavirus (group B/C) SARS-CoV-1 and MERS-CoV; alphacoronavirus NL63-CoV and 229E-CoV and betacoronavirus group A HKU1-CoV (Table 5).
they are active at least as IgG or IgA (FIGS. 3L and 3M).

Furthermore, all the antibodies (6 antibodies tested) have limited Fc effector functions, in particular Antibody-dependent-cellular-cytotoxicity (ADCC) (Table 5). All the antibodies are predicted to not react with human proteins (no off-target binding; FIG. 5E) and they are not self-reactive antibodies (FIGS. 5C, 5D and 5E). The majority of antibodies are non-polyreactive antibodies (FIGS. 5A, 5B and 5F).

The therapeutic efficacy of the human neutralizing IgG antibodies against SARS-CoV-2 was validated in two different animal models of SARS-CoV-2 infection (FIG. 6). Due to their specificity for SARS-CoV-2, these antibodies are also useful for the diagnosis of SARS-CoV-2 infection and monitoring of COVTD-19 treatment.

The disclosure further provides experimental evidence that such antibodies, and antigen-binding fragments thereof, are also potent neutralizing antibodies against new variants-of-concern (VOC) and variants-of-interest (VOI), including delta and omicron variants (FIGS. 9, 10, 11, 12, 13 and 14).

The disclosure further provides benchmark studies of such antibodies and antigen-binding fragments, in the form of competition assays, with other reference therapeutic antibodies directed against the SARS-CoV-2 Spike protein. In particular, it is demonstrated herein that such antibodies and antigen-binding fragments demonstrate a broader neutralizing effect than other therapeutic antibodies, while also targeting a distinct or partially overlapping epitope (FIGS. 10, 11, 16 and 18).

In view of these results, the SARS-CoV-2 neutralizing antibodies according to the disclosure, including antigen-binding fragments thereof, represent promising immunotherapeutic and diagnostic tools for the treatment and diagnosis of SARS-CoV-2 infection and associated disease COVID-19.

The disclosure relates to human neutralizing monoclonal antibody against Severe Acute Respiratory Syndrome-related Coronavirus 2 (SARS-CoV-2), or antigen-binding fragment thereof, which specifically binds to the viral Spike protein receptor-binding domain (RBD) and is a competitive inhibitor of binding to the RBD of at least one of the following reference antibodies:
the reference human antibody Cv2.3194 comprising (i) a heavy chain variable region comprising the amino acid sequence of SEQ ID NO: 7 and a light chain variable region comprising the amino acid sequence of SEQ ID NO: 8;
the reference human antibody Cv2.5179 comprising (i) a heavy chain variable region comprising the amino acid sequence of SEQ ID NO: 152 and (ii) a light chain variable region comprising the amino acid sequence of SEQ ID NO: 153
the reference human antibody Cv2.1169 comprising (i) a heavy chain variable region comprising the amino acid sequence of SEQ ID NO: 3 and (ii) a light chain variable region comprising the amino acid sequence of SEQ ID NO: 4;
the reference human antibody Cv2.1353 comprising (i) a heavy chain variable region comprising the amino acid sequence of SEQ ID NO: 5 and a light chain variable region comprising the amino acid sequence of SEQ ID NO: 6;
the reference human antibody Cv2.5213 comprising (i) a heavy chain variable region comprising the amino acid sequence of SEQ ID NO: 11 and a light chain variable region comprising the amino acid sequence of SEQ ID NO: 12; and
the reference human antibody Cv2.3235 comprising (i) a heavy chain variable region comprising the amino acid sequence of SEQ ID NO: 9 and a light chain variable region comprising the amino acid sequence of SEQ ID NO: 10;
   said human neutralizing antibody, or antigen-binding fragment thereof, comprising:
a) a heavy chain variable domain comprising: a heavy chain CDR1 of SEQ ID NO: 35, a heavy chain CDR2 of SEQ ID NO: 36 and a heavy chain CDR3 of SEQ ID NO: 37, and a light chain variable domain comprising: a light chain CDR1 of SEQ ID NO: 38, a light chain CDR2 of SEQ ID NO: 39, and a light chain CDR3 of SEQ ID NO: 40;
b) a heavy chain variable domain comprising: a heavy chain CDR1 of SEQ ID NO: 138, a heavy chain CDR2 of SEQ ID NO: 139, and a heavy chain CDR3 of SEQ ID NO: 140, and a light chain variable domain comprising: a light chain CDR1 of SEQ ID NO: 141, a light chain CDR2 of SEQ ID NO: 142 and a light chain CDR3 of SEQ ID NO: 143;
c) a heavy chain variable domain comprising: a heavy chain CDR1 of SEQ ID NO: 23, a heavy chain CDR2 of SEQ ID NO: 24, and a heavy chain CDR3 of SEQ ID NO: 25, and a light chain variable domain comprising: a light chain CDR1 of SEQ ID NO: 26, a light chain CDR2 of SEQ ID NO: 27 and a light chain CDR3 of SEQ ID NO: 28;
d) a heavy chain variable domain comprising: a heavy chain CDR1 of SEQ ID NO: 29, a heavy chain CDR2 of SEQ ID NO: 30 and a heavy chain CDR3 of SEQ ID NO: 31, and a light chain variable domain comprising: a light chain CDR1 of SEQ ID NO: 32, a light chain CDR2 of SEQ ID NO: 33, and a light chain CDR3 of SEQ ID NO: 34;
e) a heavy chain variable domain comprising: a heavy chain CDR1 of SEQ ID NO: 41, a heavy chain CDR2 of SEQ ID NO: 42 and a heavy chain CDR3 of SEQ ID NO: 43, and a light chain variable domain comprising: a light chain CDR1 of SEQ ID NO: 44, a light chain CDR2 of SEQ ID NO: 45, and a light chain CDR3 of SEQ ID NO: 46;
f) a heavy chain variable domain comprising: a heavy chain CDR1 of SEQ ID NO: 47, a heavy chain CDR2 of SEQ ID NO: 48 and a heavy chain CDR3 of SEQ ID NO: 49 and a light chain variable domain comprising: a light chain CDR1 of SEQ ID NO: 50, a light chain CDR2 of SEQ ID NO: 51, and a light chain CDR3 of SEQ ID NO: 52; or
g) a heavy chain variable domain comprising heavy chain CDR1, CDR2 and CDR3 which differ from SEQ ID NO: 23 to 25, or 138 to 140, respectively by up to 2 amino acid mutations in the sequence of 1, 2 or 3 CDRs; and a light chain variable domain comprising: light chain CDR1, CDR2, and CDR3 which differ from SEQ ID NO: 26 to 28, or 141 to 143, respectively by up to 2 amino acid mutations in the sequence of 1, 2 or 3 CDRs.

The disclosure further relates to an isolated antibody directed against the viral Spike protein receptor binding-domain (RBD) of SARS-CoV-2, or antigen-binding fragment thereof, characterized in that it comprises:
a heavy chain variable domain selected from:
a heavy chain variable domain comprising all three of: a heavy chain CDR1 of SEQ ID NO: 35, a heavy chain CDR2 of SEQ ID NO: 36 and a heavy chain CDR3 of SEQ ID NO: 37; or
a heavy chain variable domain comprising all three of: a heavy chain CDR1 of SEQ ID NO: 138, a heavy chain CDR2 of SEQ ID NO: 139 and a heavy chain CDR3 of SEQ ID NO: 140, or variants thereof with one or two conservative substitution(s); or
a heavy chain variable domain comprising all three of: a heavy chain CDR1 of SEQ ID NO: 23, a heavy chain CDR2 of SEQ ID NO: 24 and a heavy chain CDR3 of SEQ ID NO: 25, or variants thereof with one or two conservative substitution(s); or
a heavy chain variable domain comprising all three of: a heavy chain CDR1 of SEQ ID NO: 29, a heavy chain CDR2 of SEQ ID NO: 30 and a heavy chain CDR3 of SEQ ID NO: 31 or
a heavy chain variable domain comprising all three of: a heavy chain CDR1 of SEQ ID NO: 41, a heavy chain CDR2 of SEQ ID NO: 42 and a heavy chain CDR3 of SEQ ID NO: 43; or
a heavy chain variable domain comprising all three of: a heavy chain CDR1 of SEQ ID NO: 47, a heavy chain CDR2 of SEQ ID NO: 48 and a heavy chain CDR3 of SEQ ID NO: 49;
and
a light chain variable domain selected from:
a light chain variable domain comprising all three of: a light chain CDR1 of SEQ ID NO: 38, a light chain CDR2 of SEQ ID NO: 39 and a light chain CDR3 of SEQ ID NO: 40; or
a light chain variable domain comprising all three of: a light chain CDR1 of SEQ ID NO: 141, a light chain CDR2 of SEQ ID NO: 142 and a light chain CDR3 of SEQ ID NO: 143, or variants thereof with one or two conservative substitution(s); or
a light chain variable domain comprising all three of: a light chain CDR1 of SEQ ID NO: 26, a light chain CDR2 of SEQ ID NO: 27 and a light chain CDR3 of SEQ ID NO: 28, or variants thereof with one or two conservative substitution(s); or
a light chain variable domain comprising all three of: a light chain CDR1 of SEQ ID NO: 32, a light chain CDR2 of SEQ ID NO: 33 and a light chain CDR3 of SEQ ID NO: 34; or
a light chain variable domain comprising all three of: a light chain CDR1 of SEQ ID NO: 44, a light chain CDR2 of SEQ ID NO: 45 and a light chain CDR3 of SEQ ID NO: 46; or
a light chain variable domain comprising all three of: a light chain CDR1 of SEQ ID NO: 50, a light chain CDR2 of SEQ ID NO: 51 and a light chain CDR3 of SEQ ID NO: 52.

According to one of its main embodiments, the invention relates to an isolated antibody directed against the viral Spike protein receptor binding-domain (RBD) of SARS-CoV-2, or antigen-binding fragment thereof, characterized in that it comprises:
a heavy chain variable domain comprising all three of: a heavy chain CDR1 of SEQ ID NO: 23, a heavy chain CDR2 of SEQ ID NO: 24 and a heavy chain CDR3 of SEQ ID NO: 25, or variants thereof comprising up to 1 amino acid mutation in the sequence of 1, 2 or 3 CDRs; and a light chain variable domain comprising all three of: a light chain CDR1 of SEQ ID NO: 26, a light chain CDR2 of SEQ ID NO: 27 and a light chain CDR3 of SEQ ID NO: 28, or variants thereof comprising up to 1 amino acid mutation in the sequence of 1, 2 or 3 CDRs; or
a heavy chain variable domain comprising all three of: a heavy chain CDR1 of SEQ ID NO: 35, a heavy chain CDR2 of SEQ ID NO: 36 and a heavy chain CDR3 of SEQ ID NO: 37; and a light chain variable domain comprising all three of: a light chain CDR1 of SEQ ID NO: 38, a light chain CDR2 of SEQ ID NO: 39 and a light chain CDR3 of SEQ ID NO: 40.

In some particular embodiments, the invention relates to an isolated antibody directed against the viral Spike protein receptor binding-domain (RBD) of SARS-CoV-2, or antigen-binding fragment thereof, characterized in that it comprises:
a heavy chain variable domain comprising all three of: a heavy chain CDR1 of SEQ ID NO: 23, a heavy chain CDR2 of SEQ ID NO: 24 and a heavy chain CDR3 of SEQ ID NO: 25, or variants thereof comprising up to 1 amino acid mutation in the sequence of 1, 2 or 3 CDRs; and a light chain variable domain comprising all three of: a light chain CDR1 of SEQ ID NO: 26, a light chain CDR2 of SEQ ID NO: 27 and a light chain CDR3 of SEQ ID NO: 28, or variants thereof comprising up to 1 amino acid mutation in the sequence of 1, 2 or 3 CDRs.

In some particular embodiments, the invention relates to an isolated antibody directed against the viral Spike protein receptor binding-domain (RBD) of SARS-CoV-2, or antigen-binding fragment thereof, characterized in that it comprises:
a heavy chain variable domain comprising all three of: a heavy chain CDR1 of SEQ ID NO: 23, a heavy chain CDR2 of SEQ ID NO: 24 and a heavy chain CDR3 of SEQ ID NO: 25, or variants thereof comprising up to 1 amino acid mutation in the sequence of 1 or 2 CDRs; and a light chain variable domain comprising all three of: a light chain CDR1 of SEQ ID NO: 26, a light chain CDR2 of SEQ ID NO: 27 and a light chain CDR3 of SEQ ID NO: 28, or variants thereof comprising up to 1 amino acid mutation in the sequence of 1 or 2 CDRs.

In some particular embodiments, the invention relates to an isolated antibody directed against the viral Spike protein receptor binding-domain (RBD) of SARS-CoV-2, or antigen-binding fragment thereof, characterized in that it comprises:
a heavy chain variable domain comprising all three of: a heavy chain CDR1 of SEQ ID NO: 23, a heavy chain CDR2 of SEQ ID NO: 24 and a heavy chain CDR3 of SEQ ID NO: 25, or variants thereof comprising up to 1 amino acid mutation in the sequence of 1 CDR; and a light chain variable domain comprising all three of: a light chain CDR1 of SEQ ID NO: 26, a light chain CDR2 of SEQ ID NO: 27 and a light chain CDR3 of SEQ ID NO: 28, or variants thereof comprising up to 1 amino acid mutation in the sequence of 1 CDR.

In some particular embodiments, the invention relates to an isolated antibody directed against the viral Spike protein receptor binding-domain (RBD) of SARS-CoV-2, or antigen-binding fragment thereof, characterized in that it comprises:
a heavy chain variable domain comprising all three of: a heavy chain CDR1 of SEQ ID NO: 23, a heavy chain CDR2 of SEQ ID NO: 24 and a heavy chain CDR3 of SEQ ID NO: 25; and a light chain variable domain comprising all three of: a light chain CDR1 of SEQ ID NO: 26, a light chain CDR2 of SEQ ID NO: 27 and a light chain CDR3 of SEQ ID NO: 28.

In some particular embodiments, the invention relates to an isolated antibody directed against the viral Spike protein receptor binding-domain (RBD) of SARS-CoV-2, or antigen-binding fragment thereof, characterized in that it comprises:
a heavy chain variable domain comprising all three of: a heavy chain CDR1 of SEQ ID NO: 35, a heavy chain CDR2 of SEQ ID NO: 36 and a heavy chain CDR3 of SEQ ID NO: 37; and a light chain variable domain comprising all three of: a light chain CDR1 of SEQ ID NO: 38, a light chain CDR2 of SEQ ID NO: 39 and a light chain CDR3 of SEQ ID NO: 40.

According to a second main embodiment, the invention relates to a human neutralizing monoclonal antibody against Severe Acute Respiratory Syndrome-related Coronavirus 2 (SARS-CoV-2), or antigen-binding fragment thereof, which specifically binds to the viral Spike protein receptor-binding domain (RBD) and is a competitive inhibitor of binding to the RBD of at least one of the following reference antibodies:
the reference human antibody Cv2.1169 comprising (i) a heavy chain variable region comprising the amino acid sequence of SEQ ID NO: 3 and (ii) a light chain variable region comprising the amino acid sequence of SEQ ID NO: 4; and
the reference human antibody Cv2.3194 comprising (i) a heavy chain variable region comprising the amino acid sequence of SEQ ID NO: 7 and a light chain variable region comprising the amino acid sequence of SEQ ID NO: 8;
   said human neutralizing antibody, or antigen-binding fragment thereof, comprising:
a) a heavy chain variable domain comprising: a heavy chain CDR1 of SEQ ID NO: 23, a heavy chain CDR2 of SEQ ID NO: 24, and a heavy chain CDR3 of SEQ ID NO: 25, and a light chain variable domain comprising: a light chain CDR1 of SEQ ID NO: 26, a light chain CDR2 of SEQ ID NO: 27 and a light chain CDR3 of SEQ ID NO: 2; or
b) a heavy chain variable domain comprising: a heavy chain CDR1 of SEQ ID NO: 35, a heavy chain CDR2 of SEQ ID NO: 36 and a heavy chain CDR3 of SEQ ID NO: 37, and a light chain variable domain comprising: a light chain CDR1 of SEQ ID NO: 38, a light chain CDR2 of SEQ ID NO: 39, and a light chain CDR3 of SEQ ID NO: 40.

In some particular embodiments, the invention relates to a human neutralizing monoclonal antibody against Severe Acute Respiratory Syndrome-related Coronavirus 2 (SARS-CoV-2), or antigen-binding fragment thereof, which specifically binds to the viral Spike protein receptor-binding domain (RBD) and is a competitive inhibitor of binding to the RBD of the following reference antibody:
the reference human antibody Cv2.1169 comprising (i) a heavy chain variable region comprising the amino acid sequence of SEQ ID NO: 3 and (ii) a light chain variable region comprising the amino acid sequence of SEQ ID NO: 4;
   said human neutralizing antibody, or antigen-binding fragment thereof, comprising:
a heavy chain variable domain comprising: a heavy chain CDR1 of SEQ ID NO: 23, a heavy chain CDR2 of SEQ ID NO: 24, and a heavy chain CDR3 of SEQ ID NO: 25, and a light chain variable domain comprising: a light chain CDR1 of SEQ ID NO: 26, a light chain CDR2 of SEQ ID NO: 27 and a light chain CDR3 of SEQ ID NO: 2.

In some particular embodiments, the invention relates to a human neutralizing monoclonal antibody against Severe Acute Respiratory Syndrome-related Coronavirus 2 (SARS-CoV-2), or antigen-binding fragment thereof, which specifically binds to the viral Spike protein receptor-binding domain (RBD) and is a competitive inhibitor of binding to the RBD of the following reference antibody:
the reference human antibody Cv2.3194 comprising (i) a heavy chain variable region comprising the amino acid sequence of SEQ ID NO: 7 and a light chain variable region comprising the amino acid sequence of SEQ ID NO: 8;
   said human neutralizing antibody, or antigen-binding fragment thereof, comprising:
a heavy chain variable domain comprising: a heavy chain CDR1 of SEQ ID NO: 35, a heavy chain CDR2 of SEQ ID NO: 36 and a heavy chain CDR3 of SEQ ID NO: 37, and a light chain variable domain comprising: a light chain CDR1 of SEQ ID NO: 38, a light chain CDR2 of SEQ ID NO: 39, and a light chain CDR3 of SEQ ID NO: 40.

### Definitions

As used herein, the term “SARS-CoV-2 Spike (S) protein or glycoprotein” has its general meaning in the art and refers to a trimeric class I viral fusion protein (S trimer or tri-S) having the canonical sequence reported under accession number UniProtK PODTC2 or SEQ ID NO: 1. Based on structure predictions, signal peptide (SP) is from positions 1 to 12; ectodomain (extracellular domain) from positions 13 to 1213; transmembrane domain I from positions 1214 to 1234 and cytoplasmic domain from positions 1235 to 1273 of SEQ ID NO: 1. S1 sub-unit is from positions 13 to 685, receptor-binding domain (RBD or RBD domain) from positions 319 to 541 and S2 sub-unit from positions 686 to 1273. However, the positions of the domains or sub-units may vary slightly (+1 to +15 and −1 to −15) relative to the indicated positions. For example, the signal peptide may be from positions 1 to 15, the ectodomain from positions 13 to 1208, S1 protein from positions 16 to 681, and RBD from positions 331 to 530 of the reference sequence SEQ ID NO: 1. The RBD domain which is recognized by anti-SARS-CoV-2 antibodies according to the present disclosure may typically be SEQ ID NO: 2.

As used herein, SARS-CoV-2 refers to SARS-CoV-2 isolate Wuhan-Hu-1 and any isolate, strain, lineage, sublineage or variant thereof that is neutralized by the antibodies according to the invention. SARS-CoV-2 isolate Wuhan-Hu-1, which is used as SARS-CoV-2 reference is also referred to as BetaCoV_Wuhan_WIV04_2019 (EPI_ISL_402124) or BetaCoV_Wuhan_IVDC-HB-05_2019 EPI_ISL_402121. Non-limiting examples of SARS-CoV-2 variant or lineage which may be neutralized by the antibodies according to the present invention include SARS-CoV-2 variant comprising one or more mutations in the RBD selected from the group consisting of: K417N, K417T, N440K, L452R, G446S, S477N, T478K, E484A, E484K, E484Q, Q493R, G496S, Q498R and N501Y substitutions; for example, N501Y, E484K, K417T, and K417N. The variant may comprise other mutations in the RBD, the Spike protein or any other viral proteins, which may not prevent neutralization by the antibodies according to the present disclosure.

The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies.

The term “antibody”, as used herein and unless stated otherwise, may thus encompass whole antibody molecules, but also antigen-binding fragments thereof.

In natural antibodies of rodents and primates, two heavy chains are linked to each other by disulfide bonds, and each heavy chain is linked to a light chain by a disulfide bond. There are two types of light chains, lambda (λ) and kappa (κ). There are five main heavy chain classes (or isotypes) which determine the functional activity of an antibody molecule: IgM, IgD, IgG, IgA and IgE. In humans there are four subclasses of IgG: IgG1, IgG2, IgG3 and IgG4 (numbered in order of decreasing concentration in serum). IgA exists in two subclasses, IgA1 and IgA2. Both IgA1 and IgA2 have been found in external secretions (secretory IgA), where IgA2 is more prominent than in the blood (serum IgA). Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). Secretory IgA are polymeric: 2-4 IgA monomers are linked by two additional chains: the immunoglobulin joining (J) chain(s) and secretory component (SC). The J chain binds covalently to two IgA molecules through disulfide bonds between cysteine residues. The secretory component is a proteolytic cleavage product of the extracellular part of the polymeric immunoglobulin receptor (pIgR) which binds to J-chain containing polymeric Ig. Polymeric IgA (mainly the secretory dimer) is produced by plasma cells in the lamina propria adjacent to mucosal surfaces. It binds to the polymeric immunoglobulin receptor on the basolateral surface of epithelial cells, and is taken up into the cell via endocytosis. The receptor-IgA complex passes through the cellular compartments before being secreted on the luminal surface of the epithelial cells, still attached to the receptor. Proteolysis of the receptor occurs, and the dimeric IgA molecule, along with a portion of the receptor known as the secretory component—known as sIgA, are free to diffuse throughout the lumen.

The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) can participate in the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. The predicted CDRs of some anti-SARS-CoV-2 antibodies, such as Cv2.1169, Cv2.5213, Cv2.3235, Cv2.1353 and Cv2.3194 are described herein.

The term “monoclonal antibody” as used herein refers to a preparation of antibody molecules of single specificity. A monoclonal antibody displays a single binding specificity and affinity for a particular epitope. Accordingly, the term “human monoclonal antibody” refers to an antibody displaying a single binding specificity which has variable and constant regions derived from or based on human germline immunoglobulin sequences or derived from completely synthetic sequences. The method of preparing the monoclonal antibody is not relevant for the binding specificity.

As used herein, the term “recombinant antibody” refers to antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies. In some embodiments a recombinant human antibody of this invention has the same amino acid sequence as a naturally-occurring human antibody but differs structurally from the naturally occurring human antibody. For example, in some embodiments the glycosylation pattern is different as a result of the recombinant production of the recombinant human antibody. In some embodiments the recombinant human antibody is chemically modified by addition or subtraction of at least one covalent chemical bond relative to the structure of the human antibody that occurs naturally in humans.

As used herein, the term “non-native human glycosylation pattern” refers to a glycosylation pattern (i.e. of an antibody according to the present disclosure) which is characterized in that it is produced in human cells (i.e. in vitro production; for example in vitro production in HEK cells), and which may or may not correspond to the native glycosylation pattern of a reference human antibody.

As used herein, the term “non-human glycosylation pattern” refers to a glycosylation pattern which is characterized in that it is produced in non-human cells (i.e. in vitro production in CHO cells).

The term “antigen-binding fragment” of an antibody (or simply “antibody fragment”), as used herein, refers to full length or one or more fragments of an antibody that retain the ability to specifically bind to an antigen (e.g., Spike glycoprotein of SARS-CoV-2, preferably RBD domain). It has been shown that the antigen-binding function of an antibody can be performed by fragments of a full-length antibody. Examples of binding fragments encompassed within the term “antigen-binding fragment” of an antibody include a Fab fragment, a monovalent fragment consisting of the VL, VH, CL and CH1 domains; a F(ab′)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989 Nature 341:544-546), which consists of a VH domain, or any fusion proteins comprising such antigen-binding fragments. Furthermore, although the two domains of the Fv fragment, VL and VH, are coded for by separate genes, they can be joined, using recombinant methods, by a synthetic linker that enables them to be made as a single chain protein in which the VL and VH regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding fragment” of an antibody. These antibody fragments are obtained using conventional techniques known to those of skill in the art, and the fragments are screened for utility in the same manner as are intact antibodies.

The expression “variable domain” or “variable region” of an antibody heavy or light chain are used interchangeably as the variable region of an antibody consists of a variable domain.

The phrases “an antibody recognizing an antigen (X)”, “an antibody having specificity for an antigen (X)”, “an anti-X antibody”, “an antibody against X”, and an “antibody directed against” are used interchangeably herein with the term “an antibody which binds specifically to an antigen (X)”.

As used herein, “antibody” or “nucleic acid” refers to an isolated antibody or nucleic acid.

As used herein a “class 2 anti-SARS-CoV2 Spike protein antibody” refers to a neutralizing antibody which may specifically bind to, both, the viral Spike protein receptor-binding domain (RBD) in the ‘up’ conformation and the viral Spike protein receptor-binding domain (RBD) in the ‘down’ conformation.

The “up” conformation of the RBD corresponds to the RBD conformation exposing the receptor-binding site to the peptidase domain (PD) of the angiotensin-converting enzyme 2 (ACE2). The “down” conformation of the RBD corresponds to the closed RBD conformation for which the ACE2 receptor-binding site is not accessible.

As used herein a “class 3 anti-SARS-CoV2 Spike protein antibody” refers to a neutralizing antibody which binds outside the ACE2-binding site of the RBD. The classification into class 2 or class 3 anti-SARS-CoV2 Spike protein antibodies corresponds to the Barnes classification which is developed in Barnes et al. (“SARS-CoV-2 neutralizing antibody structures inform therapeutic strategies”; Nature; 588, 682-687 (2020)).

The present disclosure encompasses the therapeutic use of both the antibody or antigen-binding fragment according to the present disclosure (antibody therapy) and a nucleic acid or vector encoding said antibody or antigen-binding fragment, in particular mRNA such as modified mRNA (nucleic acid therapy).

The term “Kassoc” or “Ka”, as used herein, is intended to refer to the association rate of a particular antibody-antigen interaction, whereas the term “Kdis” or “Kd,” as used herein, is intended to refer to the dissociation rate of a particular antibody-antigen interaction.

The term “KD”, as used herein, is intended to refer to the dissociation constant, which is obtained from the ratio of Kd to Ka (i.e. Kd/Ka) and is expressed as a molar concentration (M). KD values for antibodies are determined by Surface plasmon resonance using Biacore® system (Biacore assay).

As used herein, the term “specificity” refers to the ability of an antibody to detectably bind an epitope presented on an antigen, such as the SARS-CoV-2 Spike glycoprotein (S trimer or tri-S) which is a trimeric class I viral fusion protein, particularly the S1 subunit of S protein monomer, more particularly the SARS-CoV-2 Spike receptor-binding domain (RBD or S-RBD) while not detectably binding to (i.e., cross-reacting with) other epitopes. The specific binding of the antibody of the present disclosure to the SARS-CoV-2 Spike receptor-binding domain (RBD or S-RBD) refers to its binding to at least one of a SARS-CoV-2 Spike (S trimer or tri-S) protein, S1 subunit protein and S-RBD protein, in particular chosen from SARS-CoV-2 tri-S(SEQ ID NO: 106), S1 sub-unit (SEQ ID NO: 107), S-RBD (SEQ ID NO: 108) proteins. As the RBD is present in the Spike and S1 proteins, the specificity of an antibody for the RBD protein also implies its specificity for the Spike and S1 proteins.

An antibody specifically binds to its target when it has a KD of 1 pM or less for its target in a Biacore assay. The target is in particular chosen from SARS-CoV-2 tri-S(SEQ ID NO: 106), S1 sub-unit (SEQ ID NO: 107) and S-RBD (SEQ ID NO: 108) (FIG. 3A). The definition is met if the criteria occurs for at least S-RBD target. Antibodies are covalently coupled to CM5 sensor chips (Biacore) using amino-coupling kit (Biacore) according to the manufacturer's procedure. Recombinant ACE2 ectodomain may also be coupled to the sensor chip in the same conditions, for comparison. All analyses are performed using HBS-EP buffer (10 mM HEPES pH 7.2; 150 mM NaCl; 3 mM EDTA, and 0.005% Tween 20). The flow rate of buffer during all real-time interaction measurements is set at 30 μl/min. All interactions are performed at temperature of 25° C. SARS CoV-2 tri-S and S1 proteins are serially diluted (two-fold step) in HBS-EP in the range of 40-0.156 nM. Same range of concentrations is used for RBD with exception of low affinity interactions where the concentration range 1280-10 nM was applied. The association and dissociation phases of the binding of viral proteins to the immobilized antibodies and ACE2 are monitored for 3 and 4 minutes, respectively. The binding of the proteins to reference channel containing carboxymethylated dextran only is used as negative control and is subtracted from the binding during data processing. The evaluation kinetic parameters of the studied interactions are performed by using BIAevaluation version 4.1.1 Software (Biacore). In these conditions, the antibodies Cv2.3235, Cv2.5213, Cv2.1353, Cv2.3194 and Cv2.1169 of the present disclosure have a KD of less than 1 pM for the three targets: tri-S(SEQ ID NO: 106), S1 sub-unit (SEQ ID NO: 107) and S-RBD (SEQ ID NO: 108) as shown in FIG. 3A. Their KD for the S-RBD target is lower than that of the ACE2 ectodomain (FIG. 3A).

Specificity can further be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a SARS-CoV-2 Spike glycoprotein (tri-S), S1 sub-unit or S-RBD, particularly chosen from SARS-CoV-2 tri-S(SEQ ID NO: 106), S1 sub-unit (SEQ ID NO: 107) and S-RBD (SEQ ID NO: 108) proteins. Specificity demonstrated experimentally for at least the S-RBD protein and one non-specific antigen means that the antibody is specific to the antigen. The term “affinity”, as used herein, means the strength of the binding of an antibody to an epitope.

As used herein, the term “Avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

The antibodies according to the invention compete with each other for binding to SARS-CoV-2 Spike protein (S trimer or tri-S) and S-RBD protein in a competition ELISA binding assay. “Cross-compete with” is used herein interchangeably with “competitively inhibit the binding of”, or “is a competitive inhibitor of”. For competition ELISAs, ELISA plates are coated with 250 ng/well of StrepTag-free SARS-CoV-2 tri-S and S-RBD proteins (SEQ ID NO: 106, 108 with deletion of C-term StrepTag sequence WSHPQFEK (SEQ IN NO: 121) and incubated with biotinylated antibodies (at a concentration of 100 ng/ml for tri-S competition and 25 ng/ml for RBD competition) in 1:2 serially diluted solutions of antibody competitors in PBS (IgG concentration ranging from 0.39 to 50 μg/ml). Antibody incubation step is for 2 h.

For all ELISA assays, the coating step is performed overnight in PBS buffer. Washings with 0.05% Tween 20-PBS buffer are performed between each step. A blocking step of 2 h with 2% BSA, 1 mM EDTA, 0.05% Tween 20-PBS (Blocking buffer) is performed after the coating step. Antibody dilution and incubation are performed in PBS. Optical densities are measured at appropriate OD and background values given by incubation of PBS alone in coated are subtracted. OD>0.5 (cut-off value) are considered as positive.

FIGS. 3B and 4D show that the antibodies Cv2.3235, Cv2.5213, Cv2.1353, Cv2.3194 and Cv2.1169 of the present disclosure competitively inhibit the binding of each other to SARS-CoV-2 tri-S and S-RBD proteins. There is no competitive inhibition with anti-S control antibody which binds to an epitope outside the RBD (Cv2.2396) (FIG. 4D). The percentage of competitive inhibition of the antibodies may be determined by measuring the area under curve (AUC) (FIG. 3B). The antibodies according to the present disclosure inhibit at least 30%, preferably 50% or more (60%; 70%; 80%; 90%) of binding of at least one of the reference antibodies Cv2.3235, Cv2.5213, Cv2.1353, Cv2.3194 and Cv2.1169 to SARS-CoV-2 tri-S and/or S-RBD protein in the competition ELISA binding assay according to the present disclosure.

The present disclosure encompasses anti-SARS-CoV-2 antibodies which inhibit at least 30% of binding to SARS-CoV-2 Spike and/or S-RBD protein of at least one of the reference antibodies Cv2.1169, Cv2.5213, Cv2.3235, Cv2.1353 and Cv2.3194 in the competition ELISA binding assay according to the present disclosure.

Test antibodies may first be screened for their binding affinity to SARS-CoV-2 Spike (S trimer or tri-S), S1 sub-unit and S-RBD in a direct ELISA binding assay. ELISA plates are coated with 250 ng/well of purified recombinant SARS-CoV-2 tri-S(SEQ ID NO: 106), S1 (SEQ ID NO: 107), and S-RBD (SEQ ID NO: 108) and incubated with recombinant monoclonal IgG1 or IgA antibodies at 4 or 10 μg/ml, and 4 to 7 consecutive 1:4 dilutions in PBS, Antibody incubation step is for 2 h. Coating, washings, revelation and buffers are as disclosed above. An OD value>0.5 in ELISA binding assay to SARS-CoV-2 Spike (S trimer or tri-S), S1 sub-unit and S-RBD (SEQ ID NO: 106 to 108) according to the present disclosure indicates the presence of binding affinity (Table 5).

The ability of a test antibody to cross-compete with, or competitively inhibit the binding of antibodies of the present disclosure to SARS-CoV-2 Spike (S) (S trimer or tri-S) and S-RBD proteins in competitive ELISA assay as disclosed above, demonstrates that the test antibody can compete with that antibody for binding to SARS-CoV-2 Spike (S) (S trimer or tri-S) and RBD proteins; such an antibody may, according to non-limiting theory, bind to the same or a related (e.g., a structurally similar or spatially proximal) epitope on SARS-CoV-2 Spike receptor-binding domain as the antibody with which it competes.

The antibody according to the invention inhibits the binding of SARS-CoV-2 Spike (S trimer or tri-S) and/or S-RBD proteins to angiotensin-converting enzyme 2 (ACE2) in a competition ELISA binding assay using a biotinylated SARS-CoV-2 tri-S(SEQ ID NO: 106) or S-RBD (SEQ ID NO: 108) protein and ACE2 ectodomain protein (SEQ ID NO: 103). Plates are coated with purified ACE2 ectodomain protein (250 ng/well) and incubated for 2h with recombinant monoclonal antibody at 2 μg/ml and consecutive dilutions (1:2) in presence of biotinylated tri-S protein at 1 μg/ml, or recombinant monoclonal IgG1 antibodies at 10 or 100 μg/ml and consecutive dilutions (1:2) in presence of biotinylated RBD at 0.5 μg/ml. Antigen-antibody complexes are detected using streptavidin conjugate, such as streptavidin-HRP and appropriate chromogenic substrate.

The inhibitory activity of the antibody is expressed as the half maximal effective concentration (EC₅₀), e.g., the concentration which inhibits 50% of tri-S or S-RBD protein binding to ACE2-ectodomain protein. EC₅₀ values (pg/ml) are calculated based on a reconstructed curve of the percentage of inhibition at the various concentrations indicated, as shown in the present examples (see FIGS. 3C and 3L). Alternatively, it may be expressed as the percentage of inhibition (or blocking) of SARS-CoV-2 tri-S or S-RBD protein binding to ACE2-ectodomain protein by measuring the area under curve (AUC) (Table 5). A competitive inhibitor of binding of SARS-CoV-2 S-RBD or tri-S protein to ACE2 ectodomain has an EC₅₀ of less than 5 μg/mL and/or inhibits at least 70% of binding of SARS-CoV-2 S-RBD or tri-S protein to ACE2 ectodomain in competitive ELISA binding assay.

The ability of the antibodies according to the present disclosure to bind to the RBD of SARS-CoV-2 variants including in particular P.1, B.1.1.7 and B.1.351 and block RBD binding to ACE2-ectodomain is assayed in the direct and competition ELISA binding assay according to the present disclosure, using S-RBD protein of SARS-CoV-2 variant P.1, B.1.1.7 and B.1.351 (SEQ ID NO: 109 to 111). RBD binding and blocking of RBD binding to ACE2 ectodomain of the Cv2.1169, Cv2.5213, Cv2.3235, Cv2.1353 and Cv2.3194 are shown in FIGS. 3F, 4E to 4J.

Alternatively, the ability of the antibodies according to the present disclosure to bind to the RBD of SARS-CoV-2 variants including in particular B.1.617 and B.1.1.529 and block RBD binding to ACE2-ectodomain is assayed in the direct and competition ELISA binding assay according to the present disclosure, using S-RBD protein of SARS-CoV-2 variant B.1.617 and B.1.1.529 (SEQ ID NO: 122 to 125).

The antibodies according to the invention do not cross-react with other coronavirus including other human pathogenic betacoronavirus SARS-CoV-1 and MERS-CoV), alphacoronavirus 229E-CoV and NL63-CoV and betacoronavirus group A HKU1-CoV in ELISA binding assay using coronavirus Spike ectodomain (tri-S) protein (SEQ ID NO: 115 to 120). ELISA plates are coated with 250 ng/well of purified recombinant coronavirus tri-S proteins comprising a foldon trimerization motif and C-terminal tags (8×HisTag, StrepTag, and AviTag) and incubated with recombinant monoclonal IgG1 or IgA antibodies at 4 or 10 μg/ml, and 4 to 7 consecutive 1:4 dilutions in PBS, Antibody incubation step is for 2 h. Coating, washings, revelation and buffers are as disclosed above. An OD value<0.5 in ELISA binding assay to SARS-CoV-1, MERS-CoV, 229E-CoV, NL63-CoV, HKU1-CoV or OC43-CoV Spike protein (SEQ ID NO: 115 to 120) according to the present disclosure indicates no cross-reactivity (Table 5).

The absence of polyreactivity of the antibodies according to the present disclosure is determined in ELISA binding assay. ELISA plates are coated with 500 ng/well of purified double stranded (ds)-DNA, KLH, LPS, Lysozyme, Thyroglobulin, Peptidoglycan from B. subtilis, 250 ng/well of insulin, flagellin from B. subtilis, MAPK14 (9), and 125 ng/well of YU2 HIV-1 Env gp140 protein in PBS. After blocking and washing steps, recombinant monoclonal IgG antibodies are tested at 4 μg/ml and 7 consecutive 1:4 dilutions in PBS. Control antibodies, mGO53 (negative) (3), and ED38 (high positive) (4) are included in each experiment. ELISA binding is developed as described above. OD>0.5 (cut-off value) are considered as positive. Results obtained with antibodies Cv2.1169, Cv2.5213, Cv2.3235, Cv2.1353 and Cv2.3194 according to the present disclosure are illustrated in FIG. 5 (A, B, D and F).

The absence of reactivity to human proteins of the antibodies according to the present disclosure is determined in Protein microarray binding assay. Experiments are performed at 4° C. using ProtoArray Human Protein Microarrays (Thermo Fisher Scientific). Microarrays are blocked for 1 h in blocking solution (Thermo Fisher), washed and incubated for 1h30 with IgG antibodies at 2.5 μg/ml as previously described (9). After washings, arrays are incubated for 1h30 with AF647-conjugated goat anti-human IgG antibodies (at 1 μg/ml in PBS; Thermo Fisher Scientific), and revealed using GenePix 4000B microarray scanner (Molecular Devices) and GenePix Pro 6.0 software (Molecular Devices) as previously described (9). Fluorescence intensities are quantified using Spotxel® software (SICASYS Software GmbH, Germany), and mean fluorescence intensity (MFI) signals for each antibody (from duplicate protein spots) are plotted against the reference antibody mGO53 (non-polyreactive isotype control) using GraphPad Prism software (v8.1.2, GraphPad Prism Inc.). For each antibody, Z-scores are calculated using ProtoArray® Prospector software (v5.2.3, Thermo Fisher Scientific), and deviation (σ) to the diagonal, and polyreactivity index (PI) values are calculated as previously described (9). Antibodies were defined as polyreactive when PI>0.21.

The absence of self-reactivity of the antibodies according to the present disclosure is determined in indirect immuno-fluorescence assay (IFA) on HEp-2 cells. Recombinant SARS-CoV-2 S-specific and control IgG antibodies (mGO53 and ED38) at 100 μg/ml are testedin indirect immuno-fluorescence assay (IFA) on HEp-2 cells sections (AnA HEp-2 AeskuSlides®, Aesku.Diagnostics, Wendelsheim, Germany) using the kit's controls and FTTC-conjugated anti-human TgG antibodies as the tracer according to the manufacturer' instructions. HEp-2 sections are examined using fluorescence microscope and pictures are taken at magnification×40. Results obtained with antibodies Cv2.1169, Cv2.5213, Cv2.3235, Cv2.1353 and Cv2.3194 according to the present disclosure are illustrated in FIG. 5C.

As used herein, the term “neutralizing antibody” refers to an antibody that inhibits virus infection, in particular that inhibits or blocks virus entry into host cells by competing with SARS-CoV-2 Spike (S) protein for binding to angiotensin-converting enzyme 2 ACE2 receptor on host cells and blocking RBD interaction with ACE2 through binding to the RBD. The neutralizing activity of the antibody is measured by SARS-CoV-2 S-Fuse Assay. SARS-CoV-2 virus (Multiplicity of infection (MOI) of 0.1) is incubated with recombinant monoclonal IgA or IgG antibodies at 10 μg/ml or 5 μg/ml, and consecutive 1:4 dilutions in culture medium for 30 min at room temperature and added to S-Fuse cell culture (U2OS-ACE2 GFP1-10 and U20S-ACE2 GFP 11; ratio 1:1; 8×10³ per well). After 18h of incubation, cells are fixed and nuclei stained. The area displaying GFP expression and the number of nuclei are quantified by confocal microscopy. The percentage neutralization is calculated from the GFP-positive area as follows: 100×(1−(value with IgA/IgG−value in “non-infected”)/(value in “no IgA/IgG”−value in “non-infected”)) (Table 5). The neutralizing activity of each isotype is expressed as the half maximal effective concentration (EC₅₀). EC₅₀ values (ng/ml) are calculated based on a reconstructed curve of the percentage neutralization at the various concentrations indicated (see FIGS. 3D, 3G, and 3L). A neutralizing antibody has an EC₅₀ of less than 1000 ng/mL and/or neutralizes at least 90% of SARS-CoV-2 in SARS-CoV-2 S-Fuse Assay.

As used herein, the term “ADCC” or “antibody dependent cell cytotoxicity” and “CDC” or “Complement-dependent-cytotoxicity” activity refers to cell depleting activity. ADCC and CDC activities can be measured by standard methods that are well-known in the art and disclosed in the examples The ADCC activity of the antibody of the present disclosure is quantified using the ADCC Reporter Bioassay (Promega). Raji-Spike cells (5×10⁴) are co-cultured with Jurkat-CD16-NFAT-rLuc cells (5×10⁴) in presence or absence of SARS-CoV2 S-specific or control mGO53 IgG antibody (3) at 10 μg/ml or 50 μg/ml and 10 consecutive 1:2 dilutions in PBS. Luciferase activity is measured after 18 h of incubation. ADCC is measured as the fold induction of Luciferase activity compared to the control antibody. A low ADCC activity is less than 2-fold higher compared to control antibody. The CDC activity of the antibody of the present disclosure is quantified using SARS-CoV-2 Spike-expressing Raji cells as previously described (10). Raji-Spike cells (5×10⁴) are cultivated in the presence of 5000 normal (NHIS) or heat-inactivated (HIHS) human serum and with or without recombinant IgG antibodies (at 10 pig/ml or 50 pig/ml and 10 consecutive 1:2 dilutions in PBS). After 24h, cells are washed with PBS and the live/dead fixable aqua dead cell marker (1:1,000 in PBS; Life Technologies) is added for 30 min at 4° C. before fixation. Cells are analysed by fluorescence microscopy. CDC is calculated using the following formula: 100×(% of dead cells with serum−% of dead cells without serum)/(100−% of dead cells without serum). A low CDC activity is less than 3%.

As used herein “preventing” SARS-CoV-2 infection and/or associated disease means reducing the risk of SARS-CoV-2 infection and/or associated disease.

### Neutralizing Antibodies

Neutralizing antibodies or antigen-binding fragments thereof according to the present disclosure include the selected recombinant anti-SARS-CoV-2 antibodies Cv2.3235, Cv2.5213, Cv2.1169, Cv2.1353 and Cv2.3194 and Cv2.5179 which are structurally characterized by their heavy chain variable domain and light chain variable domain amino acid sequences as described in Table 1 below:

TABLE 1
Variable heavy and light chain amino acid sequences of Cv2.1169,
Cv2.1353, Cv2.3194, Cv2.3235 and Cv2.5213 and Cv2.5179.

| | | |
|---|---|---|
| Cv2.1169 | VH | QVQLVQSGPEVKKPGTSVKVSCKASGFTFTTSAVQWVRQA |
| | (SEQ ID NO: 3) | RGQRLEWIGWIVVGSGNTNYAQKFQERVTITRDMSTTTAY |
| | | MELSSLRSEDTAVYYCAAPYCSGGTCLDGFDIWGQGTMVT |
| | | VSS |
| | VL | EIVLTQSPGTLSLSPGERATLSCRASQSVSRSYLAWYQQKPG |
| | (SEQ ID NO: 4) | QAPRLLIYSASSRATGIPDRESGSGSGTDFTLTISRLEPEDFAV |
| | | YYCQQYGSSPWTFGQGTKVEIK |
| Cv2.1353 | VH | EVQLVESGGGLVQPGGSLRLSCAASGVTVSSNYMNWVRQA |
| | (SEQ ID NO: 5) | PGKGLEWVSVIYSGGSTFYADSVKGRFIISRDNSKNTLYLQ |
| | | MNSLKTEDTAVYYCARDLGPMGFDIWGQGTMVTVSS |
| | VL | DIQMTQSPSFLSASVGDRVTITCRASQGISSYLAWYQQKPGK |
| | (SEQ ID NO: 6) | APKLLIYAASTLQSGVPSRFSGSGSGTEFTLTISSLQPEDFAT |
| | | YYCQQLNSDPPVTFGPGTKVDIK |
| Cv2.3194 | VH | EVQLVESGGGLIQPGGSLRLSCAASGITVTSNYMSWVRQAP |
| | (SEQ ID NO: 7) | GKGLEWVSVIYPGGSTFYADSVKGRFTISRDNSKNTLYLQM |
| | | NSLRAEDTAVYYCARDLVVYGMDVWGQGTTVTVSS |
| | VL | EIVLTQSPGTLSLSPGERATLSCRASQSVSSSYLAWYQQKPG |
| | (SEQ ID NO: 8) | QAPRLLIYGASSRATGIPGRFSGSGSGTDFTLTISRLEPEDFAI |
| | | YYCQQGVTFGGGTKVEIK |
| Cv2.3235 | VH | EVQLVESGGGLIQPGGSLRLSCAASGFTVSRNYMSWVRQAP |
| | (SEQ ID NO: 9) | GKGLEWVSVIYSGGSTFYADSVKGRFTISRDNSKNTLYLQM |
| | | NSLRAEDTAVYYCARDGDYYGMDVWGQGTTVTVSS |
| | VL | DIQMTQSPSFLSASVGDRVTITCRASQGISSFLAWYQQKPGK |
| | (SEQ ID NO: 10) | APKLLIYGASTLQSGVTSRFSGSGSGTEFTLTISSLQPEDFAT |
| | | YYCQRLDSYPPITFGQGTRLEIK |
| Cv2.5213 | VH | EVQLVESGGGLIQPGGSLRLSCAASGFTVSSNYMSWVRQAP |
| | (SEQ ID NO: 11) | GKGLEWVSLIYSGGSTYYADSVKGRFTISRDNSKNTLYLQM |
| | | NSLRAEDTAVYYCARDLNYYGMDVWGQGATVTVSS |
| | VL | DIQMTQSPSSLSASVGDRVTITCRASQGISSYLAWYQQKPGK |
| | (SEQ ID NO: 12) | APKLLIYAASTLQSGVPSRFSGSGSGTDFTLTISSLQPEDFAT |
| | | YYCQQLDSYSPFTFGPGTKVDIK |
| Cv2.5179 | VH | QVQLVQSGPEVKKPGTSVKVSCKASGFTFTSSAVQWVRQA |
| | (SEQ ID NO: 152) | RGQRLEWIGWIVVGSGNTNYAQKFQERVTITRDMSTSTAY |
| | | MELSSLRSEDTAVYYCAAPHCSSTSCYDAFDIWGQGTMVT |
| | | VSS |
| | VL | EIVLTQSPGTLSLSPGERATLSCRASQSVSSSYLAWYQQKPG |
| | (SEQ ID NO: 153) | QAPRLLIYGASSRATGIPDRFSGSGSGTDFTLTISRLEAEDFA |
| | | VYYCQQYGRSPWTFGQGTKLEIK |

Neutralizing antibody or antigen-binding fragments thereof according to the present disclosure also includes human antibody which specifically binds to SARS-CoV-2-Spike protein receptor-binding domain (RBD) and is a competitive inhibitor of binding to the RBD of at least one of the following reference antibodies:
the reference human antibody Cv2.1169 comprising (i) a heavy chain variable region comprising the amino acid sequence of SEQ ID NO: 3 and (ii) a light chain variable region comprising the amino acid sequence of SEQ ID NO: 4;
the reference human antibody Cv2.3194 comprising (i) a heavy chain variable region comprising the amino acid sequence of SEQ ID NO: 7 and a light chain variable region comprising the amino acid sequence of SEQ ID NO: 8;
the reference human antibody Cv2.1353 comprising (i) a heavy chain variable region comprising the amino acid sequence of SEQ ID NO: 5 and a light chain variable region comprising the amino acid sequence of SEQ ID NO: 6;
the reference human antibody Cv2.5213 comprising (i) a heavy chain variable region comprising the amino acid sequence of SEQ ID NO: 11 and a light chain variable region comprising the amino acid sequence of SEQ ID NO: 12; and
the reference human antibody Cv2.3235 comprising (i) a heavy chain variable region comprising the amino acid sequence of SEQ ID NO: 9 and a light chain variable region comprising the amino acid sequence of SEQ ID NO: 10; and
the reference human antibody Cv2.5179 comprising (i) a heavy chain variable region comprising the amino acid sequence of SEQ ID NO: 152 and (ii) a light chain variable region comprising the amino acid sequence of SEQ ID NO: 153.

According to particular embodiments, a neutralizing antibody or antigen-binding fragments thereof according to the present disclosure includes human antibody which specifically binds to SARS-CoV-2-Spike protein receptor-binding domain (RBD) and is a competitive inhibitor of binding to the RBD of at least one of the following reference antibodies:
the reference human antibody Cv2.1169 comprises (i) a heavy chain comprising the amino acid sequence of SEQ ID NO: 133 and (ii) a light chain comprising the amino acid sequence of SEQ ID NO: 14; or
the reference human antibody Cv2.3194 comprises (i) a heavy chain comprising the amino acid sequence of SEQ ID NO: 135 and a light chain comprising the amino acid sequence of SEQ ID NO: 18.

Competitive inhibition with a reference antibody is assayed in a competition ELISA binding assay according to the present disclosure (see definitions). The present disclosure encompasses anti-SARS-CoV-2 antibodies which inhibit at least 30% of binding to SARS-CoV-2 Spike and/or S-RBD protein to at least one of the reference antibodies Cv2.1169, Cv2.5213, Cv2.3235, Cv2.1353, Cv2.3194 and Cv2.5179 in the competition ELISA binding assay according to the present disclosure. The reference antibody Cv2.1169, Cv2.5213, Cv2.3235, Cv2.1353, Cv2.3194 or Cv2.5179 comprises the above disclosed heavy and light chain variable region amino acid sequences as presented in Table 1. The reference antibody is preferably an IgA or IgG1. The IgG1 reference antibody preferably comprises the full-length heavy and light chain amino acid sequences as presented in Table 2: Cv2.1169 (SEQ ID NO: 13-14); Cv2.1353 (SEQ ID NO: 15-16); Cv2.3194 (SEQ ID NO: 17-18); Cv2.3235 (SEQ ID NO: 19-20); Cv2.5213 (SEQ ID NO: 21-22); Cv2.5179 (SEQ ID NO: 154-155).

According to an alternative preferred embodiment, the IgG1 reference antibody preferably comprises the following full-length heavy and light chain amino acid sequences: Cv2.1169 “prime” (SEQ ID NO: 133-14); Cv2.1353 “prime” (SEQ ID NO: 134-16); Cv2.3194 “prime” (SEQ ID NO: 135-18); Cv2.3235 “prime” (SEQ ID NO: 136-20); Cv2.5213 “prime” (SEQ ID NO: 137-22).

In some preferred embodiments, the antibody is a competitive inhibitor of the reference human antibody Cv2.1169 or Cv2.1169 “prime” or Cv2.3194 or Cv2.3194 “prime”; preferably the reference human antibody Cv2.1169. Preferably, the reference antibody Cv2.1169 or Cv2.1169 “prime” or Cv2.3194 or Cv2.3194 “prime” is an IgA or IgG1; the IgG1 reference antibody Cv2.1169 or Cv2.1169 “prime” or Cv2.3194 or Cv2.3194 “prime” preferably comprises the full-length heavy and light chain amino acid sequences SEQ ID NO: 13-14, SEQ ID NO: 133-14, SEQ ID NO: 17-18 and SEQ ID NO: 135-18, respectively.

In some particular embodiments, the antibody binds to at least one recombinant SARS-CoV-2 S protein selected from a S-trimer, a S1 sub-unit, and a S-RBD domain with a KD of from 600 nM to 100 pM or less in the Biacore assay according to the present disclosure (see definitions). In some preferred embodiments, the S-trimer protein comprises or consists of the sequence SEQ ID NO: 106, the S1 sub-unit protein comprises or consists of the sequence SEQ ID NO: 107, the S-RBD protein comprises or consists of any one of SEQ ID No: 108 to 111 and 122 to 125, and/or the ACE2 ectodomain protein comprises or consists of SEQ ID NO: 103. In some preferred embodiments, it binds to recombinant SARS-CoV-2 S-trimer, preferably comprising SEQ ID NO: 106, with a KD of from 50 nM to 300 pM or less; preferably a KD of from 10 nM to 300 pM; in particular a KD selected from 5 nM, 1 nM, 500 pM, and 300 pM or less (see FIG. 3A). In some preferred embodiments, it binds to recombinant S1 sub-unit, preferably comprising SEQ ID NO: 107, with a KD of from 600 nM to 1 nM or less; preferably a KD of from 100 nM to 1 nM; more preferably a KD of from 25 nM to 1 nM; in particular a KD selected from 10 nM, 5 nM, 2.5 nM, and 1 nM or less (see FIG. 3A). In some preferred embodiments, it binds to recombinant RBD domain, preferably comprising any one of SEQ ID NO: 108 to 111, with a KD of from 500 nM to 100 pM or less; preferably a KD of from 100 nM to 100 pM; more preferably a KD of from 25 nM to 100 pM; in particular a KD selected from 10 nM, 1 nM, 500 pM, 400 pM, 300 pM, and 100 pM or less (see FIG. 3A).

In some particular embodiments, the antibody binds to at least one recombinant SARS-CoV-2 S protein selected from a S-trimer, a S1 subunit and a S-RBD, with a binding affinity which is higher than that of ACE2 ectodomain protein; preferably at least 5, 10, 25, 50, 100, 250, 500 or 1000 folds higher (which means that the KD of the antibody for S-trimer, S1 subunit, and/or S-RBD is at least 5, 10, 25, 50, 100, 250, 500 or 1000 folds lower than that of ACE2 ectodomain protein); preferably wherein the binding affinity of the antibody for the recombinant S-RBD protein is at least 10, 25, 50, 100, 250, 500 or 1000 folds higher compared to that of the recombinant ACE2 ectodomain protein; more preferably wherein the binding affinity of the recombinant S-trimer, S1 subunit and S-RBD proteins is at least 10, 25, 50, 100, 250, 500 or 1000 folds higher compared to that of the recombinant ACE2 ectodomain protein. Preferably, wherein the S-trimer protein comprises SEQ ID NO: 106, the S1 subunit protein comprises SEQ ID NO: 107, the S-RBD protein comprises any one of SEQ ID NO: 108 to 111, and/or the recombinant ACE2 ectodomain protein comprises SEQ ID NO: 103.

In some particular embodiments, the antibody competitively inhibits the binding of recombinant SARS-CoV-2 Spike protein (S trimer or tri-S) to recombinant angiotensin-converting enzyme 2 (ACE2) ectodomain protein with an EC₅₀ of from 1 μg/mL to 0.1 μg/mL or less in the competition ELISA binding assay according to the present disclosure (see definitions). In some embodiments the antibody competitively inhibits the binding of recombinant SARS-CoV-2 Spike protein (S trimer or tri-S) to recombinant angiotensin-converting enzyme 2 (ACE2) ectodomain protein with an EC₅₀ selected from 1 μg/mL or less, 0.5 μg/mL or less, 0.4 μg/mL or less, 0.3 μg/mL or less, 0.2 μg/mL or less, and 0.1 μg/mL or less as determined in the competition ELISA binding assay according to the present disclosure. (see FIGS. 3C and 3L). In some embodiments, the antibody blocks at least 70%, 80% or 90% of binding of recombinant SARS-CoV-2 tri-S and/or RBD proteins to recombinant ACE2 ectodomain protein in the competition ELISA binding assay as disclosed above (Table 5). Preferably, wherein the S-trimer protein comprises SEQ ID NO: 106, the S-RBD protein comprises any one of SEQ ID NO: 108 to 111, and/or the ectodomain protein comprises SEQ ID NO: 103.

In some of the above embodiments, the recombinant SARS-CoV-2 S-trimer, Sl, and/or RBD protein is from isolate Wuhan-Hu-1 or a variant thereof comprising mutation(s) in the RBD domain. The mutation(s) in the RBD domain are preferably substitutions, more preferably selected from one or more of N501Y, E484K, K417N and K417T mutations. In some preferred embodiments, the SARS-CoV-2 variant is chosen from B.1.1.7, P.1 and B.1.351 lineages. In some more preferred embodiments the recombinant SARS-CoV-2 S-trimer, S1, and/or RBD protein is selected from the group consisting of: SEQ ID NO: 106 to 111.

In some particular embodiments, the antibody neutralizes SARS-CoV-2 with a half maximal effective concentration (EC₅₀) of 20 ng/mL to 1 ng/mL and/or neutralizes at least 90% of SARS-CoV-2 in the SARS-CoV-2 S-Fuse Assay according to the present disclosure (see definitions). In some preferred embodiments, the antibody neutralizes SARS-CoV-2 with a half maximal effective concentration (EC₅₀) selected from 20 ng/mL or less, 15 ng/mL or less, 10 ng/mL or less, 5 ng/mL or less, and 1 ng/mL or less.

In some particular embodiments, the antibody neutralizes at least one SARS-CoV-2 chosen from: isolate Wuhan-Hu-1, SARS-CoV-2 variant D614G, and a SARS-CoV-2 variant comprising mutation(s) in the RBD domain. The mutation(s) in the RBD domain are preferably substitutions, more preferably selected from one or more of N501Y, E484K, K417N and K417T substitutions. In some preferred embodiments, the SARS-CoV-2 variant is chosen from B.1.1.7, P.1 and B.1.351 lineages. In some preferred embodiments, the antibody neutralizes SARS-CoV-2 isolate Wuhan-Hu-1 and at least one SARS-CoV-2 variant chosen from B.1.1.7, P.1 and B.1.351 lineages; preferably the antibody neutralizes SARS-CoV-2 isolate Wuhan-Hu-1 and SARS-CoV-2 variant lineages B.1.1.7, P.1 and B.1.351.

In some particular embodiments, the antibody does not cross-react with other coronavirus in the ELISA binding assay according to the present disclosure (see definitions). In some preferred embodiments, the antibody does not react with one or more coronavirus selected from the group consisting of: human pathogenic betacoronavirus (group B/C) SARS-CoV-1 and MERS-CoV; alphacoronavirus NL63-CoV and 229E-CoV; and betacoronavirus group A HKU1-CoV (Spike protein of SEQ ID NO: 115 to 120). In some preferred embodiments, the antibody does not cross-react with SARS-CoV-1, MERS-CoV, NL63-CoV, OC43-CoV, HKU1-CoV and 229E-CoV.

In some particular embodiments, the antibody has no polyreactivity in the ELISA binding assay according to the present disclosure, as compared to a control antibody (see definitions). In some particular embodiments, the antibody has no self-reactivity in the indirect immuno-fluorescence assay (IFA) on HEp-2 cells according to the present disclosure, as compared to a control antibody (see definitions). In some particular embodiments, the antibody has no predicted reactivity to human proteins in the Protein microarray binding assay according to the present disclosure (see definitions).

In some particular embodiments, the antibody has low levels of CDC activity (e.g., less than 3%) in the CDC assay on SARS-CoV-2 Spike-expressing Raji cells according to the present disclosure (see definitions). In some particular embodiments, the antibody has low levels of ADCC activity (e.g., less than 2-fold higher compared to control antibody) in the ADCC Reporter Bioassay according to the present disclosure (see definitions).

In some particular embodiments, the antibody or antigen-binding fragment thereof according to the present disclosure comprises a heavy chain variable region comprising an amino acid sequence selected from the group consisting of: SEQ ID NO: 3, 5, 7, 9, 11 and 152, for example SEQ NO: 3 and 7, preferably SEQ ID NO: 3.

In some particular embodiments, the antibody or antigen-binding fragment thereof according to the present disclosure comprises:
a) a heavy chain variable region comprising an amino acid sequence having at least 90% identity with SEQ ID NO: 3, and a light chain variable region comprising an amino acid sequence having at least 90% identity with SEQ ID NO: 4;
b) a heavy chain variable region comprising an amino acid sequence having at least 90% identity with SEQ ID NO: 5, and a light chain variable region comprising an amino acid sequence having at least 90% identity with SEQ ID NO: 6;
c) a heavy chain variable region comprising an amino acid sequence having at least 90% identity with SEQ ID NO: 7, and a light chain variable region comprising an amino acid sequence having at least 90% identity with SEQ ID NO: 8;
d) a heavy chain variable region comprising an amino acid sequence having at least 90% identity with SEQ ID NO: 9, and a light chain variable region comprising an amino acid sequence having at least 90% identity with SEQ ID NO: 10; or
e) a heavy chain variable region comprising an amino acid sequence having at least 90% identity with SEQ ID NO: 11, and a light chain variable region comprising an amino acid sequence having at least 90% identity with SEQ ID NO: 12; or
f) a heavy chain variable region comprising an amino acid sequence having at least 90% identity with SEQ ID NO: 152, and a light chain variable region comprising an amino acid sequence having at least 90% identity with SEQ ID NO: 153.

In some particular embodiments, the heavy and light chain variable region in any one of a) to f), such as in any one of a) to e), comprise an amino acid sequence having at least 95%, 96%, 97%, 98%, 99%, or 100% identity with the above disclosed sequences.

Anti-SARS-CoV-2 antibodies or antigen-binding fragments thereof with amino acid sequences having at least 90%, for example, at least 95%, 96%, 97%, 98%, or 99% identity to any one of the above defined amino acid sequences are part of the present disclosure, typically anti-SARS-CoV-2 antibodies or antigen-binding fragments thereof have at least equal or higher neutralizing activities than said anti-SARS-CoV-2 antibodies consisting of heavy chain SEQ ID NO:3 and light chain of SEQ ID NO:4 in the S fuse assay according to the present disclosure.

In some particular embodiments, the antibody or antigen-binding fragment thereof according to the present disclosure comprises:
a) a heavy chain variable region comprising an amino acid sequence having at least 90% identity with SEQ ID NO: 3, and a light chain variable region comprising an amino acid sequence having at least 99% identity with SEQ ID NO: 4;
b) a heavy chain variable region comprising an amino acid sequence having at least 90% identity with SEQ ID NO: 5, and a light chain variable region comprising an amino acid sequence having at least 99% identity with SEQ ID NO: 6;
c) a heavy chain variable region comprising an amino acid sequence having at least 90% identity with SEQ ID NO: 7, and a light chain variable region comprising an amino acid sequence having at least 99% identity with SEQ ID NO: 8;
d) a heavy chain variable region comprising an amino acid sequence having at least 90% identity with SEQ ID NO: 9, and a light chain variable region comprising an amino acid sequence having at least 99% identity with SEQ ID NO: 10;
e) a heavy chain variable region comprising an amino acid sequence having at least 90% identity with SEQ ID NO: 11, and a light chain variable region comprising an amino acid sequence having at least 99% identity with SEQ ID NO: 12; or
f) a heavy chain variable region comprising an amino acid sequence having at least 90% identity with SEQ ID NO: 152, and a light chain variable region comprising an amino acid sequence having at least 99% identity with SEQ ID NO: 153.

In particular, the antibody or antigen-binding fragment thereof comprises: a heavy chain variable region comprising an amino acid sequence having at least 90% identity with SEQ ID NO: 3 or SEQ ID N07, and a light chain variable region comprising an amino acid sequence having at least 90% identity with SEQ ID NO: 4 or SEQ ID NO: 8; more preferably a heavy chain variable region of SEQ ID NO: 3, and a light chain variable region of SEQ ID NO: 4

Preferably, the antibody or antigen-binding fragment thereof comprises: a heavy chain variable region comprising an amino acid sequence having at least 90% identity with SEQ ID NO: 3, and a light chain variable region comprising an amino acid sequence having at least 90% identity with SEQ ID NO: 4; more preferably a heavy chain variable region of SEQ ID NO: 3, and a light chain variable region of SEQ ID NO: 4.

Preferably, the antibody or antigen-binding fragment thereof comprises:
a) a heavy chain variable domain comprising an amino acid sequence having at least 99% identity with SEQ ID NO: 3 and a light chain variable domain comprising an amino acid sequence having at least 99% identity with SEQ ID NO: 4; or
b) a heavy chain variable domain comprising an amino acid sequence having at least 99% identity with SEQ ID NO: 7 and a light chain variable domain comprising an amino acid sequence having at least 99% identity with SEQ ID NO: 8.

As used herein, the percent identity between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences or nucleotide sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. Alternatively, the percent identity between two amino acid sequences or nucleotide sequences can be determined using the Needleman and Wunsch (J. Mol, Biol. 48:444-453, 1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

The percent identity between two nucleotide or amino acid sequences may also be determined using for example algorithms such as the BLASTN program for nucleic acid or amino acid sequences using as defaults a word length (W) of 11, an expectation (E) of 10, M=5, N=4, and a comparison of both strands.

Full length light and heavy chains amino acid sequences are shown in Table 2.

TABLE 2
Full length heavy and light chain amino acid sequences of Cv2.1169,
Cv2.1353, Cv2.3194, Cv.3235 and Cv2.5213 and Cv2.5179.
Amino acid sequences of constant isotype regions
(human IgG1) are indicated in bold.

| | | |
|---|---|---|
| Cv2.1169 | Heavy chain | QVQLVQSGPEVKKPGTSVKVSCKASGFTFTTSAVQWVRQARG |
| | (SEQ ID NO: 13) | QRLEWIGWIVVGSGNTNYAQKFQERVTITRDMSTTTAYMELSS |
| | | LRSEDTAVYYCAAPYCSGGTCLDGFDIWGQGTMVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKKVEPKSCDKTHTCPPCPAPELLGGPSVFLFPPKPK |
| | | DTLMISRTPEVTCVVVDVSHEDPEVKFNWYVDGVEVHNAK |
| | | TKPREEQYNSTYRVVSVLTVLHQDWLNGKEYKCKVSNKAL |
| | | PAPIEKTISKAKQPREPQVYTLPPSRDELTKNQVSLTCLVKG |
| | | FYPSDIAVEWESNGQPENNYKTTPPVLDSDGSFFLYSKLTVD |
| | | KSRWQQGNVFSCSVMHEALHNHYTQKSLSLSPGK |
| | Light chain | EIVLTQSPGTLSLSPGERATLSCRASQSVSRSYLAWYQQKPGQA |
| | (SEQ ID NO: 14) | PRLLIYSASSRATGIPDRFSGSGSGTDFTLTISRLEPEDFAVYYCQ |
| | | QYGSSPWTFGQGTKVEIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSFNRGEC |
| Cv2.1353 | Heavy chain | EVQLVESGGGLVQPGGSLRLSCAASGVTVSSNYMNWVRQAPG |
| | (SEQ ID NO: 15) | KGLEWVSVIYSGGSTFYADSVKGRFIISRDNSKNTLYLQMNSLK |
| | | TEDTAVYYCARDLGPMGFDIWGQGTMVTVSSASTKGPSVFPL |
| | | APSSKSTSGGTAALGCLVKDYFPEPVTVSWNSGALTSGVHTF |
| | | PAVLQSSGLYSLSSVVTVPSSSLGTQTYICNVNHKPSNTKVD |
| | | KKVEPKSCDKTHTCPPCPAPELLGGPSVFLFPPKPKDTLMIS |
| | | RTPEVTCVVVDVSHEDPEVKFNWYVDGVEVHNAKTKPREE |
| | | QYNSTYRVVSVLTVLHQDWLNGKEYKCKVSNKALPAPIEKT |
| | | ISKAKQPREPQVYTLPPSRDELTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPGK |
| | Light chain | DIQMTQSPSFLSASVGDRVTITCRASQGISSYLAWYQQKPGKAP |
| | (SEQ ID NO: 16) | KLLIYAASTLQSGVPSRFSGSGSGTEFTLTISSLQPEDFATYYCQQ |
| | | LNSDPPVTFGPGTKVDIKRTVAAPSVFIFPPSDEQLKSGTASVV |
| | | CLLNNFYPREAKVQWKVDNALQSGNSQESVTEQDSKDSTYS |
| | | LSSTLTLSKADYEKHKVYACEVTHQGLSSPVTKSFNRGEC |
| Cv2.3194 | Heavy chain | EVQLVESGGGLIQPGGSLRLSCAASGITVTSNYMSWVRQAPGK |
| | (SEQ ID NO: 17) | GLEWVSVIYPGGSTFYADSVKGRFTISRDNSKNTLYLQMNSLRA |
| | | EDTAVYYCARDLVVYGMDVWGQGTTVTVSSASTKGPSVFPL |
| | | APSSKSTSGGTAALGCLVKDYFPEPVTVSWNSGALTSGVHTF |
| | | PAVLQSSGLYSLSSVVTVPSSSLGTQTYICNVNHKPSNTKVD |
| | | KKVEPKSCDKTHTCPPCPAPELLGGPSVFLFPPKPKDTLMIS |
| | | RTPEVTCVVVDVSHEDPEVKFNWYVDGVEVHNAKTKPREE |
| | | QYNSTYRVVSVLTVLHQDWLNGKEYKCKVSNKALPAPIEKT |
| | | ISKAKQPREPQVYTLPPSRDELTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPGK |
| | Light chain | EIVLTQSPGTLSLSPGERATLSCRASQSVSSSYLAWYQQKPGQA |
| | (SEQ ID NO: 18) | PRLLIYGASSRATGIPGRFSGSGSGTDFTLTISRLEPEDFAIYYCQ |
| | | QGVTFGGGTKVEIKRTVAAPSVFIFPPSDEQLKSGTASVVCLL |
| | | NNFYPREAKVQWKVDNALQSGNSQESVTEQDSKDSTYSLSS |
| | | TLTLSKADYEKHKVYACEVTHQGLSSPVTKSFNRGEC |
| Cv2.3235 | Heavy chain | EVQLVESGGGLIQPGGSLRLSCAASGFTVSRNYMSWVRQAPGK |
| | (SEQ ID NO: 19) | GLEWVSVIYSGGSTFYADSVKGRFTISRDNSKNTLYLQMNSLRA |
| | | EDTAVYYCARDGDYYGMDVWGQGTTVTVSSASTKGPSVFPL |
| | | APSSKSTSGGTAALGCLVKDYFPEPVTVSWNSGALTSGVHTF |
| | | PAVLQSSGLYSLSSVVTVPSSSLGTQTYICNVNHKPSNTKVD |
| | | KKVEPKSCDKTHTCPPCPAPELLGGPSVFLFPPKPKDTLMIS |
| | | RTPEVTCVVVDVSHEDPEVKFNWYVDGVEVHNAKTKPREE |
| | | QYNSTYRVVSVLTVLHQDWLNGKEYKCKVSNKALPAPIEKT |
| | | ISKAKQPREPQVYTLPPSRDELTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPGK |
| | Light chain | DIQMTQSPSFLSASVGDRVTITCRASQGISSFLAWYQQKPGKAP |
| | (SEQ ID NO: 20) | KLLIYGASTLQSGVTSRFSGSGSGTEFTLTISSLQPEDFATYYCQR |
| | | LDSYPPITFGQGTRLEIKRTVAAPSVFIFPPSDEQLKSGTASVVC |
| | | LLNNFYPREAKVQWKVDNALQSGNSQESVTEQDSKDSTYSL |
| | | SSTLTLSKADYEKHKVYACEVTHQGLSSPVTKSFNRGEC |
| Cv2.5213 | Heavy chain | EVQLVESGGGLIQPGGSLRLSCAASGFTVSSNYMSWVRQAPGK |
| | (SEQ ID NO: 21) | GLEWVSLIYSGGSTYYADSVKGRFTISRDNSKNTLYLQMNSLR |
| | | AEDTAVYYCARDLNYYGMDVWGQGATVTVSSASTKGPSVFP |
| | | LAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKKVEPKSCDKTHTCPPCPAPELLGGPSVFLFPPKPKDTLMI |
| | | SRTPEVTCVVVDVSHEDPEVKFNWYVDGVEVHNAKTKPRE |
| | | EQYNSTYRVVSVLTVLHQDWLNGKEYKCKVSNKALPAPIEK |
| | | TISKAKQPREPQVYTLPPSRDELTKNQVSLTCLVKGFYPSDI |
| | | AVEWESNGQPENNYKTTPPVLDSDGSFFLYSKLTVDKSRWQ |
| | | QGNVFSCSVMHEALHNHYTQKSLSLSPGK |
| | Light chain | DIQMTQSPSSLSASVGDRVTITCRASQGISSYLAWYQQKPGKAP |
| | (SEQ ID NO: 22) | KLLIYAASTLQSGVPSRFSGSGSGTDFTLTISSLQPEDFATYYCQ |
| | | QLDSYSPFTFGPGTKVDIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSFNRGEC |
| Cv2.1169 | Heavy chain | QVQLVQSGPEVKKPGTSVKVSCKASGFTFTTSAVQWVRQARG |
| | (SEQ ID NO: 133) | QRLEWIGWIVVGSGNTNYAQKFQERVTITRDMSTTTAYMELSS |
| | | LRSEDTAVYYCAAPYCSGGTCLDGFDIWGQGTMVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKKVEPKSCDKTHTCPPCPAPELLGGPSVFLFPPKPK |
| | | DTLMISRTPEVTCVVVDVSHEDPEVKENWYVDGVEVHNAK |
| | | TKPREEQYNSTYRVVSVLTVLHQDWLNGKEYKCKVSNKAL |
| | | PAPIEKTISKAKGQPREPQVYTLPPSRDELTKNQVSLTCLVK |
| | | GFYPSDIAVEWESNGQPENNYKTTPPVLDSDGSFFLYSKLTV |
| | | DKSRWQQGNVFSCSVMHEALHNHYTQKSLSLSPGK |
| «prime» | Light chain | EIVLTQSPGTLSLSPGERATLSCRASQSVSRSYLAWYQQKPGQA |
| | (SEQ ID NO: 14) | PRLLIYSASSRATGIPDRFSGSGSGTDFTLTISRLEPEDFAVYYCQ |
| | | QYGSSPWTFGQGTKVEIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSENRGEC |
| Cv2.1353 | Heavy chain | EVQLVESGGGLVQPGGSLRLSCAASGVTVSSNYMNWVRQAPG |
| | (SEQ ID NO: 134) | KGLEWVSVIYSGGSTFYADSVKGRFIISRDNSKNTLYLQMNSLK |
| | | TEDTAVYYCARDLGPMGFDIWGQGTMVTVSSASTKGPSVFPL |
| | | APSSKSTSGGTAALGCLVKDYFPEPVTVSWNSGALTSGVHTF |
| | | PAVLQSSGLYSLSSVVTVPSSSLGTQTYICNVNHKPSNTKVD |
| | | KKVEPKSCDKTHTCPPCPAPELLGGPSVFLFPPKPKDTLMIS |
| | | RTPEVTCVVVDVSHEDPEVKFNWYVDGVEVHNAKTKPREE |
| | | QYNSTYRVVSVLTVLHQDWLNGKEYKCKVSNKALPAPIEKT |
| | | ISKAKGQPREPQVYTLPPSRDELTKNQVSLTCLVKGFYPSDI |
| | | AVEWESNGQPENNYKTTPPVLDSDGSFFLYSKLTVDKSRWQ |
| | | QGNVFSCSVMHEALHNHYTQKSLSLSPGK |
| « prime » | Light chain | DIQMTQSPSFLSASVGDRVTITCRASQGISSYLAWYQQKPGKAP |
| | (SEQ ID NO: 16) | KLLIYAASTLQSGVPSRFSGSGSGTEFTLTISSLQPEDFATYYCQQ |
| | | LNSDPPVTFGPGTKVDIKRTVAAPSVFIFPPSDEQLKSGTASVV |
| | | CLLNNFYPREAKVQWKVDNALQSGNSQESVTEQDSKDSTYS |
| | | LSSTLTLSKADYEKHKVYACEVTHQGLSSPVTKSFNRGEC |
| Cv2.3194 | Heavy chain | EVQLVESGGGLIQPGGSLRLSCAASGITVTSNYMSWVRQAPGK |
| | (SEQ ID NO: 135) | GLEWVSVIYPGGSTFYADSVKGRFTISRDNSKNTLYLQMNSLRA |
| | | EDTAVYYCARDLVVYGMDVWGQGTTVTVSSASTKGPSVFPL |
| | | APSSKSTSGGTAALGCLVKDYFPEPVTVSWNSGALTSGVHTF |
| | | PAVLQSSGLYSLSSVVTVPSSSLGTQTYICNVNHKPSNTKVD |
| | | KKVEPKSCDKTHTCPPCPAPELLGGPSVFLFPPKPKDTLMIS |
| | | RTPEVTCVVVDVSHEDPEVKFNWYVDGVEVHNAKTKPREE |
| | | QYNSTYRVVSVLTVLHQDWLNGKEYKCKVSNKALPAPIEKT |
| | | ISKAKGQPREPQVYTLPPSRDELTKNQVSLTCLVKGFYPSDI |
| | | AVEWESNGQPENNYKTTPPVLDSDGSFFLYSKLTVDKSRWQ |
| | | QGNVFSCSVMHEALHNHYTQKSLSLSPGK |
| « prime » | Light chain | EIVLTQSPGTLSLSPGERATLSCRASQSVSSSYLAWYQQKPGQA |
| | (SEQ ID NO: 18) | PRLLIYGASSRATGIPGRFSGSGSGTDFTLTISRLEPEDFAIYYCQ |
| | | QGVTFGGGTKVEIKRTVAAPSVFIFPPSDEQLKSGTASVVCLL |
| | | NNFYPREAKVQWKVDNALQSGNSQESVTEQDSKDSTYSLSS |
| | | TLTLSKADYEKHKVYACEVTHQGLSSPVTKSFNRGEC |
| Cv2.3235 | Heavy chain | EVQLVESGGGLIQPGGSLRLSCAASGFTVSRNYMSWVRQAPGK |
| | (SEQ ID NO: 136) | GLEWVSVIYSGGSTFYADSVKGRFTISRDNSKNTLYLQMNSLRA |
| | | EDTAVYYCARDGDYYGMDVWGQGTTVTVSSASTKGPSVFPL |
| | | APSSKSTSGGTAALGCLVKDYFPEPVTVSWNSGALTSGVHTF |
| | | PAVLQSSGLYSLSSVVTVPSSSLGTQTYICNVNHKPSNTKVD |
| | | KKVEPKSCDKTHTCPPCPAPELLGGPSVFLFPPKPKDTLMIS |
| | | RTPEVTCVVVDVSHEDPEVKFNWYVDGVEVHNAKTKPREE |
| | | QYNSTYRVVSVLTVLHQDWLNGKEYKCKVSNKALPAPIEKT |
| | | ISKAKGQPREPQVYTLPPSRDELTKNQVSLTCLVKGFYPSDI |
| | | AVEWESNGQPENNYKTTPPVLDSDGSFFLYSKLTVDKSRWQ |
| | | QGNVFSCSVMHEALHNHYTQKSLSLSPGK |
| « prime » | Light chain | DIQMTQSPSFLSASVGDRVTITCRASQGISSFLAWYQQKPGKAP |
| | (SEQ ID NO: 20) | KLLIYGASTLQSGVTSRFSGSGSGTEFTLTISSLQPEDFATYYCQR |
| | | LDSYPPITFGQGTRLEIKRTVAAPSVFIFPPSDEQLKSGTASVVC |
| | | LLNNFYPREAKVQWKVDNALQSGNSQESVTEQDSKDSTYSL |
| | | SSTLTLSKADYEKHKVYACEVTHQGLSSPVTKSFNRGEC |
| Cv2.5213 | Heavy chain | EVQLVESGGGLIQPGGSLRLSCAASGFTVSSNYMSWVRQAPGK |
| | (SEQ ID NO: 137) | GLEWVSLIYSGGSTYYADSVKGRFTISRDNSKNTLYLQMNSLR |
| | | AEDTAVYYCARDLNYYGMDVWGQGATVTVSSASTKGPSVFP |
| | | LAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKKVEPKSCDKTHTCPPCPAPELLGGPSVFLFPPKPKDTLMI |
| | | SRTPEVTCVVVDVSHEDPEVKFNWYVDGVEVHNAKTKPRE |
| | | EQYNSTYRVVSVLTVLHQDWLNGKEYKCKVSNKALPAPIEK |
| | | TISKAKGQPREPQVYTLPPSRDELTKNQVSLTCLVKGFYPSD |
| | | IAVEWESNGQPENNYKTTPPVLDSDGSFFLYSKLTVDKSRW |
| | | QQGNVFSCSVMHEALHNHYTQKSLSLSPGK |
| « prime » | Light chain | DIQMTQSPSSLSASVGDRVTITCRASQGISSYLAWYQQKPGKAP |
| | (SEQ ID NO: 22) | KLLIYAASTLQSGVPSRFSGSGSGTDFTLTISSLQPEDFATYYCQ |
| | | QLDSYSPFTFGPGTKVDIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSFNRGEC |
| Cv2.5179 | Heavy chain | QVQLVQSGPEVKKPGTSVKVSCKASGFTFTSSAVQWVRQARG |
| | (SEQ ID NO: 154) | QRLEWIGWIVVGSGNTNYAQKFQERVTITRDMSTSTAYMELSS |
| | | LRSEDTAVYYCAAPHCSSTSCYDAFDIWGQGTMVTVSSASTKG |
| | | PSVFPLAPSSKSTSGGTAALGCLVKDYFPEPVTVSWNSGALTSG |
| | | VHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYN |
| | | STYRVVSVLTVLHQDWLNGKEYKCKVSNKALPAPIEKTISKAK |
| | | GQPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGK |
| | Light chain | EIVLTQSPGTLSLSPGERATLSCRASQSVSSSYLAWYQQKPGQA |
| | (SEQ ID NO: 155) | PRLLIYGASSRATGIPDRFSGSGSGTDFTLTISRLEAEDFAVYYCQ |
| | | QYGRSPWTFGQGTKLEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |

Thus, in some particular embodiments, the antibody or antigen-binding fragment thereof according to the present disclosure comprises a heavy chain amino acid sequence selected from the group consisting of: SEQ ID NO: 13, 15, 17, 19 and 21, in particular SEQ ID NO: 13 and 17, preferably SEQ ID NO: 13.

In some other particular embodiments, the antibody or antigen-binding fragment thereof according to the present disclosure comprises a heavy chain amino acid sequence selected from the group consisting of: SEQ ID NO: 133, 134, 135, 136 and 137 and 154, preferably SEQ ID NO: 13 or SEQ ID NO: 133.

In some more particular embodiments, said antibody or antigen-binding fragment thereof comprises:
a) a heavy chain amino acid sequence having at least 90% identity with SEQ ID NO: 13 and a light chain amino acid sequence having at least 90% identity with SEQ ID NO: 14,
b) a heavy chain amino acid sequence having at least 90% identity with SEQ ID NO: 15 and a light chain amino acid sequence having at least 90% identity with SEQ ID NO: 16,
c) a heavy chain amino acid sequence having at least 90% identity with SEQ ID NO: 17 and a light chain amino acid sequence having at least 90% identity with SEQ ID NO: 18,
d) a heavy chain amino acid sequence having at least 90% identity with SEQ ID NO: 19 and a light chain amino acid sequence having at least 90% identity with SEQ ID NO: 20, or
e) a heavy chain amino acid sequence having at least 90% identity with SEQ ID NO: 21 and a light chain amino acid sequence having at least 90% identity with SEQ ID NO: 22;
f) a heavy chain amino acid sequence having at least 90% identity with SEQ ID NO: 154 and a light chain amino acid sequence having at least 90% identity with SEQ ID NO: 155.

In some particular embodiments, the heavy and light chain amino acid sequences in any one of a) to f), such as in any one of a) to e), have at least 95%, 96%, 97%, 98%, 99%, or 100% identity with the above disclosed sequences

In some other more particular embodiments, said antibody or antigen-binding fragment thereof comprises: a heavy chain amino acid sequence having at least 90% identity with SEQ ID NO: 154 and a light chain amino acid sequence having at least 90% identity with SEQ ID NO: 155. In some particular embodiments, the heavy and light chain amino acid sequences have at least 95%, 96%, 97%, 98%, 99%, or 100% identity with the above disclosed sequences.

In some other more particular embodiments, said antibody or antigen-binding fragment thereof comprises:
a) a heavy chain amino acid sequence having at least 90% identity with SEQ ID NO: 133 and a light chain amino acid sequence having at least 90% identity with SEQ ID NO: 14,
b) a heavy chain amino acid sequence having at least 90% identity with SEQ ID NO: 134 and a light chain amino acid sequence having at least 90% identity with SEQ ID NO: 16,
c) a heavy chain amino acid sequence having at least 90% identity with SEQ ID NO: 135 and a light chain amino acid sequence having at least 90% identity with SEQ ID NO: 18,
d) a heavy chain amino acid sequence having at least 90% identity with SEQ ID NO: 136 and a light chain amino acid sequence having at least 90% identity with SEQ ID NO: 20, or
e) a heavy chain amino acid sequence having at least 90% identity with SEQ ID NO: 137 and a light chain amino acid sequence having at least 90% identity with SEQ ID NO: 22;
f) a heavy chain amino acid sequence having at least 90% identity with SEQ ID NO: 154 and a light chain amino acid sequence having at least 90% identity with SEQ ID NO: 155.

In some particular embodiments, the heavy and light chain amino acid sequences in any one of a) to f), such as in any one of a) to e) have at least 95%, 96%, 97%, 98%, 99%, or 100% identity with the above disclosed sequences

Preferably said antibody or antigen-binding fragment thereof comprises a heavy chain amino acid sequence having at least 90% identity with SEQ ID NO: 13 or SEQ ID NO: 133 and a light chain amino acid sequence having at least 90% identity with SEQ ID NO: 14; more preferably said antibody or antigen-binding fragment comprises a heavy chain amino acid sequence of SEQ ID NO: 13 or SEQ ID NO: 133 and a light chain amino acid sequence of SEQ ID NO: 14.

Anti-SARS-CoV-2 antibodies or antigen-binding fragments thereof with amino acid sequences having at least 90%, for example, at least 95%, 96%, 97%, 98%, 99%, or 100% identity to any one of the above defined amino acid sequences are part of the present disclosure, typically anti-SARS-CoV-2 antibodies have at least equal or higher neutralizing activities than said anti-SARS-CoV-2 antibodies consisting ofheavy chain SEQ ID NO:13 or SEQ ID NO:133 and light chain of SEQ ID NO:14.

Other neutralizing anti-SARS-CoV-2 antibodies or antigen-binding fragments thereof according to the present disclosure which may be used include any antibodies comprising the 6 CDRs of Cv2.1169, Cv2.1353, Cv2.3194, Cv2.3235 or Cv2.5213 or Cv2.5179 as described in the Table 3 below.

TABLE 3
CDR regions of Cv2.1169, Cv2.1353, Cv2.3194,
Cv2.3235, Cv2.5213 and Cv2.5179
according to Kabat numbering.

| | | | | |
|---|---|---|---|---|
| Cv2.1169 | Heavy | CDR1 | TSAVQ | SEQ ID NO: 23 |
| | chain | CDR2 | WIVVGSGNTNYAQKFQE | SEQ ID NO: 24 |
| | | CDR3 | PYCSGGTCLDGFDI | SEQ ID NO: 25 |
| | Light | CDR1 | RASQSVSRSYLA | SEQ ID NO: 26 |
| | chain | CDR2 | SASSRAT | SEQ ID NO: 27 |
| | | CDR3 | QQYGSSPWT | SEQ ID NO: 28 |
| Cv2.1353 | Heavy | CDR1 | SNYMN | SEQ ID NO: 29 |
| | chain | CDR2 | VIYSGGSTFYADSVKG | SEQ ID NO: 30 |
| | | CDR3 | DLGPMGFDI | SEQ ID NO: 31 |
| | Light | CDR1 | RASQGISSYLA | SEQ ID NO: 32 |
| | chain | CDR2 | AASTLQS | SEQ ID NO: 33 |
| | | CDR3 | QQLNSDPPVT | SEQ ID NO: 34 |
| Cv2.3194 | Heavy | CDR1 | SNYMS | SEQ ID NO: 35 |
| | chain | CDR2 | VIYPGGSTFYADSVKG | SEQ ID NO: 36 |
| | | CDR3 | DLVVYGMDV | SEQ ID NO: 37 |
| | Light | CDR1 | RASQSVSSSYLA | SEQ ID NO: 38 |
| | chain | CDR2 | GASSRAT | SEQ ID NO: 39 |
| | | CDR3 | QQGVT | SEQ ID NO: 40 |
| Cv2.3235 | Heavy | CDR1 | RNYMS | SEQ ID NO: 41 |
| | chain | CDR2 | VIYSGGSTFYADSVKG | SEQ ID NO: 42 |
| | | CDR3 | DGDYYGMDV | SEQ ID NO: 43 |
| | Light | CDR1 | RASQGISSFLA | SEQ ID NO: 44 |
| | chain | CDR2 | GASTLQS | SEQ ID NO: 45 |
| | | CDR3 | QRLDSYPPIT | SEQ ID NO: 46 |
| Cv2.5213 | Heavy | CDR1 | SNYMS | SEQ ID NO: 47 |
| | chain | CDR2 | LIYSGGSTYYADSVKG | SEQ ID NO: 48 |
| | | CDR3 | DLNYYGMDV | SEQ ID NO: 49 |
| | Light | CDR1 | RASQGISSYLA | SEQ ID NO: 50 |
| | chain | CDR2 | AASTLQS | SEQ ID NO: 51 |
| | | CDR3 | QQLDSYSPFT | SEQ ID NO: 52 |
| Cv2.5179 | Heavy | CDR1 | SSAVQ | SEQ ID NO: 138 |
| | chain | CDR2 | WIVVGSGNTNYAQKFQE | SEQ ID NO: 139 |
| | | CDR3 | PHCSSTSCYDAFDI | SEQ ID NO: 140 |
| | Light | CDR1 | RASQSVSSSYLA | SEQ ID NO: 141 |
| | chain | CDR2 | GASSRAT | SEQ ID NO: 142 |
| | | CDR3 | QQYGRSPWT | SEQ ID NO: 143 |

In some particular embodiments, the anti-SARS-CoV-2 antibody or antigen-binding fragment thereof according to the present disclosure comprises:
a) a heavy chain variable domain comprising a heavy chain CDR1 of SEQ ID NO: 23, a heavy chain CDR2 of SEQ ID NO: 24 and a heavy chain CDR3 of SEQ ID NO: 25,
b) a heavy chain variable domain comprising: a heavy chain CDR1 of SEQ ID NO: 29, a heavy chain CDR2 of SEQ ID NO: 30 and a heavy chain CDR3 of SEQ ID NO: 31,
c) a heavy chain variable domain comprising a heavy chain CDR1 of SEQ ID NO: 35, a heavy chain CDR2 of SEQ ID NO: 36 and a heavy chain CDR3 of SEQ ID NO: 37,
d) a heavy chain variable domain comprising: a heavy chain CDR1 of SEQ ID NO: 41, a heavy chain CDR2 of SEQ ID NO: 42 and a heavy chain CDR3 of SEQ ID NO: 43, or
e) a heavy chain variable domain comprising: a heavy chain CDR1 of SEQ ID NO: 47, a heavy chain CDR2 of SEQ ID NO: 48 and a heavy chain CDR3 of SEQ ID NO: 49,
f) a heavy chain variable domain comprising: a heavy chain CDR1 of SEQ ID NO: 138, a heavy chain CDR2 of SEQ ID NO: 139 and a heavy chain CDR3 of SEQ ID NO: 140.

Preferably said antibody or antigen-binding fragment thereof comprises a heavy chain variable domain comprising: a heavy chain CDR1 of SEQ ID NO: 23, a heavy chain CDR2 of SEQ ID NO: 24 and a heavy chain CDR3 of SEQ ID NO: 25.

In a more particular embodiment, said anti-SARS-CoV-2 antibody or antigen-binding fragment thereof comprises:
a) a heavy chain variable domain comprising: a heavy chain CDR1 of SEQ ID NO: 23, a heavy chain CDR2 of SEQ ID NO: 24, and a heavy chain CDR3 of SEQ ID NO: 25, and a light chain variable domain comprising: a light chain CDR1 of SEQ ID NO: 26, a light chain CDR2 of SEQ ID NO: 27 and a light chain CDR3 of SEQ ID NO: 28,
b) a heavy chain variable domain comprising: a heavy chain CDR1 of SEQ ID NO: 29, a heavy chain CDR2 of SEQ ID NO: 30 and a heavy chain CDR3 of SEQ ID NO: 31, and a light chain variable domain comprising: a light chain CDR1 of SEQ ID NO: 32, a light chain CDR2 of SEQ ID NO: 33, and a light chain CDR3 of SEQ ID NO: 34,
c) a heavy chain variable domain comprising: a heavy chain CDR1 of SEQ ID NO: 35, a heavy chain CDR2 of SEQ ID NO: 36 and a heavy chain CDR3 of SEQ ID NO: 37, and a light chain variable domain comprising: a light chain CDR1 of SEQ ID NO: 38, a light chain CDR2 of SEQ ID NO: 39, and a light chain CDR3 of SEQ ID NO: 40,
d) a heavy chain variable domain comprising: a heavy chain CDR1 of SEQ ID NO: 41, a heavy chain CDR2 of SEQ ID NO: 42 and a heavy chain CDR3 of SEQ ID NO: 43, and a light chain variable domain comprising: a light chain CDR1 of SEQ ID NO: 44, a light chain CDR2 of SEQ ID NO: 45, and a light chain CDR3 of SEQ ID NO: 46,
e) a heavy chain variable domain comprising: a heavy chain CDR1 of SEQ ID NO: 47, a heavy chain CDR2 of SEQ ID NO: 48 and a heavy chain CDR3 of SEQ ID NO: 49 and a light chain variable domain comprising: a light chain CDR1 of SEQ ID NO: 50, a light chain CDR2 of SEQ ID NO: 51, and a light chain CDR3 of SEQ ID NO: 52 or
f) a heavy chain variable domain comprising: a heavy chain CDR1 of SEQ ID NO: 138, a heavy chain CDR2 of SEQ ID NO: 139 and a heavy chain CDR3 of SEQ ID NO: 140 and a light chain variable domain comprising: a light chain CDR1 of SEQ ID NO: 141, a light chain CDR2 of SEQ ID NO: 142, and a light chain CDR3 of SEQ ID NO: 143.

Preferably said antibody or antigen-binding fragment thereof comprises:
a heavy chain CDR1 of SEQ ID NO: 23, a heavy chain CDR2 of SEQ ID NO: 24 and a heavy chain CDR3 of SEQ ID NO: 25, and
a light chain CDR1 of SEQ ID NO: 26, a light chain CDR2 of SEQ ID NO: 27, and a light chain CDR3 of SEQ ID NO: 28.

It is further contemplated that antibodies or antigen-binding fragment thereof may be further screened or optimized for their neutralizing properties as above defined. In particular, it is contemplated that monoclonal antibodies or antigen-binding fragment thereof may have 1, 2, 3, 4, 5, 6, or more alterations in the amino acid sequence of 1, 2, 3, 4, 5, or 6 CDRs of monoclonal antibodies provided herein, in particular in the CDR of SEQ ID NO: 23-52. It is contemplated that the amino acid in position 1, 2, 3, 4, 5, 6, 7, 8, 9, or 10 of CDR1, CDR2, CDR3, CDR4, CDR5, or CDR6 of the VJ or VDJ region of the light or heavy variable region of antibodies may have an insertion, deletion, or substitution with a conserved or non-conserved amino acid. Such amino acids that can either be substituted or constitute the substitution are disclosed below. In some particular embodiments, the monoclonal antibodies or antigen-binding fragment have 1 or 2 conservative substitutions in the amino acid sequence of 1, 2, 3, 4, 5, or 6 CDRs of monoclonal antibodies provided herein, in particular in the CDR of SEQ ID NO: 23-52.

In some embodiments, the amino acid differences are conservative substitutions, i.e., substitutions of one amino acid with another having similar chemical or physical properties (size, charge or polarity), which substitution generally does not adversely affect the biochemical, biophysical and/or biological properties of the antibody. In particular, the substitution does not disrupt the interaction of the antibody with the spike glycoprotein antigen and neutralizing properties. Said conservative substitution(s) are advantageously chosen within one of the following five groups: Group 1-small aliphatic, non-polar or slightly polar residues (A, S, T, P, G); Group 2-polar, negatively charged residues and their amides (D, N, E, Q); Group 3-polar, positively charged residues (H, R, K); Group 4-large aliphatic, nonpolar residues (M, L, I, V, C); and Group 5-large, aromatic residues (F, Y, W).

Neutralizing antibodies or antigen-binding fragments thereof according to the present disclosure defined by their CDR domains may comprise framework regions FR1, FR2, FR3 and FR4 of Cv2.1169, Cv2.1353, Cv2.3194, Cv2.3235, Cv2.5213 and Cv2.5179 antibodies as defined in the table below.

TABLE 4
Framework regions of Cv2.1169, Cv2.1353,
Cv2.3194, Cv2.3235, Cv2.5213 and Cv2.5179.

| | | | | |
|---|---|---|---|---|
| Cv2.1169 | Heavy | FR1 | QVQLVQSGPEVKKPGTSVKVSCKASGFTFT | SEQ ID NO: 53 |
| | chain | FR2 | WVRQARGQRLEWIG | SEQ ID NO: 54 |
| | | FR3 | RVTITRDMSTTTAYMELSSLRSEDTAVYYCAA | SEQ ID NO: 55 |
| | | FR4 | WGQGTMVTVSS | SEQ ID NO: 56 |
| | Light | FR1 | EIVLTQSPGTLSLSPGERATLSC | SEQ ID NO: 57 |
| | chain | FR2 | WYQQKPGQAPRLLIY | SEQ ID NO: 58 |
| | | FR3 | GIPDRFSGSGSGTDFTLTISRLEPEDFAVYYC | SEQ ID NO: 59 |
| | | FR4 | FGQGTKVEIK | SEQ ID NO: 60 |
| Cv2.1353 | Heavy | FR1 | EVQLVESGGGLVQPGGSLRLSCAASGVTVS | SEQ ID NO: 61 |
| | chain | FR2 | WVRQAPGKGLEWVS | SEQ ID NO: 62 |
| | | FR3 | RFIISRDNSKNTLYLQMNSLKTEDTAVYYCAR | SEQ ID NO: 63 |
| | | FR4 | WGQGTMVTVSS | SEQ ID NO: 64 |
| | Light | FR1 | DIQMTQSPSFLSASVGDRVTITC | SEQ ID NO: 65 |
| | chain | FR2 | WYQQKPGKAPKLLIY | SEQ ID NO: 66 |
| | | FR3 | GVPSRFSGSGSGTEFTLTISSLQPEDFATYYC | SEQ ID NO: 67 |
| | | FR4 | FGPGTKVDIK | SEQ ID NO: 68 |
| Cv2.3194 | Heavy | FR1 | EVQLVESGGGLIQPGGSLRLSCAASGITVT | SEQ ID NO: 69 |
| | chain | FR2 | WVRQAPGKGLEWVS | SEQ ID NO: 70 |
| | | FR3 | RFTISRDNSKNTLYLQMNSLRAEDTAVYYCAR | SEQ ID NO: 71 |
| | | FR4 | WGQGTTVTVSS | SEQ ID NO: 72 |
| | Light | FR1 | EIVLTQSPGTLSLSPGERATLSC | SEQ ID NO: 73 |
| | chain | FR2 | WYQQKPGQAPRLLIY | SEQ ID NO: 74 |
| | | FR3 | GIPGRFSGSGSGTDFTLTISRLEPEDFAIYYC | SEQ ID NO: 75 |
| | | FR4 | FGGGTKVEIK | SEQ ID NO: 76 |
| Cv2.3235 | Heavy | FR1 | EVQLVESGGGLIQPGGSLRLSCAASGFTVS | SEQ ID NO: 77 |
| | chain | FR2 | WVRQAPGKGLEWVS | SEQ ID NO: 78 |
| | | FR3 | RFTISRDNSKNTLYLQMNSLRAEDTAVYYCAR | SEQ ID NO: 79 |
| | | FR4 | WGQGTTVTVSS | SEQ ID NO: 80 |
| | Light | FR1 | DIQMTQSPSFLSASVGDRVTITC | SEQ ID NO: 81 |
| | chain | FR2 | WYQQKPGKAPKLLIY | SEQ ID NO: 82 |
| | | FR3 | GVTSRFSGSGSGTEFTLTISSLQPEDFATYYC | SEQ ID NO: 83 |
| | | FR4 | FGQGTRLEIK | SEQ ID NO: 84 |
| Cv2.5213 | Heavy | FR1 | EVQLVESGGGLIQPGGSLRLSCAASGFTVS | SEQ ID NO: 85 |
| | chain | FR2 | WVRQAPGKGLEWVS | SEQ ID NO: 86 |
| | | FR3 | RFTISRDNSKNTLYLQMNSLRAEDTAVYYCAR | SEQ ID NO: 87 |
| | | FR4 | WGQGATVTVSS | SEQ ID NO: 88 |
| | Light | FR1 | DIQMTQSPSSLSASVGDRVTITC | SEQ ID NO: 89 |
| | chain | FR2 | WYQQKPGKAPKLLIY | SEQ ID NO: 90 |
| | | FR3 | GVPSRFSGSGSGTDFTLTISSLQPEDFATYYC | SEQ ID NO: 91 |
| | | FR4 | FGPGTKVDIK | SEQ ID NO: 92 |
| Cv2.5179 | Heavy | FR1 | QVQLVQSGPEVKKPGTSVKVSCKASGFTFT | SEQ ID NO: 144 |
| | chain | FR2 | WVRQARGQRLEWIG | SEQ ID NO: 145 |
| | | FR3 | RVTITRDMSTSTAYMELSSLRSEDTAVYYCAA | SEQ ID NO: 146 |
| | | FR4 | WGQGTMVTVSS | SEQ ID NO: 147 |
| | Light | FR1 | EIVLTQSPGTLSLSPGERATLSC | SEQ ID NO: 148 |
| | chain | FR2 | WYQQKPGQAPRLLIY | SEQ ID NO: 149 |
| | | FR3 | GIPDRFSGSGSGTDFTLTISRLEAEDFAVYYC | SEQ ID NO: 150 |
| | | FR4 | FGQGTKLEIK | SEQ ID NO: 151 |

In a particular aspect of the disclosure, it is disclosed an antibody or antigen-binding fragment thereof as defined above by their CDR domains, wherein:
a) the variable heavy domain comprises the amino acid sequence of the framework FR1 selected from the group consisting of: SEQ ID NO: 53, 61, 69, 77, 85 and 144, the amino acid sequence of the framework FR2 selected from the group consisting of: SEQ ID NO: 54, 62, 70, 78, 86 and 145, the amino acid sequence of the framework FR3 selected from the group consisting of: SEQ ID NO: 55, 63, 71, 79, 87 and 146 and/or the amino acid sequence of the framework FR4 selected from the group consisting of: SEQ ID NO: 56, 64, 72, 80, 88 and 147; and/or,
b) the variable light domain comprises the amino acid sequence of the framework FR1 selected from the group consisting of: SEQ ID NO: 57, 65, 73, 81, 89 and 148, the amino acid sequence of the framework FR2 selected from the group consisting of: SEQ ID NO: 58, 66, 74, 82, 90 and 149, the amino acid sequence of the framework FR3 selected from the group consisting of: SEQ ID NO: 59, 67, 75, 83, 91 and 150, and/or the amino acid sequence of the framework FR4 selected from the group consisting of: SEQ ID NO: 60, 68, 76, 84, 92 and 151.

In a particular aspect of the disclosure, it is disclosed an antibody or antigen-binding fragment thereof as defined above by their CDR domains, wherein:
a) the variable heavy domain comprises the amino acid sequence of the framework FR1 selected from the group consisting of: SEQ ID NO: 53, 61, 69, 77 and 85, the amino acid sequence of the framework FR2 selected from the group consisting of: SEQ ID NO: 54, 62, 70, 78 and 86, the amino acid sequence of the framework FR3 selected from the group consisting of: SEQ ID NO: 55, 63, 71, 79 and 87 and/or the amino acid sequence of the framework FR4 selected from the group consisting of: SEQ ID NO: 56, 64, 72, 80 and 88; and/or,
b) the variable light domain comprises the amino acid sequence of the framework FR1 selected from the group consisting of: SEQ ID NO: 57, 65, 73, 81 and 89, the amino acid sequence of the framework FR2 selected from the group consisting of: SEQ ID NO: 58, 66, 74, 82 and 90, the amino acid sequence of the framework FR3 selected from the group consisting of: SEQ ID NO: 59, 67, 75, 83 and 91 and/or the amino acid sequence of the framework FR4 selected from the group consisting of: SEQ ID NO: 60, 68, 76, 84 and 92.

In particular, it is contemplated that monoclonal antibodies or antigen-binding fragment thereof may have 1, 2, 3, 4, 5, 6, 7, 8, 9, 10 or more alterations in the amino acid sequence of 1, 2, 3, 4, 5, 6, 7, 8 FRs of monoclonal antibodies provided herein, in particular in the FR of SEQ ID NO: 53-92. It is contemplated that the FR sequences have an insertion, deletion, or substitution with a conserved or non-conserved amino acid. Such amino acids that can either be substituted or constitute the substitution are disclosed above. In some particular embodiments, the monoclonal antibodies or antigen-binding fragment have 1, 2, 3, 4, 5; preferably 1 or 2 conservative substitutions in the amino acid sequence of 1, 2, 3, 4, 5, 6, 7, 8 FRs of monoclonal antibodies provided herein, in particular in the FR of SEQ ID NO: 53-92 and 144-151, in particular in the FR of SEQ ID NO: 53-92.

Variant antibodies according to the present disclosure are functional antibodies that specifically bind to SARSV-CoV-2 Spike protein RBD and exhibit functional properties that are substantially equal or superior to the corresponding functional properties of the corresponding reference antibody human antibody Cv2.1169, Cv2.1353, Cv2.3194, Cv2.3235 or Cv2.5213 as described above. By substantially equal it is herein intended that the functional variant retains at least about 50%, 60%, 70%, 80%, 90%, 95%, 96%, 97%, 98%, 99% or 100% of the corresponding functional property of the reference human antibody.

In a more particular embodiment, said antibody or antigen-binding fragment thereof defined by their CDR domains comprises the following frameworks domains:
VHFR1 of SEQ ID NO: 53
VHFR2 of SEQ ID NO: 54,
VHFR3 of SEQ ID NO: 55
VHFR4 of SEQ ID NO: 56,
VLFR1 of SEQ ID NO: 57,
VLFR2 of SEQ ID NO: 58
VLFR3 of SEQ ID NO: 59, and
VLFR4 of SEQ ID NO: 60.

In another particular embodiment, said antibody or antigen-binding fragment thereof defined by their CDR domains comprises the following frameworks domains:
VHFR1 of SEQ ID NO: 69
VHFR2 of SEQ ID NO: 70,
VHFR3 of SEQ ID NO:71
VHFR4 of SEQ ID NO: 72,
VLFR1 of SEQ ID NO: 73,
VLFR2 of SEQ ID NO: 74
VLFR3 of SEQ ID NO: 75, and
VLFR4 of SEQ ID NO: 76.

In a particular embodiment of the disclosure, the variable regions of the antibody as described above may be associated with antibody constant regions, like IgA, IgM, IgE, IgD or IgG such as Igg1, IgG2, IgG3, IgG4. Said variable regions of the antibody is preferably associated with IgG or IgA constant region; preferably IgG1 or IgA (IgA1, IgA2) constant regions. These constant regions may be further mutated or modified, by methods known in the art, in particular for modifying their binding capability towards Fc receptor or enhancing antibody half-life. The antibody comprising IgA constant region may further comprise a J chain and/or a secretory component to generate a polymeric or secretory IgA.

As used herein, the term “IgG Fc region” is used to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat. The C-terminal lysine (residue K447) of the Fc region may be removed, for example, during production or purification of the antibody. Accordingly, a composition of antibodies of the disclosure may comprise antibody populations with all K447 residues removed, antibody populations with no K447 residues removed, and antibody populations having a mixture of antibodies with and without the K447 residue.

In a particular embodiment, the anti-SARS-CoV-2 antibody according to the present disclosure is a silent antibody. As used herein, the term “silent” antibody refers to an antibody that exhibits no or low ADCC activity. Silenced effector functions can be obtained by mutation in the Fc region of the antibodies and have been described in the Art: Strohl 2009 (LALA & N297A); Baudino 2008, D265A (Baudino et al., J. Immunol. 181 (2008): 6664-69, Strohl, CO Biotechnology 20 (2009): 685-91). Examples of silent Fc IgG1 antibodies comprise N297A or L234A and L235A mutations in the IgG1 Fc amino acid sequence.

In another particular embodiment of any of the antibodies or antigen-binding fragments described herein, the variant human Fc constant region comprises the M428L and N434S substitutions (LS) in EU index of Kabat to enhance antibody half-life. In some embodiments, the antibody of the present disclosure does not comprise an Fc domain capable of substantially binding to a FcgRIIIA (CD16) polypeptide. In some embodiments, the antibody of the present disclosure lacks an Fc domain (e.g. lacks a CH2 and/or CH3 domain).

Other mutations are G236A/A330L/I332E, herein termed “GAALIE which improves antiviral efficacy against pathogens (Bournazos et al., Nature, 2020, 588, 485-490). The M428L and N434S substitutions (LS) are advantageously combined with G236A/A330L/I332E.

Another modification of the antibodies herein that is contemplated by the present disclosure is pegylation or hesylation or related technologies. An antibody can be pegylated to, for example, increase the biological (e.g., serum) half-life of the antibody. To pegylate an antibody, the antibody, or fragment thereof, typically is reacting with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the disclosure. See for example, EP 0 154 316 by Nishimura et al. and EP 0 401 384 by Ishikawa et al.

Another modification of the antibodies that is herein contemplated is a conjugate or a fusion protein. The antibody or antigen-binding fragment thereof may be fused to another protein moiety of interest or conjugated to an agent of interest. The agent may be for example a therapeutic agent; a label for antibody detection or a protein which increases the half-life of the antibody. In some embodiments, at least the antigen-binding region of the antibody of the present disclosure is fused to a serum protein, such as human serum albumin or a fragment thereof to increase half-life of the resulting molecule. In other embodiments, the antibody or antigen binding fragment according to the present disclosure further comprises a detectable label. Preferred labels include fluorophores such as umbelliferone, fluorescein, fluorescein isothiocyanate (FITC), rhodamine, tetramethyl rhodamine, eosin, green fluorescent protein, erythrosin, coumarin, methyl coumarin, pyrene, malachite green, stilbene, lucifer yellow, Cascade Blue, Texas Red, dichlorotriazinylamine fluorescein, dansyl chloride, phycoerythrin, fluorescent lanthanide complexes such as those including Europium and Terbium, cyanine dye family members, such as Cy3 and Cy5, molecular beacons and fluorescent derivatives thereof, a chromophore label; a luminescent label such as luminol; a radioactive label such as ¹⁴C, ¹²³I, ¹²⁴I, ¹²⁵I, ³²P, ³³P, ³⁵S, or ³H and others; an affinity-ligand label, such as streptavidin/biotin, avidin/biotin or anti-isotype antibody; an enzyme label, such as alkaline phosphatase, horseradish peroxidase, luciferase, β galactosidase or acetylcholinesterase; an enzyme cofactor label; a hapten conjugate label, such as digoxigenin or dinitrophenyl; a Raman signal generating label; a magnetic label; a spin label; an epitope label, such as the FLAG or HA epitope; a heavy atom label; a nanoparticle label, an electrochemical label; a light scattering or plasmon resonant materials such as gold or silver particles or quantum dots; a spherical shell label; semiconductor nanocrystal label; as well as others known in the art, wherein the label can allow visualization with or without a secondary detection molecule.

In some embodiments, said antibody is polymeric. The polymeric antibody comprises or consists of Ig polymers. The polymeric antibody is preferably a polymeric monoclonal antibody derived from a monoclonal antibody as defined above. The Ig polymers comprise or consist of dimers. The polymeric antibody usually comprises immunoglobulin joining (J) chain(s) in addition to Ig molecules. The J chain is a 137 amino acid polypeptide expressed by plasma or myeloma cells which regulate Ig polymer formation by binding covalently to two Ig molecules through disulfide bonds between cysteine residues. In particular, dimeric antibodies are formed by two monomeric Ig molecules, which covalently bind to a J chain. In a preferred embodiment, said antibody is a polymeric IgA, preferably a polymeric IgA monoclonal antibody derived from a monoclonal antibody as defined above.

In some embodiments the antibody is a secretory antibody. A secretory antibody can be transported across epithelial cells to the luminal surface of serosal tissues. The secretory antibody is usually a polymeric antibody, preferably a polymeric IgA, comprising a complex of J-chain-containing polymer of Ig and secretory component (SC). The secretory component is a proteolytic cleavage product of the extracellular part of the polymeric immunoglobulin receptor (pIgR) which binds to J-chain containing polymeric Ig. The secretory antibody is preferably a secretory IgA monoclonal antibody derived from a monoclonal antibody as defined above.

In some preferred embodiment, the neutralizing antibody is a recombinant human monoclonal antibody, preferably of IgG1 or IgA isotype. The IgA may be monomeric, polymeric or secretory IgA; it is preferably a polymeric or secretory IgA. In some more preferred embodiments, the recombinant antibody is a silent antibody that may further comprise mutations and/or or modifications to enhance antibody half-life, as described above.

The present invention also relates to an antigen binding fragment of an antibody that contain the variable domains comprising the CDRs domains as described above such as Fv, dsFv, scFv, Fab, Fab′, F(ab′)2. In particular, said antigen binding fragment is a F(ab′)2 fragment. The F(ab′)2 fragment can be produced by pepsin digestion of an antibody below the hinge disulfide; it comprises two Fab′ fragments, and additionally a portion of the hinge region of the immunoglobulin molecule. Fab fragments are monomeric fragments obtainable by papain digestion of an antibody; they comprise the entire L chain, and a VH-CH1 fragment of the H chain, bound together through a disulfide bond. The Fab′ fragments are obtainable from F(ab′)2 fragments by cutting a disulfide bond in the hinge region. F(ab′)2 fragments are divalent, i.e. they comprise two antigen binding sites, like the native immunoglobulin molecule; on the other hand, Fv (a VHVL dimer constituting the variable part of Fab), dsFv, scFv, Fab, and Fab′ fragments are monovalent, i.e. they comprise a single antigen-binding site. These basic antigen-binding fragments of the disclosure can be combined together to obtain multivalent antigen-binding fragments, such as diabodies, tribodies or tetrabodies. These multivalent antigen-binding fragments are also part of the present invention. Fv fragments consist of the VL and VH domains of an antibody associated together by hydrophobic interactions; in dsFv fragments, the VH:VL heterodimer is stabilized by a disulphide bond; in scFv fragments, the VL and VH domains are connected to one another via a flexible peptide linker thus forming a single-chain protein.

Another aspect of the disclosure relates to an isolated antibody directed against the viral Spike protein receptor binding-domain (RBD) of SARS-CoV-2, or an antigen-binding fragment thereof characterized in that it comprises:
a heavy chain variable domain selected from:
a heavy chain variable domain comprising: a heavy chain CDR1 of SEQ ID NO: 138, a heavy chain CDR2 of SEQ ID NO: 139 and a heavy chain CDR3 of SEQ ID NO: 140;
or
a heavy chain variable domain comprising at least one of, preferably all three of: a heavy chain CDR1 of SEQ ID NO: 23, a heavy chain CDR2 of SEQ ID NO: 24 and a heavy chain CDR3 of SEQ ID NO: 25, or a variant with one or two conservative substitution(s); or
a heavy chain variable domain comprising at least one of, preferably all three of: a heavy chain CDR1 of SEQ ID NO: 29, a heavy chain CDR2 of SEQ ID NO: 30 and a heavy chain CDR3 of SEQ ID NO: 31, or a variant with one or two conservative substitution(s); or
a heavy chain variable domain comprising at least one of, preferably all three of: a heavy chain CDR1 of SEQ ID NO: 35, a heavy chain CDR2 of SEQ ID NO: 36 and a heavy chain CDR3 of SEQ ID NO: 37, or a variant with one or two conservative substitution(s); or
a heavy chain variable domain comprising at least one of, preferably all three of: a heavy chain CDR1 of SEQ ID NO: 41, a heavy chain CDR2 of SEQ ID NO: 42 and a heavy chain CDR3 of SEQ ID NO: 43, or a variant with one or two conservative substitution(s); or
a heavy chain variable domain comprising at least one of, preferably all three of: a heavy chain CDR1 of SEQ ID NO: 47, a heavy chain CDR2 of SEQ ID NO: 48 and a heavy chain CDR3 of SEQ ID NO: 49, or a variant with one or two conservative substitution(s);
   and
a light chain variable domain selected from:
a light chain variable domain comprising at least one of, preferably all three of: a light chain CDR1 of SEQ ID NO: 141, a light chain CDR2 of SEQ ID NO: 142 and a light chain CDR3 of SEQ ID NO: 143, or a variant with one or two conservative substitution(s); or
a light chain variable domain comprising at least one of, preferably all three of: a light chain CDR1 of SEQ ID NO: 26, a light chain CDR2 of SEQ ID NO: 27 and a light chain CDR3 of SEQ ID NO: 28, or a variant with one or two conservative substitution(s); or
a light chain variable domain comprising at least one of, preferably all three of: a light chain CDR1 of SEQ ID NO: 32, a light chain CDR2 of SEQ ID NO: 33 and a light chain CDR3 of SEQ ID NO: 34, or a variant with one or two conservative substitution(s); or
a light chain variable domain comprising at least one of, preferably all three of: a light chain CDR1 of SEQ ID NO: 38, a light chain CDR2 of SEQ ID NO: 39 and a light chain CDR3 of SEQ ID NO: 40, or a variant with one or two conservative substitution(s); or
a light chain variable domain comprising at least one of, preferably all three of: a light chain CDR1 of SEQ ID NO: 44, a light chain CDR2 of SEQ ID NO: 45 and a light chain CDR3 of SEQ ID NO: 46, or a variant with one or two conservative substitution(s); or
a light chain variable domain comprising at least one of, preferably all three of: a light chain CDR1 of SEQ ID NO: 50, a light chain CDR2 of SEQ ID NO: 51 and a light chain CDR3 of SEQ ID NO: 52, or a variant with one or two conservative substitution(s).

In some particular embodiments, the antibody is a whole antibody or an antigen-binding fragment.

In some embodiments, the antibody, or antigen-binding fragment thereof, further comprises framework regions FR1, FR2, FR3 and FR4 of Cv2.1169, Cv2.1353, Cv2.3194, Cv2.3235 and Cv2.5213 and Cv2.5179 antibodies as defined in Table 4 above or variant thereof as disclosed above. In some embodiments, the antibody comprises heavy and/or light chain variable domain of Cv2.1169, Cv2.1353, Cv2.3194, Cv2.3235 and Cv2.5213 and Cv2.5179 antibodies as defined in Table 1 above or variant thereof as disclosed above. The heavy and/or light chain variable domain of the antibody are preferably associated with IgG or IgA constant region that may be further modified as disclosed above.

In some particular embodiments, the antibody, or antigen-binding fragment thereof, comprises a heavy and/or light chain of Cv2.1169, Cv2.1353, Cv2.3194, Cv2.3235 and Cv2.5213 and Cv2.5179 antibodies as defined in Table 2 above or variant thereof as disclosed above.

In some particular embodiments, the antibody, or antigen-binding fragment thereof, comprises a variable region which is a product of at least one of the following V(D)J recombination events:
(i) V-gene allele IGHV1-58*01 with J-gene allele IGHJ3*02 and V-gene allele IGKV3-20*01 with J-gene allele IGKJ1*01 (e.g. Cv2.5179 and Cv2.1169);
(ii) V-gene allele IGHV3-53*01 with J-gene allele IGHJ6*02 and V-gene allele IGKV1-9*01 with J-gene allele IGKJ3*01 (e.g. Cv2.5213);
(iii) V-gene allele IGHV3-53*01 with J-gene allele IGHJ6*02 and V-gene allele IGKV3-20*01 with J-gene allele IGKJ4*01 (e.g. Cv2.3194);
(iv) V-gene allele IGHV3-66*02 with J-gene allele IGHJ3*02 and V-gene allele IGKV1-9*01 with G-gene allele IGKJ3*01 (e.g. Cv2.1353); or
(v) V-gene allele IGHV3-53*01 with J-gene allele IGHJ6*02 and V-gene allele IGKV1-9*01 with J-gene allele IGKJ5*01 (e.g. Cv2.3235).

In some particular embodiments, the antibody, or antigen-binding fragment thereof, comprises a variable region which is a product of the following V(D)J recombination events:
(i) V-gene allele IGHV1-58*01 with J-gene allele IGHJ3*02; or
(ii) V-gene allele IGKV3-53*01 with J-gene allele IGHJ6*02.

In some particular embodiments, the antibody, or antigen-binding fragment thereof, comprises a variable region which is a product of the following V(D)J recombination events:
(i) V-gene allele IGKV3-20*01 with J-gene allele IGKJ1*01; or
(ii) V-gene allele TGKV3-20*01 with J-gene allele TGKJ4*01.

In some preferred embodiments, the antibody, or antigen-binding fragment thereof, comprises a variable region which is a product of at least one of the following V(D)J recombination events:
(i) V-gene allele IGHV1-58*01 with J-gene allele IGHJ3*02 and V-gene allele IGKV3-20*01 with J-gene allele IGKJ1*01; or
(ii) V-gene allele IGHV3-53*01 with J-gene allele IGHJ6*02 and V-gene allele IGKV3-20*01 with J-gene allele IGKJ4*01.

### Nucleic Acid, Host Cell, Antibody Production

Also disclosed herein are nucleic acid molecule(s) that encode(s) the anti-SARS-CoV-2 antibody of the present disclosure.

Typically, said nucleic acid is recombinant, synthetic or semi-synthetic nucleic acid which is expressible in a host cell suitable for antibody expression or production, in particular human antibody production. The host cell may a cell for recombinant antibody production or a patient cell for antibody production in vivo. Typically, the nucleic acid may be DNA, RNA or mixed molecule, which may further be modified and/or included in any suitable expression vector. As used herein, the terms “vector” and “expression vector” mean the vehicle by which a DNA or RNA sequence (e.g. a foreign gene) can be introduced into a host cell, so as to transform the host and promote expression (e.g. transcription and translation) of the introduced sequence. The recombinant vector can be a vector for eukaryotic or prokaryotic expression, such as a plasmid, a phage for bacterium introduction, a YAC able to transform yeast, a viral vector and especially a retroviral vector, or any expression vector. An expression vector as defined herein is chosen to enable the production of an antibody, either in vitro or in vivo.

So, a further object of the disclosure relates to a vector comprising a nucleic acid as described herein.

Examples of nucleic acid molecules are those encoding the variable light and heavy chain amino acid sequences of the anti-SARS-CoV-2 antibody as disclosed in the previous section, and using the genetic code and, optionally taking into account the codon bias depending on the host cell species.

The nucleic acid molecule or construct sequence is advantageously codon-optimized for expression in a host cell suitable for antibody production in host cell, in particular mammalian cells. Codon optimization is used to improve protein expression level in living organism by increasing translational efficiency of target gene. Appropriate methods and softwares for codon optimization in the desired host are well-known in the art and publically available (see for example the GeneOptimizer software suite in Raab et al., Systems and Synthetic Biology, 2010, 4, (3), 215-225).

The host cell for antibody production may be eukaryote or prokaryote cell. Prokaryote cell is in particular bacteria. Eukaryote cell includes yeast, insect cell and mammalian cell.

Typically, nucleic acid encoding the variable heavy and light chain of Cv2.1169 antibodies comprises or consists of the sequence SEQ ID NO: 93 and SEQ ID NO: 94 respectively, nucleic acid encoding the variable heavy and light chain of Cv2.1353 antibodies comprises or consists of the sequence SEQ ID NO: 95 and SEQ ID NO: 96 respectively, nucleic acid encoding the variable heavy and light chain of Cv2.3194 antibodies comprises or consists of the sequence SEQ ID NO: 97 and SEQ ID NO: 98 respectively, nucleic acid encoding the variable heavy and light chain of Cv2.3235 antibodies comprises or consists of the sequence SEQ ID NO: 99 and SEQ ID NO: 100 respectively and nucleic acid encoding the variable heavy and light chain of Cv2.5213 antibodies comprises or consists of the sequence SEQ ID NO: 101 and SEQ ID NO: 102 respectively, nucleic acid encoding the variable heavy and light chain of Cv2.5179 antibodies comprises or consists of the sequence SEQ ID NO: 156 and SEQ ID NO: 157 respectively.

Nucleic acids encoding anti-SARS-CoV-2 antibody of the disclosure with nucleotide sequences having at least 80%, for example, at least 85%, 90%, 95%, 96%, 97%, 98%, or 99% identity to any one of the above defined nucleotides sequences are also part of the present disclosure.

The present disclosure also pertains to nucleic acid molecules that derive from the latter sequences having been optimized for protein expression in host cells, in particular eukaryotic cells, preferably mammalian cells, for example, CHO or HEK cell lines or human cells.

In some embodiments, said nucleic acid molecule is a eukaryotic, preferably mammalian, expression cassette, wherein the antibody coding sequence(s) is operably linked to appropriate regulatory sequence(s) for their expression in an antibody producing cell or a patient cell. Such sequences which are well-known in the art include in particular a promoter, and further regulatory sequences capable of further controlling the expression of a transgene, such as without limitation, enhancer, terminator and intron. The promoter may be a tissue-specific, ubiquitous, constitutive or inducible promoter that is functional in the antibody producing cell. Such promoters are well-known in the art and their sequences are available in public sequence data bases.

In some particular embodiments, the nucleic acid is RNA, preferably mRNA, wherein the coding sequence of the antibody light and/or heavy chain is operably linked to appropriate regulatory sequence(s) for their expression in an individual's target cells or tissue(s). mRNA therapy is well-known in the art. mRNA is delivered into the host cell cytoplasm where expression generates the therapeutic protein of interest. mRNA construct comprises a cap structure, 5′ and 3′untranslated regions (UTRs), and open reading frame (ORF), and a 3′poly(A) tail. mRNA construct may be non-replicating mRNA (MRM) or self-amplifying mRNA (SAM). SAM comprises the inclusion of genetic replication machinery derived from positive-strand mRNA viruses, most commonly alphaviruses such as Sindbis and Semliki-Forest viruses. In SAM constructs, the ORF encoding viral structural protein is replaced by the transcript encoding the therapeutic protein of interest, and the viral RNA-dependent RNA polymerase is retained to direct cytoplasmic amplification of the replicon construct. Trans-replicating RNA are disclosed for example in WO 2017/162461. RNA replicon from alphavirus suitable for gene expression are disclosed in WO 2017/162460. mRNA manufacturing process uses plasmid DNA (pDNA) containing a DNA-dependent RNA polymerase promoter, such as T7, and the corresponding sequence for the mRNA construct. The pDNA is linearized to serve as a template for the DNA-dependent RNA polymerase to transcribe the mRNA, and subsequently degraded by a DNase process step. The addition of the 5′cap and the 3′poly(A) tail can be achieved during the in vitro transcription step or enzymatically after transcription. Enzymatic addition of the cap can be accomplished by using guanylyl transferase and 2′-O-methyltransferase to yield a Cap0(^{N7Me}GpppN) or Cap1 (N^{7Me}GpppN^{2′-oMe}) structure, respectively, while the poly-A tail can be achieved through enzymatic addition via poly-A polymerase. mRNA is then purified using standard methods suitable for mRNA purification such as high-pressure liquid chromatography (HPLC) and others. Methods for producing mRNA are disclosed for example in WO 2017/182524.

To improve translation efficiency in treated subject cells, the mRNA according to the invention comprises a sequence which is codon-optimized for expression in human. Further improvements of the mRNA construct according to the invention to improve its stability and translation efficiency in vivo include optimization the length and regulatory element sequences of 5′-UTR and 3′UTR; base and/or sugar modifications in the cap structure to increase ribosomal interaction and/or mRNA stability; and modified nucleosides. Modified nucleosides may be in the 5′-UTR, 3′-UTR or ORF. Examples of modified nucleosides include pseudouridine and N-1-methylpseudouridine that remove intracellular signalling triggers for protein kinase R activation. Examples of modified nucleosides that reduce RNA degradation into cells are disclosed in WO 2013/039857. Modified cap structures are disclosed in WO 2011/015347 and WO 2019/175356. Optimized 3′-UTR sequences are disclosed in WO 2017/059902. Modified polyA sequences which improve RNA stability and translation efficiency are disclosed in US 2020/0392518. Modified mRNA with improved stability and translation efficiency are also disclosed in WO 2007/036366.

The invention may use any vector suitable for the delivery and expression of nucleic acid into individual's cells, in particular suitable for nucleic acid therapy. Such vectors that are well-known in the art include viral and non-viral vectors. Non-viral vector includes the various (non-viral) agents which are commonly used to either introduce or maintain nucleic acid into individual's cells. Agents which are used to introduce nucleic acid into individual's cells by various means include in particular polymer-based, particle-based, lipid-based, peptide-based delivery vehicles or combinations thereof, such as with no limitations cationic polymer, dendrimer, micelle, liposome, lipopolyplex, exosome, microparticle and nanoparticle including lipid nanoparticle (LNP) and viral-like particles; and cell penetrating peptides (CPP). Agents which are used to maintain nucleic acid into individual's cells include in particular naked nucleic acid vectors such as plasmids, transposons and mini-circles. Viral vectors are by nature capable of penetrating into cells and delivering nucleic acid(s) of interest into cells, according to a process named as viral transduction. As used herein, the term “viral vector” refers to a non-replicating, non-pathogenic virus engineered for the delivery of genetic material into cells. In viral vectors, viral genes essential for replication and virulence are replaced with an expression cassette for the transgene of interest. Thus, the viral vector genome comprises the transgene expression cassette flanked by the viral sequences required for viral vector production. As used herein, the term “recombinant virus” refers to a virus, in particular a viral vector, produced by standard recombinant DNA technology techniques that are known in the art. As used herein, the term “virus particle” or “viral particle” is intended to mean the extracellular form of a non-pathogenic virus, in particular a viral vector, composed of genetic material made from either DNA or RNA surrounded by a protein coat, called the capsid, and in some cases an envelope derived from portions of host cell membranes and including viral glycoproteins. As used herein, a viral vector refers to a viral vector particle. These vectors have minimal eukaryotic sequences to minimize the possibility of chromosomal integration. In addition, these approaches can advantageously be combined to introduce and maintain the nucleic acid of the invention into individual's cells.

In some embodiments, a mRNA according to the present invention as disclosed above is combined with a nucleic-acid delivery agent suitable for delivery of mRNA into mammalian host cells that are well-known in the art. The mRNA delivery agent may be a polymeric carrier, polycationic protein or peptide, lipid nanoparticle or other. For example, the mRNA (non-replicating or self-amplifying) may be delivered into cells using polymers, in particular cationic polymers, such as polyethylenimine (PEI), poly-L-Lysin (PEL), polyvinylamine (PVA) or polyallylamine (PAA), wherein the mRNA is preferentially present in the form of monomers, dimers, trimers or oligomers as disclosed in WO 2021/001417. Alternatively, the mRNA may be combined with polyalkyleneimine in the form of polyplex particles, suitable for intramuscular administration as disclosed in WO 2019/137999 or WO 2018/011406. The mRNA may also be combined with a polycation, in particular protamine, as disclosed in WO 2016/000792. One or more mRNA molecules may be formulated within a cationic lipid nanoparticle (LNP); for example the formulation may comprise 20-60% cationic lipid; 5-25% non-cationic lipid, 25-55% sterol and 0.5-15% PEG-modified lipid as disclosed WO 2015/164674. The mRNA may also be formulated in RNA decorated particles such as RNA decorated lipid particles, preferably RNA decorated liposomes as disclosed in WO 2015/043613.

In particular embodiments, the vector is a particle or vesicle, in particular lipid-based micro- or nano-vesicle or particle such as liposome or lipid nanoparticle (LNP). In more particular embodiments, the nucleic acid is RNA, in particular mRNA and the vector is a particle or vesicle, in particular LNP as described above. The LNP: mRNA mass ratio can be around 10:1 to 30:1.

In other embodiments, the nucleic acid is DNA, preferably included in an expression vector such as plasmid or viral vector. The invention also relates to a vector comprising the nucleic acid according to the present disclosure. Preferably, the vector is a recombinant integrating or non-integrating viral vector. Examples of recombinant viral vectors include, but not limited to, vectors derived from retrovirus, adenovirus, adeno-associated virus (AAV), herpes virus, poxvirus, and other virus. Retrovirus includes in particular lentivirus vector such as human immunodeficiency virus, including HIV type 1 (HIV-1) and HIV type 2 (HIV-2) vectors.

In some preferred embodiments, the expression vector comprises a pair of nucleic acid sequences selected from: a sequence having at least 90% identity with SEQ ID NO: 93 and a sequence having at least 90% identity with SEQ ID NO: 94; a sequence having at least 90% identity with SEQ ID NO: 95 and a sequence having at least 90% identity with SEQ ID NO: 96; a sequence having at least 90% identity with SEQ ID NO: 97 and a sequence having at least 90% identity with SEQ ID NO: 98; a sequence having at least 90% identity with SEQ ID NO: 99 and a sequence having at least 90% identity with SEQ ID NO: 100; and a sequence having at least 90% identity with SEQ ID NO: 101 and a sequence having at least 90% identity with SEQ ID NO: 102.

In some more preferred embodiments, the recombinant vector for expression of the antibody of the present disclosure is plasmid Cv2.1169_pIgH encoding Cv2.1169 antibody heavy chain in expressible form, particularly as contained in the E. coli bacteria (DH10B, C3019, NEB) transformed with Cv2.1169_pIgH. These bacteria Cv2.1169_pIgH were deposited under the terms of the Budapest Treaty at the Collection Nationale de Cultures de Microorganismes (CNCM) at the Institut Pasteur, 25 rue du Docteur Roux, 75724 Paris, FR, on Jan. 28, 2021 under the number I-5651.

In some preferred embodiments, the recombinant vector for expression of the antibody of the present disclosure is plasmid Cv2.1169_pIgL encoding Cv2.1169 antibody light chain in expressible form, particularly as contained in the E. coli bacteria (DH10B, C3019, NEB) transformed with Cv2.1169_pIgL. These bacteria Cv2.1169_pIgL were deposited under the terms of the Budapest Treaty at the Collection Nationale de Cultures de Microorganismes (CNCM) at the Institut Pasteur, 25 rue du Docteur Roux, 75724 Paris, FR, on Jan. 28, 2021 under the number I-5652.

In some preferred embodiments, the recombinant vector for expression of the antibody of the present disclosure is plasmid Cv2.1353_pIgH encoding Cv2.1353 antibody heavy chain in expressible form, particularly as contained in the E. coli bacteria (DH10B, C3019, NEB) transformed with Cv2.1353_pIgH. These bacteria Cv2.1353_IgH were deposited under the terms of the Budapest Treaty at the Collection Nationale de Cultures de Microorganismes (CNCM) at the Institut Pasteur, 25 rue du Docteur Roux, 75724 Paris, FR, on Apr. 2, 2021 under the number I-5668.

In some preferred embodiments, the recombinant vector for expression of the antibody of the present disclosure is plasmid Cv2.1353_pIgL encoding Cv2.1353 antibody light chain in expressible form, particularly as contained in the E. coli bacteria (DH10B, C3019, NEB) transformed with Cv2.1353_pIgL. These bacteria Cv2.1353_IgL were deposited under the terms of the Budapest Treaty at the Collection Nationale de Cultures de Microorganismes (CNCM) at the Institut Pasteur, 25 rue du Docteur Roux, 75724 Paris, FR, on Apr. 2, 2021 under the number I-5669.

In some preferred embodiments, the recombinant vector for expression of the antibody of the present disclosure is plasmid Cv2.3194_pIgH encoding Cv2.3194 antibody heavy chain in expressible form, particularly as contained in the E. coli bacteria (DH10B, C3019, NEB) transformed with Cv2.3194_pIgH. These bacteria Cv2.3194_IgH were deposited under the terms of the Budapest Treaty at the Collection Nationale de Cultures de Microorganismes (CNCM) at the Institut Pasteur, 25 rue du Docteur Roux, 75724 Paris, FR, on Apr. 2, 2021 under the number I-5670.

In some preferred embodiments, the recombinant vector for expression of the antibody of the present disclosure is plasmid Cv2.3194_pIgL encoding Cv2.3194 antibody light chain in expressible form, particularly as contained in the E. coli bacteria (DH10B, C3019, NEB) transformed with Cv2.3194_pIgL. These bacteria Cv2.3194_IgL were deposited under the terms of the Budapest Treaty at the Collection Nationale de Cultures de Microorganismes (CNCM) at the Institut Pasteur, 25 rue du Docteur Roux, 75724 Paris, FR, on Apr. 2, 2021 under the number I-5671.

In some preferred embodiments, the recombinant vector for expression of the antibody of the present disclosure is plasmid Cv2.3235_pIgH encoding Cv2.3235 antibody heavy chain in expressible form, particularly as contained in the E. coli bacteria (DH10B, C3019, NEB) transformed with Cv2.3235_pIgH. These bacteria Cv2.3235_IgH were deposited under the terms of the Budapest Treaty at the Collection Nationale de Cultures de Microorganismes (CNCM) at the Institut Pasteur, 25 rue du Docteur Roux, 75724 Paris, FR, on Apr. 2, 2021 under the number I-5672.

In some preferred embodiments, the recombinant vector for expression of the antibody of the present disclosure is plasmid Cv2.3235_pIgL encoding Cv2.3235 antibody light chain in expressible form, particularly as contained in the E. coli bacteria (DH10B, C3019, NEB) transformed with Cv2.3235_pIgL. These bacteria Cv2.3235_IgL were deposited under the terms of the Budapest Treaty at the Collection Nationale de Cultures de Microorganismes (CNCM) at the Institut Pasteur, 25 rue du Docteur Roux, 75724 Paris, FR, on Apr. 2, 2021 under the number I-5673.

In some preferred embodiments, the recombinant vector for expression of the antibody of the present disclosure is plasmid Cv2.5213_pIgH encoding Cv2.5213 antibody heavy chain in expressible form, particularly as contained in the E. coli bacteria (DH10B, C3019, NEB) transformed with Cv2.5213_pIgH. These bacteria Cv2.5213_IgH were deposited under the terms of the Budapest Treaty at the Collection Nationale de Cultures de Microorganismes (CNCM) at the Institut Pasteur, 25 rue du Docteur Roux, 75724 Paris, FR, on Apr. 2, 2021 under the number I-5674.

In some preferred embodiments, the recombinant vector for expression of the antibody of the present disclosure is plasmid Cv2.5213_pIgL encoding Cv2.5213 antibody light chain in expressible form, particularly as contained in the E. coli bacteria (DH10B, C3019, NEB) transformed with Cv2.5213_pIgL. These bacteria Cv2.5213_IgL were deposited under the terms of the Budapest Treaty at the Collection Nationale de Cultures de Microorganismes (CNCM) at the Institut Pasteur, 25 rue du Docteur Roux, 75724 Paris, FR, on Apr. 2, 2021 under the number I-5675.

The polynucleotide according to the disclosure is prepared by the conventional methods known in the art. For example, it is produced by amplification of a nucleic sequence by PCR or RT-PCR, by screening genomic DNA libraries by hybridization with a homologous probe, or else by total or partial chemical synthesis. The recombinant vectors are constructed and introduced into host cells by the conventional recombinant DNA and genetic engineering techniques, which are known in the art.

A further object of the present disclosure relates to a host cell which has been transfected, infected or transformed by a nucleic acid and/or a vector according to the invention. As used herein, the term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. The transformation may be transient or stable over time. Stable transformation may be by integration of the nucleic acid into the host cell genome. A host cell that receives and expresses introduced DNA or RNA has been “transformed”.

Said host cells may be prokaryotic cells such as bacteria or eukaryotic cells such as yeasts, insect cells or mammalian cells. Mammalian cells may be simian, human, dog and rodent cells. Mammalian host cells for expressing the antibodies of the disclosure include in particular Chinese Hamster Ovary (CHO cells) including dhfr-CHO cells (described in Urlaub and Chasin, 1980) used with a DHFR selectable marker (as described in Kaufman and Sharp, 1982), CHOK1 dhfr+ cell lines, NSO myeloma cells, COS cells and SP2 cells, for example GS CHO cell lines together with GS Xceed™ gene expression system (Lonza), HEK-293 cells (ATCC CRL-1573). In a preferred embodiment, said host cells are CHO cells, or HEK-293.

The polynucleotide, vector or cell of the disclosure are useful for the production of the protein of the invention using well-known recombinant DNA techniques. The polynucleotide or vector are also useful for nucleic acid therapy as disclosed below.

Antibodies of the present disclosure can be produced in a host cell transfectoma using, for example, a combination of recombinant DNA techniques and gene transfection methods as is well known in the art (Morrison, 1985). For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. When recombinant expression vectors encoding antibody genes are introduced into host cells, in particular eukaryotic cells such as mammalian cells, the antibodies are produced by culturing the host cells for a period of time sufficient for expression of the antibody in the host cells and, optionally, secretion of the antibody into the culture medium in which the host cells are grown. Antibodies can be recovered and purified for example from the culture medium after their secretion using standard protein purification methods (Shukla et al., 2007).

### Pharmaceutical Composition and Therapeutic Use

In another aspect, the present disclosure provides a composition, e.g., a pharmaceutical composition, containing an antibody, antigen-binding fragment, nucleic acid or vector disclosed herein, formulated together with at least one of a pharmaceutically acceptable carrier, an adjuvant, and a preservative. For example, the composition comprises an antibody selected from the group consisting of Cv2.1169, Cv2.1353, Cv2.3194, Cv2.3235, Cv2.5179 and Cv2.5213, their antigen-binding fragments or nucleic acid or vector encoding said antibody or antigen-binding fragment as disclosed herein.

In some embodiments, the nucleic acid is mRNA, preferably modified mRNA as disclosed herein; the mRNA including modified mRNA is advantageously formulated in a particle or vesicle, in particular LNP, as disclosed herein.

As used herein, the term “pharmaceutically acceptable” means approved by a regulatory agency or recognized pharmacopeia such as European Pharmacopeia, for use in animals and/or humans. The term “excipient” refers to a diluent, adjuvant, carrier, or vehicle with which the therapeutic agent is administered.

Any suitable pharmaceutically acceptable carrier, diluent or excipient can be used in the preparation of a pharmaceutical composition (See e.g., Remington: The Science and Practice of Pharmacy, Alfonso R. Gennaro (Editor) Mack Publishing Company, April 1997). Pharmaceutical compositions are typically sterile and stable under the conditions of manufacture and storage. Pharmaceutical compositions may be formulated as solutions (e.g. saline, dextrose solution, or buffered solution, or other pharmaceutically acceptable sterile fluids), microemulsions, liposomes, or other ordered structure suitable to accommodate a high product concentration (e.g. microparticles or nanoparticles). The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition.

In some embodiments, the pharmaceutical composition is for systemic, local or systemic combined with local administration. Parenteral pharmaceutical composition includes a composition suitable for intravenous, intraarterial, subcutaneous, intraperitoneal or intramuscular administration. Local administration is preferably respiratory such as by nasal administration, inhalation, insufflation. or broncho-alveolar lavage. The administration may be parenteral injection or infusion, local delivery, or inhalation or sustained delivery. Preferably, the administration is by injection, inhalation, or injection combined with inhalation. Preferably the injection is intravenous, subcutaneous or intramuscular. The inhalation is advantageously done by nebulisation.

In some embodiments the pharmaceutical composition comprises a preservative. Non-limiting examples of preservatives are anti-oxidation agents and anti-bacterial agents. In some embodiments the preservative is present at a known concentration. The presence of a preservative in the composition distinguishes the composition from any composition that occurs in nature. It also imbues the composition with unique functions that are not present in any naturally occurring composition, such as in certain embodiments the ability to be used therapeutically under certain conditions in which the preservative is useful.

In some embodiments the pharmaceutical composition comprises a defined concentration of a recombinant human antibody of this invention. Such compositions do not occur naturally and have a different structure than any naturally occurring composition. The known concentration of the recombinant antibody imbues the compositions with unique functions that are not present in any naturally occurring composition, such as in certain embodiments the ability to be used therapeutically under certain conditions in which the defined concentration of the recombinant antibody is useful.

In some embodiments, a pharmaceutical composition comprising IgA, in particular polymeric or secretory IgA as disclosed herein is used for mucosal application, in particular to the respiratory tract, preferably by nebulisation or inhalation. Pharmaceutical composition comprising IgA, in particular polymeric (e.g., J-chain dimerization of IgA) or secretory IgA are preferred as prophylactic treatment, to prevent SARS-CoV-2 infection.

In some embodiments, a pharmaceutical composition comprising IgG, preferably IgG1, is used for injection, in particular intravenous, subcutaneous or intramuscular.

These pharmaceutical compositions are exemplary only and do not limit the pharmaceutical compositions suitable for other parenteral and non-parenteral administration routes. The pharmaceutical compositions described herein can be packaged in single unit dosage or in multidosage forms.

Preferably, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. Sustained release formulations, such as PLA or PLGA or other polymers, for inhalation or injection can be used.

In certain aspects, the disclosure relates to an antibody, antigen-binding fragment thereof, or pharmaceutical composition according to any one of the preceding embodiments, for use as a medicament.

In certain aspects, the disclosure provides the therapeutic use of an antibody, antigen-binding fragment thereof or a composition according to any one of the preceding embodiments, preferably for treating, preventing or alleviating the symptoms of a SARS-CoV-2-associated or -mediated disorder in a subject in need thereof.

The term “subject” or “patient” as used herein, refers to mammals. Mammalian species that can benefit from the disclosed methods of treatment include, but are not limited to, humans, non-human primates such as apes, chimpanzees, monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease, such as according to the present disclosure the reduction of the viral burden and/or levels of inflammation in the lungs. In other embodiments, this term refers to minimizing the spread or worsening of the disease resulting from the administration of one or more therapeutic agents to a subject with such a disease.

In a further aspect, the disclosure relates to a method of treating and/or reducing the risk of developing SARS-CoV-2-associated disorder, in a subject in need thereof that comprises administering to the subject a therapeutically effective amount of an antibody, an antigen-binding fragment thereof, a nucleic acid or vector, or a pharmaceutical composition as described above.

In some embodiments, the subject is not infected with SARS-CoV-2 and the treatment is a prophylactic treatment.

In some particular embodiments, the method is a method of reducing the risk of developing a SARS-CoV-2-associated COVID-19 disease, wherein the risk of hospitalization or the risk of death is reduced by the treatment. In some particular embodiments, the reduction of the risk of developing a SARS-CoV-2 associated COVID-19 disease lasts at least 3 or 4 months, preferably 5 or 6 months, more preferably 7 to 9 months, after administration to the subject of a therapeutically effective amount of an antibody, an antigen-binding fragment thereof, a nucleic acid or vector, or a pharmaceutical composition as described above.

In some embodiments, the subject is a COVID-19 patient and the treatment is a curative treatment.

In some particular embodiments, the method is a method of treating SARS-CoV-2-associated COVID-19 disease, wherein the likelihood of developing severe disease is reduced by the treatment; wherein the likelihood of hospitalization is reduced by the treatment; wherein the subject is hospitalized.

In some particular embodiments, the method is a method of treating SARS-CoV-2-associated COVID-19 disease, wherein the subject is at risk of developing a SARS, more particularly a subject with concurrent underlying conditions such as obesity, diabetes, cancer, under immunosuppressive therapy, primary immune deficiency or unresponsive to vaccines. Non-limiting examples of subjects with concurrent underlying conditions include a subject receiving anti-CD20 antibody therapy, a subject having a lymphoid hemopathy, a solid organ transplant recipient or an allogeneic hematopoietic stem cell transplant recipient.

In the context of the invention, an “effective amount” means a therapeutically effective amount. As used herein a “therapeutically effective amount” refers to an amount effective, at dosages and for periods of time necessary to achieve the desired therapeutic result, such as prophylaxis, or treatment of SARS-CoV-2-infection and in particular the reduction of the viral burden and/or levels of inflammation in the lungs. The therapeutically effective amount of the product of the invention, or pharmaceutical composition that comprises it may vary according to factors such as the disease state, age, sex, and weight of the individual, and the ability of the product or pharmaceutical composition to elicit a desired response in the individual. Dosage regimens may be adjusted to provide the optimum therapeutic response. A therapeutically effective amount is also typically one in which any toxic or detrimental effect of the product or pharmaceutical composition is outweighed by the therapeutically beneficial effects.

The product of the disclosure will be typically included in a pharmaceutical composition or medicament, optionally in combination with a pharmaceutical carrier, diluent and/or adjuvant. Such composition or medicinal product comprises the product of the disclosure in an effective amount, sufficient to provide a desired therapeutic effect, and a pharmaceutically acceptable carrier or excipient.

In one embodiment the antibody or antigen-binding fragment or the pharmaceutical composition for its therapeutic use is administered to the subject or patient by a parenteral route, in particularly by intravenous, intraarterial, subcutaneous, intraperitoneal, or intramuscular route. In another particular embodiment, the antibody or antigen-binding fragment or the pharmaceutical composition for its therapeutic use is administered to the subject or patient by inhalation.

The amount of product of the invention that is administered to the subject or patient may vary depending on the particular circumstances of the individual subject or patient including, age, sex, and weight of the individual; the nature and stage of the disease, the aggressiveness of the disease; the route of administration; and/or concomitant medication that has been prescribed to the subject or patient. Dosage regimens may be adjusted to provide the optimum therapeutic response.

For any particular subject, specific dosage regimens may be adjusted over time according to the individual needs and the professional judgment of the person administering or supervising the administration of the compositions. Dosage ranges set forth herein are exemplary only and do not limit the dosage ranges that may be selected by medical practitioners.

In one embodiment, the antibody or antigen-binding fragment according to the disclosure can be administered to the subject or patient for the treatment of SARS-CoV-2 associated disease in an amount or dose comprised within a range of 2.5 mg/kg to 40 mg/kg (kg: subject's or patient's body weight). In a more particular embodiment, the antibody or antigen-binding fragment is administered in an amount comprised within a range of 5 to 30 mg/kg. In a more particular embodiment, the antibody or antigen-binding fragment is administered in an amount comprised within a range of 8.5 to 28.5 mg/kg for a person weighing 70 kg. In a more particular embodiment, the antibody or antigen-binding fragment is administered at a dosage of at least 5 mg/kg, preferably 10 mg/kg, more preferably 15 mg/kg, and more preferably 30 mg/kg.

In some embodiments, the pharmaceutical composition is included in a kit that may further comprise instructions or packaging materials that describe how to administer the product contained within the kit to a patient. Containers of the kit can be of any suitable material, e.g., glass, plastic, metal, etc., and of any suitable size, shape, or configuration. In certain embodiments, the kits may include one or more ampoules or syringes that contain the products of the invention in a suitable liquid or solution form.

Another aspect of the invention relates to a medical device, comprising the pharmaceutical composition according to the present disclosure. The medical device is in a form suitable for the administration of the composition. In some embodiments, the medical device is suitable for the injection of the composition; said medical device is advantageously chosen from a syringe, infusion bag, injection port, and others that are well-known in the art. In other embodiments, the medical device is suitable for the respiratory tract administration of the composition; said medical device is advantageously chosen from an inhaler, a nebulizer, such as small-volume nebulizer, and others that are well-known in the art.

Another aspect of the invention relates to the use of a pharmaceutical composition according to the present disclosure for the manufacture of a medicament for the prevention or treatment of SARS-CoV-2 infection and associated COVID-19 disease.

### Diagnostic Reagents, Kits and Methods

The antibodies or fragment thereof comprising the antigen-binding site according to the present disclosure are specific for SARS-CoV-2 and in particular do not cross-react with with other coronavirus including human pathogenic betacoronavirus (group B/C) SARS-CoV-1 and MERS-CoV; alphacoronavirus NL63-CoV and 229E-CoV and betacoronavirus group A HKU1-CoV (Table 5). Therefore, they are useful as reagent for the detection of SARS-CoV-2 infection or contamination in various samples, including in particular biological or environmental samples.

The sample is any sample suspected of containing SARS-CoV-2 such as in particular biological or environmental samples. A biological sample may be any tissue, body fluid or stool. Non-limiting examples of body fluids include whole-blood, serum, plasma, urine, cerebral spinal fluid (CSF), and mucosal secretions, such as with no limitations oral and respiratory tract secretions (sputa, saliva and the like). Samples include swabs such as oral or nasopharyngeal (NP) swabs, aspirate, wash or lavage. Samples for diagnostic tests for SARS-CoV-2 can be taken from the upper (nasopharyngeal/oropharyngeal swabs, nasal aspirate, nasal wash or saliva) or lower respiratory tract (sputum or tracheal aspirate or bronchoalveolar lavage (BAL). Preferred biological samples include nasopharyngeal swab and saliva sample. Samples also include environmental samples that may contain SARS-CoV-2 such as air, water, soil, food, beverages, feed, water (e.g., fresh water, salt water, waste water, and drinking water), sewage, sludge, environmental surfaces and others. The environmental surface sample is for example a surface swab or swipe.

The detection or diagnosis is performed by immunoassay technique which is well-known in the art and rely on the detection of antigen-antibody complexes using an appropriate label. The method of the invention may use any immunoassay such as with no limitations, immunoblotting, immunoprecipitation, ELISA, immunocytochemistry or immunohistochemistry, and immunofluorescence like flow cytometry assay, and FACS. Flow cytometry, also known as flow virometry, nanoscale flow cytometry or simply small-particle flow cytometry is a rapid, high-throughput, and effective method to quantify intact viral particles released by an infected cell.

The invention encompasses a method for the detection of a SARS-CoV-2 in a sample comprising: contacting said sample with an antibody according to the present disclosure and detecting the antigen-antibody complexes formed.

The method of the invention may use any appropriate label used in immunoassays such as enzymes, chemiluminescent, fluorescent dyes/proteins or radioactive agents, or others. The label may be on the antibody or fragment thereof which binds to the antigen or on a binding-partner such as secondary antibody or avidin/streptavidin conjugated to a label.

The antibody is preferably labelled, in the form of a conjugate or fusion protein, and the antigen-antibody complexes are detected by measuring the signal from the label by any appropriate means available for that purpose as disclosed above.

In some embodiments, antigen detection is performed by ELISA, lateral flow immunoassay, or bead-based immunoassay.

The detection step may be qualitative or semi-quantitative, and may comprise detecting the presence or level of viral antigen in the sample. In some embodiments, the detecting step comprises the determination of the amount of bound antigen in the mixture, and optionally, comparing the amount of bound antigen in the mixture with at least one predetermined value.

The detection of the presence or level of viral antigen in a biological sample from an individual using the methods of the invention is indicative of whether the individual is suffering from SARS-CoV-2 infection or associated COVID-19 disease.

Therefore, the above method of the invention is useful for the diagnosis of SARS-CoV-2 infection or associated COVID-19 disease in an individual, as well as monitoring of treatment in a COVID-19 patient.

The treatment may be an antiviral treatment or immunotherapeutic treatment using SARS-CoV-2 neutralizing antibodies.

In some embodiments, the above method is a method of diagnosis comprising the step of deducing therefrom whether the individual is suffering from SARS-CoV-2 infection or associated disease.

In some embodiments, the above method is a method of monitoring of treatment in a COVID-19 patient, comprising the step of deducing therefrom whether the treatment is efficient is not. Treatment efficacy is determined by a decrease of viral antigen level compared to previous viral antigen level determined in the patient, before treatment or during the treatment course.

In some embodiments in connection with this aspect of the invention, the above method of diagnosis comprises a further step of administering an appropriate treatment to the individual depending on whether or not the individual is diagnosed with SARS-CoV-2 virus infection and in particular COVID-19 associated disease.

In some embodiments in connection with this aspect of the invention, the above method of monitoring of treatment in a COVID-19 patient, comprises a further step of modifying the COVID-19 treatment when said treatment is determined as not being efficient in the patient.

In another aspect, the disclosure further relates to a kit for the detection or diagnosis of SARS-CoV-2 infection or contamination, comprising at least an antibody or antigen-binding fragment thereof, preferably further including a detectable label. The kit optionally comprises reagents for the detection of the antigen/antibody complex. Reagents available for this purpose are well-known in the art and include with no limitation buffers, secondary antibody conjugated to a label, avidin/streptavidin conjugated to a label. In some preferred embodiments of the kit of the invention, the antibody, and optional reagents are in lyophilised form to allow ambient storage. The components of the kits are packaged together into any of the various containers suitable for antigen/antibody complex detection such as plates, slides, wells, dishes, beads, particles, cups, strands, chips, strips and others. The kit optionally includes instructions for performing at least one specific embodiment of the method of the invention. In some advantageous embodiments, the kit comprises micro-well plates or microtubes, preferably in a dried format, i.e., wherein the wells of the plates or microtubes comprise a dried composition containing at least the antibody, and preferably further comprising all the reagents for the detection of antigen/antibody complex. In some other advantageous embodiments, the antibody and optional reagents are included into any of the devices available for immunoassay.

The practice of the present invention will employ, unless otherwise indicated, conventional techniques, which are within the skill of the art. Such techniques are explained fully in the literature.

The invention will now be exemplified with the following examples, which are not limitative, with reference to the attached drawings in which:

### FIGURE LEGENDS

FIG. 1. (Wuhan) SARS-CoV-2 spike-specific memory antibodies cloned from convalescent COVID-19 individuals.
(A) Dot plots showing the IgG antibody binding to SARS-CoV-2 tri-S as area under the curve (AUC) values determined by ELISA with serially-diluted sera from convalescent COVID-19 individuals in the CORSER (n=212; two timepoints t1 and t2) and French COVID-19 cohorts (n=159; with a follow-up overtime for some samples). All dots show selected samples tested in (B) and (C), respectively.
(B) Heatmap showing the IgG, IgG subclass and IgA seroreactivity of selected convalescent COVID-19 individuals from the CORSER (n=8) and French COVID-19 (n=34) cohorts against SARS-CoV-2 tri-S and RBD proteins as measured in FIG. 2B. Samples were also tested against MERS tri-S to assay for cross-reactivity against another β-coronavirus.
(C) Heatmap showing the antibody binding of serum IgG and IgA antibodies purified from selected convalescent donors against SARS-CoV-2 antigens and trimeric spike proteins from other coronaviruses (α-coronaviruses; β-coronaviruses) as measured in FIGS. 2D and 2E. RBD, receptor binding domain; FP, fusion peptide.
(D) Graph showing the in vitro SARS-CoV-2 neutralizing activity of purified serum IgG and IgA antibodies from selected COVID-19 convalescents (top). Calculated IC₅₀ values are presented in the heatmap on the bottom.
(E) Flow-cytometric plots showing the SARS-CoV-2 S-binding IgG⁺ and IgA⁺ memory B cells in the blood from convalescent donors. Flow-cytometric histograms in the upper left-hand corner show the proportion of RBD⁺ cells among SARS-CoV-2 S-binding IgG⁺ and IgA⁺ memory B lymphocytes.
(F) Bubble plots showing the reactivity of human monoclonal IgG antibodies cloned from SARS-CoV-2 S-binding IgG⁺ and IgA⁺ memory B cells of convalescent donors against SARS-CoV-2 S protein as measured by S-Flow (Y axis), tri-S ELISA (X axis) and tri-S-capture ELISA (bubble size). For each donor, the pie chart shows the proportion of SARS-CoV-2 S-specific antibodies from cloned antibodies (top; total number indicated in the pie chart center) and the number (n) of variants in each SARS-CoV-2 S-specific B-cell clonal family.

FIG. 2. (Wuhan) SARS-CoV-2 reactivity of sera, purified polyclonal and monoclonal antibodies from COVID-19 convalescents.
(A) Graph comparing the single-dilution OD measurements (1:400) (X-axis) and AUC values (Y-axis) measured with serially-diluted sera from convalescent COVID-19 individuals in the CORSER (n=212) and French COVID-19 cohorts (n=159), and pre-epidemic donors (n=100) for the ELISA IgG antibody binding to SARS-CoV-2 tri-S as previously reported (2).
(B) ELISA graphs showing the reactivity of serum IgG and IgA antibodies from selected convalescent COVID-19 individuals in the CORSER (n=8) and French COVID-19 (n=34) cohorts against SARS-CoV-2 tri-S and RBD proteins. Samples were also tested against MERS tri-S to assay for cross-reactivity against another β-coronavirus. Means of duplicate values are shown. DF, dilution factor.
(C) Correlation plots comparing the AUC binding values of serum IgG and IgA antibodies to SARS-CoV-2 tri-S, MERS-CoV tri-S and RBD proteins as determined in (A). p values were calculated using two-tailed Pearson correlation test.
(D) ELISA graphs showing the reactivity of purified IgG and IgA serum antibodies from selected donors (n=10) against SARS-CoV-2 protein and protein sub-units. Means of duplicate values are shown.
(E) Same as in (D) but for trimeric spike proteins from other coronaviruses.
(F) ELISA graphs showing the reactivity of antibodies cloned from SARS-CoV-2 S-captured memory B cells (n=133) against the SARS-CoV-2 tri-S protein. Means of duplicate values are shown.

FIG. 3. Binding and neutralizing properties of potent anti-RBD antibody neutralizers—Related to FIGS. 12 and 13.
(A) SPR sensorgrams comparing the relative affinity of neutralizing anti-RBD IgG antibodies for the binding to (Wuhan) SARS-CoV-2 S trimers, S1 and RBD proteins. Calculated K_{D} values are indicated at the bottom.
(B) Competition ELISA graphs (left) comparing the IgG binding to (Wuhan) SARS-CoV-2 tri-S(top) and RBD (bottom) of selected biotinylated anti-RBD antibodies in presence of Cv2.1169 as potential competitor. Error bars indicate the SD of duplicate values. Heatmaps (right) showing the competition of selected anti-RBD antibodies for tri-S and RBD binding as measured in FIG. 4D. Black colors indicate stronger inhibition by competitors; grey colors indicate low or no competition.
(C) Competition ELISA graphs showing the binding of biotinylated (Wuhan) SARS-CoV-2 tri-S protein to the immobilized soluble ACE2 ectodomain in presence of anti-RBD antibodies as competitors. Error bars indicate the SD of duplicate values
(D) Graphs showing the neutralization curves of (Wuhan) SARS-CoV-2 by selected anti-RBD IgG antibodies as determined with the pseudo-neutralization (top) and S-Fuse neutralization (bottom) assays. Error bars indicate the SD of assay triplicates. IC₅₀ values are indicated in the top left-hand corner.
(E) Heatmap comparing the binding of RBD-specific IgG antibodies to the cell-expressed spike proteins of SARS-CoV-2 and selected viral variants as measured by flow cytometry. Geometric means of duplicate log 10 ΔgMFI values are shown in each cell.
(F) Heatmaps comparing the binding (left) and RBD-ACE2 blocking capacity (right) of RBD-specific IgG antibodies for the RBD proteins of SARS-CoV-2 and selected viral variants (B.1.1.7, B.1.351 and P.1) as measured in FIGS. 4E-4G. Black colors indicate high binding or competition while grey colors show moderate binding or competition (white=no binding). AUC values are shown in each cell.
(G) Graphs showing the neutralization curves of SARS-CoV-2 viral variants by anti-RBD IgG antibodies as determined by the S-Fuse neutralization assay. Error bars indicate the SD of assay duplicates.
(H) Heatmaps comparing the IC₅₀ neutralizing values of the selected anti-RBD antibodies against SARS-CoV-2 and selected VOCs with the pseudo-neutralization (bottom) and S-Fuse neutralization (top) assays as measured in FIGS. 14D and 14E.
(I) Heatmap showing binding to spike (top) and RBD proteins (middle), RBD-ACE2 blocking capacity (middle), and neutralizing activity (bottom) but for Cv2.5179 antibody as measured in FIG. 7B-E.
(J) Radar plot comparing the binding of Cv2.1169 IgG and IgA antibodies to SARS-CoV-2 tri-S, S1 and RBD, and to RBD proteins from SARS-CoV-2 viral variants (bold; B.1.1.7, B.1.351 and P.1).
(K) Radar plot comparing the binding of monomeric Cv2.1169 IgG and IgA antibodies to SARS-CoV-2 tri-S, S1 and RBD proteins, and to RBD from selected viral variants (in bold) as measured in FIG. 4J
(L) Competition ELISA graphs (left) comparing the binding of biotinylated (Wuhan) SARS-CoV-2 tri-S protein to the immobilized soluble ACE2 ectodomain in presence of Cv2.1169 IgG or IgA as a competitor. Error bars indicate the SD of duplicate values. Graphs (right) comparing the SARS-CoV-2 neutralizing activity of Cv2.1169 IgG, IgA and IgA Fab as determined with the pseudo-neutralization assay. Error bars indicate the SD of duplicate values.
(M) Graphs comparing the (Wuhan) SARS-CoV-2 neutralizing activity of monomeric and dimeric IgA (dIgA) Cv2.1169 antibodies as determined with the S-Fuse neutralization assay. Error bars indicate the SD of triplicate values. n.dIgA, normalized values according to the number of binding sites.

FIG. 4. Binding characteristics of potent anti-RBD antibody neutralizers
(A) Infrared immunoblot showing the reactivity of SARS-CoV-2 S-specific IgG antibodies (n=101) to denatured (Wuhan) SARS-CoV-2 tri-S protein. Immunoreactive bands (top image) correspond to denatured SARS-CoV-2 tri-S protein revealed with an anti-6×His tag antibody. The band in the bottom image indicates the SARS-CoV-2 antibody Cv2.3132 recognizing denatured tri-S protein, which is characterized by a heavy chain variable region of SEQ ID NO 164 and a light chain variable region of SEQ ID NO 165.
(B) Infrared dot blot showing the reactivity of Cv2.3132 antibody to denatured (Wuhan) SARS-CoV-2 tri-S protein at various concentrations. mGO53 is a non-SARS-CoV-2 isotype control. Cv2.1169 was included for comparison.
(C) Graphs showing the reactivity of Cv2.3132 IgG antibody against 15-mer S2 overlapping 5-amino acid peptides (n=52). mGO53 is a non-SARS-CoV-2 isotype control. Means±SD of duplicate values are shown.
(D) Competition ELISA graphs showing the IgG binding to (Wuhan) SARS-CoV-2 tri-S (top) and RBD (bottom) of selected biotinylated SARS-CoV-2 S-specific antibodies in presence of the corresponding non-biotinylated IgG antibodies as potential competitors. Means±SD of duplicate values are shown.
(E) ELISA graphs showing the reactivity of SARS-CoV-2 RBD-specific IgG antibodies to RBD proteins from SARS-CoV-2 viral variants (B.1.1.7 (α), B.1.351 (β) and P.1 (γ)). Means±SD of duplicate OD_{405 nm} values are shown.
(F) Competition ELISA graphs showing the binding of biotinylated RBD proteins from SARS-CoV-2 and viral variants (B.1.1.7 (α), B.1.351 (β) and P.1 (γ)) to soluble ACE2 ectodomain in presence of SARS-CoV-2 S-specific IgG antibodies as potential competitors. Means±SD of duplicate values are shown.
(G) Same as in (F) but selected IgG competitors tested against B.1.1.7 and B.1.351 for RBD proteins at a higher concentration. Means of duplicate values are shown.
(H) Same as in (E) but for RBD proteins from SARS-CoV-2 viral variants κ, δ and δ+.
(I) Same as in (F) but for RBD proteins from SARS-CoV-2 viral variants κ, δ and δ+.
(J) ELISA graphs comparing the reactivity of the monomeric IgG/IgA and dimeric IgA (dIgA) antibody forms of Cv2.1169 to SARS-CoV-2 tri-S, S1 and RBD, and to RBD proteins from SARS-CoV-2 viral variants (α, β, γ, δ, δ+ and κ). Means±SD of duplicate OD_{405 nm} values are shown.

FIG. 5. Poly- and self-reactivity assessment of potent SARS-CoV-2 neutralizing antibodies
(A) ELISA graphs showing the reactivity of selected SARS-CoV-2 neutralizing antibodies against dsDNA (DNA), flagellin (Fla), YU2 HIV-1 Env (gp140), insulin (INS), keyhole limpet hemocyanin (KLH), lipopolysaccharide (LPS), lysozyme (LZ), MAPK-14 (MAPK), proteoglycan (PG), and thyroglobulin (Tg); mGO53 (3) and ED38 (4) are negative and positive control antibody, respectively. Anti-SARS-CoV-2 S antibody Cv2.3132 showing HEp-2 reactivity in (C) was included for comparison. Mean of duplicate values are shown.
(B) Heatmap comparing the area under the curve (AUC) values determined from the ELISA binding analyses shown in (A). Darker colors indicate high binding while light colors show moderate binding (white=no binding).
(C) Microscopic images showing the reactivity of selected SARS-CoV-2 antibodies to HEp2-expressing self-antigens assayed by indirect immunofluorescence assay. The negative (mGO53), low-positive (ED38), and kit's positive (Ctr+) controls were included in the experiment. HEp-2-reactive anti-SARS-CoV-2 S antibody Cv2.3132 was also included for comparison. The scale bars represent 40 μm.
(D) Bar graph showing the HEp-2 reactivity of selected SARS-CoV-2 antibodies as measured by ELISA. Means±SD of duplicate values are shown. Ctr+ and Ctr− are the positive and negative control of the kit, respectively.
(E) Representative microarray plots showing the reactivity of selected SARS-CoV-2 antibodies to human proteins. Plots (top) showing the mean fluorescence intensity (MFI) values given on each protein spot by the reference (Ref: mGO53) and test antibody on the y and x axis, respectively. Each dot represents the average of duplicate array proteins. Plots (bottom) showing the z-scores given on a single protein by the reference (Ref: mGO53, y axis) and test antibody (x axis).
(F) Frequency histograms showing the log₁₀ protein displacement (a) of the MFI signals for the selected SARS-CoV-2 antibodies compared to non-reactive antibody mGO53 obtained from two independent experiments (Array #1 and #2). The polyreactivity index (PI) corresponds to the Gaussian mean of all array protein displacements. Light grey (5213, 1169 and 1353) and dark grey (3235 and 3194) histograms indicate non-polyreactive and polyreactive antibodies, respectively.

FIG. 6. In vivo therapeutic activity of potent SARS-CoV-2 neutralizer Cv2.1169
(A) Schematic diagram showing the experimental design of Cv2.1169 antibody therapy in (Wuhan) SARS-CoV-2-infected K18-hACE2 mice (top). Animals were infected intranasally (i.n.) with 10⁴ plaque forming units (PFU) of (Wuhan) SARS-CoV-2 virus and received 6 h later an intraperitoneal (i.p.) injection of Cv2.1169 or isotypic control IgG antibody at ˜10 mg/kg (0.25 mg) and ˜20 mg/kg (0.5 mg). Graphs showing the evolution of initial body weight (% A weight, bottom left) and survival rate (bottom right) in animal groups. Groups of mice were compared in the Kaplan-Meier analysis using Log-rank Mantel-Cox test.
(B) Same as in (A) but with K18-hACE2 mice infected with 10⁵ PFU and treated 22 h later with 1 mg i.p. of Cv2.1169 IgG antibody (˜40 mg/kg) or with 1 mg+0.4 mg of Cv2.1169 IgG antibody, i.p and i.n, respectively.
(C) Same as in (A) but with infected mice treated with Cv2.1169 IgG and IgA antibodies at ˜5 mg/kg (0.125 mg) i.p.
(D) Schematic diagram shows the experimental design of Cv2.1169 antibody therapy in (Wuhan) SARS-CoV-2-infected golden Syrian hamsters (top). Animals were infected intranasally (i.n.) with 6×10⁴ plaque forming units (PFU) of SARS-CoV-2 virus and received 24 h later an intraperitoneal (i.p.) injection of PBS, Cv2.1169 or isotypic control IgG antibody at ˜10 mg/kg (1 mg). Dot plots showing the lung weight/body weight ratio (LW/BW)×100 (left), infectivity (center) and RNA load (right) measured in animal groups at 5 dpi. Groups of hamsters were compared using two-tailed Mann-Whitney test.
(E) Same as in (D) but with infected animals treated 4 h later with Cv2.1169 IgG and IgA antibodies at ˜5 mg/kg (0.5 mg) i.p.
(F) Same as in (A) but with K18-hACE2 mice infected with 10⁴ PFU of the SARS-CoV-2 variant β (B.1.351), and either pre-treated 6h before infection with ˜5 mg/kg (0.5 mg) of Cv2.1169 IgA or treated 6h post-infection with ˜5 mg/kg (0.5 mg) of Cv2.1169 IgG or isotype control (ctr).

FIG. 7. V_{H}1-58-encoded SARS-CoV-2 spike-captured memory B-cell antibodies.
(A) Amino acid alignment of the heavy chains (IgH, left) and light chains (IgL, right) of the V_{H}1-58-encoded human antibodies produced from SARS-CoV-2 spike-captured memory B-cell antibodies. Dendrograms showing the relationship between V_{H}1-58-encoded human antibodies generated from the IgH and IgL sequence alignments are shown at the bottom.
(B) ELISA graphs showing the reactivity of V_{H}1-58-encoded antibodies against the (Wuhan)SARS-CoV-2 tri-S protein. Means±SD of duplicate OD_{405 nm} values are shown.
(C) ELISA graphs comparing the binding of Cv2.5179 and Cv2.1169 antibodies to RBD proteins. Means±SD of duplicate OD_{405 nm} values are shown.
(D) Competition ELISA graphs showing the binding of biotinylated RBD proteins to the immobilized soluble ACE2 ectodomain in presence of Cv2.5179 or Cv2.1169 antibody as a competitor. Means±SD of duplicate values are shown.
(E) Graphs showing the neutralization curves of SARS-CoV-2 and VOCs by Cv2.5179 IgG antibody as determined with the S-Fuse neutralization assay. Means±SD of duplicate values are shown. IC₅₀ values are indicated in the top left-hand corner.

FIG. 8. ELISA binding of biotinylated Cv2.5179 to Wuhan triS protein in the presence of the other neutralizing antibodies as potential competitors.

Competition ELISA graph showing the binding of biotinylated Cv2.5179 antibody to (Wuhan) SARS-CoV-2 tri-S in presence of selected anti-RBD antibodies as potential competitors. The binding curves show that all the tested anti-RBD nAbs inhibit/block the binding of Cv2.5179 to the SARS-Cov-2 triS protein.

FIG. 9. Cv2.1169 binding to tri-S and RBD proteins of VOC Beta and Omicron (BA.1)—Related to FIGS. 17A and 17C.

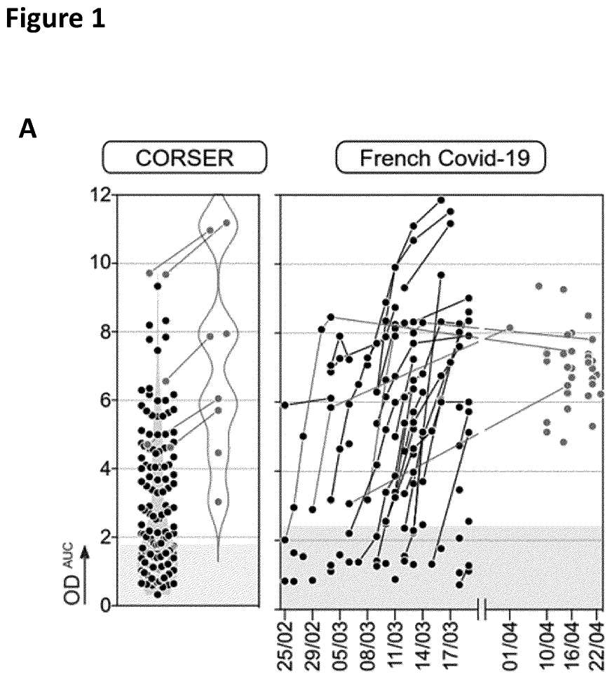

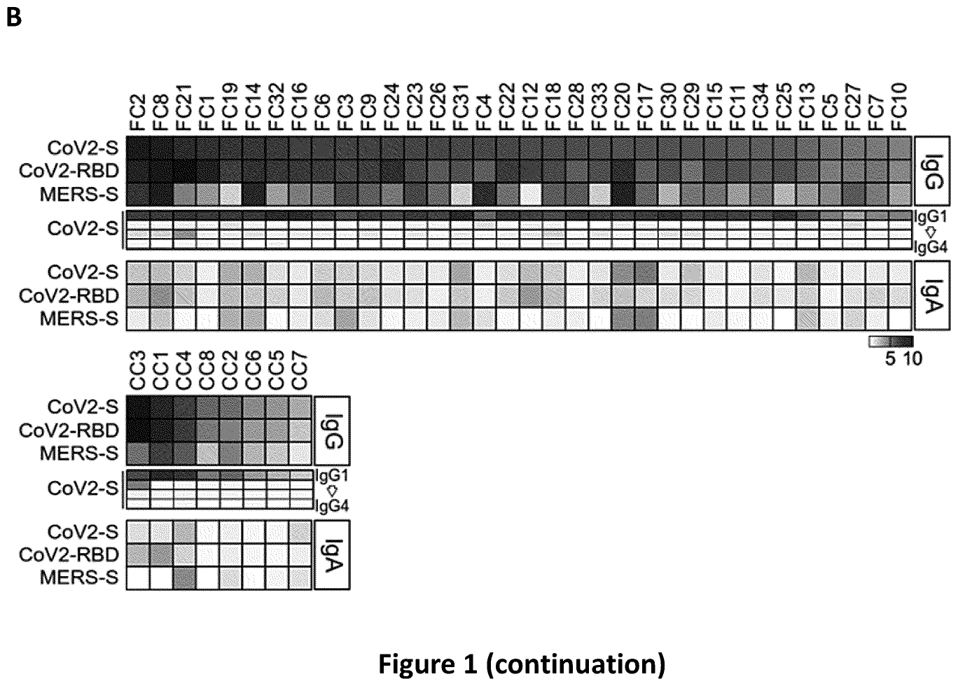

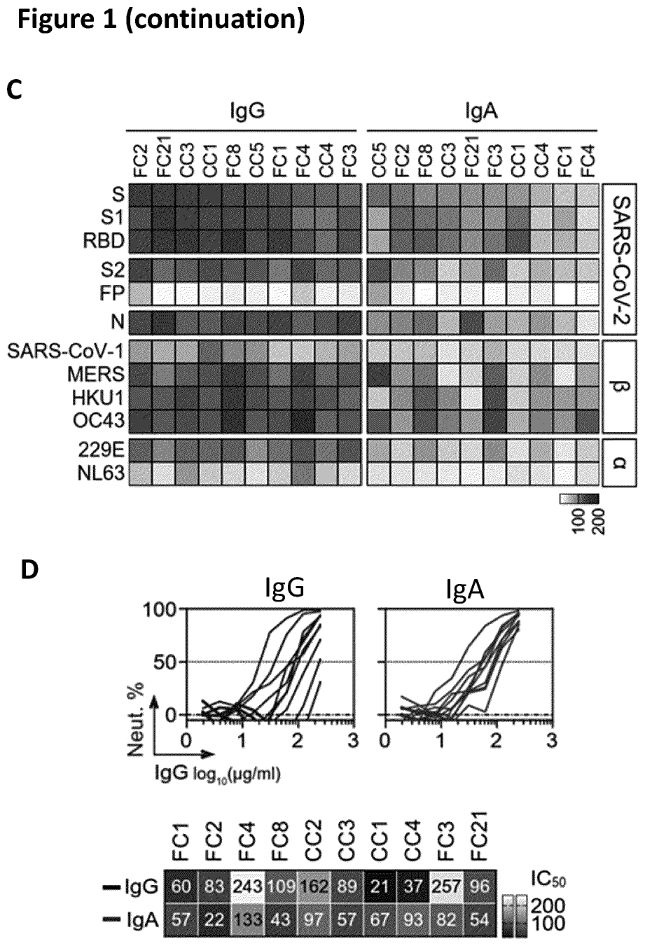

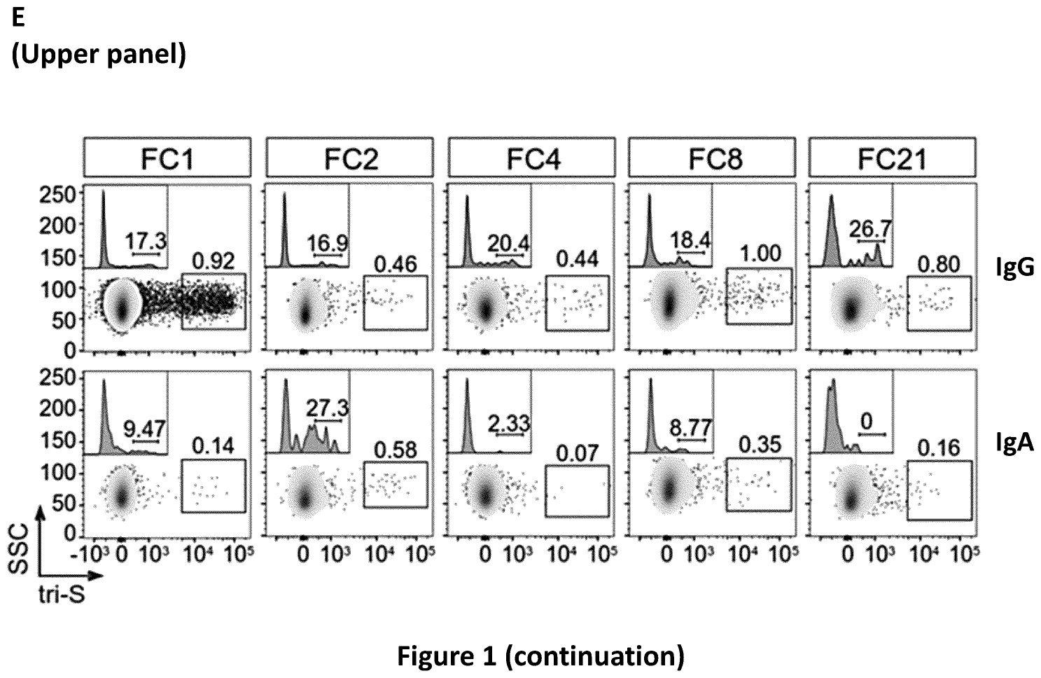

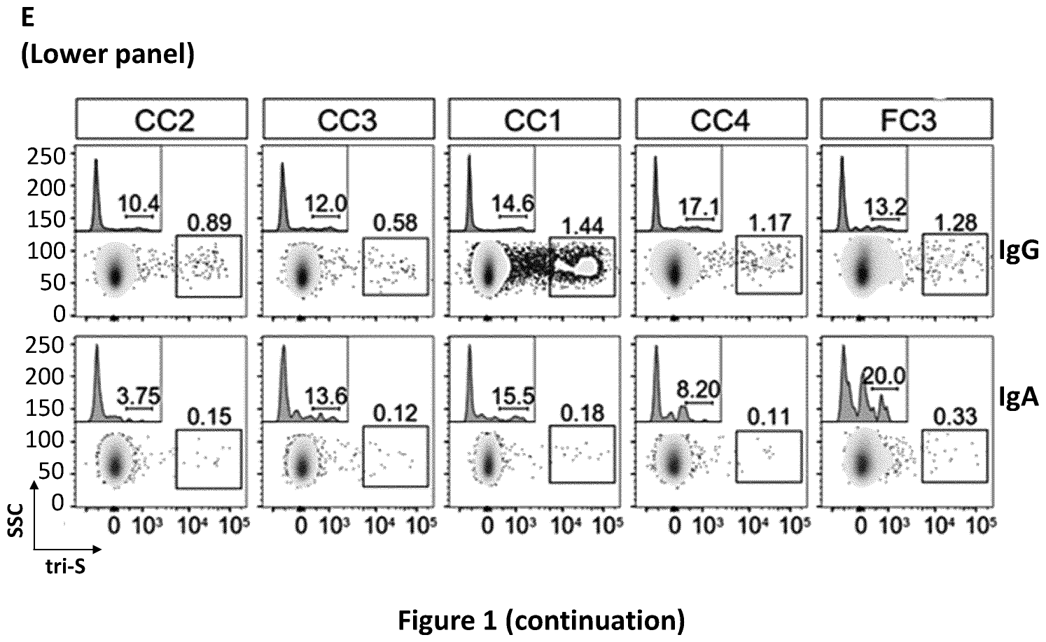

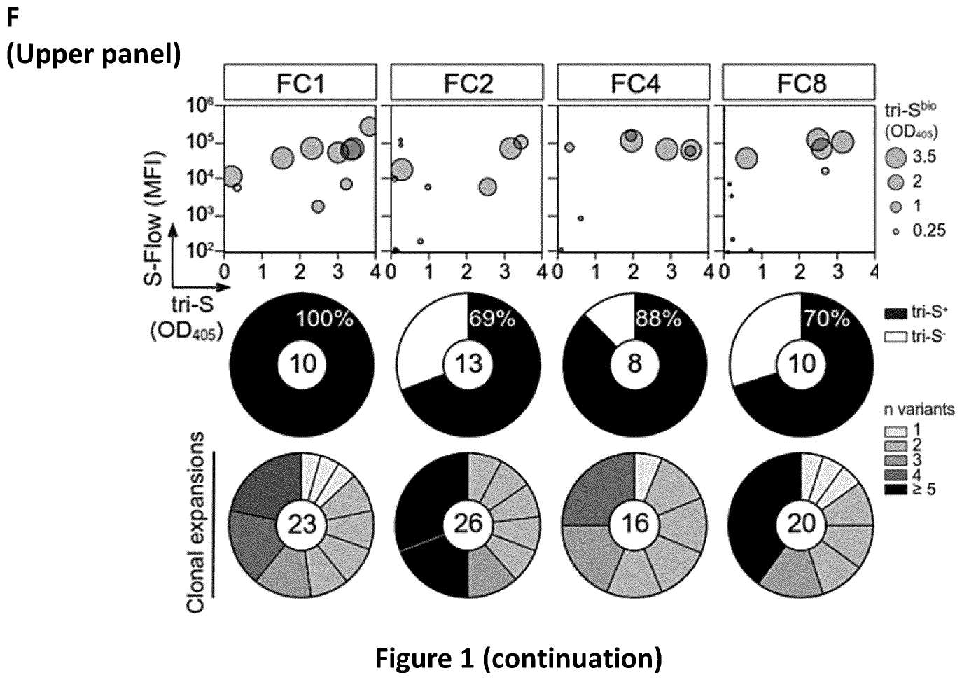

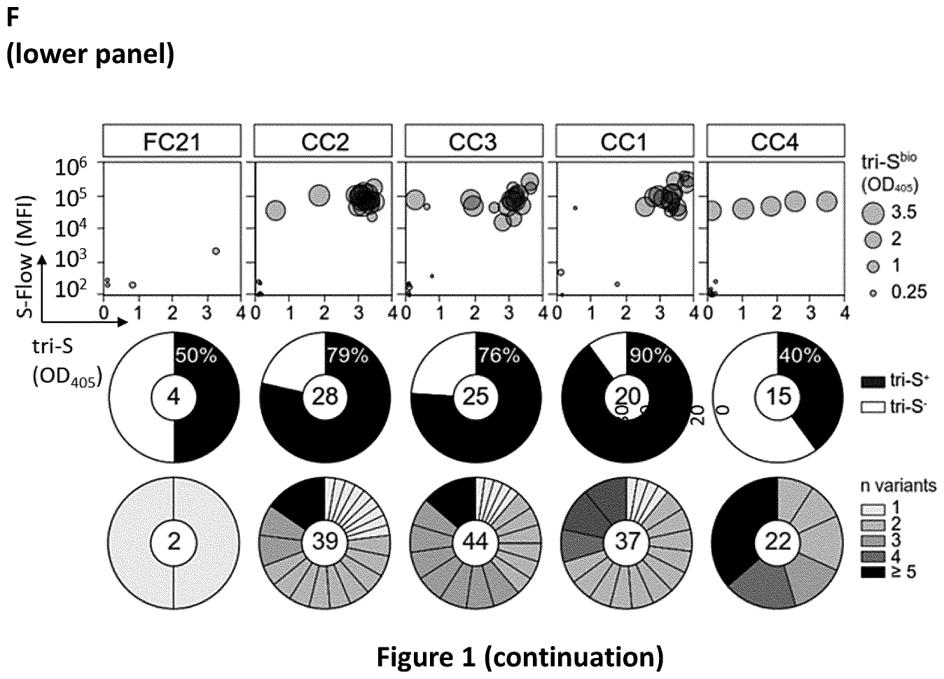

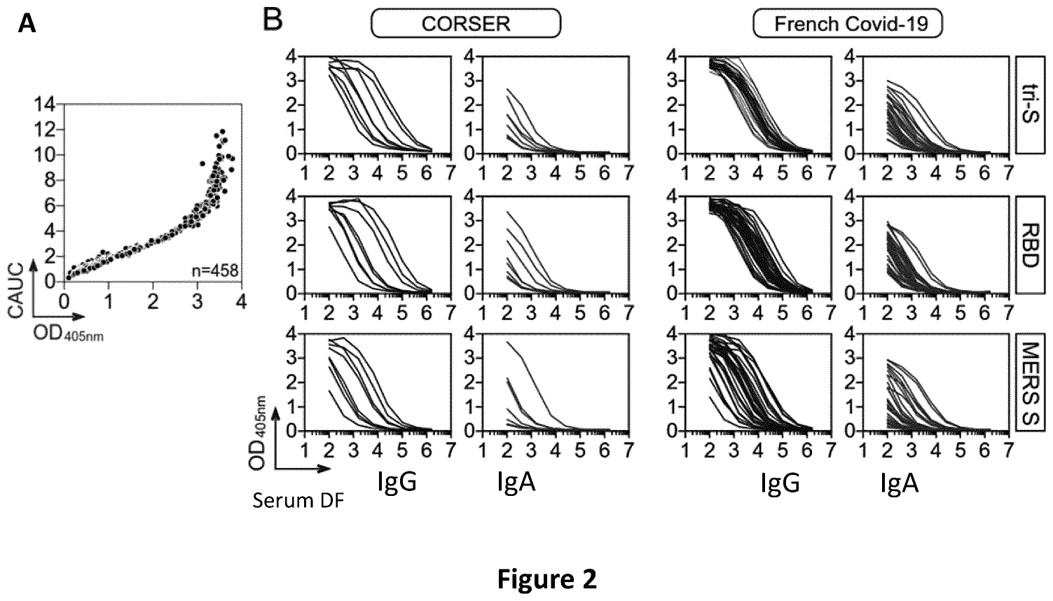

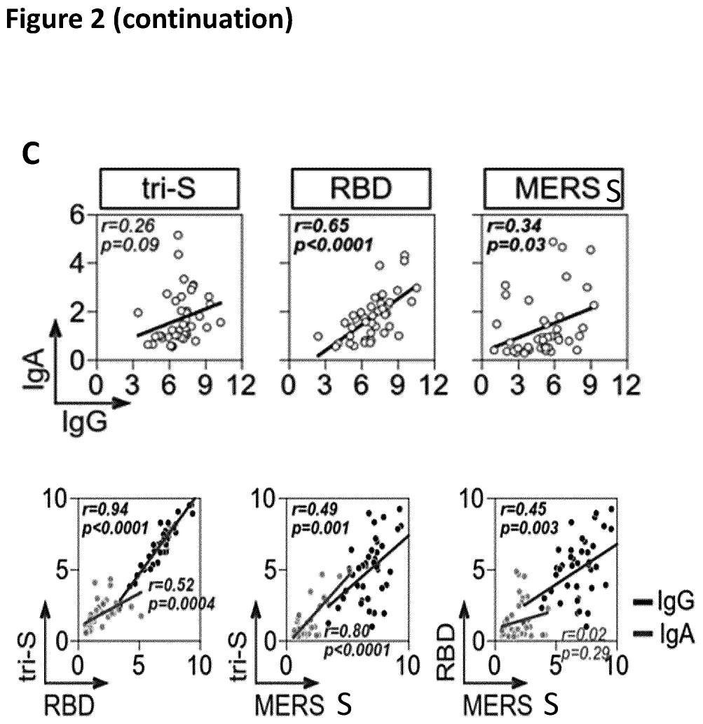

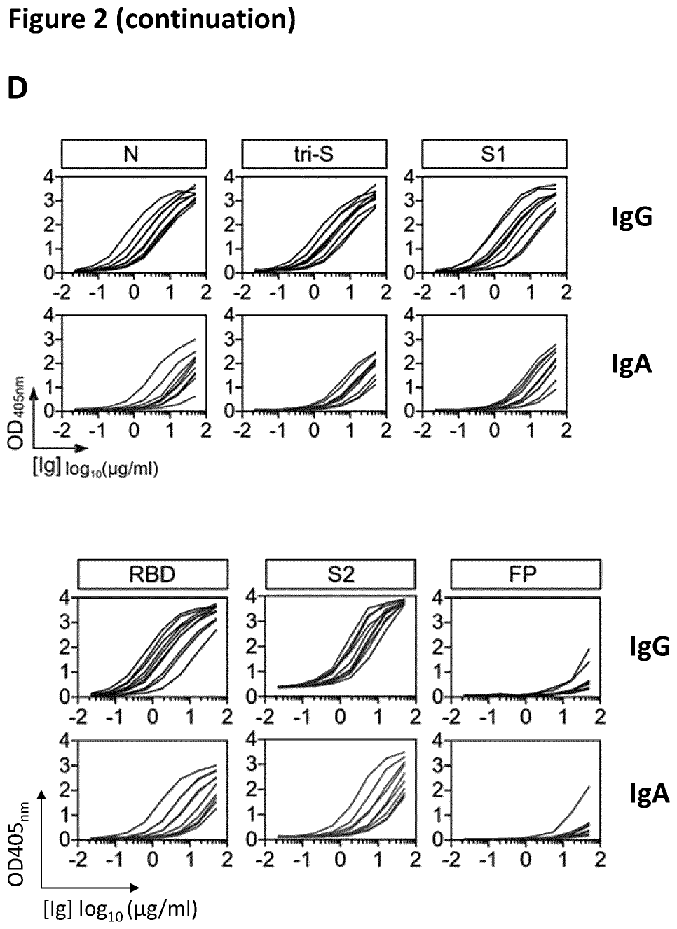

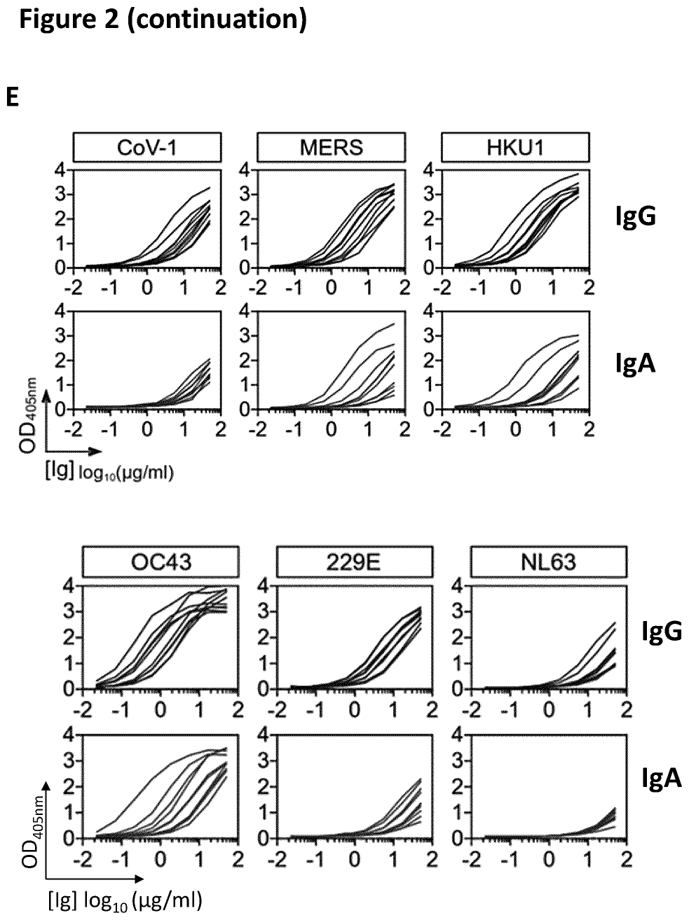

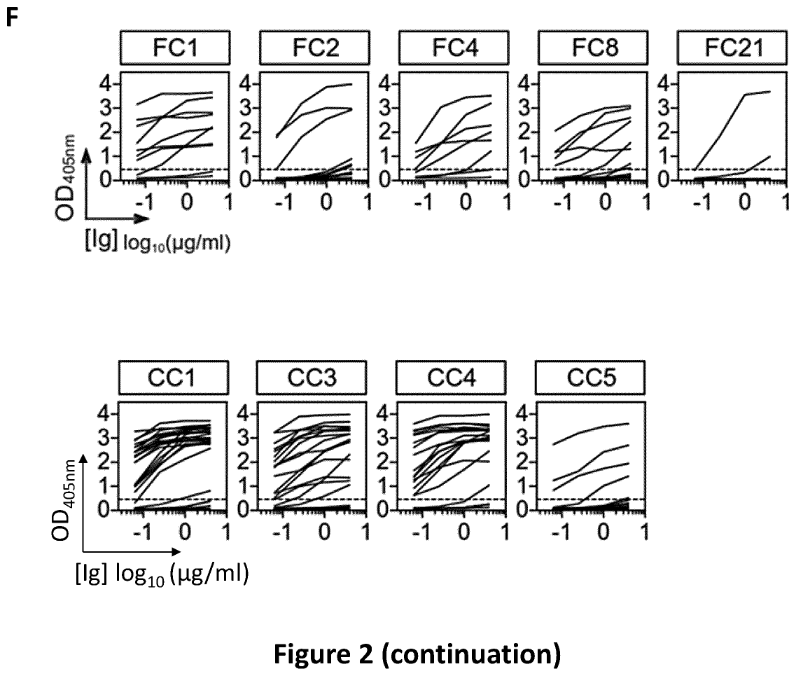

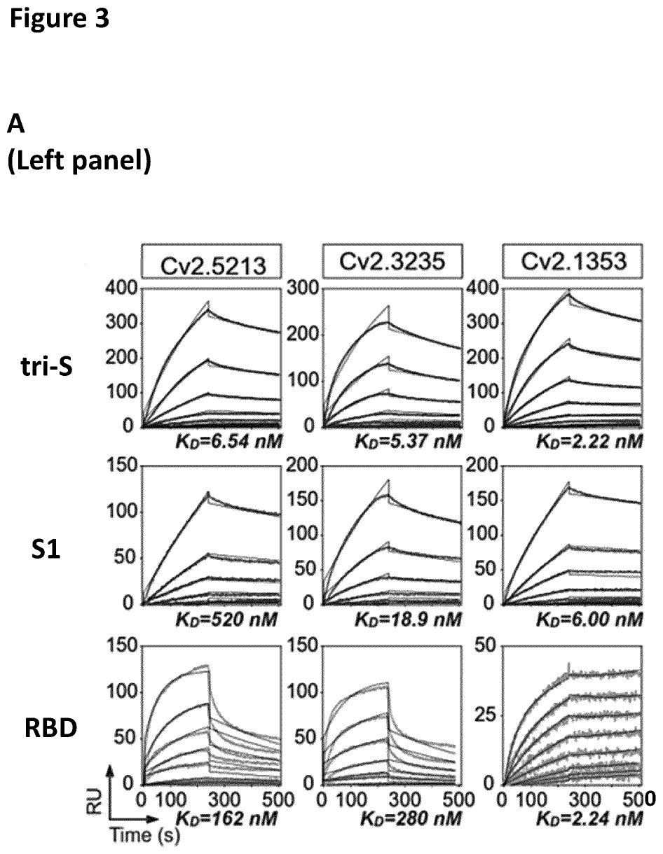

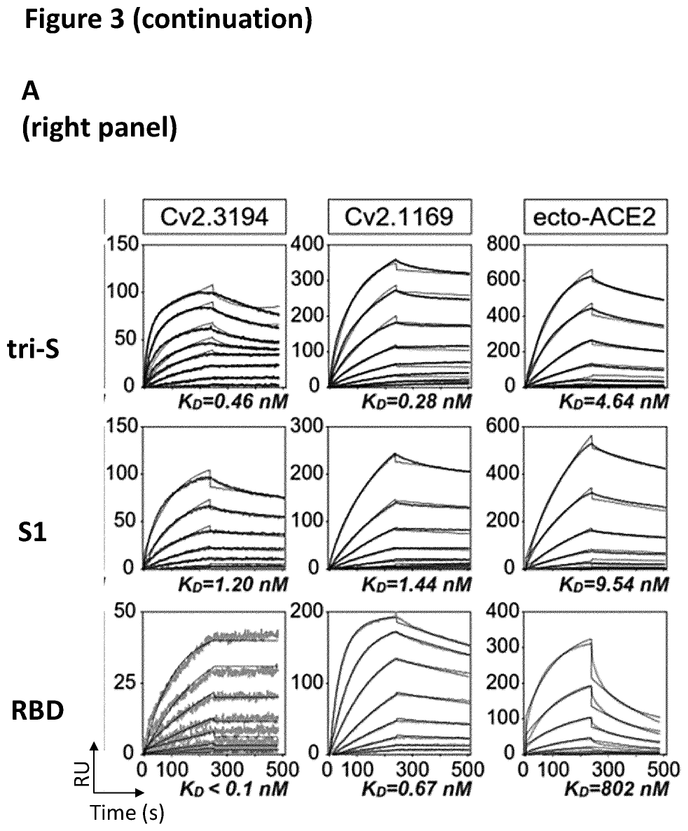

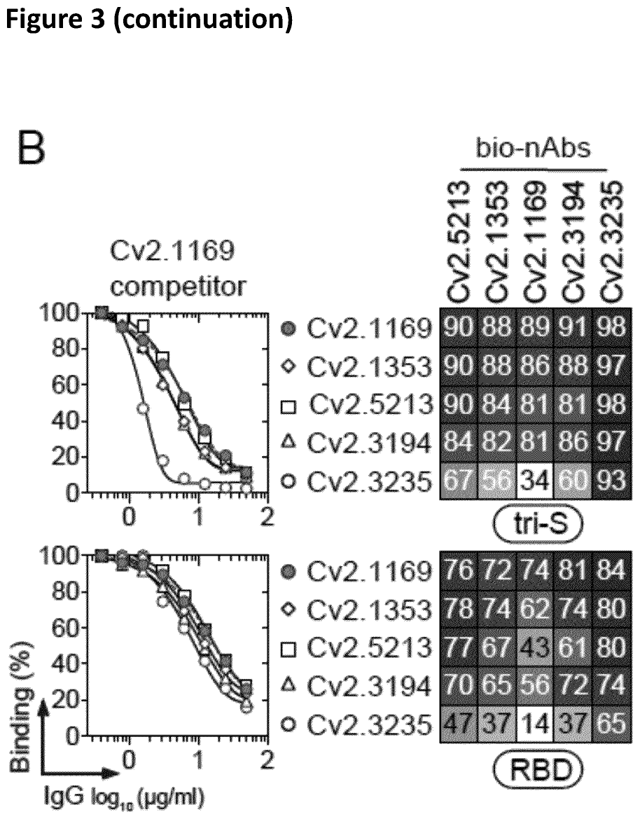

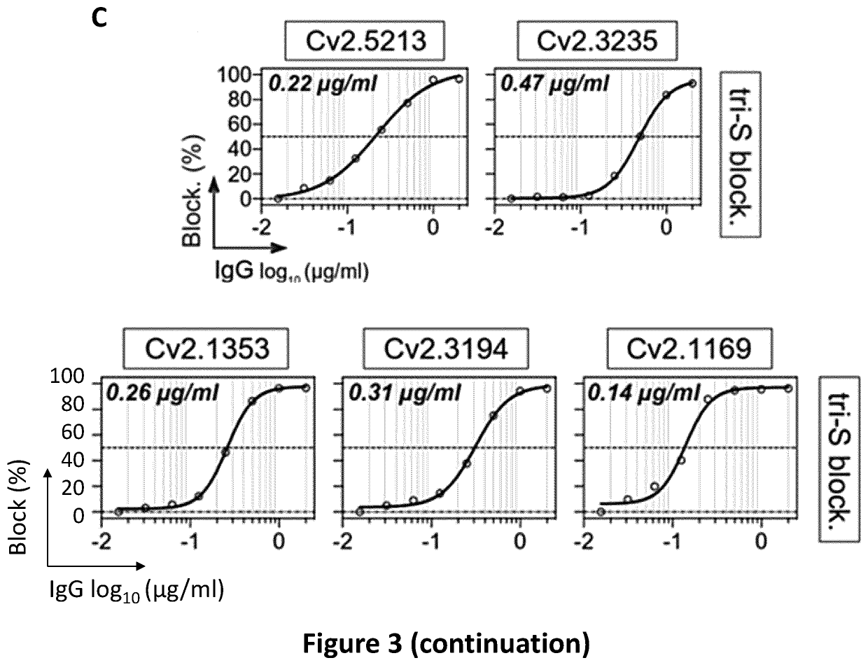

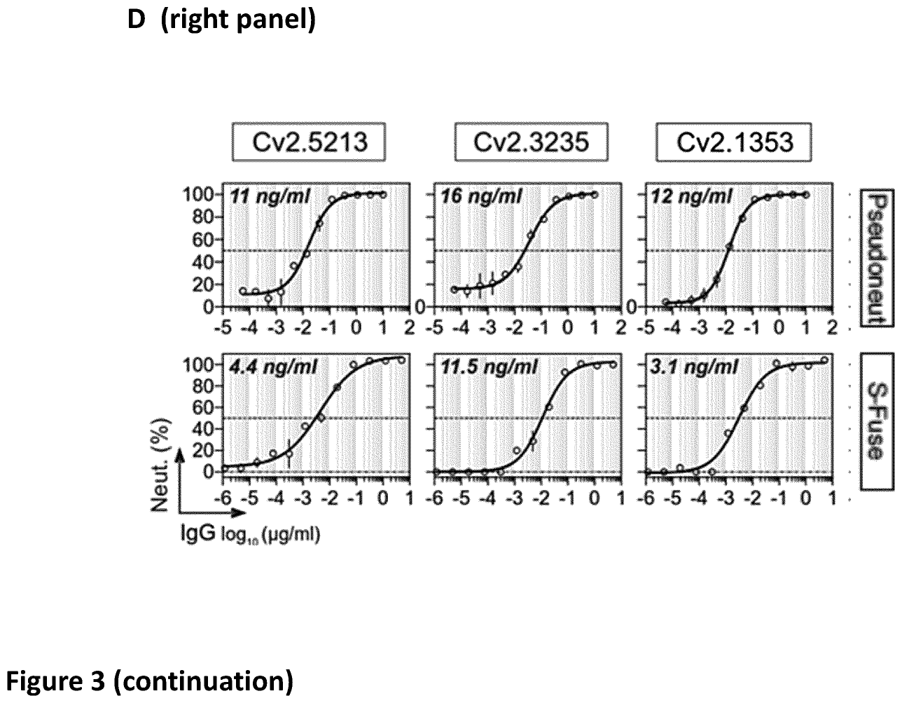

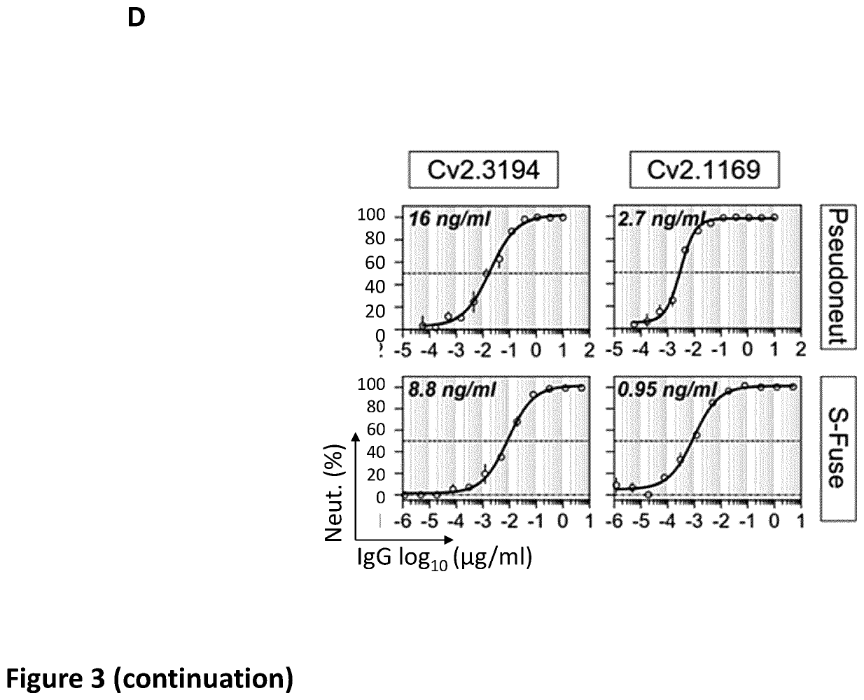

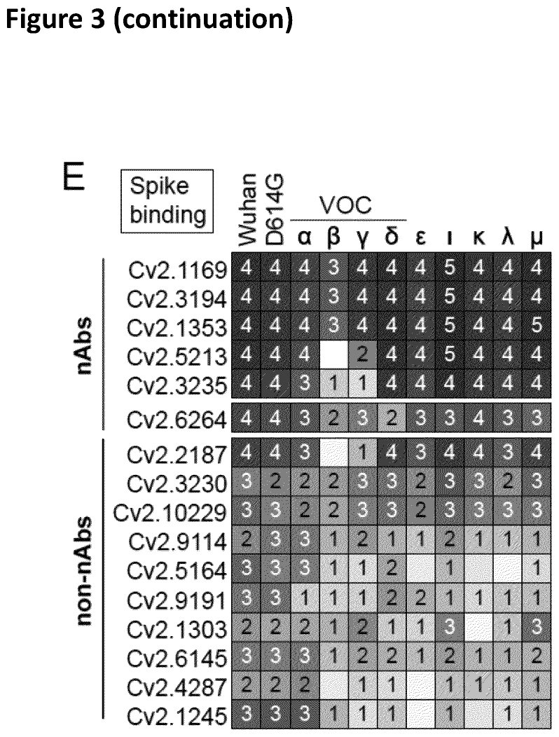

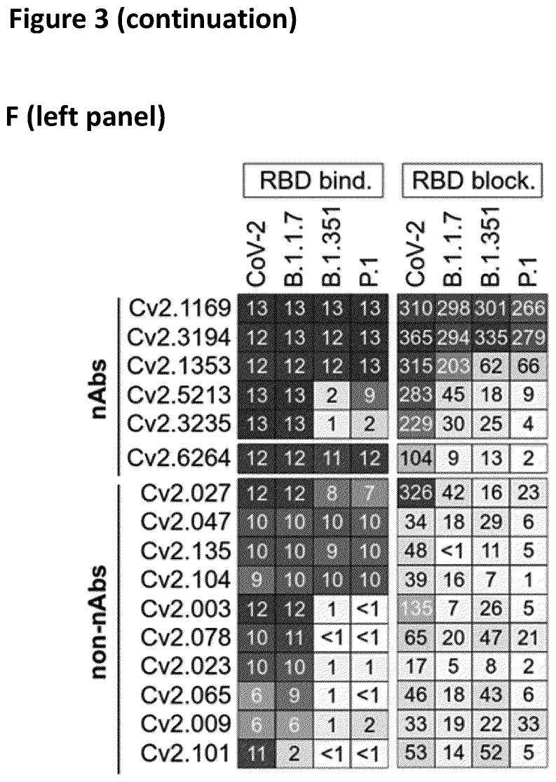

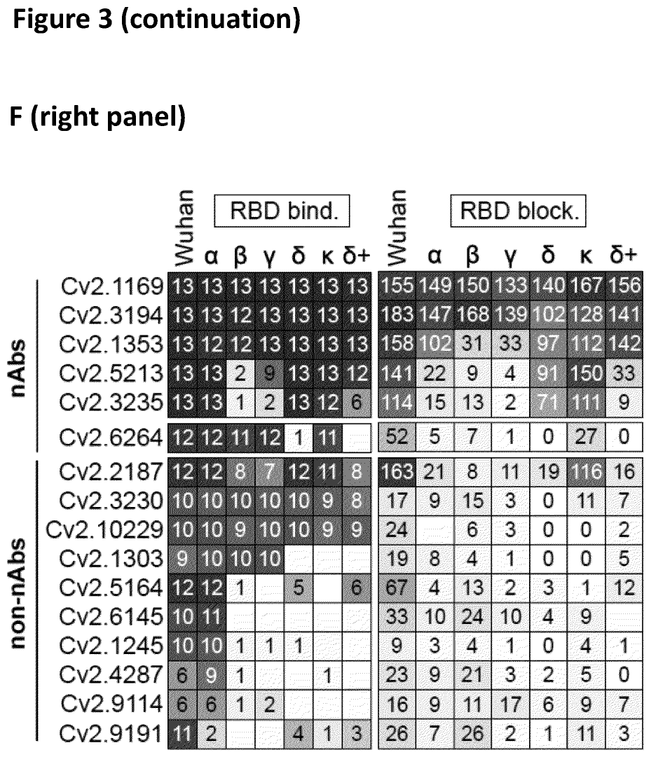

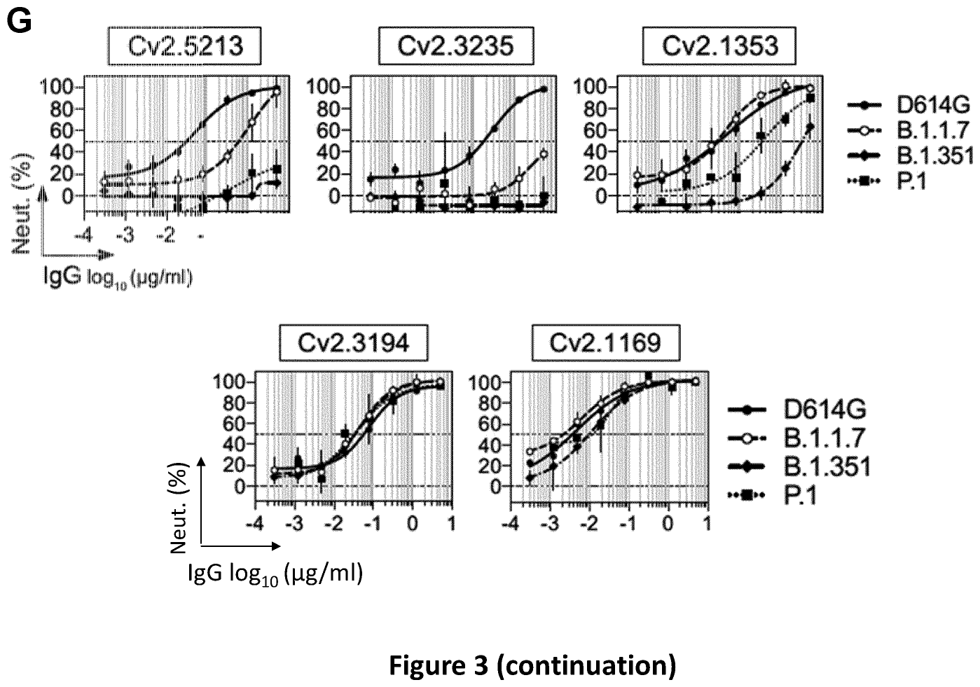

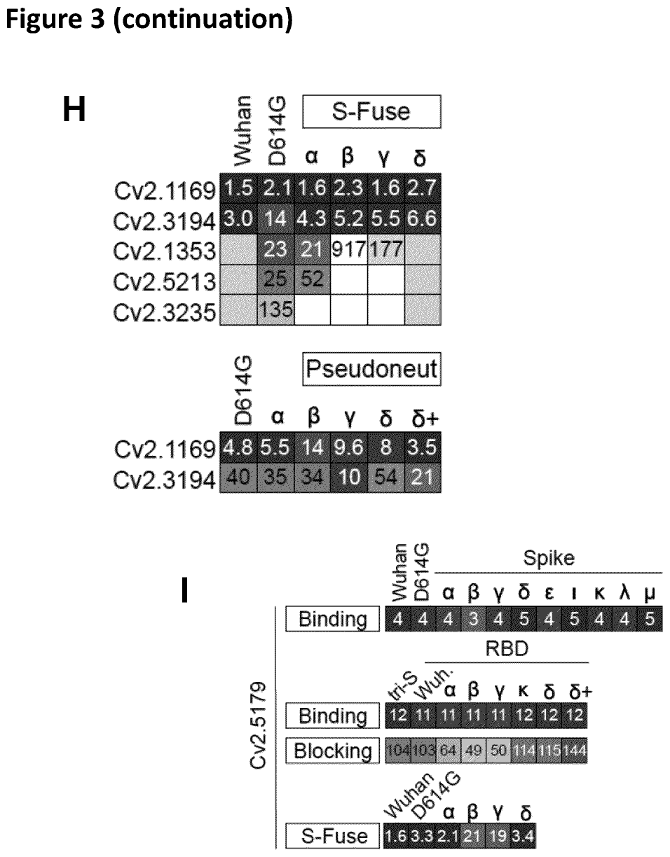

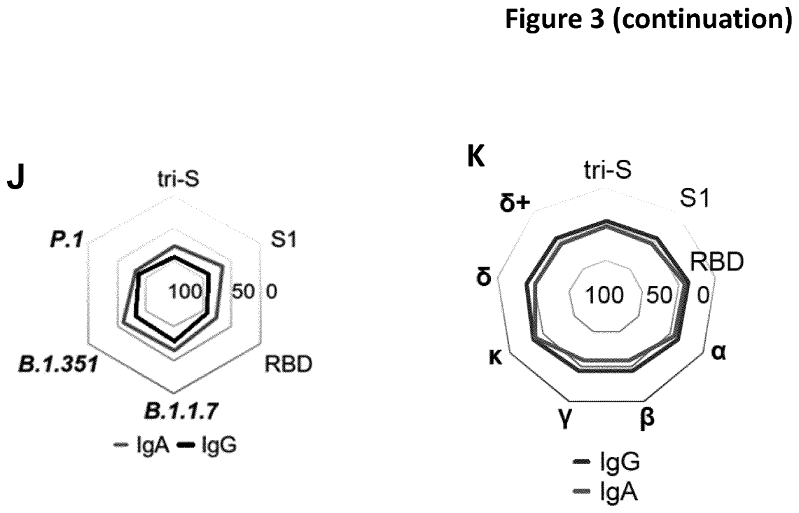

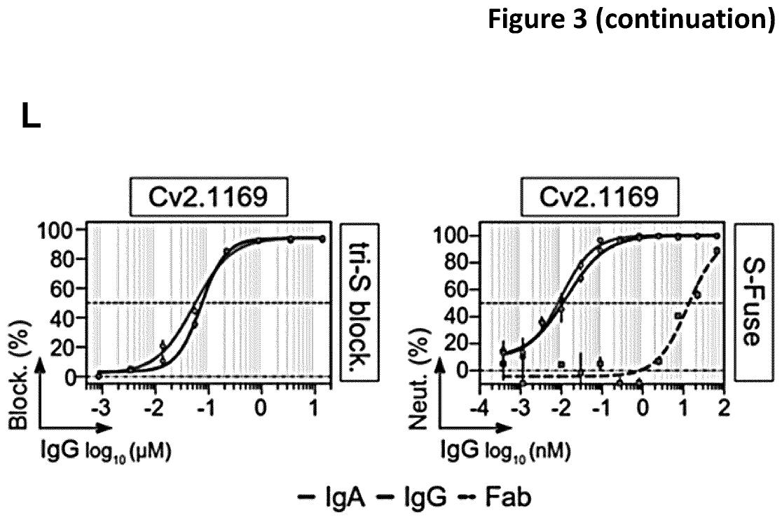

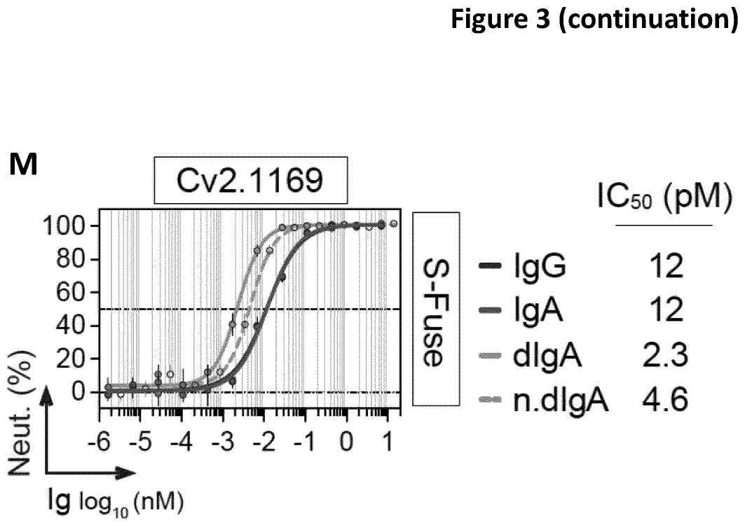

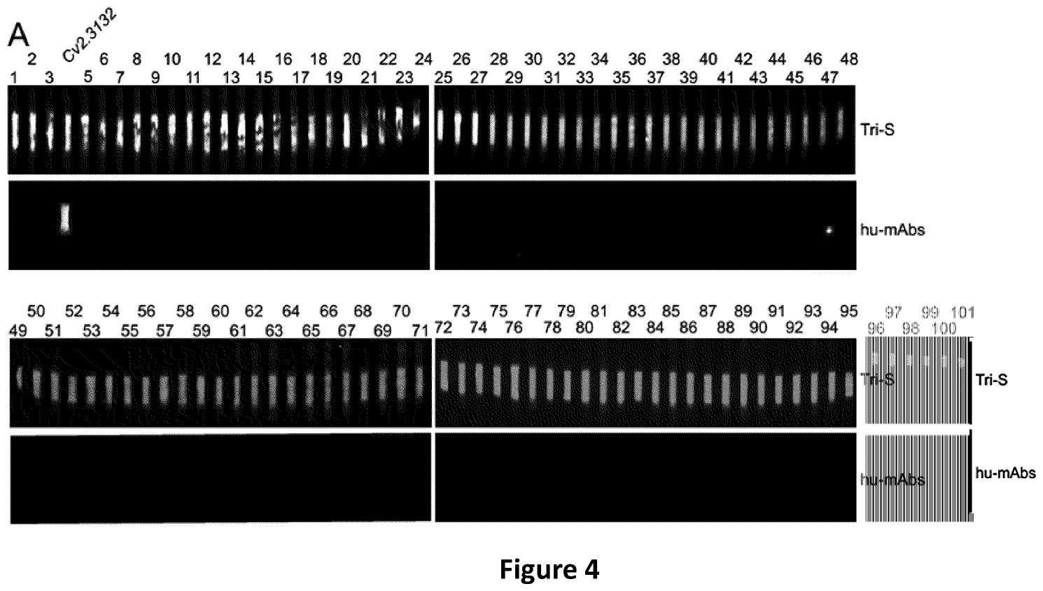

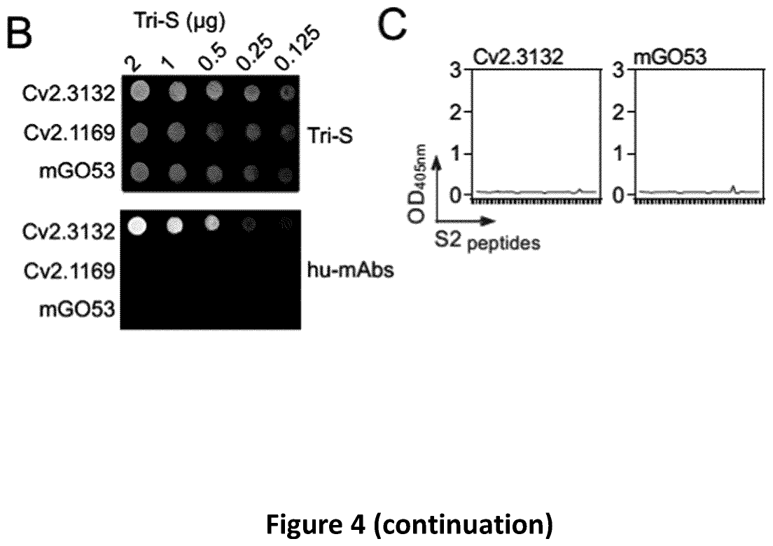

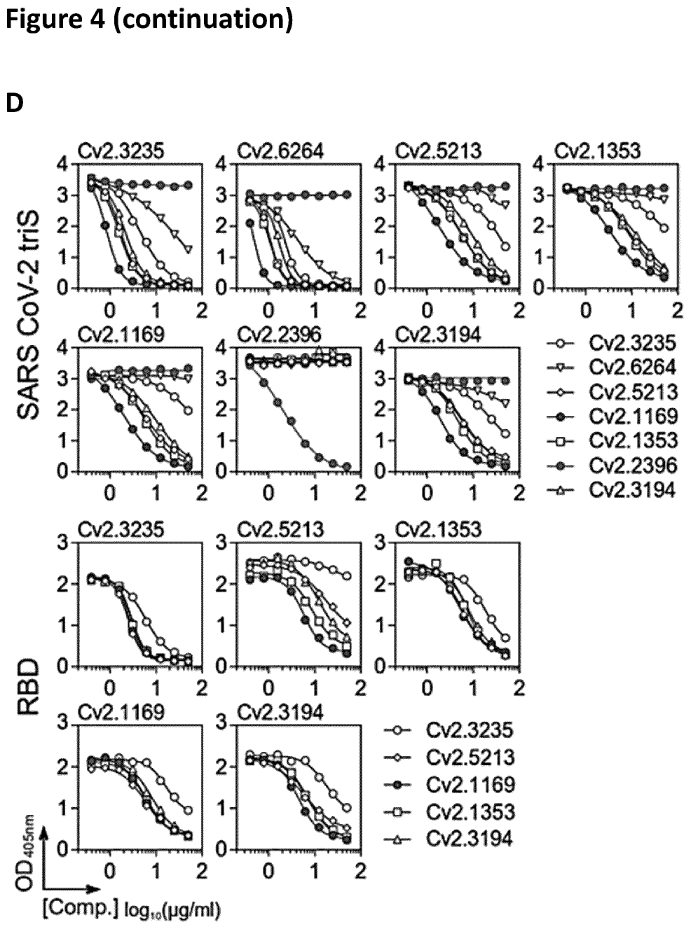

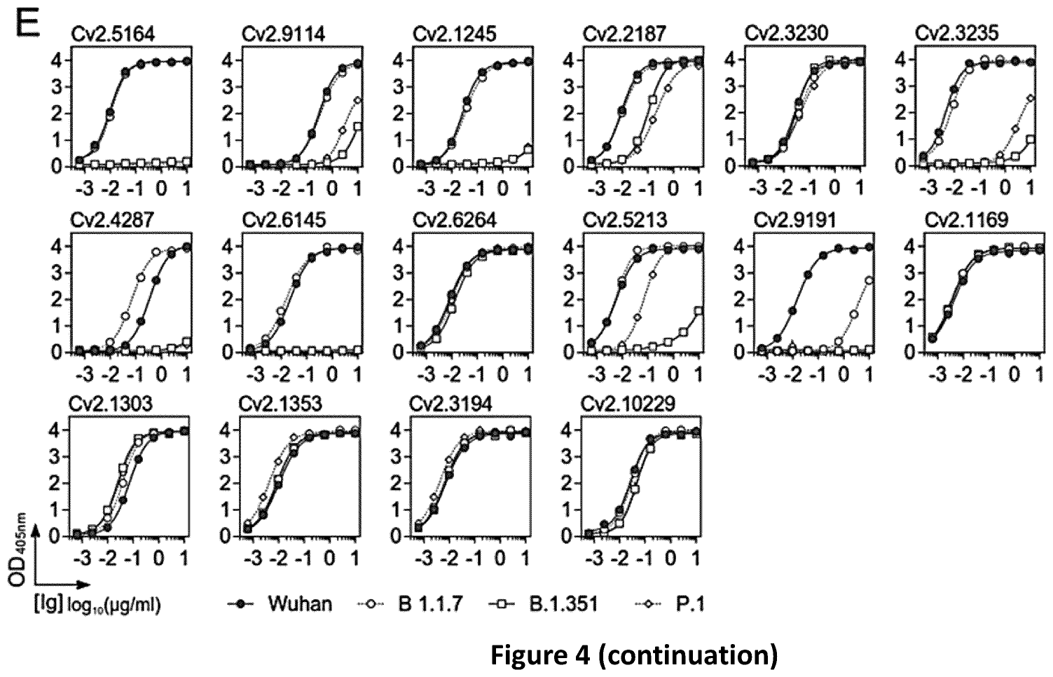

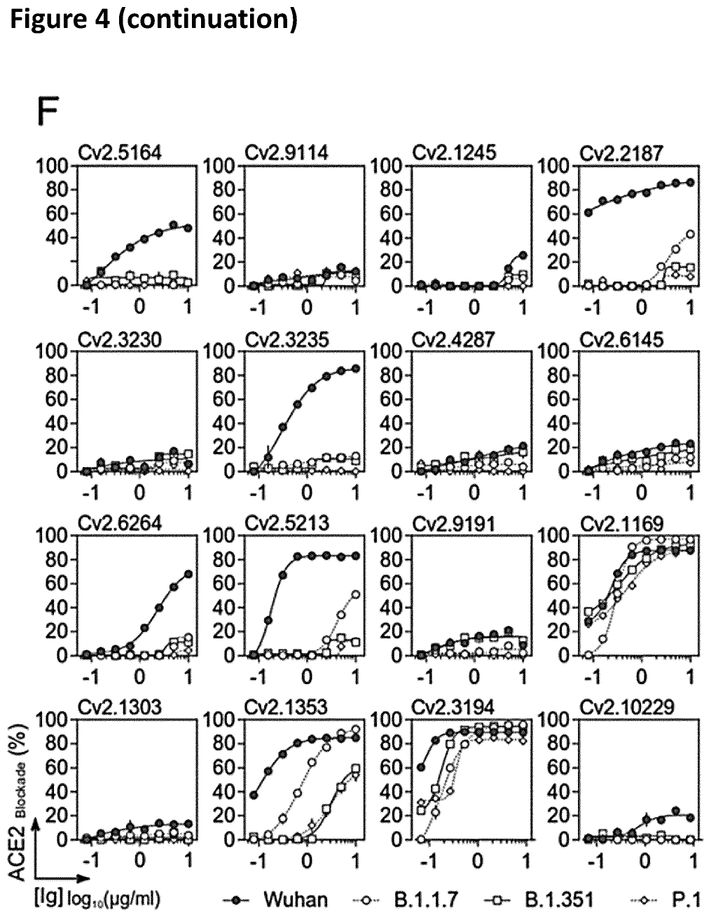

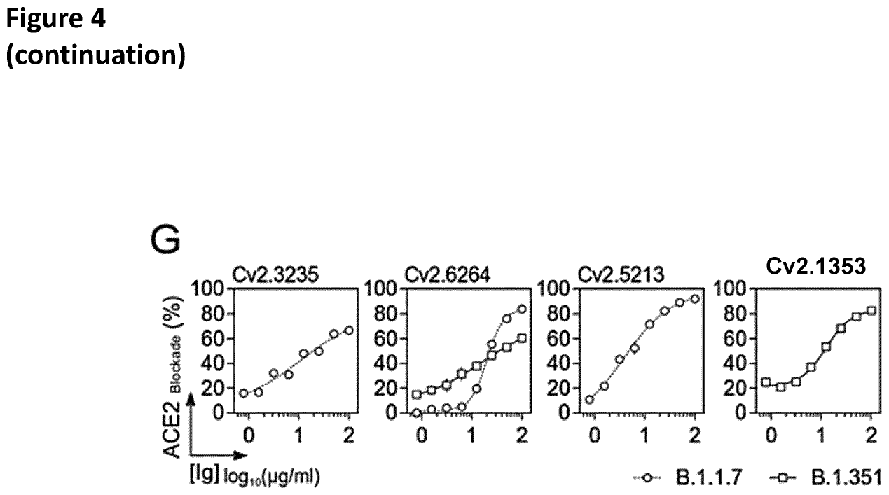

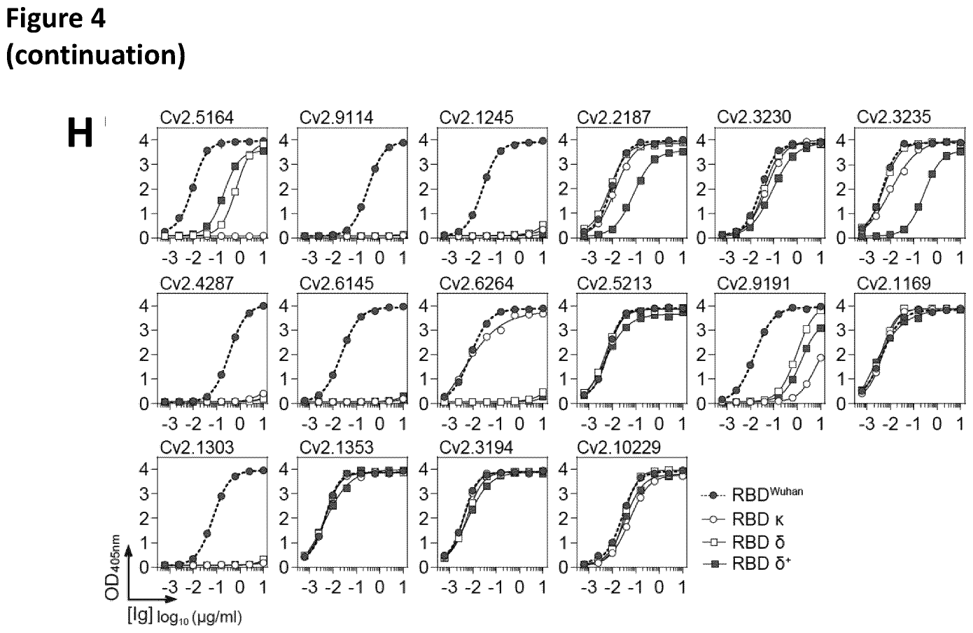

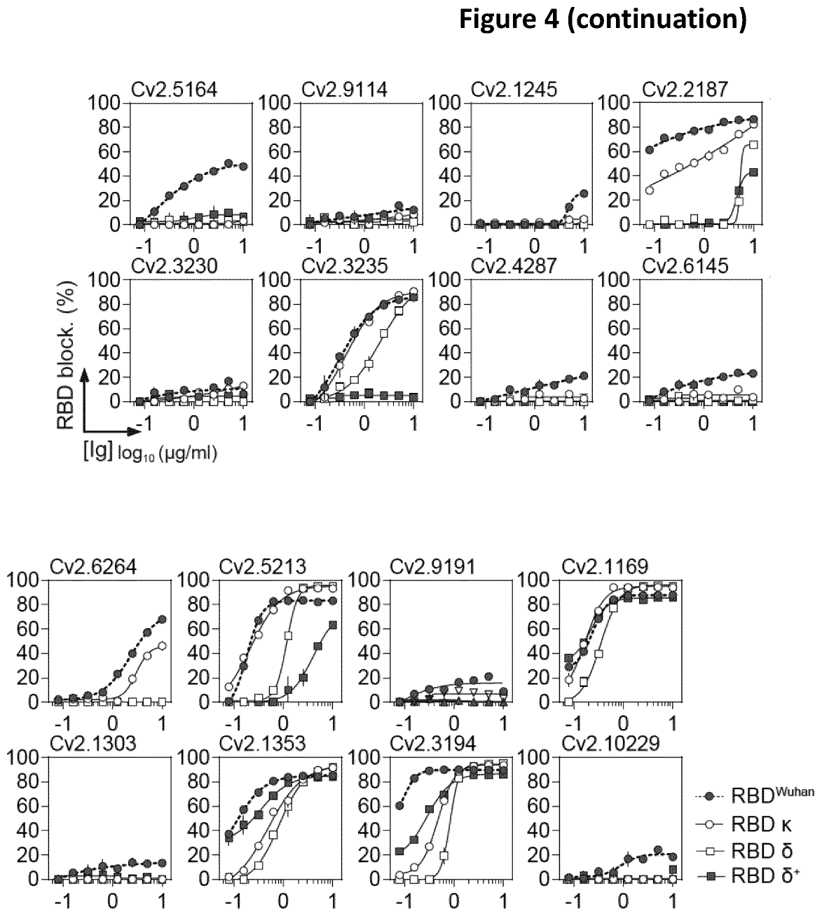

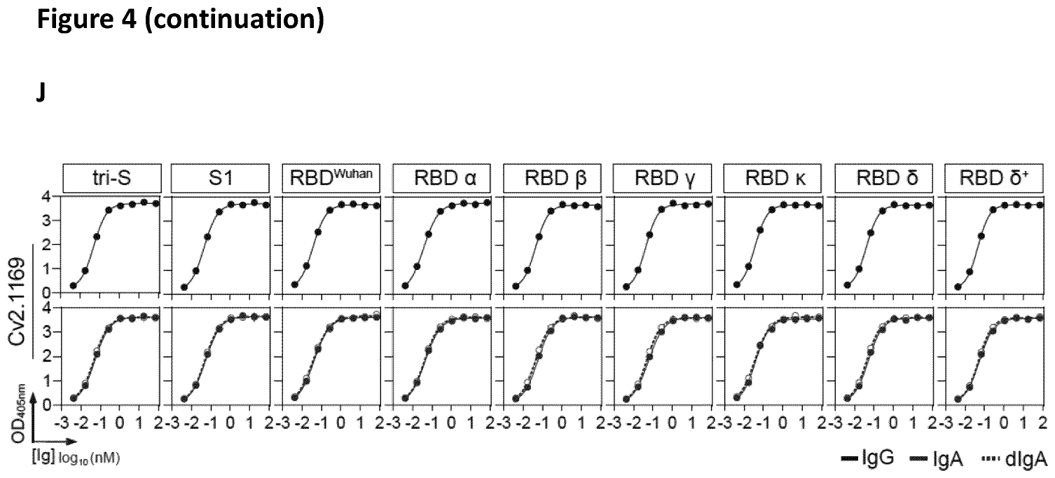

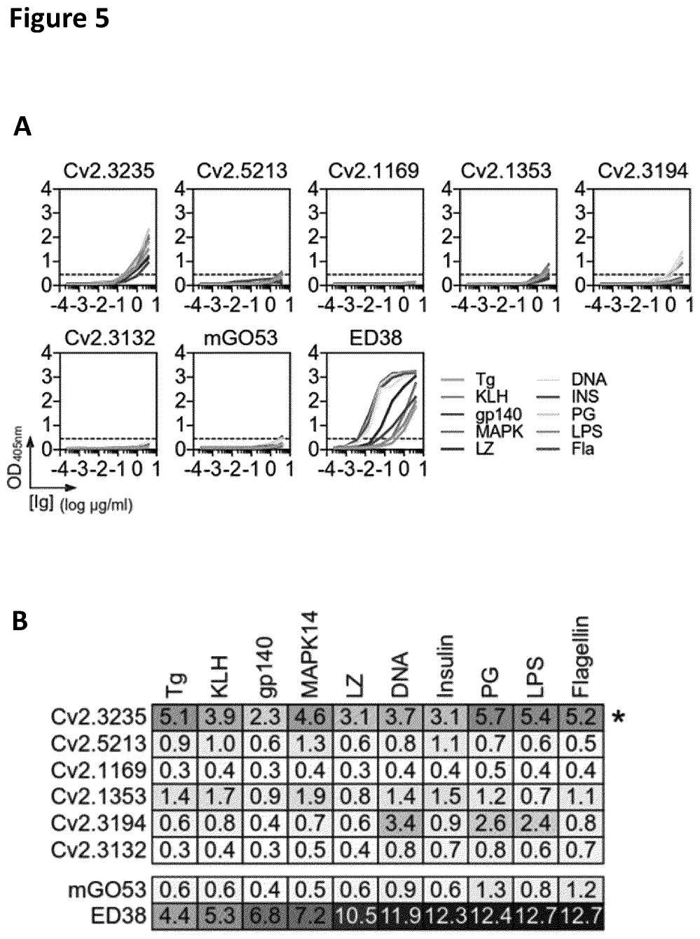

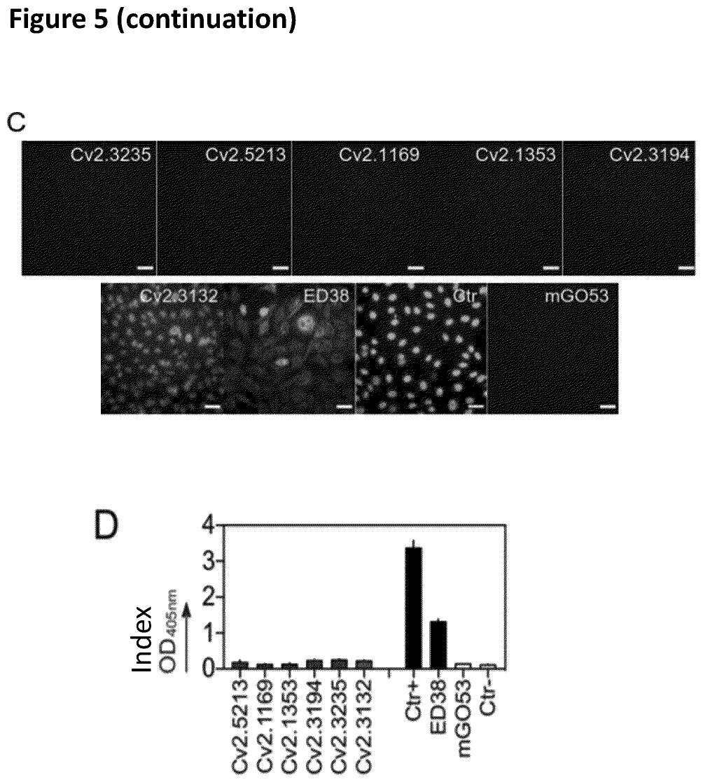

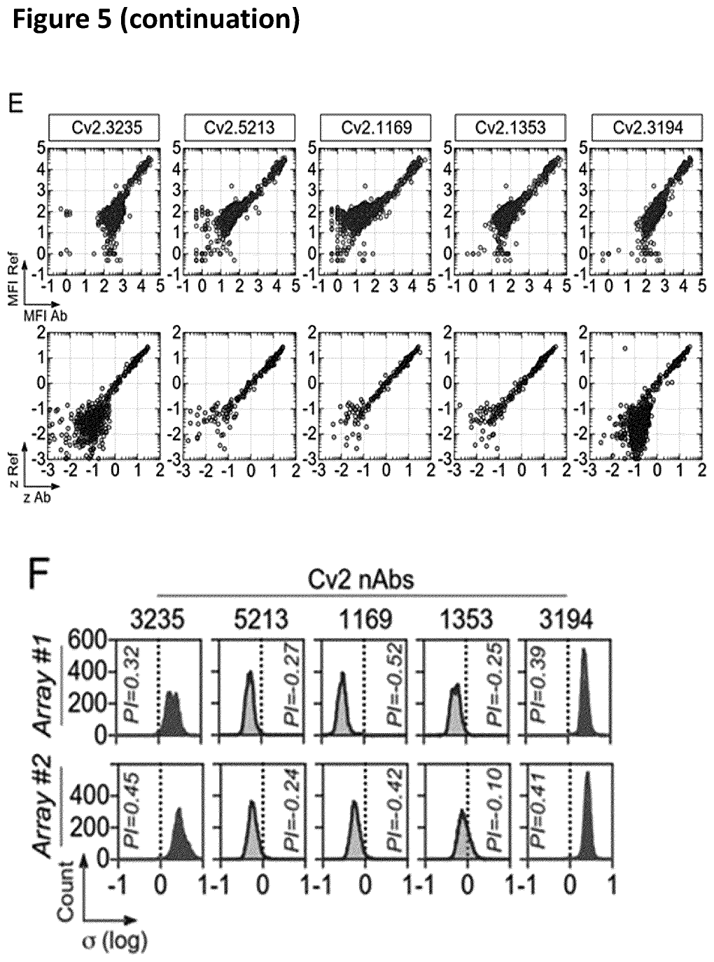

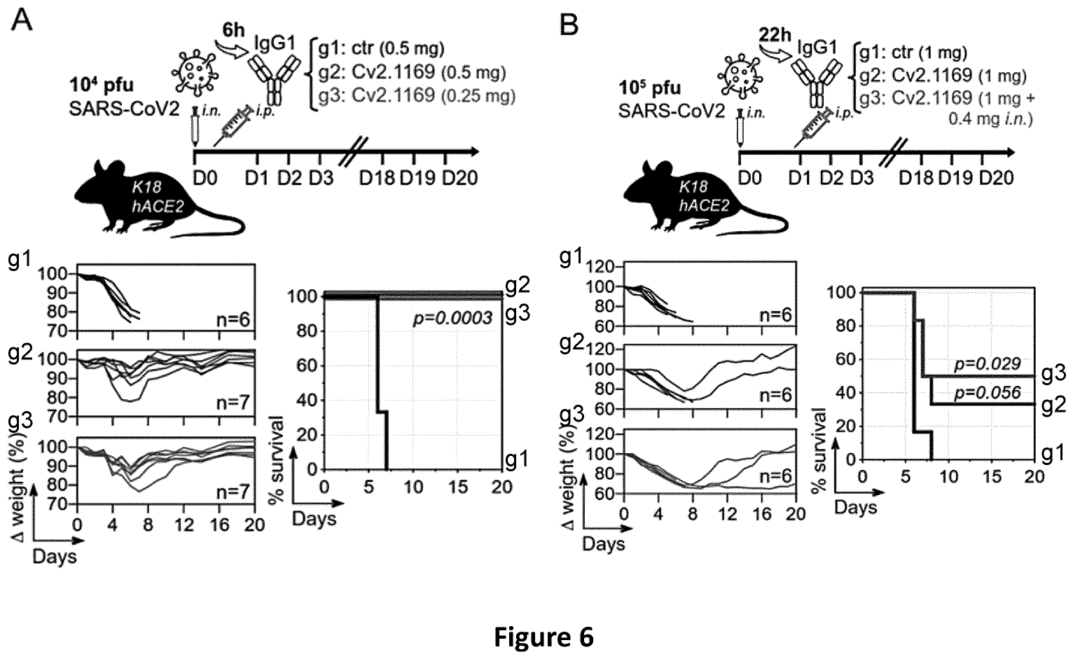

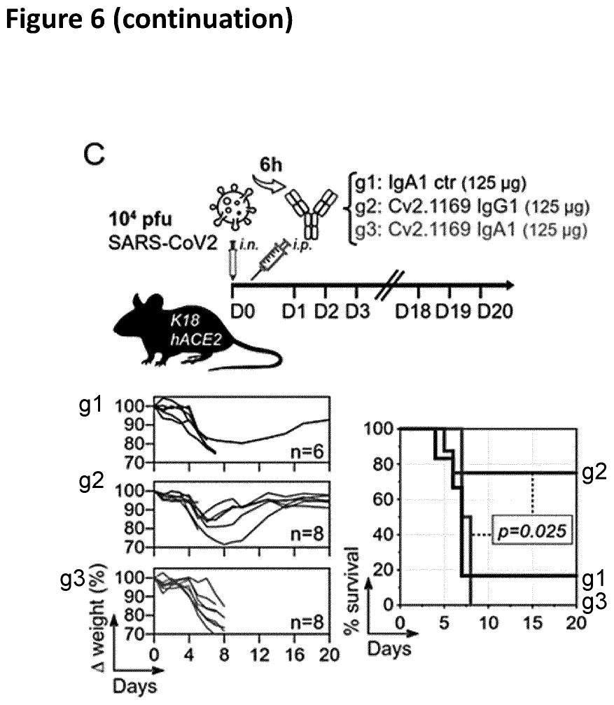

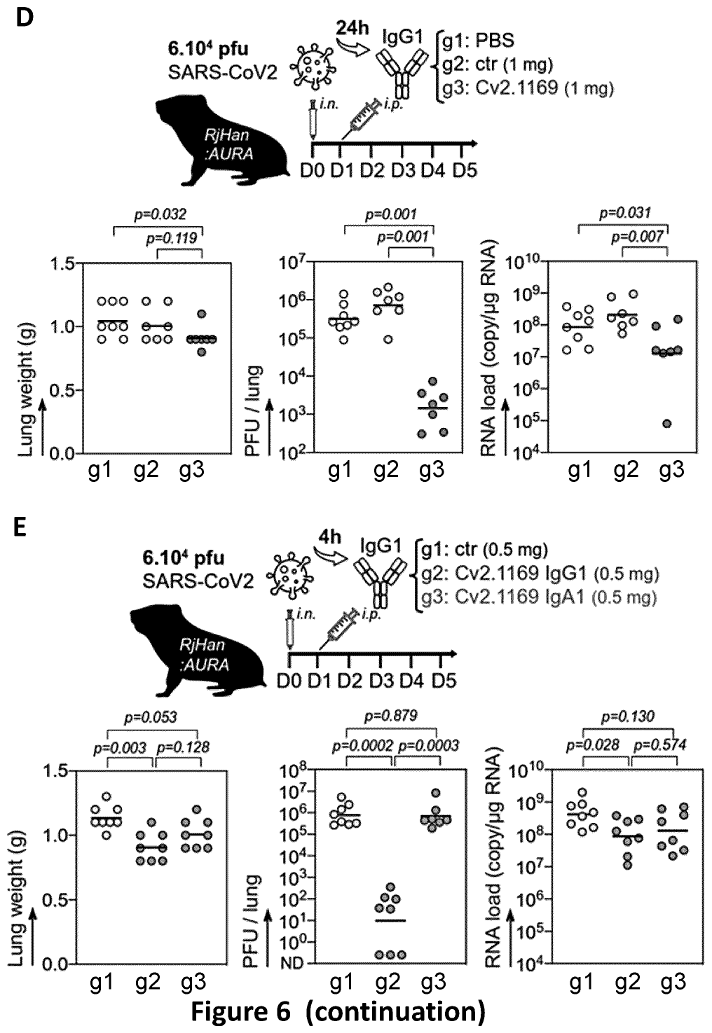

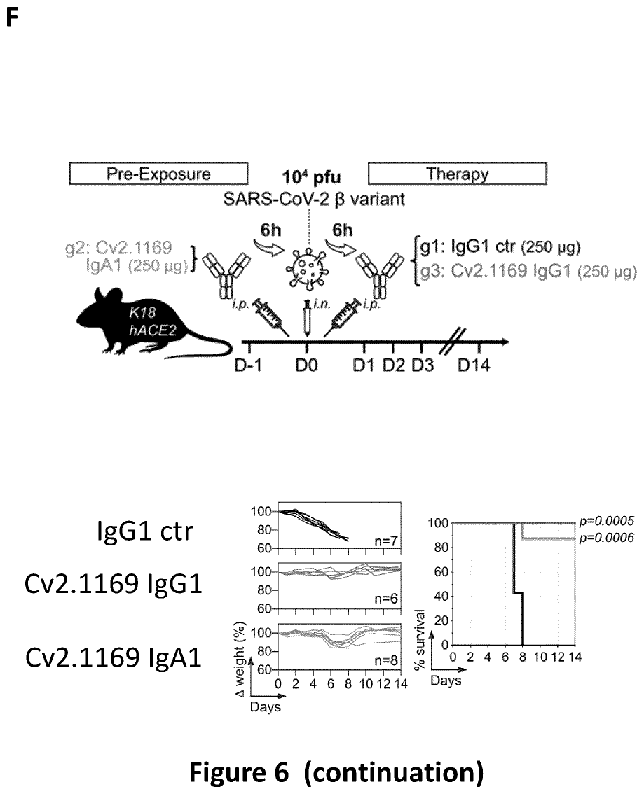

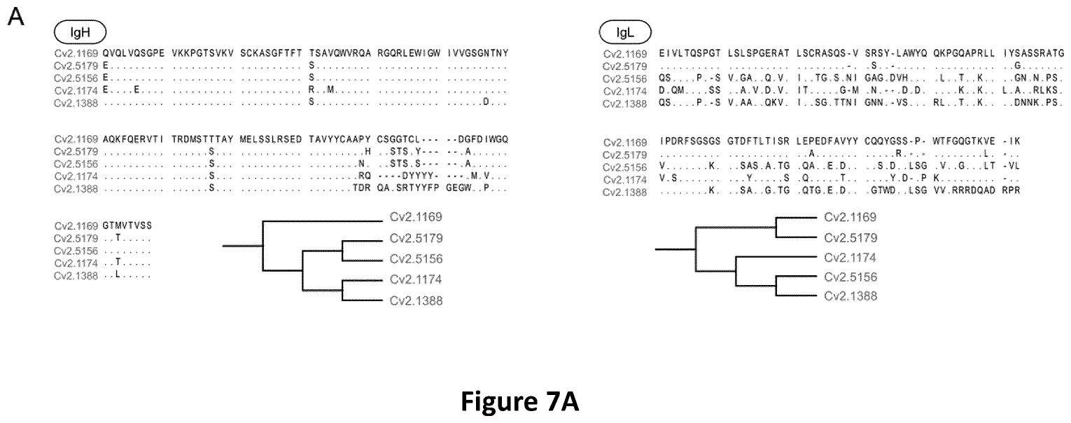

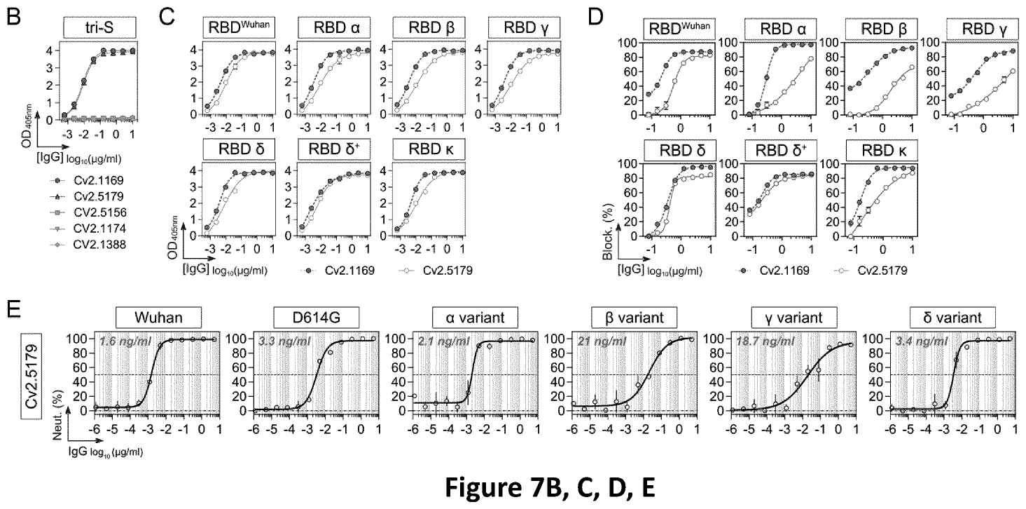

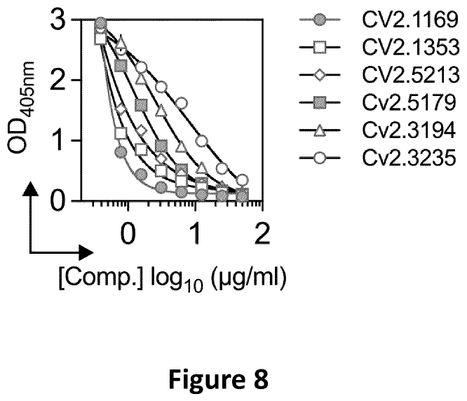

ELISA graphs showing the reactivity of Cv2.1169 antibody to SARS-CoV-2 Wuhan (historical data) and BA.1 tri-S(A); and RBD proteins from SARS-CoV-2 Wuhan and viral variants (P and BA.1) (B). Means±SD of duplicate OD_{405 nm} values are shown. (C) IC₅₀ values for Cv2.1169 and selected benchmark antibodies are indicated in the heatmap. Empty cells in grey indicate that the antibodies did not bind and thus, IC₅₀ values could not be determined.

FIG. 10. Binding of SARS-CoV-2 S protein (tri-S) to immobilized ACE-2 in the presence of Cv2.1169 and benchmark antibodies
(A) Competition ELISA showing the binding of biotinylated SARS-CoV-2 Wuhan and BA.1 tri-S proteins to soluble ACE2 ectodomain in presence of Cv2.1169 antibody. Means±SD of duplicate values are shown.
(B) IC₅₀ values for Cv2.1169 and selected benchmark antibodies (comprising variable regions SEQ ID No 158 to 163) are indicated in the heatmap. The value of 2.0 (in grey) indicates that 50% binding inhibition was not reached at an IgG concentration of 2 μg/ml.

FIG. 11. Neutralization of SARS-CoV-2 Delta and Omicron (BA.1) by Cv2.1169 and benchmark antibodies as determined with the S-Fuse neutralization assay
(A) Graph shows neutralization curve of SARS-CoV-2 Delta and BA.1 viral variants by Cv2.1169 antibody as determined by the S-Fuse neutralization assay. Error bars indicate the SD of assay duplicates.
(B) IC₅₀ values (ng/ml) for Cv2.1169 and selected benchmark antibodies are indicated in the heatmap on the right. The values in grey indicate that the 50% binding inhibition was not reached at the maximum concentration (>9000 ng/ml).

FIG. 12. Binding and neutralizing properties of potent anti-RBD neutralizers toward SARS-CoV-2 viral variants κ, δ and δ⁺—Related to FIG. 3F (right panel).

Heatmaps comparing the binding (left) and RBD-ACE2 blocking capacity (right) of RBD-specific IgG antibodies for RBD proteins of SARS-CoV-2 and selected viral variants (including δ, δ⁺ and κ) as measured in FIGS. 4E-4H. Darker colors indicate high binding or competition while light colors show moderate binding or competition (white=no binding or competition). AUC values are shown in each cell.

FIG. 13. Binding characteristics of potent anti-RBD antibody neutralizer Cv2.5179 toward SARS-CoV-2 viral variants κ, δ and δ⁺. —Related to FIG. 3L.

Heatmaps comparing the binding (top) and RBD-ACE2 blocking capacity (bottom) of RBD-specific IgG antibody Cv2.5179 to RBD proteins of SARS-CoV-2 and selected viral variants (including δ, δ⁺ and κ) as in FIG. 12. Means±SD of duplicate values are shown.

FIG. 14. Cross neutralization and Fc-dependent effectors functions of potent SARS-CoV2 neutralizers.
(A) Graphs comparing the ADCC activity of selected neutralizing (nAbs) and non-neutralizing (non-nAbs) SARS-CoV-2 S-specific antibodies. Means±SD of duplicate values are shown.
(B) Same as in (A) but for the CDC activity.
(C) ADCP activity of Cv2.1169. The dot plot (left) shows the monocyte-mediated ADCP activity of Cv2.1169 IgG at a concentration of 1 and 10 μg/ml. Each dot corresponds to a donor of primary monocytes (n=6). Graph comparing the ADCP activity of Cv2.1169 expressed as recombinant IgG1, IgG1^{NA}, IgG1^{LALA}, monomeric IgA1 (mIgA1) and dimeric IgA1 (dIgA1) antibodies. mGO53 is the negative isotype control, and ADCP-induced S309 IgG1 was included for comparison. PS, phagocytic score. Means of duplicate values are shown.
(D) Graphs showing the neutralization curves of SARS-CoV-2 and selected VOCs by potent anti-RBD IgG antibodies as determined with the S-Fuse neutralization assay. Error bars indicate the SD of duplicate values. IC₅₀ values are indicated in the top left-hand corner. ND, not determined.
(E) Graphs showing the neutralization curves of SARS-CoV-2 and selected VOCs by Cv2.1169 and Cv2.3194 IgG antibodies as determined with the pseudo-neutralization assay. Error bars indicate the SD of duplicate values. IC₅₀ values are indicated in the top left-hand corner.

FIG. 15. Cv2.1169 antibody treatment in SARS-CoV-2-infected mice and hamsters.
(A) Dot plot comparing the SARS-CoV-2 RNA levels in the oral swabs (OS) of (Wuhan) SARS-CoV-2-infected K18 hACE2 mice (at 4 dpi) treated with 5 mg/kg i.p. of Cv2.1169 IgG or IgA (n=8/group) or mGO53 control (ctr) IgA antibody (n=6) as shown in FIG. 6C. Each dot corresponds to a mouse. Means of duplicate values are shown.
(B) Dot plot comparing the human IgG concentrations in the serum of SARS-CoV-2-infected K18 hACE2 mice (at 20 dpi) receiving once 5, 10 or 20 mg/kg i.p. of antibody Cv2.1169 (n=7/group) as shown in FIGS. 6A and 6C. Each dot corresponds to a mouse. Means of duplicate values are shown.
(C) Dot plot comparing the human IgG and IgA concentrations in the serum of K18 hACE2 mice infected with the SARS-CoV-2 β variant, and pre-treated (IgA, n=8) or treated (IgG, n6) with Cv2.1169, or with mGO53 IgG control (ctr, n=7) as shown in FIG. 6F. Each dot corresponds to a mouse. Means of duplicate values are shown.
(D) Dot plot showing the ELISA SARS-CoV-2 tri-S binding of serum murine IgG antibodies in K18 hACE2 mice infected with the SARS-CoV-2 β variant, and pre-treated (IgA, n=8) or treated (IgG, n=6) with Cv2.1169 as shown in FIG. 6F. AUC, area under the curve. Each dot corresponds to a mouse. Means of duplicate values are shown.
(E) Dot plot comparing the human IgG and IgA concentrations in the serum of SARS-CoV-2-infected golden Syrian hamsters (at 5 dpi) treated once with Cv2.1169 or mGO53 control (5 mg/kg i.p., n=8; or 10 mg/kg i.p., n=7) as shown in FIGS. 6D and 6E (left and right, respectively). Each symbol corresponds to a mouse. Means of duplicate values are shown.
(F) Dot plot showing the ELISA SARS-CoV-2 tri-S binding of serum hamster IgG antibodies in SARS-CoV-2-infected hamsters (at 5 dpi) treated once with Cv2.1169 or mGO53 control (5 mg/kg i.p., n=8; or 10 mg/kg i.p., n=7) as shown in FIGS. 6D and 6E (left and right, respectively). AUC, area under the curve. Each symbol corresponds to a mouse. Means of duplicate values are shown.

FIG. 16. Benchmarking of Cv2.169 and Cv2.3194 with other anti-Spike antibodies.

Heatmaps comparing the ELISA binding (A) and the flow cytometry binding (B) of RBD-specific antibodies against selected viral variants. Darker colors indicate high binding while light colors show moderate binding (white=no binding). Means of duplicate OD_{405 nm} values and geometric means of duplicate log 10 ΔgMFI values are shown in each cell for (A) and (B), respectively. The following reference RBD-specific antibodies are reported: Adintrevimab (ADI), Bamlaivimab (BAM), Casirivimab (CAS), Cilgavimab (CIL), Etesevimab (ETE), Imdevimab (IMD), Regdanvimab (REG), Sotrovimab (SOT), Tixagevimab (TIX). Variable regions for each antibody are provided as SEQ ID NO: 164 to 183.
(C) (D) (E) Heatmaps corresponding to the binding competition of a selection of RBD-specific antibodies against the RBD domain of the Wuhan strain (RBD Wuhan—16C), of the β-strain (16D) and of the δ-strain (16E). Darker colors indicate high competition while light colors show moderate competition (white=no competition). For each experiment, the competitor is defined in the Y-axis.

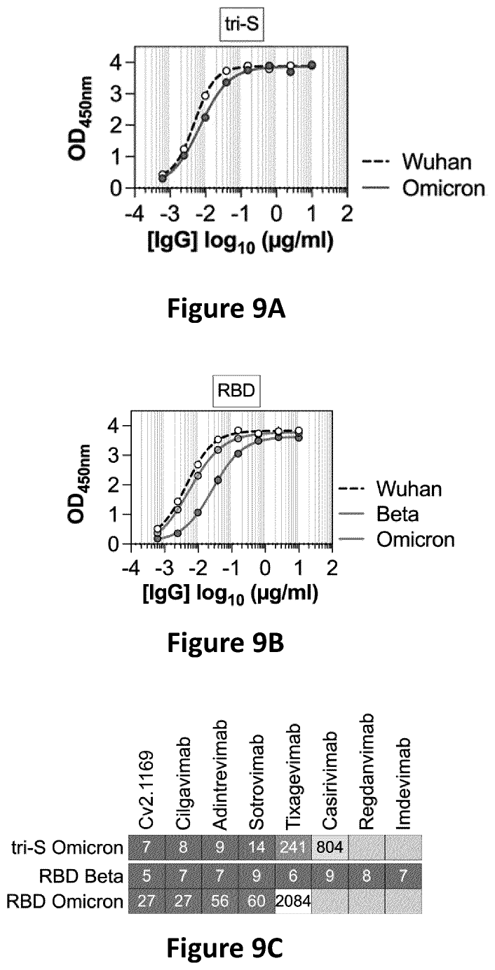

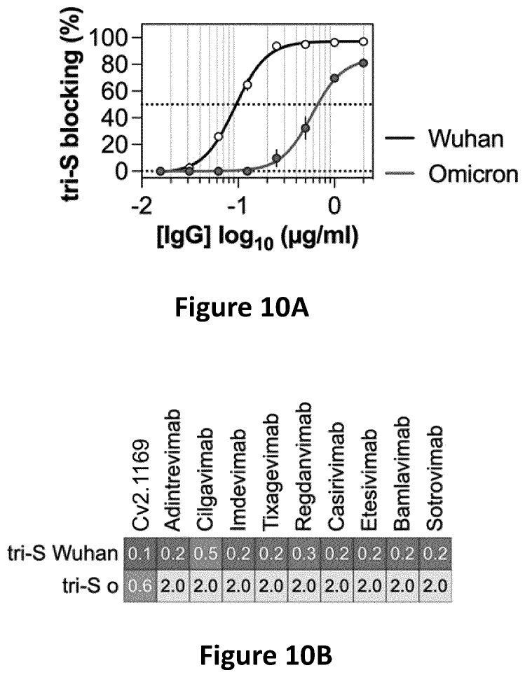

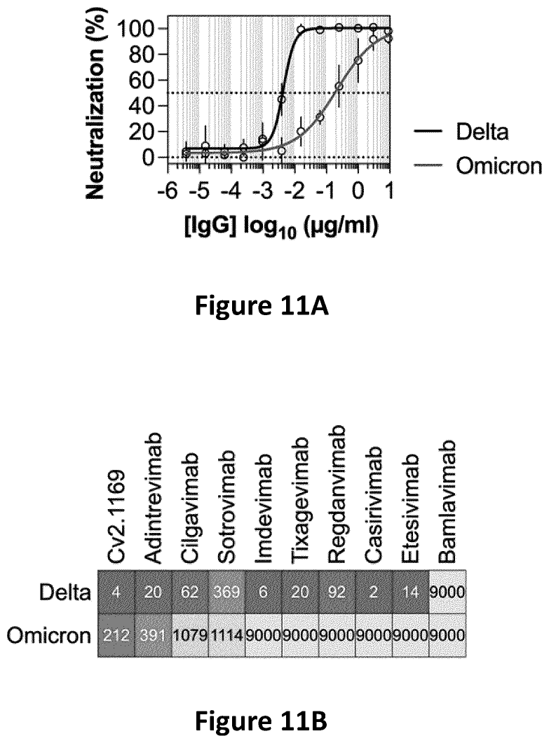

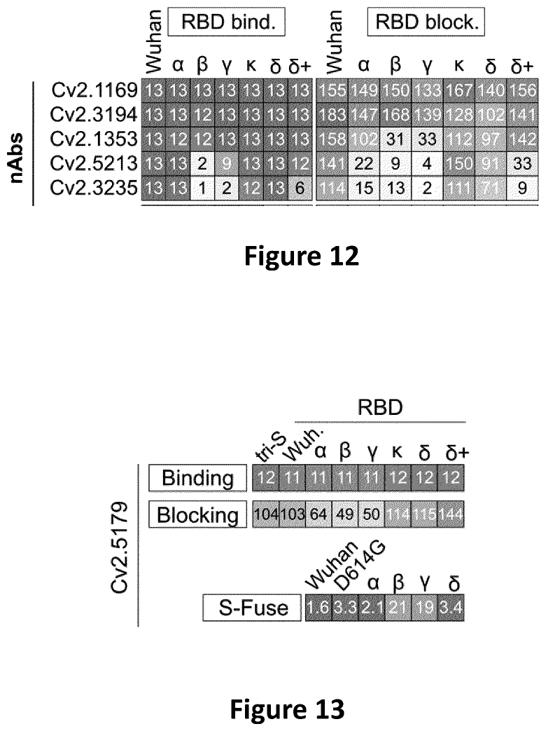

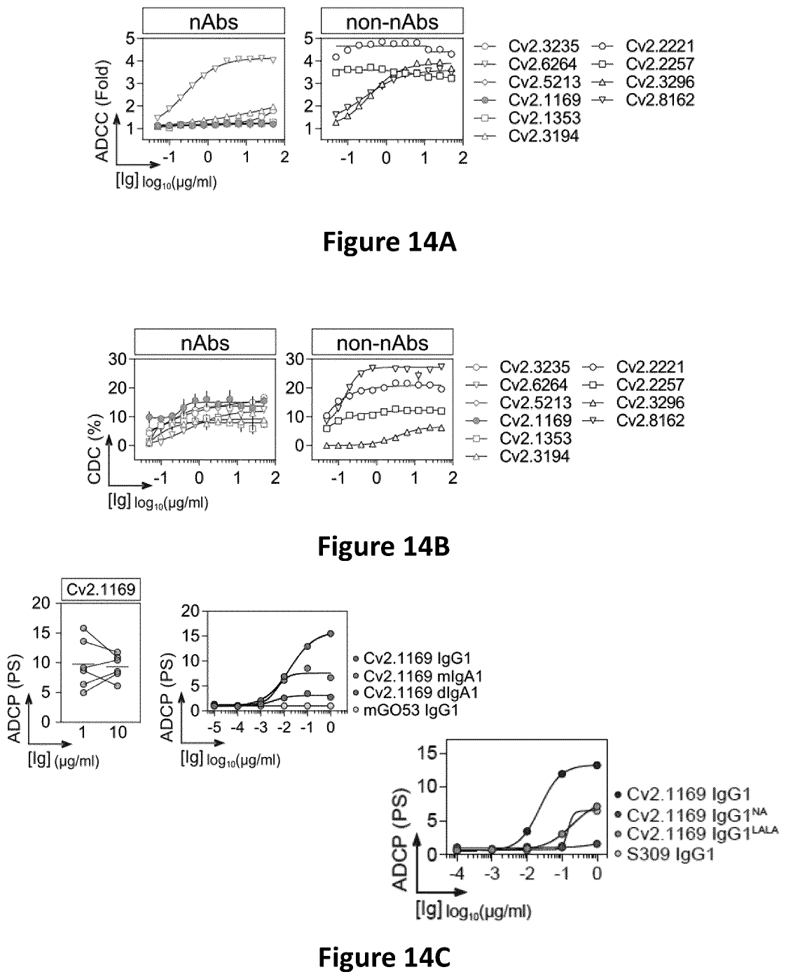

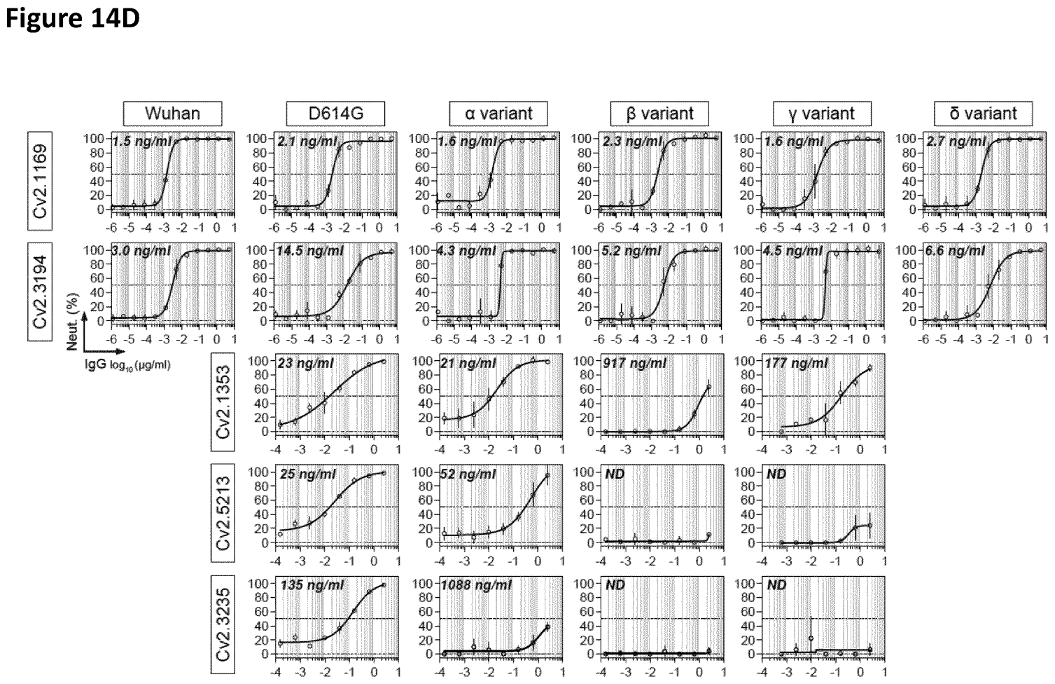

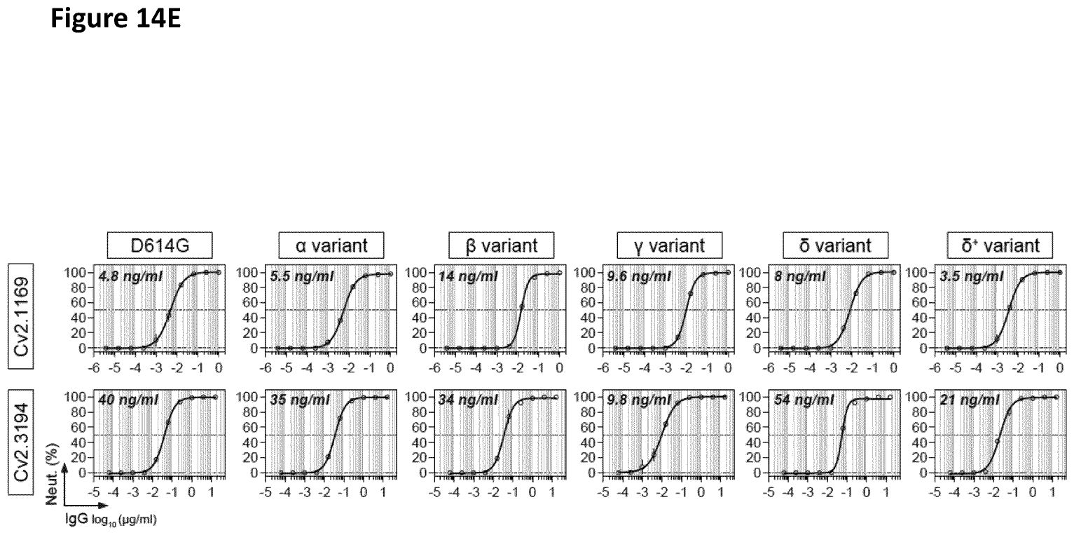

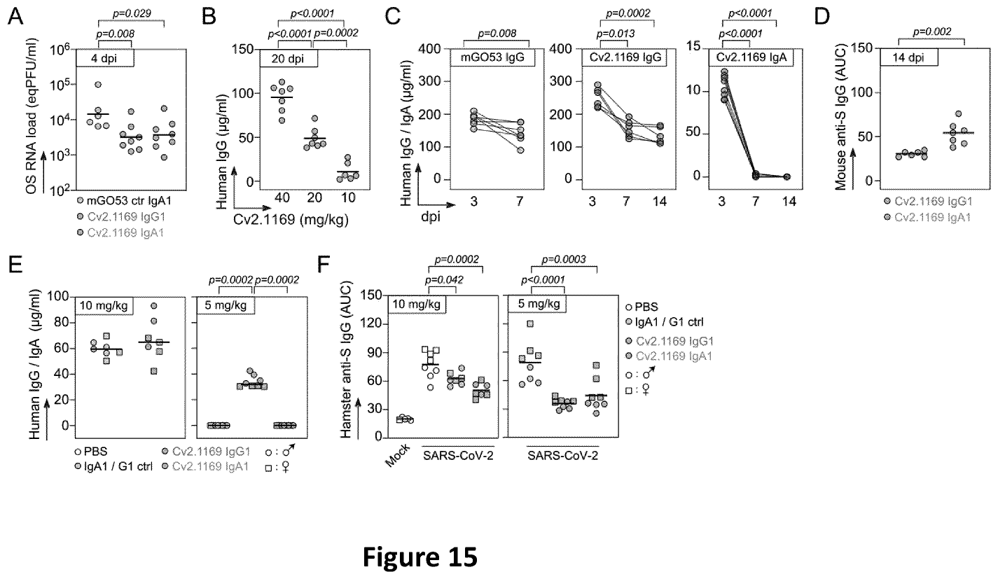

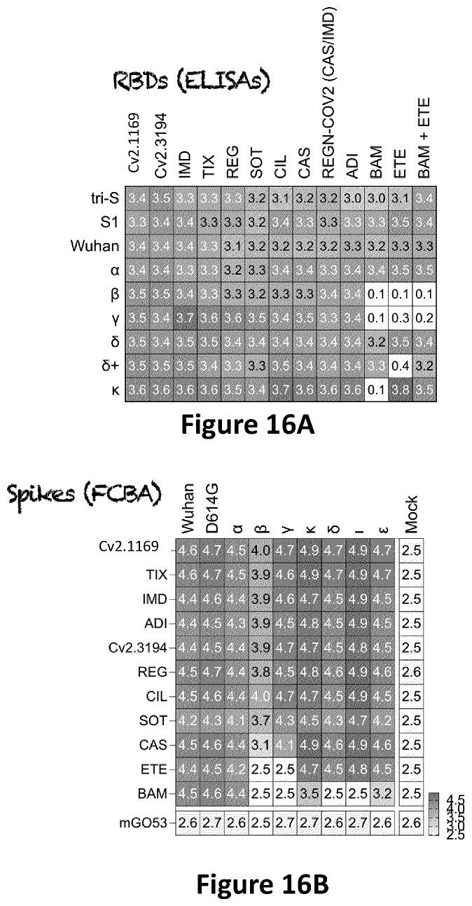

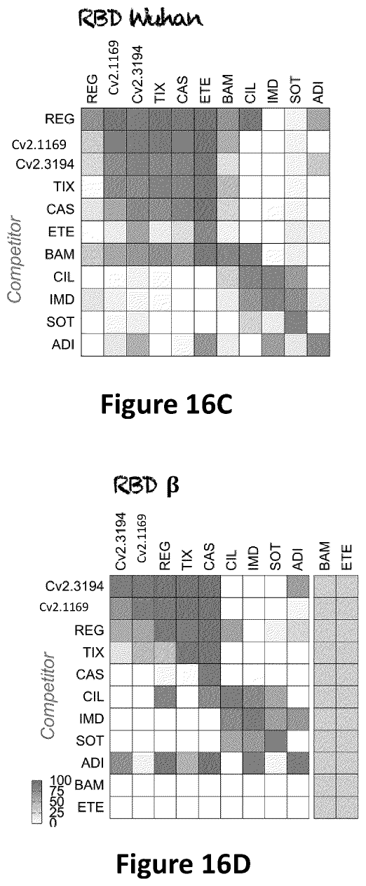

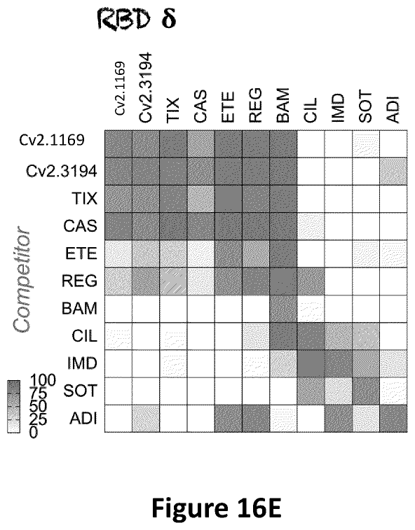

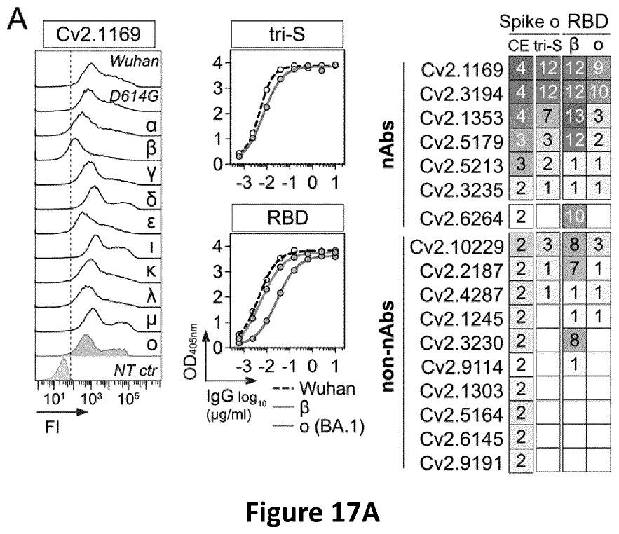

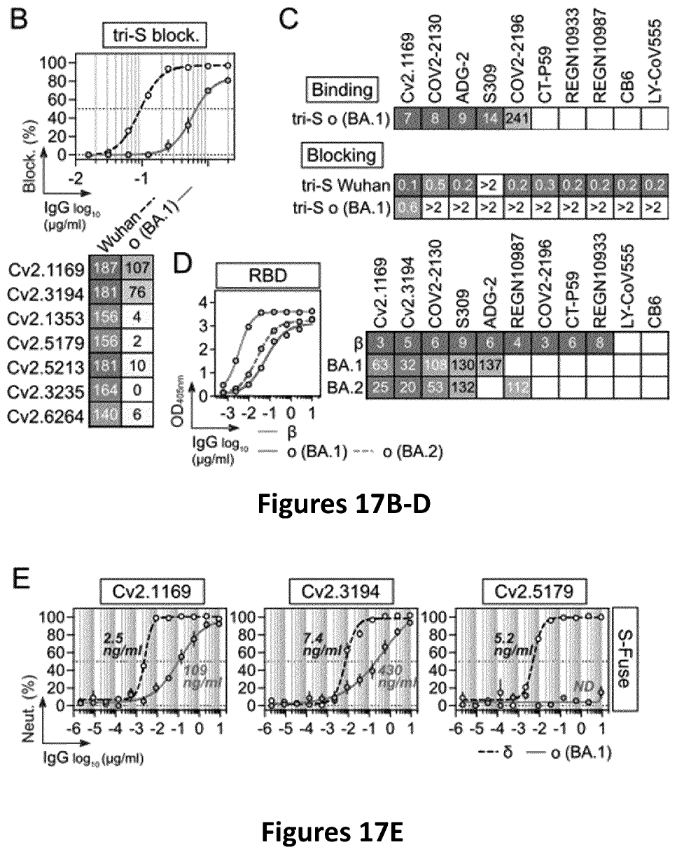

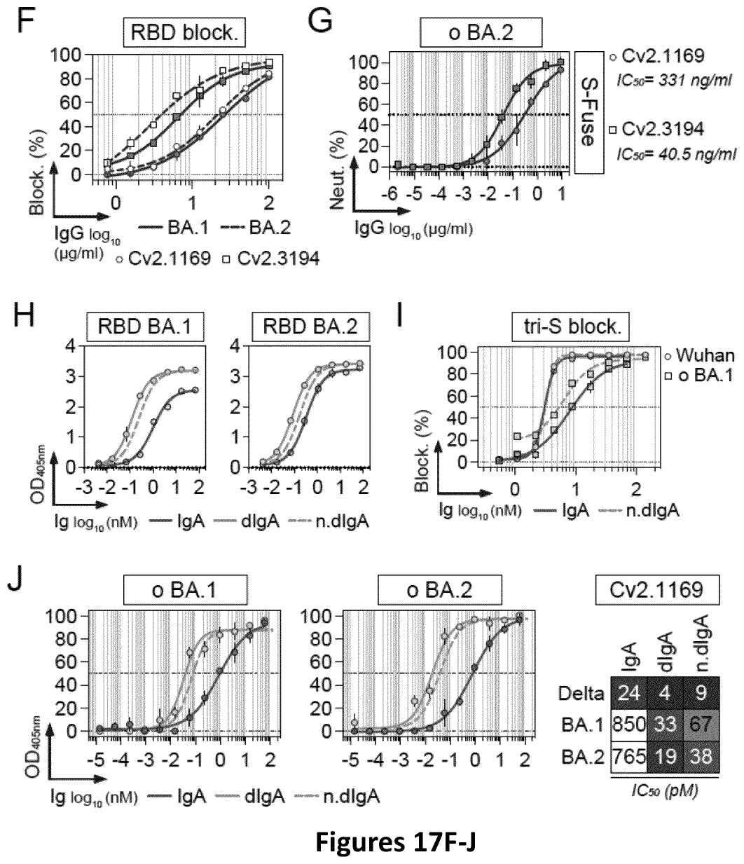

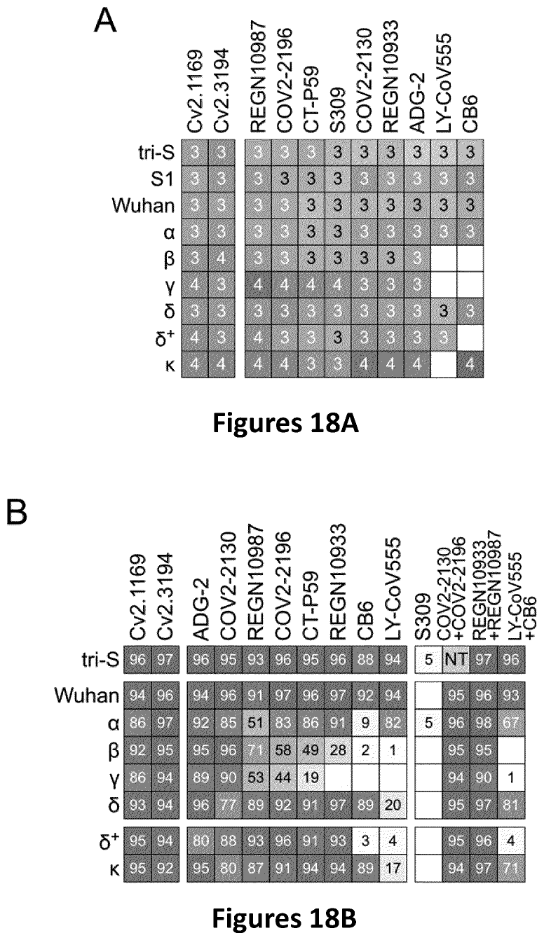

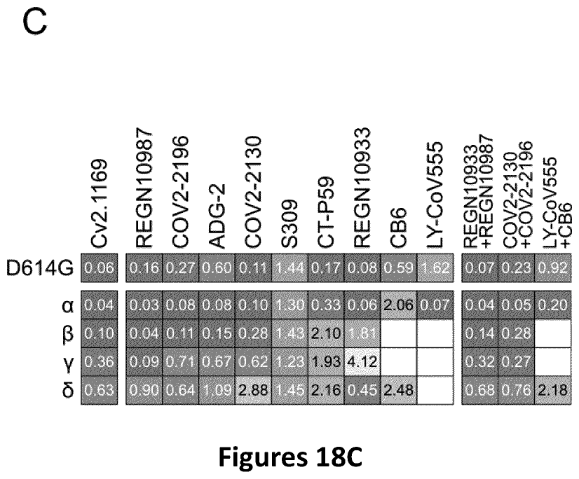

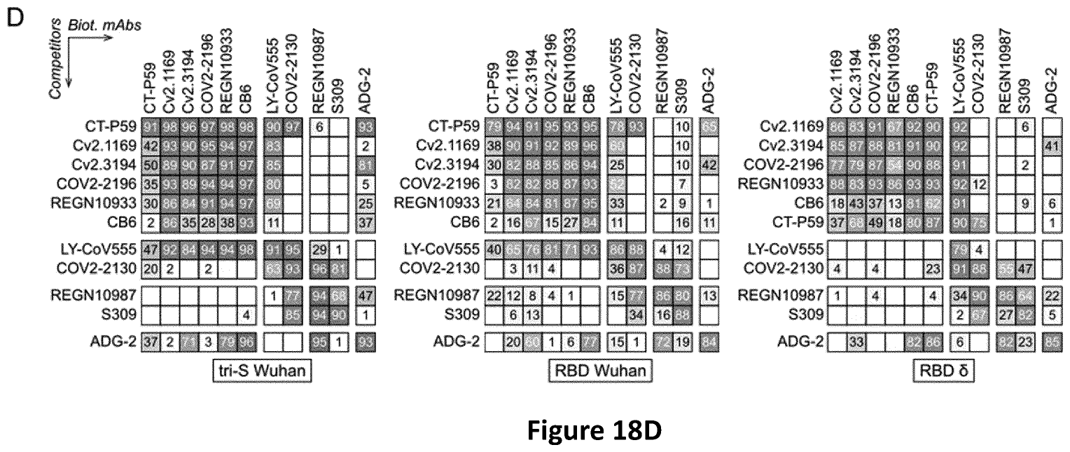

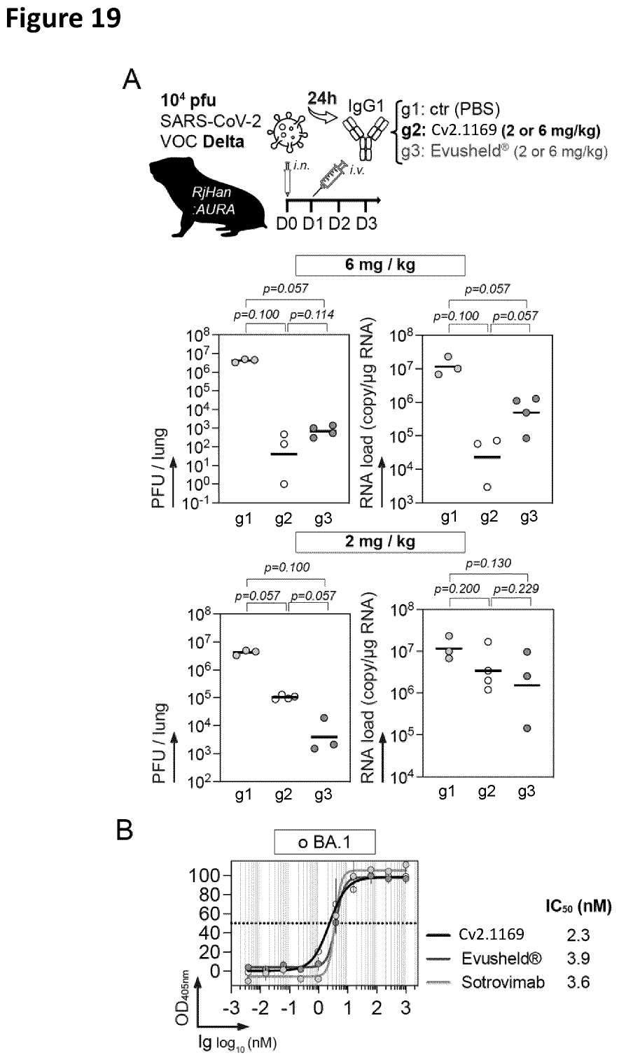

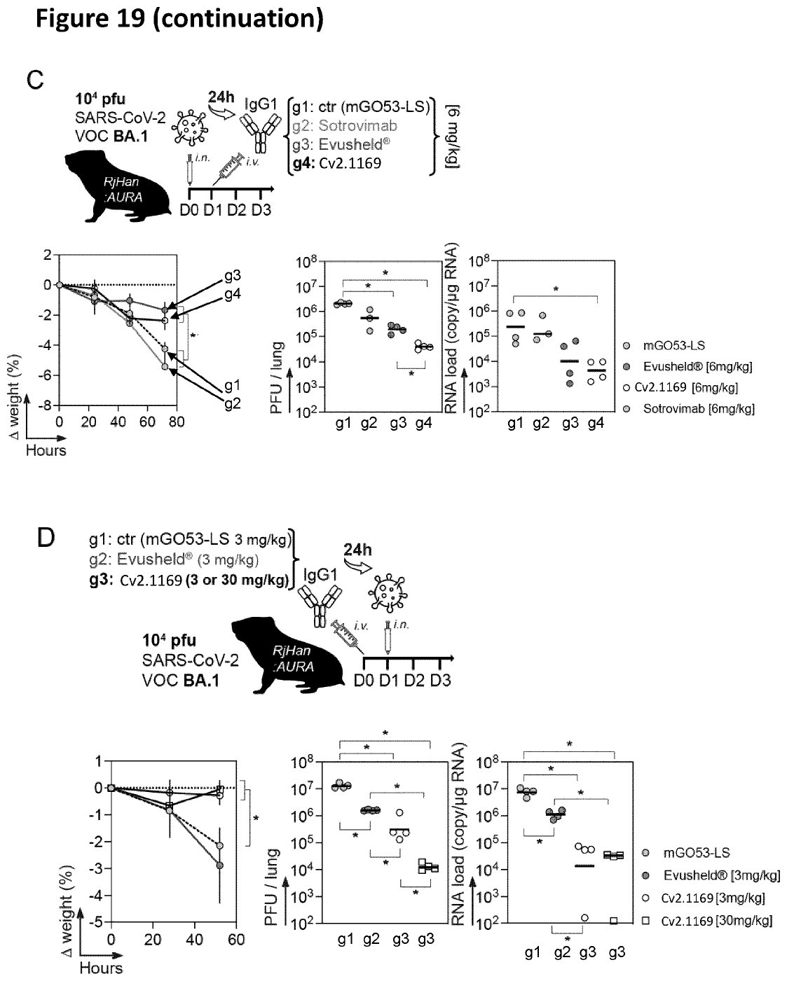

FIG. 17. Activity of Cv2.1169, Cv2.3194 and Cv2.5179 against SARS-CoV-2 Omicron variants with other benchmarked SARS-CoV-2 neutralizers—Related to FIG. 9.
(A) Heatmap (right) comparing the binding of RBD-specific IgG antibodies to the cell-expressed (CE) and soluble (tri-S) Omicron (o) SARS-CoV-2 spike proteins as measured by flow cytometry (mean log₁₀ ΔMFI from duplicate values) and ELISA (mean AUC from duplicate values), respectively, as shown on the left for Cv2.1169. NT ctr, non-transfected cell control. The heatmap also presents the comparative antibody reactivity (AUC values) against β and o RBD proteins. White indicates no binding.
(B) Heatmap (bottom) comparing the RBD-ACE2 blocking capacity of neutralizing anti-RBD antibodies for the RBD proteins of SARS-CoV-2 and o variant BA.1 as shown for Cv2.1169 (top). Darker colors indicate high competition while light colors show moderate competition (white=no binding or competition). Mean AUC from duplicate values are shown in each cell.
(C) Heatmaps comparing the tri-S binding (top) and tri-S-ACE2 blocking capacity (bottom) of Cv2.1169 with benchmarked SARS-CoV-2 neutralizers RBD-specific IgG antibodies to the SARS-CoV-2 proteins of the o variant BA.1. Darker colors indicate high binding or competition while light colors show moderate binding or competition (white=no binding or competition). Mean EC₅₀ from duplicate values are shown in each cell.
(D) Heatmap (right) comparing the binding of Cv2.1169 and Cv2.3194 with benchmarked SARS-CoV-2 neutralizers for the RBD proteins of the o variant BA.1 and BA.2 as measured ELISA (means of duplicate AUC values) as shown on the left for Cv2.1169. Darker colors indicate high binding while light colors show moderate binding (white=no binding). Mean EC₅₀ from duplicate values are shown in each cell.
(E) Graphs showing the neutralization curves of SARS-CoV-2 δ and o BA.1 by potent anti-RBD IgG antibodies as determined with the S-Fuse neutralization assay. Error bars indicate the SD of duplicate values from 2 (Cv2.5179) or 5 (Cv2.1169 and Cv2.3194) independent experiments. IC₅₀ values are indicated (in grey for o BA.1). ND, not determined.
(F) Competition ELISA graphs showing the binding of biotinylated RBD proteins from SARS-CoV-2 o BA.1 and BA.2 variants to soluble ACE2 ectodomain in presence of Cv2.1169 and Cv2.3194 antibodies as competitors. Means±SD of duplicate values are shown.
(G) Graphs showing the neutralization curves of SARS-CoV-2 o BA.2 by Cv2.1169 and Cv2.3194 as determined with the S-Fuse neutralization assay. Error bars indicate the SD of duplicate values.
(H) Graphs comparing the ELISA binding of monomeric and dimeric Cv2.1169 IgA antibodies to the RBD proteins of SARS-CoV-2 o BA.1 and BA.2 variants. Means±SD of duplicate values are shown. n.dIgA, normalized values according to the number of binding sites.
(I) Same as in (E) but for Wuhan and o BA.1 tri-S proteins with monomeric and dimeric Cv2.1169 IgA antibodies. Means±SD of duplicate values are shown. n.dIgA, normalized values according to the number of binding sites.
(J) Same as in (F) but for Cv2.1169 IgA monomers and J-chain dimers (dIgA) against BA.1 and BA.2. Error bars indicate the SD of duplicate values. Heatmap (right) presents the IC₅₀ values calculated from the curves (left). n.dIgA, normalized values according to the number of binding sites.

FIG. 18. Comparative analyses of Cv2.1169 with benchmarked antibodies. As in FIGS. 9, 16 and 17.
(A) Heatmap comparing the ELISA binding to the selected SARS-CoV-2 proteins of Cv2.1169, Cv2.3194 and benchmarked neutralizing antibodies in clinical use or in development. Darker colors indicate high binding while light colors show moderate binding or competition (white=0, no binding). Means of duplicate AUC values are shown in each cell.
(B) Heatmap comparing the tri-S- and RBD-ACE2 blocking capacity of Cv2.1169, Cv2.3194 and benchmarked neutralizing antibodies. Darker colors indicate high competition while light colors show moderate competition (white=0, no competition). Means of duplicate values (% binding inhibition) are shown in each cell. NT, not tested.
(C) Heatmap comparing the in vitro neutralizing activity of Cv2.1169 and benchmarked neutralizing antibodies against the selected SARS-CoV-2 viral variants. Means of triplicate IC₅₀ values in pM are shown in each cell. White color indicates that 50% neutralization was not reached at the maximum antibody concentration of 25 nM.
(D) Heatmaps showing the competition potential of Cv2.1169, Cv2.3194 and benchmarked neutralizing antibodies for the ELISA binding to tri-S and RBD proteins. Darker colors indicate high competition while light colors show moderate competition (white=0, no competition). Means of duplicate values (% binding inhibition) are shown in each cell.

FIG. 19. In vivo therapeutic and prophylactic activity of Cv2.1169 and benchmarked antibodies in hamsters infected with SARS-CoV-2 Delta and Omicron BA.1.
(A) Schematic diagram showing the experimental design for the antibody therapy with Cv2.1169 and Evusheld in golden Syrian hamsters infected with SARS-CoV-2 δ variant. Animals were infected intranasally (i.n.) with 10⁴ plaque forming units (PFU) of SARS-CoV-2 δ and received 24 h later an intraperitoneal (i.p.) injection of PBS, Cv2.1169 or Evusheld at 6 mg/kg (top) or 2 mg/kg (bottom). Dot plots showing the intra-lung infectivity (left) and RNA load (right) measured in animal groups (n=3) at 3 dpi. Groups of hamsters were compared using two-tailed Mann-Whitney test.
(B) Graphs comparing the neutralization curves of SARS-CoV-2 Omicron (o) BA.1 by Cv2.1169, Evusheld, and Sotrovimab as determined with the neutralization assay on Vero cells with qRT-PCR determination. Error bars indicate the SD of triplicate values. IC₅₀ values are indicated (right).
(C) Same as in (A) but with animals infected with SARS-CoV-2 Omicron BA.1 and treated with Cv2.1169, Evusheld, Sotrovimab or control (mGO53-LS) antibodies at 6 mg/kg. Plots showing the animal weight variation compared to baseline (Δweight) overtime (left), intra-lung infectivity (center) and RNA load (right). Groups of hamsters (n=4) were compared using two-tailed Mann-Whitney test. Asterisks indicate p=0.029.
(D) Same as in (C) but with animals (n=4) pre-treated with Cv2.1169 (3 or 30 mg/kg), Evusheld (3 mg/kg), or control (mGO53-LS, 3 mg/kg) antibodies before a i.n. challenge with 10⁴ PFU of SARS-CoV-2 Omicron BA.1.

### EXAMPLES

### Material and Methods

### Human Samples

Blood samples from COVID-19 convalescent donors were obtained as part of the CORSER and REACTing French COVID-19 cohorts in accordance with and after ethical approval from all the French legislation and regulation authorities. The CORSER study was registered with ClinicalTrials.gov (NCT04325646), and received ethical approval by the Comit6 de Protection des Personnes Ile de France III. The REACTing French Covid-19 study was approved by the regional investigational review board (IRB; Comit6 de Protection des Personnes Ile-de-France VII, Paris, France) and performed according to the European guidelines and the Declaration of Helsinki. All participants gave written consent to participate in this study, and data were collected under pseudo-anonymized conditions using subject coding.

### Serum IgG and IgA Purification

All human sera were heat-inactivated at 56° C. for 60 minutes. Human IgG and IgA antibodies were purified from donors' sera by affinity chromatography using Protein G Sepharose® 4 Fast Flow (GE Healthcare) and peptide M-coupled agarose beads (Invivogen), respectively. Purified serum antibodies were dialyzed against PBS using Slide-A-Lyzer® Cassettes (10K MWCO, Thermo Fisher Scientific).

### Viruses

The SARS-CoV-2 BetaCoV/France/IDF0372/2020, the hCoV-19/France/GES-1973/2020, the D614G (hCoV-19/France/GE1973/2020) and the B.1.351 (β variant; hcoV-19/France/IDF-IPP00078/2021) strains were provided by the National Reference Centre for Respiratory Viruses (Institut Pasteur France). The human sample from which strain BetaCoV/France/IDF0372/2020 was isolated has been provided by Drs. Xavier Lescure, Yazdan Yazdanpanah from the Bichat Hospital, Paris. The human sample from which the β variant was isolated has been provided by Dr. Mounira Smati-Lafarge (CHI de Créteil, Créteil, France). All variant strains were isolated from nasal swabs and amplified by one or two passages in Vero E6 cell cultures. The a variant (B.1.1.7) and the 6 variant (B.1.617.2) used for neutralization assays were isolated from individuals in Tours (France) and Hopital Européen Georges Pompidou (Assistance Publique des Hôpitaux de Paris, Paris, France), respectively. The γ variant (P.1.; hCoV-19/Japan/TY7-501/2021) was obtained from Global Health security action group Laboratory Network. Sequences ID of the GISAID database are as follow: D614G: EPI_ISL_414631; α variant: EPI_ISL_735391; β variant: EPI_ISL_964916; γ variant: EPI_ISL_833366; δ variant: EPI_ISL_2029113. All individuals provided informed consent for the use of the biological materials. The viruses were amplified by one or two passages in Vero E6 cell cultures and titrated. The sequence of the viral stocks was verified by RNAseq. All work with infectious virus was performed in biosafety level 3 containment laboratories at Institut Pasteur.

### Expression and Purification of Viral Proteins

Codon-optimized nucleotide fragments encoding stabilized versions of SARS-CoV-2, SARS-CoV-1, MERS-CoV, OC43-CoV, HKU1-CoV, 229E-CoV and NL63-CoV (2P) and BA.1 spike (HexaPro) Spike (S) ectodomains, and SARS-CoV-2 S2 domain, followed by a foldon trimerization motif and C-terminal tags (His×8-tag, Strep-tag, and AviTag) were synthesized and cloned into pcDNA3.1/Zeo(+) vector vector (thermo Fisher Scientific). For competition ELISA experiments, a SARS-CoV-2 S ectodomain DNA sequence without the StrepTag was also cloned into pcDNA3.1/Zeo(+) vector. Synthetic nucleotide fragments coding for Wuhan SARS-CoV-2 RBD, S1 subunit, S1 N-terminal domain (NTD), S1 connecting domain (CD), nucleocapsid protein (N), BA.1 and BA.2 RBDs followed by C-terminal tags (His×8-tag, Strep-tag, and AviTag) as well as human angiotensin-converting enzyme 2 (ACE2) (plus His×8- and Strep tags were cloned into pcDNA3.1/Zeo(+)vector. For SARS-CoV-2 RBD variant proteins, mutations (N501Y for the α variant, K417N, E484K and N501Y for the β variant; K471T, E484K and N501Y for the γ variant; L452R and T478K for the δ variant, K417N, L452R and T478K for the δ+ variant; L452R and E484Q for the κ variant) were introduced using the QuickChange Site-Directed Mutagenesis kit (Agilent Technologies) following the manufacturer's instructions. Glycoproteins were produced by transient transfection of exponentially growing Freestyle 293-F suspension cells (Thermo Fisher Scientific, Waltham, MA) using polyethylenimine (PEI) precipitation method as previously described (5). Proteins were purified from culture supernatants by high-performance chromatography using the Ni Sepharose® Excel Resin according to manufacturer's instructions (GE Healthcare), dialyzed against PBS using Slide-A-Lyzer® dialysis cassettes (Thermo Fisher Scientific), quantified using NanoDrop 2000 instrument (Thermo Fisher Scientific), and controlled for purity by SDS-PAGE using NuPAGE 3-8% tris-acetate gels (Life Technologies) as previously described (5). AviTagged tri-S and RBD proteins were biotinylated using BirA biotin-protein ligase bulk reaction kit (Avidity, LLC) or Enzymatic Protein Biotinylation Kit (Sigma-Aldrich). SARS-CoV-2 RBD protein was also coupled to DyLight 650 using the DyLight® Amine-Reactive Dyes kit (Thermo Fisher scientific).

For crystallographic experiments, a codon-optimized nucleotide fragment encoding the SARS-CoV-2 RBD protein (residues 331-528), followed by an enterokinase cleavage site and a C-terminal double strep-tag was cloned into a modified pMT/BiP expression vector (pT350, Invitrogen). Drosophila S2 cells were stably co-transfected with pT350 and pCoPuro (for puromycin selection) plasmids. The cell line was selected and maintained in serum-free insect cell medium (HyClone, Cytiva) supplemented with 7 μg/ml puromycin and 1% penicillin/streptomycin antibiotics. Cells were grown to reach a density of 1×10⁷ cells/ml, and protein expression was then induced with 4 μM CdCl₂. After 6 days of culture, the supernatant was collected, concentrated and proteins were purified by high-performance chromatography using a Streptactin column (IBA). The eluate was buffer-exchanged into 10 mM Tris-HCl (pH 8.0), 100 mM NaCl, 2 mM CaCl₂) using a HiPrep 26/10 Desalting column (GE Healthcare) and subsequently treated with enterokinase overnight at room temperature to remove the strep-tag. Undigested tagged proteins were removed using a Streptactin column, and monomeric untagged protein was purified by size-exclusion chromatography (SEC) using a Superdex 75 column (Cytiva) equilibrated with 10 mM Tris-HCl (pH 8.0), 100 mM NaCl. Purified monomeric untagged protein was concentrated and stored at −80° C. until used.

For Cryo-EM experiments, a codon-optimized nucleotide fragment encoding the SARS-CoV-2 spike (S) protein (residues 1-1208) was cloned with its endogenous signal peptide in pcDNA3.1(+) vector, and expressed as a stabilized trimeric prefusion construct by introduction of six proline substitutions (F817P, A892P, A899P, A942P, K986P, V987P) along with a GSAS substitution at the furin cleavage site (residues 682-685), followed by a Foldon trimerization motif, and C-terminal tags (His×8-tag, Strep-tag and AviTag). The recombinant protein, S_6P, was produced by transient transfection of Expi293FTM cells (Thermo Fisher Scientific, Waltham, MA) using FectroPRO® DNA transfection reagent (Polyplus), according to the manufacturer's instructions. After 5 days of culture, recombinant proteins were purified from the concentrated supernatant by affinity chromatography using a SrepTactin column (IBA), followed by a SEC using a Superose 6 10/300 column (Cytiva) equilibrated in 10 mM Tris-HCl, 100 mM NaCl (pH 8.0). The peak corresponding to the trimeric protein was concentrated and stored at −80° C. until used.

### Flow Cytometry Immunophenotyping

Peripheral blood mononuclear cells (PBMC) were isolated from donors' blood using Ficoll Plaque Plus (GE Healthcare). Human blood B cells and circulating T follicular helper T cells (cTfh) were analyzed using two different fluorescently-labeled antibody cocktails. For B-cell phenotyping, B cells were first isolated from donors' PBMC by MACS using human CD19 MicroBeads (Miltenyi Biotec). CD19⁺ B cells were then stained using LIVF/DEAD aqua fixable dead cell stain kit (Molecular Probes, Thermo Fisher Scientific) to exclude dead cells. B cells were incubated for 30 min at 4° C. with biotinylated tri-S and DyLight 650-coupled RBD, washed once with 1% FBS-PBS (FACS buffer), and incubated for 30 min at 4° C. with a cocktail of mouse anti-human antibodies: CD19 Alexa 700 (HIB19, BD Biosciences, San Jose, CA), CD21 BV421 (B-ly4, BD Biosciences), CD27 PE-CF594 (M-T271, BD Biosciences), IgG BV786 (G18-145, BD Biosciences), IgA FITC (IS11-8E10, Miltenyi Biotec, Bergisch Gladbach, Germany), Integrin β7 BUV395 (FIB504, BD Biosciences) and streptavidin R-PE conjugate (Invitrogen, Thermo Fisher Scientific). Cells were then washed and resuspended in FACS buffer. Following a lymphocyte and single cell gating, live cells were gated on CD19⁺ B cells. FACS analyses were performed using a FACS Aria Fusion Cell Sorter (Becton Dickinson, Franklin Lakes, NJ) and FlowJo software (v10.3, FlowJo LLC, Ashland, OR). Immunophenotyping of cTfh subsets was performed on negative fractions from the CD19 MACS. The cTfh antibody panel included: CD3 BV605 (SK7), CD4 PE-CF594 (RPA-T4), CD185/CXCR5 AF-488 (RF8B2), CD183/CXCR3 PE-Cy™5 (1C6/CXCR3), CD196/CCR6 PE-Cy™7 (11A9), CD197/CCR7 AF647 (3D12) (BD Biosciences), CD279/PD1 BV421 (EH12.2H7, BioLegend), and CD278/ICOS PE (ISA-3, Thermo Fisher Scientific). Cells were stained as described above, washed and fixed in 1% paraformaldehyde-PBS. Following a lymphocyte and single cell gating, dead cells were excluded. Flow cytometric analyses of stained cells were performed using a BD LSR Fortessa™ instrument (BD Biosciences), and the FlowJo software (v10.6, FlowJo LLC)

### Single B-Cell FACS Sorting and Expression-Cloning of Antibodies

Peripheral blood human B cells were isolated from donors' PBMCs by CD19 MACS (Miltenyi Biotec) and stained as describe above. Single SARS-CoV-2 S⁺ IgG⁺ and IgA⁺ B cells were sorted into 96-well PCR plates using a FACS Aria Fusion Cell Sorter (Becton Dickinson, Franklin Lakes, NJ) as previously described (6). Single-cell cDNA synthesis using SuperScript IV reverse transcriptase (Thermo Fisher Scientific) followed by nested-PCR amplifications of IgH, Igκ and Igλ genes, and sequences analyses for Ig gene features were performed as previously described (6, 13). Purified digested PCR products were cloned into human Igγ1-, Igκ- or Igλ-expressing vectors (GenBank #LT615368.1, LT615369.1 and LT615370.1, respectively) as previously described. Cv2.1169 were also cloned into human Igγ1NA, Igγ1LALA [N297A and L234A/L235A mutations introduced by Site-Directed Mutagenesis (QuickChange, Agilent Technologies)], Igα1 and Fab-Igα1-expressing vectors. Cv2.3235, and Cv2.6264 IgH were also cloned into a human Fab-Igγ1-expressing vector.

Recombinant antibodies were produced by transient co-transfection of Freestyle™ 293-F suspension cells (Thermo Fisher Scientific) using PEI-precipitation method as previously described (5). The dimeric form of Cv2.1169 IgA1 was produced by co-transfection of Freestyle™ 293-F cells with a human J chain pcDNA™3.1/Zeo(+) vector as previously described. Recombinant human IgG and IgA antibodies and Fab fragments were purified by affinity chromatography using Protein G Sepharose® 4 Fast Flow (GE Healthcare), peptide M-coupled agarose beads (Invivogen) and Ni Sepharose® Excel Resin (GE Healthcare), respectively. Monomeric and dimeric Cv2.1169 IgA1 antibodies were separated by SEC using a Superose 6 Increase 10/300 column (Cytiva). After equilibration of the column with PBS, purified IgA antibodies were injected into the column at a flow rate of 0.3 ml/min. Monomers, dimers and multimers were separated upon an isocratic elution with 1.2 CV of PBS. The quality/purity of the different purified fractions was evaluated by SDS-PAGE using 3-8% Tris-Acetate gels (Life Technologies) under non-reducing conditions followed by a silver staining (Siver Stain kit, Thermo Scientific). Purified antibodies were dialyzed against PBS. The purified parental IgG1 antibody versions of benchmarked monoclonals [REGN10933, REGN10987, CB6, LY-CoV555, CT-P59), COV2-2196, COV2-2130, ADG-2 and S309] were prepared as described above after cloning of synthetic DNA fragments (GeneArt, Thermo Fisher Scientific) coding for the immunoglobulin variable domains. Antibody preparations for in vivo infusions were micro-filtered (Ultrafree®-CL devices—0.1 μm PVDF membrane, Merck-Millipore, Darmstadt, Germany), and checked for endotoxins levels using the ToxinSensor™ Chromogenic LAL Endotoxin Assay Kit (GenScript).

The purified parental IgG1 antibody versions of benchmarked monoclonals [REGN10933, REGN10987 (PMID: 32540901), CB6 (PMID: 32454512), LY-CoV555 (PMID: 33820835), CT-P59 (PMID: 33436577), COV2-2196, COV2-2130 (PMID: 32668443), ADG-2 (PMID: 33495307) and S309 (PMID: 32422645)] were prepared as described above after cloning of synthetic DNA fragments (GeneArt, Thermo Fisher Scientific).

### ELISAs

ELISAs were performed as previously described (5, 6). Briefly, high-binding 96-well ELISA plates (Costar, Corning) were coated overnight with 250 ng/well of purified recombinant Coronavirus proteins and 500 ng/well of a SARS-CoV-2 fusion sequence-containing peptide

(SEQ ID NO: 105)
(KRSFIEDLLFNKVTLADAGFIK,

GenScript Biotech). After washings with 0.05% Tween 20-PBS (washing buffer), plates were blocked 2 h with 2% BSA, 1 mM EDTA, 0.05% Tween 20-PBS (Blocking buffer), washed, and incubated with serially diluted human and rodent sera, purified serum IgA/IgG or recombinant monoclonal antibodies in PBS. Total sera were diluted 1:100 (for human and golden hamster) or 1:10 (for K18 mouse) following by 7 consecutive 1:4 dilutions in PBS. Purified serum IgG and IgA antibodies were tested at 50 μg/ml and 7 consecutive 1:3 dilutions in PBS. Recombinant monoclonal IgG1 antibodies were tested at 4 or 10 μg/ml, and 4 to 7 consecutive 1:4 dilutions in PBS. Comparative ELISA binding of Cv2.1169 IgG1 and IgA1 antibodies was performed at a concentration of 70 nM, and 7 consecutive dilutions in PBS. To quantify blood-circulating human Cv2.1169 IgA1 and IgG1 in treated K18 mice and golden hamsters, high-binding 96-well ELISA plates (Costar, Corning) were coated overnight with 250 ng/well of purified goat anti-human IgA or IgG antibody (Jackson ImmunoResearch, 0.8 μg/ml final). After washings, plates were blocked, washed, and incubated for 2 h with 1:100 diluted sera from K18 mice and golden hamster and seven consecutive 1:3 dilutions in PBS. Cv2.1169 IgA1 or IgG1 antibody at 12 μg/ml and seven consecutive 1:3 dilutions in PBS were used as standards. After washings, the plates were revealed by incubation for 1 h with goat HRP-conjugated anti-mice IgG, anti-golden hamster IgG, anti-human IgG or anti-human IgA antibodies (Jackson ImmunoReseach, 0.8 μg/ml final) and by adding 100 μl of HRP chromogenic substrate (ABTS solution, Euromedex) after washing steps. Optical densities were measured at 405 nm (OD_{405 nm}), and background values given by incubation of PBS alone in coated wells were subtracted. Experiments were performed using HydroSpeed™ microplate washer and Sunrise™ microplate absorbance reader (Tecan Männedorf, Switzerland). For peptide-ELISA, binding of SARS-CoV2 and control IgG antibodies (at 1 μg/ml) to 15-mer S2 overlapping 5-amino acid peptides (n=52, GenScript Biotech, 500 ng/well) was tested using the same procedure as previously described (7). For competition ELISAs, 250 ng/well of StrepTag-free tri-S and RBD proteins were coated on ELISA plates (Costar, Corning), which were then blocked, washed, and incubated for 2 h with biotinylated antibodies (at a concentration of 100 ng/ml for tri-S competition and 25 ng/ml for RBD competition) in 1:2 serially diluted solutions of antibody competitors in PBS (IgG concentration ranging from 0.39 to 50 μg/ml). Plates were developed using HRP-conjugated streptavidin (BD Biosciences) as described above. For the competition experiments of tri-S- and RBD-binding to ACE2, ELISA plates (Costar, Corning) were coated overnight with 250 ng/well of purified ACE2 ectodomain. After washings, plates were blocked 2 h with Blocking buffer, PBST-washed, and incubated with recombinant monoclonal IgG1 antibodies at 2 μg/ml and 7 consecutive 1:2 dilutions in presence of biotinylated tri-S protein at 1 μg/ml in PBS, and at 10 or 100 μg/ml and 7 consecutive 1:2 dilutions in PBS in presence of biotinylated RBD at 0.5 μg/ml. After washings, the plates were revealed by incubation for 30 min with streptavidin HRP-conjugated (BD Biosciences) as described above.

Polyreactivity ELISA was performed as previously described (9). Briefly, high-binding 96-well ELISA plates were coated overnight with 500 ng/well of purified double stranded (ds)-DNA, KLH, LPS, Lysozyme, Thyroglobulin, Peptidoglycan from B. subtilis, 250 ng/well of insulin (Sigma-Aldrich, Saint-Louis, MO), flagellin from B. subtilis (Invivogen), MAPK14 (9), and 125 ng/well of YU2 HIV-1 Env gp140 protein in PBS. After blocking and washing steps, recombinant monoclonal IgG antibodies were tested at 4 μg/ml and 7 consecutive 1:4 dilutions in PBS. Control antibodies, mGO53 (negative) (3), and ED38 (high positive) (4) were included in each experiment. ELISA binding was developed as described above. Serum levels of human IL6, IP10, CXCL13 and BAFF were measured using DuoSet ELISA kits (R&D Systems) with undiluted plasma samples.

### HEp-2 IFA Assay

Recombinant SARS-CoV-2 S-specific and control IgG antibodies (mGO53 and ED38) at 100 μg/ml were analyzed by indirect immuno-fluorescence assay (IFA) on HEp-2 cells sections (ANA HEp-2 AeskuSlides®, Aesku.Diagnostics, Wendelsheim, Germany) using the kit's controls and FITC-conjugated anti-human IgG antibodies as the tracer according to the manufacturer' instructions. HEp-2 sections were examined using the fluorescence microscope Axio Imager 2 (Zeiss, Jena, Germany), and pictures were taken at magnification×40 with 5000 ms-acquisition using ZEN imaging software (Zen 2.0 blue version, Zeiss) at the Imagopole platform (Institut Pasteur).

### Infrared Immunoblotting

Recombinant tri-S protein was heat-denatured at 100° C. for 3 min in loading buffer (Invitrogen) containing 1× sample reducing agent (Invitrogen). Denatured tri-S protein (50 μg total) was separated by SDS-PAGE with a NuPAGE® 4-12% Bis-Tris Gel (1-well, Invitrogen), electro-transferred onto nitrocellulose membranes, and saturated in PBS-0.05% Tween 20 (PBST)-5% dry milk overnight at 4° C. Membranes were inserted into a Miniblot apparatus (Immunetics) and then incubated with human monoclonal antibodies (at a concentration of 1 μg/ml) and mouse anti-His×6 antibody (1 μg/ml, BD Biosciences) in PBS-T 5% dry milk in each channel for 2 h. For dot blotting experiments, denatured tri-S(ranging from 0.125 to 2 μg) was immobilized on dry nitrocellulose membranes for 2 h at room temperature and saturated in PBS-0.05% Tween 20 (PBST)-5% dry milk overnight at 4° C. The membranes were then incubated with human monoclonal antibodies (at a concentration of 1 μg/ml) and mouse anti-His×6 antibody (1 μg/ml, BD Biosciences) in PBS-T 5% dry milk for 2 h. After washing with PBST, membranes were incubated for 1h with 1/25,000-diluted Alexa Fluor 680-conjugated donkey anti-human IgG (Jackson ImmunoResearch) and 1/25,000-diluted IR Dye® 800CW-conjugated goat anti-mouse IgG (LI-COR Biosciences) in PBST-5% dry milk. Finally, membranes were washed, and examined with the Odyssey Infrared Imaging system (LI-COR Biosciences).

### Protein Microarray Binding Analyses

All experiments were performed at 4° C. using ProtoArray Human Protein Microarrays (Thermo Fisher Scientific). Microarrays were blocked for 1 h in blocking solution (Thermo Fisher), washed and incubated for 1h30 with IgG antibodies at 2.5 μg/ml as previously described (9). After washings, arrays were incubated for 1h30 with AF647-conjugated goat anti-human IgG antibodies (at 1 μg/ml in PBS; Thermo Fisher Scientific), and revealed using GenePix 4000B microarray scanner (Molecular Devices) and GenePix Pro 6.0 software (Molecular Devices) as previously described (9). Fluorescence intensities were quantified using Spotxel® software (SICASYS Software GmbH, Germany), and mean fluorescence intensity (MFI) signals for each antibody (from duplicate protein spots) was plotted against the reference antibody mGO53 (non-polyreactive isotype control) using GraphPad Prism software (v8.1.2, GraphPad Prism Inc.). For each antibody, Z-scores were calculated using ProtoArray® Prospector software (v5.2.3, Thermo Fisher Scientific), and deviation (a) to the diagonal, and polyreactivity index (PI) values were calculated as previously described (9). Antibodies were defined as polyreactive when PI>0.21.

### Surface Plasmon Resonance

Surface plasmon resonance (SPR)-based technology (Biacore 2000, Biacore, Uppsala, Sweden) was used to assess kinetics of interaction of monoclonal antibodies with SARS CoV2 proteins—trimer S, S1 and RBD. Antibodies (Cv2.1169, Cv2.1353, Cv2.3194, Cv2.3235 and Cv2.5213) and ACE2 ectodomain were covalently coupled to CM5 sensor chips (Biacore) using amino-coupling kit (Biacore) according to the manufacturer's procedure. In brief, IgG antibodies and ACE2 protein were diluted in 5 mM maleic acid solution, pH 4 to a final concentration of 10 μg/ml and injected over sensor surfaces pre-activated by a mixture of 1-Ethyl-3-(3-dimethylaminopropyl) carbodiimide and N-hydroxysuccinimide. Uncoupled carboxyl groups were blocked by exposure to 1M solution of ethanolamine.HCl (Biacore). Immobilization densities were 500 RU and 1000 RU for IgG antibodies and ACE2, respectively. All analyses were performed using HBS-EP buffer (10 mM HEPES pH 7.2; 150 mM NaCl; 3 mM EDTA, and 0.005% Tween 20). The flow rate of buffer during all real-time interaction measurements was set at 30 μl/min. All interactions were performed at temperature of 25° C. SARS CoV-2 tri-S and S1 proteins were serially diluted (two-fold step) in HBS-EP in the range of 40-0.156 nM. Same range of concentrations was used for RBD with exception of low affinity interactions where the concentration range 1280-10 nM was applied. The association and dissociation phases of the binding of viral proteins to the immobilized antibodies and ACE2 were monitored for 3 and 4 minutes, respectively. The binding of the proteins to reference channel containing carboxymethylated dextran only was used as negative control and was subtracted from the binding during data processing. The sensor chip surfaces were regenerated by 30 s exposure to 4M solution of guanidine.HCl (Sigma-Aldrich). The evaluation kinetic parameters of the studied interactions were performed by using BIAevaluation version 4.1.1 Software (Biacore).

### SARS-CoV-2 S-Fuse Neutralization Assay

S-Fuse cells (U20S-ACE2 GFP1-10 or GFP 11 cells) were mixed (ratio 1:1) and plated at a density of 8×10³ per well in a μClear 96-well plate (Greiner Bio-One) as previously described (1). SARS-CoV-2 and VOC viruses (MOI 0.1) were incubated with recombinant monoclonal IgG1, monomeric and dimeric IgA1 antibodies at 35 nM or 7 nM, and 11 consecutive 1:4 dilutions in culture medium for 30 min at room temperature and added to S-Fuse cells. The cells were fixed, 18 h later, in 2% paraformaldehyde, washed and stained with Hoechst stain (dilution 1:1000; Invitrogen). Images were acquired with an Opera Phenix high-content confocal microscope (Perkin Elmer). The area displaying GFP expression and the number of nuclei were quantified with Harmony software 4.8 (Perkin Elmer). The percentage neutralization was calculated from the GFP-positive area as follows: 100×(1−(value with IgA/IgG−value in “non-infected”)/(value in “no IgA/IgG”−value in “non-infected”)). IC₅₀ values were calculated using Prism software (v.9.3.1, GraphPad Prism Inc.) by fitting replicate values using the four-parameters dose-response model (variable slope). The neutralizing activity of each isotype is expressed as the half maximal effective concentration (IC₅₀). IC₅₀ values (pg/ml) were calculated based on a reconstructed curve of the percentage neutralization at the various concentrations indicated

### In Vitro SARS-CoV-2 Pseudoneutralization Assay

The SARS-CoV-2 pseudoneutralization assay was performed as previously described (10, 12). Briefly, 2×10⁴ 293T-ACE2-TMPRSS2 were plated in 96-well plates. Purified serum IgA and IgG antibodies were tested at 250 μg/ml and 7 consecutive 1:2 dilutions in PBS (or in Penicillin/Streptomycin-containing 10%-FCS DMEM), and incubated with spike-pseudotyped lentiviral particles for 15-30 minutes at room temperature before addition to the cells. Recombinant monoclonal IgG1, IgA1 or Fab-IgA fragment antibodies were also tested at 70 or 350 nM, and 11 consecutive 1:3 dilutions in PBS. After a 48h, the incubation at 37° C. in 5% CO2, the revelation was performed using the ONE-Glo™ Luciferase Assay System (Promega), and the luciferase signal was measured with EnSpire® Multimode Plate Reader (PerkinElmer). The percentage of neutralization was calculated as follow: 100×(1−mean (luciferase signal in sample duplicate)/mean (luciferase signal in virus alone)). Individual experiments were standardized using Cv2.3235 antibody. IC₅₀ values were calculated as described above.

### Antibody-Dependent Cellular Phagocytosis (ADCP) Assay

PBMC were isolated from healthy donors' blood (Etablissement Frangais du Sang) using Ficoll Plaque Plus (GE Healthcare). Primary human monocytes were purified from PBMC by MACS using Whole Blood CD14 MicroBeads (Miltenyi Biotech). Biotinylated-SARS-CoV-2 tri-S proteins were mixed with FITC-labelled NeutrAvidin beads (1 μm, Thermo Fisher Scientific) (1 μg of tri-S for 1 μl of beads), and incubated for 30 min at room temperature. After PBS washings, tri-S coupled-beads 1:500-diluted in DMEM were incubated for 1 h at 37° C. with human monoclonal IgG1 antibodies (at 3 μg/ml). tri-S-beads-antibody mixtures were then incubated with 7.5×10⁴ human monocytes for 2 h at 37° C. Following washings with 0.5% BSA, 2 mM EDTA-PBS, cells were fixed with 4% PFA-PBS and analyzed using a CytoFLEX flow cytometer (Beckman Coulter). ADCP assays were performed in two independent experiments, and analyzed using the FlowJo software (v10.6, FlowJo LLC). Phagocytic scores were calculated by dividing the fluorescence signals (% FITC-positive cells×geometric MFI FITC-positive cells) given by anti-SARS-CoV-2 spike antibodies by the one of the negative control antibody mGO53.

### Antibody-Dependent Cellular Cytotoxicity (ADCC) Assay

The ADCC activity of anti-SARS-CoV2 S IgG antibodies was determined using the ADCC Reporter Bioassay (Promega) as previously described (10). Briefly, 5×10⁴ Raji-Spike cells were co-cultured with 5×10⁴ Jurkat-CD16-NFAT-rLuc cells in presence or absence of SARS-CoV2 S-specific or control mGO53 IgG antibody at 10 μg/ml or 50 μg/ml and 10 consecutive 1:2 dilutions in PBS. Luciferase was measured after 18 h of incubation using an EnSpire plate reader (PerkinElmer). ADCC was measured as the fold induction of Luciferase activity compared to the control antibody. Experiments were performed in duplicate in two independent experiments.

### Complement-Dependent Cytotoxicity (CDC) Assay

The CDC activity of anti-SARS-CoV2 S IgG antibodies was measured using SARS-CoV-2 Spike-expressing Raji cells as previously described (10). Briefly, 5×10⁴ Raji-Spike cells were cultivated in the presence of 50% normal or heat-inactivated human serum, and with or without IgG antibodies (at 10 μg/ml or 50 μg/ml and 10 consecutive 1:2 dilutions in PBS). After 24h, cells were washed with PBS and incubated for 30 min at 4° C. the live/dead fixable aqua dead cell marker (1:1,000 in PBS; Life Technologies) before fixation. Data were acquired on an Attune N×T instrument (Life Technologies). CDC was calculated using the following formula: 100×(% of dead cells with serum−% of dead cells without serum)/(100−% of dead cells without serum). Experiments were performed in duplicate in two independent experiments.

### SARS-CoV-2 Infection and Treatment in Golden Hamsters

Golden Syrian hamsters (Mesocricetus auratus; RjHan:AURA) of 5-6 weeks of age (average weight 60-80 grams) were purchased from Janvier Laboratories (Le Genest-Saint-Isle, France) and handled under specific pathogen-free conditions. Golden hamsters were housed and manipulated in class III safety cabinets in the Pasteur Institute animal facilities accredited by the French Ministry of Agriculture for performing experiments on live rodents, with ad libitum access to water and food. Animal infection was performed as previously described (11). Briefly, anesthetized animals were intranasally infected with 6×10⁴ plaque-forming units (PFU) of SARS-CoV-2 (BetaCoV/France/IDF00372/2020; EVAg collection, Ref-SKU: 014V-03890) (50 μl/nostril). Mock-infected animals received the physiological solution only. Four or 24 h post-intranasal inoculation, hamsters received an intraperitoneal (i.p.) injection of 10 or 5 mg/kg of Cv2.1169 IgG or IgA antibody, as well as the mGO53 control antibody or PBS. All hamsters were followed-up daily when the body weight and the clinical score were noted. At day 5 post-inoculation, animals were euthanized with an excess of anesthetics (ketamine and xylazine) and exsanguination (AVMA Guidelines 2020). Blood samples were collected by cardiac puncture; after coagulation, tubes were centrifuged at 1,500×g during 10 min at 4° C., and sera were collected and frozen at −80° C. until further analyses. The lungs were weighted and frozen at −80° C. until further analyses. Frozen lungs fragments were weighted and homogenized with 1 ml of ice-cold DMEM (31966021, Gibco) supplemented with 1% penicillin/streptomycin (15140148, Thermo Fisher) in Lysing Matrix M 2 ml tubes (116923050-CF, MP Biomedicals) using the FastPrep-24™ system (MP Biomedicals), and the following scheme: homogenization at 4.0 m/s during 20 sec, incubation at 4° C. during 2 min, and new homogenization at 4.0 m/s during 20 sec. The tubes were centrifuged at 10,000×g during 1 min at 4° C. The supernatants were titrated on Vero-E6 cells by classical plaque assays using semisolid overlays (Avicel, RC581-NFDR080I, DuPont) and expressed and PFU/100 mg of tissue (12). Frozen lungs fragments were homogenized with Trizol (15596026, Invitrogen) in Lysing Matrix D 2 ml tubes (116913100, MP Biomedicals) using the FastPrep-24™ system (MP Biomedicals) and the following scheme: homogenization at 6.5 m/s during 60 sec, and centrifugation at 12,000×g during 2 min at 4° C. The supernatants were collected and the total RNA was then extracted using the Direct-zol RNA MiniPrep Kit (R²⁰⁵², Zymo Research) and quantified using NanoDrop 2000. The presence of genomic SARS-CoV-2 RNA in these samples was evaluated by one-step RT-qPCR in a final volume of 25 μl per reaction in 96-well PCR plates using a thermocycler (7500t Real-time PCR system, Applied Biosystems) as previously described (11). Viral load quantification (expressed as RNA copy number/pg of RNA) was assessed by linear regression using a standard curve of six known quantities of RNA transcripts containing the RdRp sequence (ranging from 10⁷ to 10² copies).

### SARS-CoV-2 Infection and Treatment in K18 Mice

B6.Cg-Tg(K18-ACE2)2Prlmn/J mice (stock #034860) were imported from The Jackson Laboratory (Bar Harbor, ME, USA) and bred at the Institut Pasteur under strict SPF conditions. Infection studies were performed on 6- to 16-week-old male and female mice, in animal biosafety level 3 (BSL-3) facilities at the Institut Pasteur, in Paris. All animals were handled in strict accordance with good animal practice. Animal work was approved by the Animal Experimentation Ethics Committee (CETEA 89) of the Institut Pasteur (project dap 200008 and 200023) and authorized by the French legislation (under project 24613) in compliance with the European Communities Council Directives (2010/63/UE, French Law 2013-118, Feb. 6, 2013) and according to the regulations of Pasteur Institute Animal Care Committees before experiments were initiated. Anesthetized (ketamine/xylazine) mice were inoculated intranasally (i.n.) with 1×10⁴ or 1×10⁵ PFU of SARS-CoV-2 virus (20 μl/nostril). Six or 22 h post-inoculation, mice received an intraperitoneal (i.p.) injection of 5, 10, 20 or 40 mg/kg of Cv2.1169 IgG or IgA antibody, and of mGO53 control IgG or IgA antibody. Oropharyngeal swabs were taken on day 3 post-infection. Clinical signs of disease (ruffled fur, hunched posture, reduced mobility and breathing difficulties) and weight loss were monitored daily during 20 days. Mice were euthanized when they reached pre-defined end-point criteria, and sera were harvested from collected cardiac blood punctures.

### Quantification and Statistical Analysis

The numbers of V_{H}, Vκ and Vλ mutations were compared across groups of antibodies using unpaired Student's t test with Welch's correction. Bivariate correlations were assayed using two-tailed Pearson correlation test. Statistical and analyses were performed using GraphPad Prism software (v.8.2, GraphPad Prism Inc.). Volcano plot comparing gene features (n=206 parameters) of tri-S⁺ B cells and normal memory B-cells (mB) was also performed using GraphPad Prism software (v.8.4, GraphPad Prism Inc.). The y axis indicates the statistics expressed as −log₁₀ (p-values) and the x axis represents the differences between the group means for each parameter. The Barnes-Hut implementation of t-distributed stochastic neighbor embedding (t-SNE) was computed using FlowJo software (v.10.3, FlowJo LLC, Ashland, OR) with 2000 iterations and a perplexity parameter of 200. Colors represent density of surface expression markers or cell-populations varying from low (blue) to high (red). Circos plot linking antibody sequences with at least 75% identity within their CDR_{H}3 was performed using online software at http://mkweb.bcgsc.ca/circos. Phylogenetic tree was built using CLC Main Workbench (Qiagen) on aligned V_{H} sequences using the Neighbor-Joining method with a bootstrap analysis on 100 replicates. Mouse survival were compared across groups using a Kaplan-Meier analysis and Log-rank Mantel-Cox test (GraphPad Prism, v8.2, GraphPad Prism Inc.). Groups of golden Syrian hamsters were compared across analyses using two-tailed Mann-Whitney test (GraphPad Prism, v.8.2, GraphPad Prism Inc.). Principal component analysis (PCA) was performed using the prcomp( ) function in R Studio Server (v1.4.1103). PCA plots of individuals [fviz_pca_ind( )], variables [fviz_pca_var( )], and biplots [fviz_pca_biplot( )], were generated using the factoextra package (v1.0.7, https://CRAN.R-project.org/package=factoextra). Spearman rank correlations were used to establish multiparameter associations. All correlograms and scatterplots were created using the corrplot and plot R functions, respectively. Correlation plots were generated using GraphPad Prism (v6.4, GraphPad Prism Inc.).

### Flow Cytometry Binding Assays

SARS-CoV-2 specificity validation of cloned human IgG antibodies was performed using the S-Flow assay as previously described (PMID: 32817357). To evaluate Spike cross-reactivity, Freestyle™ 293-F were transfected with pUNO1-Spike-dfur expression vectors (Spike and SpikeV1 to V11 plasmids, Invivogen) (1.2 μg plasmid DNA per 10⁶ cells) using PEI-precipitation method. Forty-eight hours post-transfection, 0.5×10⁶ transfected and non-transfected control cells were incubated with IgG antibodies for 30 min at 4° C. (1 μg/ml). After washings, cells were incubated 20 min at 4° C. with AF647-conjugated goat anti-human IgG antibodies (1:1000 dilution; Thermo Fisher Scientific) and LIVE/DEAD Fixable Viability dye Aqua (1:1000 dilution; Thermo Fisher Scientific), washed and resuspended in PBS-Paraformaldehyde 1% (Electron Microscopy Sciences). Data were acquired using a CytoFLEX flow cytometer (Beckman Coulter), and analyzed using FlowJo software (v10.7.1; FlowJo LLC). Antibodies were tested in duplicate.

### Crystallization and Structure Determinations

The Fab fragment of the anti-SARS-CoV-2 S antibody CR3022, to be used as crystallization chaperone molecules, was produced and purified as described above (section with heading Single B-cell FACS sorting and expression-cloning of antibodies). The purified RBD protein was incubated overnight at 4° C. with the Fabs with an RBD-Fab molar ratio of 2:1 (2:1:1 for the ternary complex RBD-Cv2.1169-CR3022). Each binding reaction was loaded onto a Superdex200 column (Cytiva) equilibrated in 10 mM Tris-HCl (pH 8.0), 100 mM NaCl. The fractions corresponding to the complexes were pooled, concentrated to 9-10 mg/ml and used in crystallization trials at 18° C. using the sitting-drop vapor diffusion method. The RBD-Cv2.2325 Fab complex crystalized with 0.1 M ammonium citrate (pH 7.0), 12% PEG 3350, while crystals for RBD-Cv2.6264 Fab were obtained with 0.1 M NaAc, 7% PEG 6000, 30% ethanol. The RBD-Cv2.1169-CR3022 crystals grew in the presence of 6% PEG 8000, 0.5 M Li2SO4. Crystals were flash-frozen by immersion into a cryo-protectant containing the crystallization solution supplemented with 30% (v/v) glycerol (RBD-Cv2.2325; RBD-Cv2.1169-CR3022) or 30% (v/v) ethylenglycol (RBD-Cv2.6264), followed by rapid transfer into liquid nitrogen. Data collection was carried out at SOLEIL synchrotron (St Aubin, France). Data were processed, scaled and reduced with XDS and AIMLESS. The structures were determined by molecular replacement using Phaser from the suite PHENIX (101) and search ensembles obtained from the PBDs 6M0J (RBD), 5I1E (Cv2.2325), 5VAG (Cv2.6264), 7K3Q (Cv2.1169) and 6YLA (CR3022). The final models were built by combining real space model building in Coot with reciprocal space refinement with phenix.refine. The final model was validated with Molprobity. Epitope and paratope residues, as well as their interactions, were identified by accessing PISA at the European Bioinformatics Institute (www.ebi.ac.uk/pdbe/prot_int/pistart.html). Superpositions and figures were rendered using Pymol and UCSF Chimera.

### Cryo-Electron Microscopy

The S_6P protein was incubated with the Cv2.1169 IgA Fab at a 1:3.6 (trimer:Fab) ratio and a final trimer concentration of 0.8 μM for 1h at room temperature. 3 μl aliquots of the sample were applied to freshly glow discharged R 1.2/1.3 Quantifoil grids prior to plunge freezing using a Vitrobot Mk IV (Thermo Fischer Scientific) at 8° C. and 100% humidity (blot 4s, blot force 0). Data for the complex were acquired on a Titan Krios transmission electron microscope (Thermo Fischer Scientific) operating at 300 kV, using the EPU automated image acquisition software (Thermo Fisher Scientific). Movies were collected on a Gatan K3 direct electron detector operating in Counted Super Resolution mode at a nominal magnification of 105,000× (0.85 Å/pixel) using defocus range of −1.0 μm to −3.0 μm. Movies were collected over a 2 s exposure and a total dose of ˜45 e-/Å2.

### Image Processing

All movies were motion-corrected and dose-weighted with MotionCorr2 and the aligned micrographs were used to estimate the defocus values with patchCTF within cryosparc. CryoSPARC blob picker was used for automated particle picking and the resulting particles used to obtain initial 2D references, which were then used to auto-pick the micrographs. An initial 3D model was obtained in cryosparc and used to perform a 3D classification without imposing any symmetry in Relion. The best class was selected and subjected to 3D, non-uniform refinement in cryosparc.

### Sequences of the Protein/Peptides Used in the Various Assays

SEQ ID NO: 103: Angiotensin Converting Enzyme 2 (ACE2) ectodomain
METDTLLLWVLLLWVPGSTGSTIEEQAKTFLDKFNHEAEDLFYQSSLASWNYNTNITEENVQNMNNAGDKWSAF
LKEQSTLAQMYPLQEIQNLTVKLQLQALQQNGSSVLSEDKSKRLNTILNTMSTIYSTGKVCNPDNPQECLLLEP
GLNEIMANSLDYNERLWAWESWRSEVGKQLRPLYEEYVVLKNEMARANHYEDYGDYWRGDYEVNGVDGYDYSRG
QLIEDVEHTFEEIKPLYEHLHAYVRAKLMNAYPSYISPIGCLPAHLLGDMWGRFWTNLYSLTVPFGQKPNIDVT
DAMVDQAWDAQRIFKEAEKFFVSVGLPNMTQGFWENSMLTDPGNVQKAVCHPTAWDLGKGDFRILMCTKVTMDD
FLTAHHEMGHIQYDMAYAAQPFLLRNGANEGFHEAVGEIMSLSAATPKHLKSIGLLSPDFQEDNETEINFLLKQ
ALTIVGTLPFTYMLEKWRWMVFKGEIPKDQWMKKWWEMKREIVGVVEPVPHDETYCDPASLFHVSNDYSFIRYY
TRTLYQFQFQEALCQAAKHEGPLHKCDISNSTEAGQKLENMLRLGKSEPWTLALENVVGAKNMNVRPLLNYFEP
LFTWLKDQNKNSFVGWSTDWSPYADGSGLVPRGSHHHHHHHHSAWSHPQFEK
SEQ ID NO: 104: SARS-COV-2 Nucleocapsid protein (N)
MGWSCIILFLVATATGVHSQRNAPRITFGGPSDSTGSNQNGERSGARSKQRRPQGLPNNTASWFTALTQHGKED
LKFPRGQGVPINTNSSPDDQIGYYRRATRRIRGGDGKMKDLSPRWYFYYLGTGPEAGLPYGANKDGIIWVATEG
ALNTPKDHIGTRNPANNAAIVLQLPQGTTLPKGFYAEGSRGGSQASSRSSSRSRNSSRNSTPGSSRGTSPARMA
GNGGDAALALLLLDRLNQLESKMSGKGQQQQGQTVTKKSAAEASKKPRQKRTATKAYNVTQAFGRRGPEQTQGN
FGDQELIRQGTDYKHWPQIAQFAPSASAFFGMSRIGMEVTPSGTWLTYTGAIKLDDKDPNFKDQVILLNKHIDA
YKTFPPTEPKKDKKKKADETQALPQRQKKQQTVTLLPAADLDDFSKQLQQSMSSADSTQAGSHHHHHHHHGSGL
NDIFEAQKIEWHE
SEQ ID NO: 105: SARS-COV-2 Fusion peptide (FP)
KRSFIEDLLENKVTLADAGFIK
SEQ ID NO: 106: SARS-COV-2 Spike ectodomain (tri-S)
MFVFLVLLPLVSSQCVNLTTRTQLPPAYTNSFTRGVYYPDKVFRSSVLHSTQDLFLPFFSNVTWFHAIHVSGTN
GTKRFDNPVLPENDGVYFASTEKSNIIRGWIFGTTLDSKTQSLLIVNNATNVVIKVCEFQFCNDPFLGVYYHKN
NKSWMESEFRVYSSANNCTFEYVSQPFLMDLEGKQGNFKNLREFVFKNIDGYFKIYSKHTPINLVRDLPQGFSA
LEPLVDLPIGINITRFQTLLALHRSYLTPGDSSSGWTAGAAAYYVGYLQPRTFLLKYNENGTITDAVDCALDPL
SETKCTLKSFTVEKGIYQTSNFRVQPTESIVRFPNITNLCPFGEVFNATRFASVYAWNRKRISNCVADYSFLYN
SASFSTFKCYGVSPTKLNDLCFTNVYADSFVIRGDEVRQIAPGQTGKIADYNYKLPDDFTGCVIAWNSNNLDSK
VGGNYNYLYRLFRKSNLKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKELPFQQFGRDIADTTDAVRDPQTLEILDITPCSF
GGVSVITPGTNTSNQVAVLYQDVNCTEVPVAIHADQLTPTWRVYSTGSNVFQTRAGCLIGAEHVNNSYECDIPI
GAGICASYQTQTNSPRRARSVASQSIIAYTMSLGAENSVAYSNNSIAIPTNFTISVTTEILPVSMTKTSVDCTM
YICGDSTECSNLLLQYGSFCTQLNRALTGIAVEQDKNTQEVFAQVKQIYKTPPIKDFGGFNFSQILPDPSKPSK
RSFIEDLLFNKVTLADAGFIKQYGDCLGDIAARDLICAQKENGLTVLPPLLTDEMIAQYTSALLAGTITSGWTF
GAGAALQIPFAMQMAYRENGIGVTQNVLYENQKLIANQFNSAIGKIQDSLSSTASALGKLQDVVNQNAQALNTL
VKQLSSNFGAISSVLNDILSRLDPPEAEVQIDRLITGRLQSLQTYVTQQLIRAAEIRASANLAATKMSECVLGQ
SKRVDFCGKGYHLMSFPQSAPHGVVFLHVTYVPAQEKNFTTAPAICHDGKAHFPREGVFVSNGTHWFVTQRNFY
EPQIITTDNTFVSGNCDVVIGIVNNTVYDPLQPELDSFKEELDKYFKNHTSPDVDLGDISGINASVVNIQKEID
RLNEVAKNLNESLIDLQELGKYEQGSGYIPEAPRDGQAYVRKDGEWVLLSTFLGGSHHHHHHHHSAWSHPQFEK
GTGGLNDIFEAQKIEWHE
SEQ ID NO: 107: SARS-COV-2 S1 domain
MGWSCIILFLVATATGVHSVNLTTRTQLPPAYTNSFTRGVYYPDKVFRSSVLHSTQDLFLPFFSNVTWFHAIHV
SGTNGTKRFDNPVLPFNDGVYFASTEKSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREFVFKNIDGYFKIYSKHTPINLVRDLPQ
GFSALEPLVDLPIGINITRFQTLLALHRSYLTPGDSSSGWTAGAAAYYVGYLQPRTFLLKYNENGTITDAVDCA
LDPLSETKCTLKSFTVEKGIYQTSNERVQPTESIVRFPNITNLCPFGEVFNATRFASVYAWNRKRISNCVADYS
FLYNSASFSTFKCYGVSPTKLNDLCFTNVYADSFVIRGDEVRQIAPGQTGKIADYNYKLPDDFTGCVIAWNSNN
LDSKVGGNYNYLYRLERKSNLKPFERDISTEIYQAGSTPCNGVEGFNCYFPLQSYGFQPTNGVGYQPYRVVVLS
FELLHAPATVCGPKKSTNLVKNKCVNFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYSTGSNVFQTRAGCLIGAEHVNNSYEC
DIPIGAGICASYQTQTNSPGGSHHHHHHHH
SEQ ID NO: 108: SARS-COV-2 RBD domain
METDTLLLWVLLLWVPGSTGNITNLCPFGEVFNATRFASVYAWNRKRISNCVADYSFLYNSASFSTFKCYGVSP
TKLNDLCFTNVYADSFVIRGDEVRQIAPGQTGKIADYNYKLPDDFTGCVIAWNSNNLDSKVGGNYNYLYRLFRK
SNLKPFERDISTEIYQAGSTPCNGVEGFNCYFPLQSYGFQPTNGVGYQPYRVVVLSFELLHAPATVCGPKGSGL
VPRGSHHHHHHHHSAWSHPQFEKGTGGLNDIFEAQKIEWHE
SEQ ID NO: 109: SARS-COV-2 RBD (B.1.1.7) domain
METDTLLLWVLLLWVPGSTGNITNLCPFGEVFNATRFASVYAWNRKRISNCVADYSFLYNSASFSTFKCYGVSP
TKLNDLCFTNVYADSFVIRGDEVRQIAPGQTGKIADYNYKLPDDFTGCVIAWNSNNLDSKVGGNYNYLYRLFRK
SNLKPFERDISTEIYQAGSTPCNGVEGFNCYFPLQSYGFQPTYGVGYQPYRVVVLSFELLHAPATVCGPKGSGL
VPRGSHHHHHHHHSAWSHPQFEKGTGGLNDIFEAQKIEWHE
SEQ ID NO: 110: SARS-COV-2 RBD (B.1.351) domain
METDTLLLWVLLLWVPGSTGNITNLCPFGEVFNATRFASVYAWNRKRISNCVADYSFLYNSASFSTFKCYGVSP
TKLNDLCFTNVYADSFVIRGDEVRQIAPGQTGNIADYNYKLPDDFTGCVIAWNSNNLDSKVGGNYNYLYRLFRK
SNLKPFERDISTEIYQAGSTPCNGVKGFNCYFPLQSYGFQPTYGVGYQPYRVVVLSFELLHAPATVCGPKGSGL
VPRGSHHHHHHHHSAWSHPQFEKGTGGLNDIFEAQKIEWHE
SEQ ID NO: 111: SARS-COV-2 RBD (P.1) domain
METDTLLLWVLLLWVPGSTGNITNLCPFGEVFNATRFASVYAWNRKRISNCVADYSFLYNSASFSTFKCYGVSP
TKLNDLCFTNVYADSFVIRGDEVRQIAPGQTGTIADYNYKLPDDFTGCVIAWNSNNLDSKVGGNYNYLYRLFRK
SNLKPFERDISTEIYQAGSTPCNGVKGFNCYFPLQSYGFQPTYGVGYQPYRVVVLSFELLHAPATVCGPKGSGL
VPRGSHHHHHHHHSAWSHPQFEKGTGGLNDIFEAQKIEWHE
SEQ ID NO: 112: SARS-COV-2 S1 NTD domain
MFVFLVLLPLVSSQCVNLTTRTQLPPAYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEFQFCNDPFLGVYYHKN
NKSWMESEFRVYSSANNCTFEYVSQPFLMDLEGKQGNFKNLREFVFKNIDGYFKIYSKHTPINLVRDLPQGESA
LEPLVDLPIGINITRFQTLLALHRSYLTPGDSSSGWTAGAAAYYVGYLQPRTFLLKYNENGTITDAVDCALDPL
SETKCTLKSGSGLVPRGSHHHHHHHH
SEQ ID NO: 113: SARS-COV-2 S1 connecting domain (CD)
MGWSCIILFLVATATGVHSTVEKGIYQTSNFRVQPTESIVRFPGGGSGGKSTNLVKNKCVNFNFNGLTGTGVLT
ESNKKFLPFQQFGRDIADTTDAVRDPQTLEILDITPCSFGGVSVITPGTNTSNQVAVLYQDVNCTEVPVAIHAD
QLTPTWRVYSTGSNVFQTRAGCLIGAEHVNNSYECDIPIGAGICASYQTQTNSPRRARSVASQSIIAYTMSLGG
SGLVPRGSHHHHHHHH
SEQ ID NO: 114: SARS-COV-2 S2 domain
MGWSCIILFLVATATGVHSSVASQSIIAYTMSLGAENSVAYSNNSIAIPTNFTISVTTEILPVSMTKTSVDCTM
YICGDSTECSNLLLQYGSFCTQLNRALTGIAVEQDKNTQEVFAQVKQIYKTPPIKDFGGFNFSQILPDPSKPSK
RSFIEDLLFNKVTLADAGFIKQYGDCLGDIAARDLICAQKENGLTVLPPLLTDEMIAQYTSALLAGTITSGWTF
GAGAALQIPFAMQMAYRENGIGVTQNVLYENQKLIANQFNSAIGKIQDSLSSTASALGKLQDVVNQNAQALNTL
VKQLSSNFGAISSVLNDILSRLDPPEAEVQIDRLITGRLQSLQTYVTQQLIRAAEIRASANLAATKMSECVLGQ
SKRVDFCGKGYHLMSFPQSAPHGVVFLHVTYVPAQEKNFTTAPAICHDGKAHFPREGVFVSNGTHWFVTQRNFY
EPQIITTDNTFVSGNCDVVIGIVNNTVYDPLQPELDSFKEELDKYFKNHTSPDVDLGDISGINASVVNIQKEID
RLNEVAKNLNESLIDLQELGKYEQGSGYIPEAPRDGQAYVRKDGEWVLLSTFLGGSHHHHHHHH
SEQ ID NO: 115: CoV-OC43 Spike ectodomain
MPMGSLQPLATLYLLGMLVASVLAVIGDLKCTSDNINDKDTGPPPISTDTVDVTNGLGTYYVLDRVYLNTTLFL
NGYYPTSGSTYRNMALKGSVLLSRLWFKPPFLSDFINGIFAKVKNTKVIKDRVMYSEFPAITIGSTEVNTSYSV
VVQPRTINSTQDGDNKLQGLLEVSVCQYNMCEYPQTICHPNLGNHRKELWHLDTGVVSCLYKRNFTYDVNADYL
YFHFYQEGGTFYAYFTDTGVVTKFLFNVYLGMALSHYYVMPLTCNSKLTLEYWVTPLTSRQYLLAFNQDGIIFN
AVDCMSDFMSEIKCKTQSIAPPTGVYELNGYTVQPIADVYRRKPNLPNCNIEAWLNDKSVPSPLNWERKTFSNC
NFNMSSLMSFIQADSFTCNNIDAAKIYGMCFSSITIDKFAIPNGRKVDLQLGNLGYLQSFNYRIDTTATSCQLY
YNLPAANVSVSRFNPSTWNKRFGFIEDSVFKPRPAGVLTNHDVVYAQHCFKAPKNFCPCKLNGSCVGSGPGKNN
GIGTCPAGTNYLTCDNLCTPDPITFTGTYKCPQTKSLVGIGEHCSGLAVKSDYCGGNSCTCRPQAFLGWSADSC
LQGDKCNIFANFILHDVNSGLTCSTDLQKANTDIILGVCVNYDLYGILGQGIFVEVNATYYNSWQNLLYDSNGN
LYGFRDYITNRTFMIRSCYSGRVSAAFHANSSEPALLERNIKCNYVFNNSLTRQLQPINYFDSYLGCVVNAYNS
TAISVQTCDLTVGSGYCVDYSKNGGSGGAITTGYRFTNFEPFTVNSVNDSLEPVGGLYEIQIPSEFTIGNMVEF
IQTSSPKVTIDCAAFVCGDYAACKSQLVEYGSFCDNINAILTEVNELLDTTQLQVANSLMNGVTLSTKLKDGVN
FNVDDINFSPVLGCLGSECSKASSRSAIEDLLFDKVKLSDVGFVEAYNNCTGGAEIRDLICVQSYKGIKVLPPL
LSENQFSGYTLAATSASLFPPWTAAAGVPFYLNVQYRINGLGVTMDVLSQNQKLIANAFNNALYAIQEGFDATN
SALVKIQAVVNANAEALNNLLQQLSNRFGAISASLQEILSRLDALEAEAQIDRLINGRLTALNAYVSQQLSDST
LVKFSAAQAMEKVNECVKSQSSRINFCGNGNHIISLVQNAPYGLYFIHFSYVPTKYVTARVSPGLCIAGDRGIA
PKSGYFVNVNNTWMYTGSGYYYPEPITENNVVVMSTCAVNYTKAPYVMLNTSIPNLPDFKEELDQWFKNQTSVA
PDLSLDYINVTFLDLLIKRMKQIEDKIEEIESKQKKIENEIARIKKIKLVPRGSLEWSHPQFEK
SEQ ID NO: 116: CoV-HKU1 Spike ectodomain
METDTLLLWVLLLWVPGSTGVIGDENCINSFINDYNKTIPRISEDVVDVSLGLGTYYVLNRVYLNTTLLFTGYF
PKSGANFRDLALKGSIYLSTLWYKPPFLSDENNGIFSKVKNTKLYVNNTLYSEFSTIVIGSVFVNTSYTIVVQP
HNGILEITACQYTMCEYPHTVCKSKGSIRNESWHIDSSEPLCLFKKNFTYNVSADWLYFHFYQERGVFYAYYAD
VGMPTTFLFSLYLGTILSHYYVMPLTCNAISSNTDNETLEYWVTPLSRRQYLLNFDEHGVITNAVDCSSSFLSE
IQCKTQSFAPNTGVYDLSGFTVKPVATVYRRIPNLPDCDIDNWLNNVSVPSPLNWERRIFSNCNENLSTLLRLV
HVDSFSCNNLDKSKIFGSCENSITVDKFAIPNRRRDDLQLGSSGFLQSSNYKIDISSSSCQLYYSLPLVNVTIN
NFNPSSWNRRYGFGSFNLSSYDVVYSDHCFSVNSDFCPCADPSVVNSCAKSKPPSAICPAGTKYRHCDLDTTLY
VKNWCRCSCLPDPISTYSPNTCPQKKVVVGIGEHCPGLGINEEKCGTQLNHSSCFCSPDAFLGWSFDSCISNNR
CNIFSNFIFNGINSGTTCSNDLLYSNTEISTGVCVNYDLYGITGQGIFKEVSAAYYNNWQNLLYDSNGNIIGFK
DFLINKTYTILPCYSGRVSAAFYQNSSSPALLYRNLKCSYVLNNISFISQPFYFDSYLGCVLNAVNLTSYSVSS
CDLRMGSGFCIDYALPSSGGSGSGISSPYRFVTFEPFNVSFVNDSVETVGGLFEIQIPTNFTIAGHEEFIQTSS
PKVTIDCSAFVCSNYAACHDLLSEYGTFCDNINSILNEVNDLLDITQLQVANALMQGVTLSSNLNTNLHSDVDN
IDFKSLLGCLGSQCGSSSRSLLEDLLENKVKLSDVGFVEAYNNCTGGSEIRDLLCVQSENGIKVLPPILSETQI
SGYTTAATVAAMFPPWSAAAGVPFSLNVQYRINGLGVTMDVLNKNQKLIANAFNKALLSIQNGFTATNSALAKI
QSVVNANAQALNSLLQQLFNKFGAISSSLQEILSRLDPPEAQVQIDRLINGRLTALNAYVSQQLSDITLIKAGA
SRAIEKVNECVKSQSPRINFCGNGNHILSLVQNAPYGLLFIHFSYKPTSFKTVLVSPGLCLSGDRGIAPKQGYF
IKQNDSWMFTGSSYYYPEPISDKNVVFMNSCSVNFTKAPFIYLNNSIPNLSDFEAELSLWFKNHTSIAPNLTEN
SHINATFLDLYYEMNVIQESIKSLNGSGYIPEAPRDGQAYVRKDGEWVLLSTFLGSLVPRGSHHHHHHHH
SEQ ID NO: 117: CoV-MERS Spike ectodomain
METDTLLLWVLLLWVPGSTGVDVGPDSVKSACIEVDIQQTFFDKTWPRPIDVSKADGIIYPQGRTYSNITITYQ
GLFPYQGDHGDMYVYSAGHATGTTPQKLFVANYSQDVKQFANGFVVRIGAAANSTGTVIISPSTSATIRKIYPA
FMLGSSVGNFSDGKMGRFFNHTLVLLPDGCGTLLRAFYCILEPRSGNHCPAGNSYTSFATYHTPATDCSDGNYN
RNASLNSFKEYFNLRNCTFMYTYNITEDEILEWFGITQTAQGVHLFSSRYVDLYGGNMFQFATLPVYDTIKYYS
IIPHSIRSIQSDRKAWAAFYVYKLQPLTFLLDFSVDGYIRRAIDCGFNDLSQLHCSYESFDVESGVYSVSSFEA
KPSGSVVEQAEGVECDFSPLLSGTPPQVYNFKRLVFTNCNYNLTKLLSLFSVNDFTCSQISPAAIASNCYSSLI
LDYFSYPLSMKSDLSVSSAGPISQFNYKQSFSNPTCLILATVPHNLTTITKPLKYSYINKCSRLLSDDRTEVPQ
LVNANQYSPCVSIVPSTVWEDGDYYRKOLSPLEGGGWLVASGSTVAMTEQLQMGFGITVQYGTDTNSVCPKLEF
ANDTKIASQLGNCVEYSLYGVSGRGVFQNCTAVGVRQQRFVYDAYQNLVGYYSDDGNYYCLRACVSVPVSVIYD
KETKTHATLFGSVACEHISSTMSQYSRSTRSMLKRRDSTYGPLQTPVGCVLGLVNSSLFVEDCKLPLGQSLCAL
PDTPSTLTPASVGSVPGEMRLASIAFNHPIQVDQLNSSYFKLSIPTNFSFGVTQEYIQTTIQKVTVDCKQYVCN
GFQKCEQLLREYGQFCSKINQALHGANLRQDDSVRNLFASVKSSQSSPIIPGFGGDENLILLEPVSISTGSRSA
RSAIEDLLFDKVTIADPGYMQGYDDCMQQGPASARDLICAQYVAGYKVLPPLMDVNMEAAYTSSLLGSIAGVGW
TAGLSSFAAIPFAQSIFYRLNGVGITQQVLSENQKLIANKENQALGAMQTGFTTTNEAFQKVQDAVNNNAQALS
KLASELSNTFGAISASIGDIIQRLDPPEQDAQIDRLINGRLTTLNAFVAQQLVRSESAALSAQLAKDKVNECVK
AQSKRSGFCGQGTHIVSFVVNAPNGLYFMHVGYYPSNHIEVVSAYGLCDAANPTNCIAPVNGYFIKTNNTRIVD
EWSYTGSSFYAPEPITSLNTKYVAPQVTYQNISTNLPPPLLGNSTGIDFQDELDEFFKNVSTSIPNFGSLTQIN
TTLLDLTYEMLSLQQVVKALNESYIDLKELGNYTYYNKGSGYIPEAPRDGQAYVRKDGEWVLLSTFLGSLVPRG
SHHHHHHHHGLNDIFEAQKIEWHE
SEQ ID NO: 118: SARS-COV-1 Spike ectodomain
METDTLLLWVLLLWVPGSTGSDLDRCTTFDDVQAPNYTQHTSSMRGVYYPDEIFRSDTLYLTQDLFLPFYSNVT
GFHTINHTFDNPVIPFKDGIYFAATEKSNVVRGWVFGSTMNNKSQSVIIINNSTNVVIRACNFELCDNPFFAVS
KPMGTQTHTMIFDNAFNCTFEYISDAFSLDVSEKSGNFKHLREFVFKNKDGFLYVYKGYQPIDVVRDLPSGFNT
LKPIFKLPLGINITNFRAILTAFSPAQDTWGTSAAAYFVGYLKPTTFMLKYDENGTITDAVDCSQNPLAELKCS
VKSFEIDKGIYQTSNFRVVPSGDVVRFPNITNLCPFGEVFNATKFPSVYAWERKKISNCVADYSVLYNSTFFST
FKCYGVSATKLNDLCFSNVYADSFVVKGDDVRQIAPGQTGVIADYNYKLPDDEMGCVLAWNTRNIDATSTGNYN
YKYRYLRHGKLRPFERDISNVPFSPDGKPCTPPALNCYWPLNDYGFYTTTGIGYQPYRVVVLSFELLNAPATVC
GPKLSTDLIKNQCVNFNFNGLTGTGVLTPSSKRFQPFQQFGRDVSDFTDSVRDPKTSEILDISPCSFGGVSVIT
PGTNASSEVAVLYQDVNCTDVSTAIHADQLTPAWRIYSTGNNVFQTQAGCLIGAEHVDTSYECDIPIGAGICAS
YHTVSLLRSTSQKSIVAYTMSLGADSSIAYSNNTIAIPTNFSISITTEVMPVSMAKTSVDCNMYICGDSTECAN
LLLQYGSFCTQLNRALSGIAAEQDRNTREVFAQVKQMYKTPTLKYFGGFNFSQILPDPLKPTKRSFIEDLLENK
VTLADAGFMKQYGECLGDINARDLICAQKENGLTVLPPLLTDDMIAAYTAALVSGTATAGWTFGAGAALQIPFA
MQMAYRFNGIGVTQNVLYENQKQIANQFNKAISQIQESLTTTSTALGKLQDVVNQNAQALNTLVKQLSSNFGAI
SSVLNDILSRLDPPEAEVQIDRLITGRLQSLQTYVTQQLIRAAEIRASANLAATKMSECVLGQSKRVDFCGKGY
HLMSFPQAAPHGVVFLHVTYVPSQERNFTTAPAICHEGKAYFPREGVFVFNGTSWFITQRNFFSPQIITTDNTF
VSGNCDVVIGIINNTVYDPLQPELDSFKEELDKYFKNHTSPDVDLGDISGINASVVNIQKEIDRLNEVAKNLNE
SLIDLQELGKYEQYIKGSGYIPEAPRDGQAYVRKDGEWVLLSTFLGSLVPRGSHHHHHHHH
SEQ ID NO: 119: CoV-229E Spike ectodomain
METDTLLLWVLLLWVPGSTGAGCQTTNGLNTSYSVCNGCVGYSENVFAVESGGYIPSDFAFNNWELLINTSSVV
DGVVRSFQPLLLNCLWSVSGLRFTTGFVYFNGTGRGDCKGFSSDVLSDVIRYNLNFEENLRRGTILFKTSYGVV
VFYCTNNTLVSGDAHIPFGTVLGNFYCFVNTTIGTETTSAFVGALPKTVREFVISRTGHFYINGYRYFTLGNVE
AVNFNVTTAETTDFFTVALASYADVLVNVSQTSIANIIYCNSVINRLRCDQLSFYVPDGFYSTSPIQSVELPVS
IVSLPVYHKHMFIVLYVDFKPQSGGGKCENCYPAGVNITLANFNETKGPLCVDTSHFTTKYVAVYANVGRWSAS
INTGNCPFSFGKVNNFVKFGSVCFSLKDIPGGCAMPIVANWAYSKYYTIGTLYVSWSDGDGITGVPQPVEGVSS
FMNVTLDKCTKYNIYDVSGVGVIRVSNDTFLNGITYTSTSGNLLGFKDVTKGTIYSITPCNPPDQLVVYQQAVV
GAMLSENFTSYGFSNVVELPKFFYASNGTYNCTDAVLTYSSFGVCADGSIIAVQPRNVSYDSVSAIVTANLSIP
SNWTISVQVEYLQITSTPIVVDCSTYVCNGNVRCVELLKQYTSACKTIEDALRNSARLESADVSEMLTFDKKAF
TLANVSSFGDYNLSSVIPSLPTSGSRVAGRSAIEDILFSKIVTSGLGTVDADYKNCTKGLSIADLACAQYYNGI
MVLPGVADAERMAMYTGSLIGGIALGGLTSAVSIPFSLAIQARLNYVALQTDVLQENQKILAASENKAMTNIVD
AFTGVNDAITQTSQALQTVATALNKIQDVVNQQGNSLNHLTSQLRQNFQAISSSIQAIYDRLDPPQADQQVDRL
ITGRLAALNVFVSHTLTKYTEVRASRQLAQQKVNECVKSQSKRYGFCGNGTHIFSIVNAAPEGLVFLHTVLLPT
QYKDVEAWSGLCVDGINGYVLRQPNLALYKEGNYYRITSRIMFEPRIPTMADFVQIENCNVTFVNISRSELQTI
VPEYIDVNKTLQELSYKLPNYTVPDLVVEQYNQTILNLTSEISTLENKSAELNYTVQKLQTLIDNINSTLVDLK
WLNRVETYIKSGGYIPEAPRDGQAYVRKDGEWVLLSTFLGSLVPRGSHHHHHHHH
SEQ ID NO: 120: CoV-NL63 Spike ectodomain
METDTLLLWVLLLWVPGSTGFFTCNSNANLSMLQLGVPDNSSTIVTGLLPTHWFCANQSTSVYSANGFFYIDVG
NHRSAFALHTGYYDANQYYIYVTNEIGLNASVTLKICKFSRNTTFDFLSNASSSFDCIVNLLFTEQLGAPLGIT
ISGETVRLHLYNVTRTFYVPAAYKLTKLSVKCYFNYSCVFSVVNATVTVNVTTHNGRVVNYTVCDDCNGYTDNI
FSVQQDGRIPNGFPFNNWFLLINGSTLVDGVSRLYQPLRLTCLWPVPGLKSSTGFVYFNATGSDVNCNGYQHNS
VVDVMRYNLNFSANSLDNLKSGVIVFKTLQYDVLFYCSNSSSGVLDTTIPFGPSSQPYYCFINSTINTTHVSTF
VGILPPTVREIVVARTGQFYINGFKYFDLGFIEAVNFNVTTASATDFWTVAFATFVDVLVNVSATNIQNLLYCD
SPFEKLQCEHLQFGLQDGFYSANFLDDNVLPETYVALPIYYQHTDINFTATASFGGSCYVCKPHQVNISLNGNT
SVCVRTSHFSIRYIYNRVKSGSPGDSSWHIYLKSGTCPFSFSKLNNFQKFKTICFSTVEVPGSCNFPLEATWHY
TSYTIVGALYVTWSEGNSITGVPYPVSGIREFSNLVLNNCTKYNIYDYVGTGIIRSSNQSLAGGITYVSNSGNL
LGFKNVSTGNIFIVTPCNQPDQVAVYQQSIIGAMTAVNESRYGLQNLLQLPNFYYVSNGGNNCTTAVMTYSNFG
ICADGSLIPVRPRNSSDNGISAIITANLSIPSNWTTSVQVEYLQITSTPIVVDCATYVCNGNPRCKNLLKQYTS
ACKTIEDALRLSAHLETNDVSSMLTEDSNAFSLANVTSFGDYNLSSVLPQRNIRSSRIAGRSALEDLLFSKVVT
SGLGTVDVDYKSCTKGLSIADLACAQYYNGIMVLPGVADAERMAMYTGSLIGGMVLGGLTSAAAIPFSLALQAR
LNYVALQTDVLQENQKILAASENKAINNIVASFSSVNDAITQTAEAIHTVTIALNKIQDVVNQQGSALNHLTSQ
LRHNFQAISNSIQAIYDRLDSPPADQQVDRLITGRLAALNAFVSQVLNKYTEVRGSRRLAQQKINECVKSQSNR
YGFCGNGTHIFSIVNSAPDGLLFLHTVLLPTDYKNVKAWSGICVDGIYGYVLRQPNLVLYSDNGVFRVTSRVMF
QPRLPVLSDFVQIYNCNVTFVNISRVELHTVIPDYVDVNKTLQEFAQNLPKYVKPNFDLTPFNLTYLNLSSELK
QLEAKTASLFQTTVELQGLIDQINSTYVDLKLLNRFENLIKRMKQIEDKIEEIESKQKKIENEIARIKKIKGSL
VPRGSHHHHHHHH
SEQ ID NO: 122: Synthetic polypeptide (SARS-COV-2 kappa variant B.1.617.1/3
RBD domain)
METDTLLLWVLLLWVPGSTGNITNLCPFGEVFNATRFASVYAWNRKRISNCVADYSFLYNSASFSTFKCYGVSP
TKLNDLCFTNVYADSFVIRGDEVRQIAPGQTGKIADYNYKLPDDFTGCVIAWNSNNLDSKVGGNYNYRYRLFRK
SNLKPFERDISTEIYQAGSTPCNGVQGFNCYFPLQSYGFQPTNGVGYQPYRVVVLSFELLHAPATVCGPKGSGL
VPRGSHHHHHHHHSAWSHPQFEKGTGGLNDIFEAQKIEWHE
SEQ ID NO: 123: Synthetic polypeptide (SARS-COV-2 delta variant B.1.617.2/3
RBD domain)
METDTLLLWVLLLWVPGSTGNITNLCPFGEVFNATRFASVYAWNRKRISNCVADYSFLYNSASFSTFKCYGVSP
TKLNDLCFTNVYADSFVIRGDEVRQIAPGQTGKIADYNYKLPDDFTGCVIAWNSNNLDSKVGGNYNYRYRLFRK
SNLKPFERDISTEIYQAGSKPCNGVEGFNCYFPLQSYGFQPTNGVGYQPYRVVVLSFELLHAPATVCGPKGSGL
VPRGSHHHHHHHHSAWSHPQFEKGTGGLNDIFEAQKIEWHE
SEQ ID NO: 124: Synthetic polypeptide (SARS-COV-2 delta plus variant)
METDTLLLWVLLLWVPGSTGNITNLCPFGEVFNATRFASVYAWNRKRISNCVADYSFLYNSASFSTFKCYGVSP
TKLNDLCFTNVYADSFVIRGDEVRQIAPGQTGNIADYNYKLPDDFTGCVIAWNSNNLDSKVGGNYNYRYRLFRK
SNLKPFERDISTEIYQAGSKPCNGVEGFNCYFPLQSYGFQPTNGVGYQPYRVVVLSFELLHAPATVCGPKGSGL
VPRGSHHHHHHHHSAWSHPQFEKGTGGLNDIFEAQKIEWHE
SEQ ID NO: 125: Synthetic polypeptide (SARS-COV-2 omicron variant B.1.1.529/
RBD domain)
METDTLLLWVLLLWVPGSTGNITNLCPFDEVFNATRFASVYAWNRKRISNCVADYSVLYNLAPFFTFKCYGVSP
TKLNDLCFTNVYADSFVIRGDEVRQIAPGQTGNIADYNYKLPDDFTGCVIAWNSNKLDSKVSGNYNYLYRLFRK
SNLKPFERDISTEIYQAGNKPCNGVAGFNCYFPLRSYSFRPTYGVGHQPYRVVVLSFELLHAPATVCGPKGSGL
VPRGSHHHHHHHHSAWSHPQFEKGTGGLNDIFEAQKIEWHE
SEQ ID NO: 126: Synthetic polypeptide (SARS-COV-2 omicron variant B.1.1.529/
Spike ectodomain)
MFVFLVLLPLVSSQCVNLTTRTQLPPAYTNSFTRGVYYPDKVFRSSVLHSTQDLFLPFFSNVTWFHVISGINGT
KRFDNPVLPFNDGVYFASIEKSNIIRGWIFGTTLDSKTQSLLIVNNATNVVIKVCEFQFCNDPFLDHKNNKSWM
ESEFRVYSSANNCTFEYVSQPFLMDLEGKQGNFKNLREFVFKNIDGYFKIYSKHTPIIVREPEDLPQGFSALEP
LVDLPIGINITRFQTLLALHRSYLTPGDSSSGWTAGAAAYYVGYLQPRTFLLKYNENGTITDAVDCALDPLSET
KCTLKSFTVEKGIYQTSNFRVQPTESIVRFPNITNLCPFDEVFNATRFASVYAWNRKRISNCVADYSVLYNLAP
FFTFKCYGVSPTKLNDLCFTNVYADSFVIRGDEVRQIAPGQTGNIADYNYKLPDDFTGCVIAWNSNKLDSKVSG
NYNYLYRLERKSNLKPFERDISTEIYQAGNKPCNGVAGFNCYFPLRSYSFRPTYGVGHQPYRVVVLSFELLHAP
ATVCGPKKSTNLVKNKCVNFNFNGLKGTGVLTESNKKFLPFQQFGRDIADTTDAVRDPQTLEILDITPCSFGGV
SVITPGTNTSNQVAVLYQGVNCTEVPVAIHADQLTPTWRVYSTGSNVFQTRAGCLIGAEYVNNSYECDIPIGAG
ICASYQTQTKSHGSAGSVASQSIIAYTMSLGAENSVAYSNNSIAIPTNFTISVTTEILPVSMTKTSVDCTMYIC
GDSTECSNLLLQYGSFCTQLKRALTGIAVEQDKNTQEVFAQVKQIYKTPPIKYFGGFNFSQILPDPSKPSKRSP
IEDLLFNKVTLADAGFIKQYGDCLGDIAARDLICAQKFKGLTVLPPLLTDEMIAQYTSALLAGTITSGWTFGAG
PALQIPFPMQMAYRFNGIGVTQNVLYENQKLIANQFNSAIGKIQDSLSSTPSALGKLQDVVNHNAQALNTLVKQ
LSSKFGAISSVLNDIFSRLDPPEAEVQIDRLITGRLQSLQTYVTQQLIRAAEIRASANLAATKMSECVLGQSKR
VDFCGKGYHLMSFPQSAPHGVVFLHVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPDVDLGDISGINASVVNIQKEIDRLN
EVAKNLNESLIDLQELGKYEQGSGYIPEAPRDGQAYVRKDGEWVLLSTFLGGSHHHHHHHH
SAWSHPQFEKGTGGLNDIFEAQKIEWHE
SEQ ID NO: 184: Synthetic polypeptide (Omicron subvariant BA.2)
METDTLLLWVLLLWVPGSTGNITNLCPFDEVFNATRFASVYAWNRKRISNCVADYSVLYNFAPFFAFKCYGVSP
TKLNDLCFTNVYADSFVIRGNEVSQIAPGQTGNIADYNYKLPDDFTGCVIAWNSNKLDSKVGGNYNYLYRLFRK
SNLKPFERDISTEIYQAGNKPCNGVAGFNCYFPLRSYGFRPTYGVGHQPYRVVVLSFELLHAPATVCGPKGSGL
VPRGSHHHHHHHHSAWSHPQFEKGTGGLNDIFEAQKIEWHE

### Sequence Listing

| SEQ ID | Description |
|---|---|
| 1-102 | Polypeptide sequences & Nucleic acid sequences |
| 103-120 | Polypeptide sequences from viral proteins |
| 3-12 | VH & VL (Cv2.1169 + Cv2.1353+ Cv2.3194 + Cv2.3235 + Cv2.5213) |
| 13-22 | Full length Heavy & Light Chain (Cv2.1169 + Cv2.1353 + Cv2.3194 + |
| | Cv2.3235 + Cv2.5213) |
| 23-52 | Hypervariable sequences (Cv2.1169 + Cv2.1353 + Cv2.3194 + Cv2.3235 + |
| | Cv2.5213) |
| 23 | Cv2.1169 HCDR1 (TSAVQ) |
| 24 | Cv2.1169 HCDR2 (WIVVGSGNTNYAQKFQE) |
| 25 | Cv2.1169 HCDR3 (PYCSGGTCLDGFDI) |
| 26 | Cv2.1169 LCDR1 (RASQSVSRSYLA) |
| 27 | Cv2.1169 LCDR2 (SASSRAT) |
| 28 | Cv2.1169 LCDR3 (QQYGSSPWT) |
| 39 | Cv2.1353 HCDR1 (SNYMN) |
| 30 | Cv2.1353 HCDR2 (VIYSGGSTFYADSVKG) |
| 31 | Cv2.1353 HCDR3 (DLGPMGFDI) |
| 32 | Cv2.1353 LCDR1 (RASQGISSYLA) |
| 33 | Cv2.1353 LCDR2 (AASTLQS) |
| 34 | Cv2.1353 LCDR3 (QQLNSDPPVT) |
| 35 | Cv2.3194 HCDR1 (SNYMS) |
| 36 | Cv2.3194 HCDR2 (VIYPGGSTFYADSVKG) |
| 37 | Cv2.3194 HCDR3 (DLVVYGMDV) |
| 38 | Cv2.3194 LCDR1 (RASQSVSSSYLA) |
| 39 | Cv2.3194 LCDR2 (GASSRAT) |
| 40 | Cv2.3194 LCDR3 (QQGVT) |
| 41 | Cv2.3235 HCDR1 (RNYMS) |
| 42 | Cv2.3235 HCDR2 (VIYSGGSTFYADSVKG) |
| 43 | Cv2.3235 HCDR3 (DGDYYGMDV) |
| 44 | Cv2.3235 LCDR1 (RASQGISSFLA) |
| 45 | Cv2.3235 LCDR2 (GASTLQS) |
| 46 | Cv2.3235 LCDR3 (QRLDSYPPIT) |
| 47 | Cv2.5213 HCDR1 (SNYMS) |
| 48 | Cv2.5213 HCDR2 (LIYSGGSTYYADSVKG) |
| 49 | Cv2.5213 HCDR3 (DLNYYGMDV) |
| 50 | Cv2.5213 LCDR1 (RASQGISSYLA) |
| 51 | Cv2.5213 LCDR2(AASTLQS) |
| 52 | Cv2.5213 LCDR3 (QQLDSYSPFT) |
| 53-92 | Framework regions (Cv2.1169 + Cv2.1353 + Cv2.3194 + Cv2.3235 + |
| | Cv2.5213) |
| 121 | Synthetic peptide-StrepTag |
| 122 | Synthetic polypeptide (SARS-COV-2 kappa variant B.1.617.1/3 RBD domain) |
| 123 | Synthetic polypeptide (SARS-COV-2 delta variant B.1.617.2/3 RBD domain) |
| 124 | Synthetic polypeptide (SARS-COV-2 delta plus variant) |
| 125 | Synthetic polypeptide (SARS-COV-2 omicron variant B.1.1.529/RBD |
| | domain) |
| 126 | Synthetic polypeptide (SARS-COV-2 omicron variant B.1.1.529/Spike |
| | ectodomain) |
| 127 | Endogenous Leader Sequence |
| 128 | Thrombin Cleavage Site |
| 129 | Poly His-Tag |
| 130 | Foldon Trimerization motif |
| 131 | Avi-Tag |
| 132 | Constant Heavy Chain Region |
| 133 | Cv2.1169 Full length Heavy Chain « Prime » |
| 134 | Cv2.1353 Full length Heavy Chain « Prime » |
| 135 | Cv2.3194 Full length Heavy Chain « Prime » |
| 136 | Cv2.3235 Full length Heavy Chain « Prime » |
| 137 | Cv2.5213 Full length Heavy Chain « Prime » |
| 138 | Cv2.5179 HCDR1 (SSAVQ) |
| 139 | Cv2.5179 HCDR2 (WIVVGSGNTNYAQKFQE) |
| 140 | Cv2.5179 HCDR3 (PHCSSTSCYDAFDI) |
| 141 | Cv2.5179 LCDR1 (RASQSVSSSYLA) |
| 142 | Cv2.5179 LCDR2 (GASSRAT) |
| 143 | Cv2.5179 LCDR3 (QQYGRSPWT) |
| 144 | Cv2.5179 H-FR1 |
| 145 | Cv2.5179 H-FR2 |
| 146 | Cv2.5179 H-FR3 |
| 147 | Cv2.5179 H-FR4 |
| 148 | Cv2.5179 L-FR1 |
| 149 | Cv2.5179 L-FR2 |
| 150 | Cv2.5179 L-FR3 |
| 151 | Cv2.5179 L-FR4 |
| 152 | Cv2.5179 VH |
| 153 | Cv2.5179 VL |
| 154 | Cv2.5179 Heavy chain |
| 155 | Cv2.5179 Light chain |
| 156 | Nucleic acid coding for Cv2.5179 Heavy chain |
| 157 | Nucleic acid coding for Cv2.5179 Light chain |
| 158 | Cv2.5156 VH |
| 159 | Cv2.5156 VL |
| 160 | Cv2.1174 VH |
| 161 | Cv2.1174 VL |
| 162 | Cv2.1388 VH |
| 163 | Cv2.1388 VL |
| 164 | Cv2.3132 VH |
| 165 | Cv2.3132 VL |
| 166 | Casirivimab VH |
| 167 | Casirivimab VL |
| 168 | Etesevimab VH |
| 169 | Etesevimab VL |
| 170 | Regdanvimab VH |
| 171 | Regdanvimab VL |
| 172 | Tixagevimab VH |
| 173 | Tixagevimab VL |
| 174 | Adintrevimab VH |
| 175 | Adintrevimab VH |
| 176 | Bamlanivimab VH |
| 177 | Bamlanivimab VL |
| 178 | Cilgavimab VH |
| 179 | Cilgavimab VL |
| 180 | Sotrovimab VH |
| 181 | Sotrovimab VL |
| 182 | Imdevimab VH |
| 183 | Imdevimab VL |
| 184 | Synthetic polypeptide-RBD Omicron (BA.2) |

### Results

### 1. Production of Human Monoclonal Antibodies Against SARS-CoV-2 S Protein

In convalescent COVID-19 individuals, serum antibody levels against the spike and RBD proteins have been correlated to SARS-CoV-2 IgA seroneutralizing activities.

Convalescent COVID-19 patients (infected during the first epidemic wave) with high antibody response to SARS-CoV-2 S protein were selected from COVID-19 cohorts based on the IgG and IgA seroreactivity against SARS-CoV-2 tri-S and RBD by ELISA binding experiments (FIG. 1A, 1B; 2A, 2B, 2C).

Most of convalescent COVID-19 patients had high titers of anti-tri-S IgGs, mainly IgG1, including cross-reacting antibodies against the Middle East respiratory syndrome-related coronavirus (MERS-CoV) tri-S protein (FIGS. 1A, 1B, 2A and 2B). High levels of serum anti-RBD IgGs were also detected (FIGS. 1A, 1B, 2A and 2B), and correlated with anti-tri-S antibody titers (FIG. 2C). Although the SARS-CoV-2 seroreactivity of IgA antibodies was globally weaker than for IgGs, both were correlated (FIGS. 1B, 2B and 2C).

Then, polyclonal serum IgG and IgA from selected convalescent COVID-19 patients were purified and assayed by ELISA binding experiments against various SARS-CoV-2 antigens including tri-S, Si, S2, RBD, FP and N proteins (FIG. 1C, 2D, 2E). Purified serum IgG and IgA antibodies from selected Covid-19 convalescents showed strong ELISA binding to Wuhan nucleocapsid (N), tri-S, S1 and S2 subunits, and RBD, and also cross-reacted against recombinant spike proteins from other 0-coronaviruses (SARS-CoV-1, MERS-CoV, HKU1 and OC43) as well as α-coronaviruses (229E and NL63) (FIGS. 1C, 2D, and 2E). Purified serum IgG and IgA antibodies from selected Covid-19 convalescents were also assayed for in vitro SARS-CoV-2 neutralizing activity (FIG. 1D). Fifty percent inhibitory concentrations (IC₅₀) of purified IgA antibodies were in average lower as compared to IgGs (70.4 vs 115.6 μg/ml for IgAs and IgGs, respectively, p=0.068), ranging from 43 to 133 μg/ml for IgAs, and from 21 to 257 μg/ml for IgGs (FIG. 1D). IC₅₀ values for IgA but not IgG antibodies were negatively correlated to their respective binding levels to SARS-CoV-2 Sl and RBD proteins.

Samples were also tested against MERS tri-S to assay for cross-reactivity against another beta-coronavirus (FIG. 1B, 1C, 2C).

Peripheral blood single SARS-CoV-2 S⁺ IgG⁺ and IgA⁺ B cells were stained with fluorescently-labeled RBD and tri-S, the latter being used as a bait to capture single SARS-CoV-2-reactive B cells and were sorted by flow cytometry, and their frequency was determined by flow cytometric analyses. The frequency of S⁺ RBD⁺ IgG⁺ and IgA⁺ B cells was also determined (FIG. 1E). From the 2870 SARS-CoV-2 tri-S+ IgA+/G+ memory B cells isolated, a total of 133 unique human mAbs were produced by recombinant expression cloning, with most of them being part of B-cell clonal expansions (FIG. 1F).

10 Convalescent COVID-19 patients were selected based on their Spike protein seroreactivity and neutralization.

Monoclonal antibodies were cloned from single cell sorted SARS-CoV-2 S⁺ IgG⁺ and IgA⁺ B cells of selected convalescent COVID-19 patients. The reactivity of the recombinant human monoclonal antibodies against SARS-CoV-2 S protein was analyzed by S-Flow, tri-S ELISA and tri-S capture ELISA (FIG. 1F, 2F).

A total of 101 recombinant human monoclonal antibodies specific to the SARS-CoV-2 Spike protein were isolated out of the 133 cloned, produced and verified for their SARS-CoV-2 S protein specificity.

2. Human SARS-CoV-2 Spike-Specific Memory B-Cell Antibodies from COVID-19 Convalescents (Some Data not Shown)

ELISA and flow cytometry-based (S-Flow) binding analyses showed that 101 purified mAbs specifically bind to SARS-CoV-2 S protein (76% [40-100%]; FIGS. 1F and 2F). RBD-binding cells represented 11% and 17% of the tri-S+ IgA+ and IgG+ B cells, respectively. Anti-RBD IgA titers were correlated with blood RBD+IgA+ B-cell frequencies, and inversely correlated with neutralization IC50 values of IgAs. Both, total and SARS-CoV-2 tri-S-specific class-switched memory B cells showed a resting memory B-cell phenotype (RM, CD19+CD27+CD21+). The frequency of circulating blood follicular helper T cell (cTfh) subsets was also determined. cTfh2 (CD4+CXCR5+CCR6-CXCR3−), most them being activated, were found predominant, and correlated with tri-S+IgG+RM B cells (r=0.83; p=0.0098), illustrating their capacity to promote class switching and affinity maturation of B cells as previously shown. Comparison of immunoglobulin gene features with IgG+ memory B cells from healthy controls revealed an increased usage in the SARS-CoV-2 spike-specific B-cell repertoire of rearranged VH3Vλ3 (p=0.0047) and VX3/Jλ2 (p=0.0019), JH4 (p=0.0312), and Jκ4 (p=0.0387) genes, as well as IgG1 subclass (p=0.0001). Anti-spike antibodies were also enriched in VH1-24/-69 and VH3-30/-33 genes as previously observed, and had reduced CDRH3 positive charges (p=0.0001) and somatic mutations in IgH (9.5 vs 19.2, p<0.0001) and Igλ (6.8 vs 12.4, p<0.0001). As reported, certain antibody clones were shared among several of the COVID-19 convalescents, further exemplifying the inter-individual convergence of antibody responses to SARS-CoV-2.

### 3. Binding and Antiviral Properties of Human Anti-SARS-CoV-2 Spike Antibodies (Data not Shown)

Epitope mapping analyses showed that 59% of the anti-S mAbs (n=101) bind to the S2 subunit, 16% the RBD domain, 17% the NTD domain, 1% the S1 connecting domain, and 7% undefined regions of the SARS-CoV-2 spike. Only one anti-S antibody (0.99% of the total) targeting the S2 subunit recognized the denatured tri-S protein by immunoblotting, but did not bind S-covering linear peptides, indicating that most SARS-CoV-2-S memory antibodies target conformational epitopes. To determine whether anti-spike memory antibodies neutralize the Wuhan strain, their inhibitory activity were measured using three different in vitro functional assays: a competition ELISA measuring the blockage of soluble tri-S or RBD binding to ACE2 ectodomain, a pseudoneutralization assay and a neutralizing assay using live virus called S-Fuse (19). Overall, ˜15% of the anti-S mAbs showed inhibitory activities>50% in the S-Fuse assay, many of which also neutralized pseudotyped SARS-CoV-2 virions and blocked tri-S-ACE2 interactions. Potent neutralizers targeted the RBD, but only 50% of all anti-RBD antibodies blocked SARS-CoV-2 infection with IC₅₀ values<10 μg/ml.

SARS-CoV-2 antibodies can be armed with Fc-dependent effector functions allowing the elimination of virions and infected cells, which can alter the course of infection in vivo. The in vitro capacity of anti-S mAbs to promote antibody dependent cellular cytotoxicity (ADCC), antibody dependent cellular phagocytosis (ADCP) and complement dependent cytotoxicity (CDC) were evaluated. On average, 41.6%, 74.2% and 42.6% of the IgG antibodies displayed ADCC, ADCP and CDC activity, respectively. Effector activities of SARS-CoV-2 antibodies were globally correlated. ADCC- and ADCP-inducing antibodies were directed principally against S2 (50% and 85%, respectively) and the NTD (53% and 76%, respectively. Conversely, anti-RBD antibodies as a group were less efficient at performing ADCC, and to a lesser extent ADCP. SARS-CoV-2 antibodies with CDC potential targeted mainly the NTD (59% of anti-NTD) and the RBD (56% of anti-RBD). Accordingly, CDC and tri-S-ACE2 blocking activities were correlated. Principal-component analyses (PCA) showed that neutralizing and Fc-dependent effector functions segregated into two separate clusters in the PCA of antiviral functions, with 77% of the variance reached when combining the two first principal components. The “neutralization” cluster included mainly anti-RBD antibodies, while the “effector” cluster comprised both NTD- and S2-specific IgGs.

### 4. Characterization of Human Monoclonal Antibodies Against SARS-CoV-2 S Protein

The 101 recombinant human monoclonal antibodies specific to the SARS-CoV-2 Spike protein were further characterized.

The results are presented for the 6 potent anti-RBD antibody neutralizers (Cv2.5213, Cv2.5179, Cv2.3235, Cv2.1353, Cv2.3194, Cv2.1169). Cv2.1169 originates from an IgA producing B cell, whereas Cv2.5213, Cv2.5197, Cv2.3235, Cv2.1353 and Cv2.3194 originate from an IgG producing B cell.

The amino acid sequences of the heavy and light chains of the human monoclonal antibodies Cv2.5213, Cv2.5179, Cv2.3235, Cv2.1353, Cv2.3194, Cv2.1169 are presented in Table 2. The CDR, VH, VL and FR sequences of these antibodies are presented in Table 1, Table 3 and Table 4. Cv2.1169 variable heavy and light chains are encoded by the nucleic acid sequences SEQ ID NO: 93 and SEQ ID NO: 94, respectively. Cv2.1353 variable heavy and light chains are encoded by the nucleic acid sequences SEQ ID NO: 95 and SEQ ID NO: 96, respectively. Cv2.3194 variable heavy and light chains are encoded by the nucleic acid sequences SEQ ID NO: 97 and SEQ ID NO: 98, respectively. Cv2.3235 variable heavy and light chains are encoded by the nucleic acid sequences SEQ ID NO: 99 and SEQ ID NO: 100, respectively. Cv2.5179 variable heavy and light chains are encoded by the nucleic acid sequences SEQ ID NO: 152 and SEQ ID NO: 153, respectively. Cv2.5213 variable heavy and light chains are encoded by the nucleic acid sequences SEQ ID NO: 101 and SEQ ID NO: 102, respectively.

These sequences were cloned independently in an expression vector ((pUC19 plasmids: GeneBank #LT615368.1 (IgG1); LT615369.1 (IgK); LT615370.1 (IgL)) to generate 10 recombinant plasmids for expression of the antibody heavy and light chains of the antibodies Cv2.1169, Cv2.1353, Cv2.3194, Cv2.3235, Cv2.5179 and Cv2.5213, as follows. Plasmids Cv2.1169_pIgH and Cv2.1169_pIgL (antibody Cv2.1169); plasmids Cv2.1353_pIgH and Cv2.1353_pIgL (antibody Cv2.1353); plasmids Cv2.3194_pIgH and Cv2.3194_pIgL (antibody Cv2.3194); plasmids Cv2.3235_pIgH and Cv2.3235_pIgL (antibody Cv2.3235); plasmids Cv2.5179_pIgH and Cv2.5179_pIgL (antibody Cv2.5179), plasmids Cv2.5213_pIgH and Cv2.5213_pIgL (antibody Cv2.5213). E. coli bacteria (DH10B, C3019, NEB) transformed with these plasmids were deposited at the Collection Nationale de Cultures de Microorganismes (CNCM) at the Institut Pasteur, 25 rue du Docteur Roux, 75724 Paris, FR on January 28 and Apr. 2, 2021.

The bacteria strains Cv2.1169_pIgH and Cv2.1169_pIgL containing the plasmid Cv2.1169_pIgH and Cv2.1169_pIgL encoding Cv2.1169 antibody heavy and light chain in expressible form, were deposited at the CNCM at the Institut Pasteur on Jan. 28, 2021 under the number I-5651 and I-5652, respectively.

The bacteria strains Cv2.1353_IgH and Cv2.1353_IgL containing the plasmid Cv2.1353_pIgH and Cv2.1353_pIgL encoding Cv2.1353 antibody heavy and light chain in expressible form, were deposited at the CNCM at the Institut Pasteur on Apr. 2, 2021 under the number I-5668 and I-5669, respectively.

The bacteria strains Cv2.3194_IgH and Cv2.3194_IgL containing the plasmid Cv2.3194_pIgH and Cv2.3194_pIgL encoding Cv2.3194 antibody heavy and light chain in expressible form, were deposited at the CNCM at the Institut Pasteur on Apr. 2, 2021 under the number I-5670 and I-5671, respectively.

The bacteria strains Cv2.3235_IgH and Cv2.3235_IgL containing the plasmid Cv2.3235_pIgH and Cv2.3235_pIgL encoding Cv2.3235 antibody heavy and light chain in expressible form, were deposited at the CNCM at the Institut Pasteur on Apr. 2, 2021 under the number I-5672 and I-5673, respectively.

The bacteria strains Cv2.5179_IgH and Cv2.5179_IgL containing the plasmid Cv2.5179_pIgH and Cv2.5179_pIgL encoding Cv2.5179 antibody heavy and light chain in expressible form, were deposited at the CNCM at the Institut Pasteur on Nov. 15, 2021 under the number I-5775 and I-5776, respectively.

The bacteria strains Cv2.5213_IgH and Cv2.5213_IgL containing the plasmid Cv2.5213_pIgH and Cv2.5213_pIgL encoding Cv2.5213 antibody heavy and light chain in expressible form, were deposited at the CNCM at the Institut Pasteur on Apr. 2, 2021 under the number I-5674 and I-5675, respectively.

Recombinant antibodies were produced by transient co-transfection of Freestyle™ 293-F suspension cells with the above-disclosed recombinant expression plasmids, and recombinant antibodies were purified as disclosed in the material and methods.

The reactivity of the potent anti-RBD neutralizers against SARS-CoV-2 Spike protein was analysed by S-Flow, tri-S ELISA and tri-S capture ELISA. Binding region of monoclonal antibodies on S protein was mapped. Cross-reactivity with other betacoronaviruses was tested. Antiviral function of the monoclonal antibodies was evaluated in competition ELISA assay of tri-S and RBD binding to ACE2 and SARS-CoV-2 neutralization assays (SARS-CoV-2 S-Fuse Assay and pseudoneutralization assay). Fc-dependent effector function was analysed by Complement-dependent cytotoxicity (CDC) assay and Antibody-dependent cellular cytotoxicity (ADCC) assay.

In the collection of 101 anti-S mAbs, 5 potent SARS-CoV-2 neutralizing antibodies were identified (Cv2.5213, Cv2.3235, Cv2.1353, Cv2.3194, Cv2.1169). They bound to the recombinant tri-S, S1 and RBD proteins with high affinity as measured by surface plasmon resonance (FIG. 3A). They targeted similar or spatially-close epitopes on the RBD as shown by their cross-competition for ligand binding by ELISA (FIGS. 3B and 4D). They efficiently blocked the interaction of tri-S to the soluble ACE2 ectodomain (FIG. 3C), suggesting that they recognize the receptor binding motif (RBM). IC₅₀ values for SARS-CoV-2 neutralization, determined using the pseudoneutralization and S-Fuse assays, ranged from 3 to 37 ng/ml and from 0.95 to 11.5 ng/ml, respectively (FIG. 3D). Affinity and neutralization activity Cv2.1169 and Cv2.3194 antibodies are summarized in Table 5 and Table 6 bellow.

The most potent antibody, Cv2.1169, was encoded by V_{H}1-58/D_{H}2-15/J_{H}3 and Vκ3-20/Jκ1 immunoglobulin gene rearrangements, and exhibited low levels of somatic mutation (3.1% VH and 2.1% Vx at the amino acid level).

The potential of the SARS-CoV-2 neutralizers to bind with low-affinity unrelated ligands (polyreactivity), and to cross-react with self-antigens was then evaluated in different complementary binding assays (FIG. 5). None of the antibodies displayed self-reactivity, while only Cv2.3235 and Cv2.3194 showed polyreactivity (FIG. 5 and Section 5. Polyreactivity and Self-reactivity assessment of potent SARS-CoV-2 neutralizing antibodies).

None of the potent neutralizers had ADCC potential, but showed moderate CDC and robust ADCP activities (FIGS. 14A-14C and Section 11. Fc-dependent effector functions of potent CoV-2 neutralizers). Remarkably, Cv2.1169 expressed as IgG1 antibody was one of the strongest ADCP-inducer among all the SARS-CoV-2 Spike mAbs (top 2%; FIG. 14C).

TABLE 5
Binding and neutralizing characteristics of anti-RBD neutralizing antibodies

| | | | SARS- | | triS | | | | | | SARS2 | |
|---|---|---|---|---|---|---|---|---|---|---|---|---|
| mAb | S-Flow | S-Flow | CoV-2 | triS | ELISA | SARS2 | SARS2 | SARS2 | SARS2 | SARS2 | S2 | SARS- |
| name | (%) | (MFI) | triS | Capt-ELISA | (AUC) | S1 | RBD | NTD | CD | S2 | FP | CoV-1 |
| Cv2.3235 | 97 | 1.7E+05 | 3.6 | 1.0 | 13.2 | 3.9 | 3.9 | 0.2 | 0.2 | 0.3 | 0.0 | 0.2 |
| Cv2.5213 | 95 | 2.7E+05 | 3.8 | 2.1 | 14.2 | 3.5 | 3.5 | 0.1 | 0.1 | 0.1 | 0.1 | 0.1 |
| Cv2.1169 | 96 | 2.2E+05 | 3.8 | 2.2 | 13.1 | 3.5 | 3.5 | 0.1 | 0.1 | 0.1 | 0.1 | 0.1 |
| Cv2.1353 | 95 | 2.7E+05 | 3.5 | 2.2 | 14.0 | 3.5 | 3.5 | 0.1 | 0.1 | 0.1 | 0.1 | 0.1 |
| Cv2.3194 | 94 | 2.6E+05 | 3.6 | 2.3 | 15.8 | 3.7 | 3.6 | 0.1 | 0.1 | 0.1 | 0.1 | 0.1 |

| | | | | | | ACE2 | | | | |
|---|---|---|---|---|---|---|---|---|---|---|
| mAb | MERS- | NL63- | OC43- | HKU1- | 229E- | Block | PseudoNEUT | S-FuseNEUT | ADCC | CDC |
| name | CoV | CoV | CoV | CoV | CoV | (%) | (%) | (%) | (Fold) | (%) |
| Cv2.3235 | 0.2 | 0.2 | 0.3 | 0.2 | 0.1 | 78 | 100 | 100 | 1.2 | 13.5 |
| Cv2.5213 | 0.1 | 0.1 | 0.7 | 0.1 | 0.1 | 90 | 100 | 96 | 1.1 | 12.0 |
| Cv2.1169 | 0.1 | 0.1 | 0.1 | 0.1 | 0.1 | 90 | 100 | 95 | 1.2 | 14.6 |
| Cv2.1353 | 0.1 | 0.1 | 0.8 | 0.1 | 0.1 | 92 | 100 | 100 | 1.2 | 13.6 |
| Cv2.3194 | 0.1 | 0.1 | 0.8 | 0.1 | 0.1 | 91 | 100 | 100 | 1.6 | 15.0 |

The binding affinity of the potent anti-RBD neutralizers for tri-S, S1 and RBD proteins was measured by surface plasmon resonance (SPR) and K_{D} values were determined (FIG. 3A).

Competition of the anti-RBD antibodies for binding to tri-S, and RBD was evaluated by ELISA with biotinylated anti-RBD antibodies and antibodies as potential competitors (FIGS. 3B and 4D).

Binding of biotinylated tri-S to immobilized ACE-2 in presence of anti-RBD antibodies as competitors was assessed in ELISA (FIG. 3C).

Neutralization curves of SARS-CoV-2 and SARS-CoV-2 viral variants by anti-RBD antibodies was determined with SARS-CoV-2 S-Fuse Assay and pseudoneutralization assay (FIGS. 3D and 3G).

The binding and RBD-ACE2 blocking capacity of RBD-specific IgG antibodies for RBD proteins of SARS-CoV-2 and viral variants (B.1.1.7, B.1.351 and P.1) was measured by ELISA (FIG. 3F, 4E, 4F, 4G).

Binding of biotinylated tri-S to immobilized ACE-2 in presence of Cv2.1169 IgG and IgA antibodies as competitors was assessed in ELISA (FIG. 3L).

Neutralization curves of SARS-CoV-2 by Cv2.1169 IgG and IgA were determined with SARS-CoV-2 S-Fuse Assay and pseudoneutralization assay (FIG. 3L).

Binding of Cv2.1169 IgG and IgA antibodies to SARS-CoV-2 tri-S, S1 and RBD, and to RBD proteins from SARS-CoV-2 viral variants was determined in ELISA (FIG. 3J, 4).

All potent neutralizers have high affinity to S, target the RBD and cross compete with each-others for S-binding. Furthermore, the most potent antibody, Cv2.1169 also neutralized D614G, B1.1.7, B1.351 and P1 viral variants in vitro comparably to the original Wuhan strain.

### 5. Polyreactivity and Self-Reactivity Assessment of Potent SARS-CoV-2 Neutralizing Antibodies

The polyreactivity and self-reactivity of the potent SARS-CoV-2 neutralizing antibodies was assessed by ELISA against dsDNA (DNA), flagellin (Fla), gp140 (HIV-1 YU2), insulin (INS), keyhole limpet hemocyanin (KLH), lipopolysaccharide (LPS), lysozyme (LZ), MAPK-14 (MAPK), peptidoglycan (PG), and thyroglobulin (Tg) (FIGS. 5A and 5B); immunofluorescence to Hep2-expressing self-antigens (FIG. 5C); Hep-2 reactivity by ELISA (FIG. 5D); human proteins (FIG. 5E). Polyreactivity index was determined by comparison with non-reactive antibody (FIG. 5F).

No polyreactivity, self-reactivity and off-target binding to human proteins was detected, in particular for the ultra-potent neutralizer Cv2.1169 (FIG. 5A to 5F).

### 6. In Vivo Therapeutic Activity of Potent SARS-CoV-2 Neutralizing Antibody

The therapeutic efficacy of potent SARS-CoV-2 neutralizer Cv2.1169 was tested in SARS-CoV-2 (Wuhan strain)-infected K18-hACE2 mice and golden Syrian hamsters.

K18-hACE2 mice were infected intranasally (i.n.) with 10⁴ plaque forming units (PFU) of SARS-CoV-2 virus and received Cv2.1169 antibody according to two different treatment regimen:
6 h later intraperitoneal (i.p.) injections of Cv2.1169 or isotypic control IgG antibody at ˜10 mg/kg (0.25 mg) and ˜20 mg/kg (0.5 mg) (FIG. 6A);
6 h later intraperitoneal (i.p.) injections of Cv2.1169 IgG and IgA antibodies or isotypic control IgG antibody at ˜5 mg/kg (0.125 mg) (FIG. 6C).

K18-hACE2 mice were infected intranasally (i.n.) with 10⁵ plaque forming units (PFU) of SARS-CoV-2 (Wuhan strain) virus and received 22 h later intraperitoneal (i.p.) injections of Cv2.1169 or isotypic control IgG antibody at ˜40 mg/kg (1 mg) plus (i.n.) injection of Cv2.1169 at ˜16 mg/kg (0.4 mg) (FIG. 6B).

The results in K18-hACE2 mice show that:
Cv2.1169 IgG treatment 6h pi (at both 10 and 20 mg/kg) led to 100% survival and recovery (vs 0% in the control group) (FIG. 6A).
Cv2.1169 IgG treatment 24h pi (at 40 mg/kg) still led to 50% survival and recovery (vs 0% in the control group) in mice infected with 10-fold viral inoculum (FIG. 6B).
A single injection of Cv2.1169 IgG at 5 mg/kg but not Cv2.1169 IgA still led to up to 77% survival (FIG. 6C).

Infected mice from the control group lost up to 25% of their body weight within the first 6 days post-infection (dpi) before dying at 7-8 dpi (FIG. 6A). In contrast, all animals treated with Cv2.1169 IgG survived and recovered their body weight after experiencing a transient loss during the first week (FIG. 6A). Even when infected with a higher viral inoculum (10⁵ PFU SARS-CoV-2), and treated 22 h post-infection with Cv2.1169 IgG (˜40 mg/kg i.p. plus i.n.), half of the mice survived compared to those in the control group (p=0.029) (FIG. 6B). Next, to evaluate the in vivo efficacy of Cv2.1169 IgA antibodies, a single low dose of either Cv2.1169 IgA or IgG antibodies (0.125 mg i.p., ˜5 mg/kg) was administered to SARS-CoV-2-infected mice. Despite a significant and comparable reduction of viral loads in the oral swabs of Cv2.1169 IgA- and IgG-treated mice compared to control animals at 4 dpi (2.6×10⁴ eqPFU/ml vs 5.7×10³ eqPFU/ml for Cv2.1169 IgA [p=0.008], and 4.7×10³ eqPFU/ml for Cv2.1169 IgG [p=0.029]) (FIG. 15A), all mice treated with the SARS-CoV-2 IgAs died at 7-8 dpi, whereas 75% of the Cv2-1169 IgG-treated mice survived and recovered their initial body weight after 2 weeks (FIG. 6C). This can be explained by the rapid decay of circulating human IgA as compared to IgG antibodies in mice (FIG. 15C).

SARS-CoV-2-related pathogenesis in infected Golden Syrian hamsters resemble mild COVID-19 disease in humans. Golden Syrian hamsters were infected intranasally (i.n.) with 6. 10⁴ plaque forming units (PFU) of SARS-CoV-2 (Wuhan strain) virus and received various treatment regimen:
24 h later intraperitoneal (i.p.) injections of PBS, Cv2.1169 or isotypic control IgG antibody at ˜40 mg/kg (1 mg i.p.) (FIG. 6D);
4 h later intraperitoneal (i.p.) injections of Cv2.1169 IgG and IgA antibodies or isotypic control IgG antibody at ˜20 mg/kg (0.5 mg i.p.) (FIG. 6E).

The results in Syrian Hamsters show that a single injection of Cv2.1169 IgG in infected hamsters induced a significant decrease of intra-lung viral infectivity and viremia (FIG. 6D), even 24h post-infection (FIG. 6E).

Lung weight to body weight (LW/BW) ratio, intra-lung viral infectivity and RNA load were measured at 5 dpi. Both pulmonary viral infectivity and RNA levels in hamsters treated with Cv2.1169 were significantly reduced compared to control animals (2.44×10³ vs 10×10⁵ PFU/lung, p=0.0005 and 4.3×10⁷ vs 3.4×10⁸ copies/pg RNA, p=0.013, respectively) (FIG. 6D).

IgA- and IgG-treated hamsters showed a reduction in LW/BW ratio compared to control animals (1.64 vs 1.4 for IgA [p=0.03] and 1.32 for IgG [p=0.004]) (FIG. 6E).

As expected from the rapid disappearance of circulating human IgA antibodies in treated animals (FIG. 15E), the intra-lung viral infectivity and RNA loads were comparable between SARS-CoV-2 neutralizing IgA-treated and control hamsters (FIG. 6D). In contrast, the administration of Cv2.1169 IgG antibodies reduced both SARS-CoV-2 infectivity and RNA levels in the lungs of treated hamsters (1.39×10⁶ vs 80 PFU/lung, p=0.0002; 6.14×10⁸ vs 1.51×10⁸ copies/pg RNA, p=0.028) (FIG. 6D). Cv2.1169 IgA and IgG-treated animals showed similar endogenous anti-spike IgG titers, which were reduced as compared to the control group (p<0.0001 and p=0.0003, respectively), suggesting potential early antiviral effects of Cv2.1169 IgA antibodies against SARS-CoV-2 infection (FIG. 15F).

For activity against VOCs see also Section 13. In vivo therapeutic activity of potent SARS-CoV-2 neutralizing antibody Cv2.1169 against the Beta variant and Section 15. In vivo therapeutic and prophylactic activity of Cv2.1169 and benchmarked antibodies in hamsters infected with SARS-CoV-2 Delta and Omicron BA.1 variants.

### 7. Neutralization Spectrum of Potent SARS-CoV-2 Neutralizers

Several SARS-CoV-2 variants of concern (VOCs), i.e., Alpha (α, B.1.1.7), Beta (β, B.1.351), Gamma (γ, P.1) and Delta (δ, B.1.617.2), and variants of interest (VOI) have emerged during the epidemics (https://www.who.int/). The cross-reactive potential of the 16 anti-RBD antibodies was next evaluated. Binding analyses by flow cytometry showed that 3 out of the 5 potent neutralizers bound to cells expressing the spike proteins from VOCs (α, β, γ, δ) and VOI (ε, ι, κ, λ, μ), while most non-neutralizing antibodies had narrowed cross-reactivity spectra (FIG. 3E). Only neutralizers Cv2.1169, Cv2.3194 and Cv2.1353, as well as a third of the non-neutralizing antibodies, displayed unaltered ELISA binding to RBD proteins from the VOCs α, β, γ, δ and VOI κ, δ+ (FIGS. 3G, 4E and 4H). Cv2.1169 and Cv2.3194 were the sole anti-RBD antibodies uniformly blocking the interaction of the ACE2 ectodomain with RBD proteins from the viral variants tested (FIG. 3G). Three potent neutralizers encoded by VH3-53/-66 immunoglobulin genes (Cv2.1353, Cv2.5213 and Cv2.3235), sensitive to the RBD mutations at positions 417 and 501, lost binding and/or blocking activity against SARS-CoV-2 α, β, γ, δ (FIGS. 3G and 4E-4I). Both S-Fuse and pseudo-neutralization assays showed that Cv2.1169 and Cv2.3194 neutralized SARS-CoV-2 variants α, β, γ, δ (FIGS. 3J, 14D and 14E). VH3-53 gene-expressing antibody Cv2.3194 efficiently bound and neutralized all the variants, most likely due to the usage of rearranged Vκ3-20/Jκ4 light chain genes as previously reported. Among these cross-neutralizers, Cv2.1169 was the most potent with IC₅₀ values ranging from 1.5 to 2.7 ng/ml against Wuhan, D614G variant, α, β, γ, and δ strains in the S-Fuse assay, and from 3.5 to 14 ng/ml against D614G variant, α, β, γ, δ and δ+ strains in the pseudoneutralization assay (FIGS. 3D, 3J, 14D and 14E). Cv2.1169 ranked among the strongest cross-neutralizers when compared to the parental versions of benchmarked antibodies used in clinics or in development (FIGS. 18A-18C). In addition, a Cv2.1169 IgG homolog (VH1-58/DH2/JH3 and Vκ3-20/Jκ1) from a different convalescent donor were produced based on interindividual clonal convergence analyses (FIGS. 7A and 7B), Cv2.5179, which also exhibited a potent and broad SARS-CoV-2 neutralizing activity (FIGS. 3L and 18C-18E).

Immunophenotyping of sorted B cells indicated that Cv2.1169 was originally expressed by a Spike+RBD+IgA+B cell with an activated memory phenotype (CD27+CD21-), and expressing the mucosa-homing integrin 07. Thus, Cv2.1169 was also expressed as monomeric IgA antibody, which showed equivalent binding and neutralization compared to its IgG counterpart (FIGS. 3K, 3L and 4I). In contrast, purified J-chain containing IgA dimers demonstrated a higher neutralizing capacity against the Wuhan strain (FIG. 3M), suggesting an enhanced neutralization by binging avidity effects as previously reported. Accordingly, the neutralizing activity of Cv2.1169 IgA Fab against SARS-CoV-2 was strongly impaired as compared to bivalent immunoglobulins (FIG. 3L).

### 8. Cv2.1169 Binding to Spike and RBD Proteins of VOC Omicron

SARS-CoV-2 Omicron variant B.1.1.529 or BA.1 became dominant worldwide (https://www.who.int/). Omicron BA.1 contains 15 RBD-amino acid substitutions, which conferred resistance to numerous potent anti-RBD neutralizers including those in clinical use.

The purpose of this study was to compare the binding of Cv2.1169 and selected benchmark antibodies to the SARS-CoV-2 Spike (tri-S) from the VOC Omicron and RBD proteins from VOC Beta (0) and Omicron (o) viral strains.

The selected benchmark antibodies are reported in FIG. 7A as Cv2.1174, Cv2.5156 and Cv2.1388. Polypeptide sequences corresponding to their variable regions (heavy chain variable region VH and light chain variable region VL) are reported as sequences SEQ ID No 158 to SEQ ID No 163.

ELISA plates were coated with purified recombinant SARS-CoV-2 Spike VOC o and RBD proteins from β (used as control) and o. After washing, plates were blocked with a BSA-containing solution, incubated with various concentrations of Cv2.1169 or benchmark IgG antibodies, and then revealed by goat peroxidase-conjugated anti-human IgG antibodies. Optical densities were measured at 405 nm (OD_{405 nm}).

FIG. 9 and FIGS. 17A and 17C show that Cv2.1169 binds to purified recombinant SARS-CoV-2 Spike and RBD domain from Omicron variant (o) in a dose-dependent manner. Plateau values were reached around 150 ng/ml for tri-S and 625 ng/ml for RBD. EC₅₀ values expressed in ng/ml (mean±SD, n=2) vary from 5.4 (0) to 27 (o) for the RBDs indicating that the binding of Cv2.1169 to RBD o is 5-fold decreased as compared to RBD 3.

Cv2.1169 and Cv2.3194, but not the other anti-RBD antibodies, bound well to cell-expressed and soluble Omicron BA.1 spike proteins as well as to the o BA.1 RBD (FIG. 17A). Both antibodies blocked Omicron BA.1 tri-S binding to ACE2, although less efficiently than for the Wuhan viral spike (FIG. 17B). Cv2.1169 and Cv2.3194 also had the highest binding and spike-ACE2-blocking capacity to Omicron BA.1 viral proteins by ELISA as compared to benchmarked antibodies (FIGS. 17C and 17D). Cv2.1169 and Cv2.3194, but not Cv2.5179, neutralized BA.1 in the S-Fuse assay with IC₅₀ of 253 ng/ml and 24.2 ng/ml, respectively (FIG. 17E). Thus, Cv2.1169 and Cv2.3194 presented, respectively, a 79- and 2.2-fold decreased neutralization efficacy on Omicron BA.1 as compared to Delta (FIG. 17E). In contrast, Cv2.1169 and Cv2.3194 showed a slightly stronger RBD-binding against Omicron BA.2 as compared to o BA.1 (FIG. 17D). Consistently, both antibodies blocked more efficiently the binding of the RBD BA.2 to soluble ACE2 (FIG. 17F). Nonetheless, Cv2.1169 and Cv2.3194 showed comparable neutralizing activities against Omicron BA.1 and BA.2 in the S-Fuse assay (FIG. 17G). As compared to their monomeric counterpart, dimeric Cv2.1169 IgA antibodies had enhanced RBD-binding and spike-ACE2 blocking activities to Omicron variants especially BA.1 (FIGS. 17H and 17I). This translated into an increased neutralizing potency of Cv2.1169 IgA dimers against Omicron BA.1 and BA.2 by a 13- and 20-fold, respectively when normalized for the number of binding sites (FIG. 17J).

9. Cv2.1169 Inhibit the Binding of SARS-CoV-2 Spike and RBD from Wuhan and VOC o to ACE-2

The ability of Cv2.1169 and benchmark antibodies to inhibit the Spike protein interaction with its receptor ACE-2 was studied by ELISA using Spike (tri-S) proteins from Wuhan and VOC Omicron viral strains.

ELISA plates were coated overnight with 250 ng/well of purified ACE-2 ectodomain. After blocking, biotinylated tri-S and RBD (Wuhan and variant o) were added in the presence of Cv2.1169 or benchmark antibodies. After washing, the plates were revealed by incubation for 30 min with streptavidin peroxydase-conjugated (BD Biosciences). Optical densities were measured at 405 nm (OD_{405 nm}).

FIG. 10 shows that Cv2.1169 inhibits the interaction between tri-S and ACE-2 in a dose dependent manner. Plateau values were obtained from 92.5 ng/ml (Wuhan) to 583.4 ng/ml (o). IC₅₀ expressed in mean±SD (n=2) vary from 250 ng/ml (Wuhan) to ˜ 2000 ng/ml (o). Cv2.1169 still inhibit Spike Omicron interactions with its ligand ACE-2. This functional property suggests that Cv2.1169 could block Omicron BA.1 virus attachment to its target and consequently, and would still diminish the viral infection mediated by SARS-CoV-2 Omicron variant.

### 10. In Vitro SARS-CoV-2 Neutralization by Antibodies of the Present Disclosure, Including Cv2.3194, Cv2.5179, Cv2.1169, Against δ, δ+ and o Strains Using the S-Fuse Assay.

The ability of Cv2.1169 and other benchmark antibodies to neutralize SARS-CoV-2 Delta (used as control) and Omicron viruses was evaluated in vitro using the S-Fuse neutralization assay.

S-Fuse cells (U20S-ACE-2 GFP1-10 or GFP 11 cells) were incubated with SARS-CoV-2 viral variants in the presence of various dilutions of IgG antibodies. After 18h, the cells were fixed and images were acquired with confocal microscope. Based on the area displaying GFP expression and the number of nuclei, the percentage neutralization was calculated. EC₅₀ values (ng/ml) were calculated based on the dose-response curve.

FIG. 11 shows dose response SARS-CoV-2 neutralization by Cv2.1169. Plateau values were obtained from 15.6 ng/ml (Delta) and 1000 ng/ml (o). IC₅₀ values are comprised between 4.46 ng/ml (Delta) and 212.2 ng/ml (o) with the S-Fuse assay, indicating that the neutralizing activity of Cv2.1169 against Omicron o is ˜ 50-fold decreased as compared to Delta.

Despite that Cv2.1169 shows decreased RBD-binding and RBD-ACE2-blocking capacities as well as a reduced neutralizing activity against SARS-CoV-2 VOC Omicron, the antibody still neutralize SARS-CoV-2 Omicron with an IC_{50˜0.25} μg/ml, and ranks first among all the benchmark antibodies tested across the described assays.

FIGS. 12 and 13 further confirm that all the tested neutralizing antibodies according to the present disclosure, in particular those including Cv2.3194, Cv2.5179, and Cv2.1169 have broad neutralization properties against a plurality of variants of concern.

TABLE 6
Affinity and neutralization activity
Cv2.1169 and Cv2.3194 antibodies.

| Affinity | | Cv2.1169 | | Cv2.3194 | |
|---|---|---|---|---|---|
| (SPR) | | | K_{D} (M) | | K_{D} (M) |
| Wuhan | ka (M⁻¹s⁻¹) | 6.36E+05 | | 2.45E+06 | |
| tri-S | kd (s⁻¹) | 1.80E−04 | 2.84E−10 | 1.12E−03 | 4.59E−10 |
| Wuhan | ka (M⁻¹s⁻¹) | 2.46E+05 | | 4.39E+05 | |
| S1 | kd (s⁻¹) | 3.54E−04 | 1.44E−09 | 5.25E−04 | 1.20E−09 |
| Wuhan | ka (M⁻¹s⁻¹) | 1.12E+06 | | 4.87E+05 | |
| RBD | kd (s⁻¹) | 7.52E−04 | 6.72E−10 | 3.06E−06 | 6.27E−12 |

| | IC₅₀ | IC₉₀ | IC₅₀ | IC₉₀ |
|---|---|---|---|---|
| | (ng/ml) | (ng/ml) | (ng/ml) | (ng/ml) |
| triS/RBD-ACE2 blocking | | | | |
| Wuhan tri-S | 53 | 115 | 93 | 212 |
| Omicron BA.1 tri-S | 584 | 1314 | 876 | 1204 |
| Wuhan RBD | 234 | 523 | 99 | 185 |
| Alpha RBD | 295 | 601 | 212 | 737 |
| Beta RBD | 351 | 2159 | 206 | 406 |
| Gamma RBD | 479 | 2376 | 412 | 591 |
| Delta RBD | 344 | 908 | 827 | 1310 |
| Delta⁺ RBD | 216 | 518 | 280 | 967 |
| Kappa RBD | 168 | 446 | 469 | 1099 |
| Omicron BA.1 RBD | 25491 | 242267 | 6530 | 37554 |
| Omicron BA.2 RBD | 20427 | 136446 | 2713 | 29066 |
| Pseudoneutralization | | | | |
| Wuhan | 3.0 | 12.4 | 18.9 | 240.6 |
| D614G | 4.8 | 24.0 | 40.5 | 159.8 |
| Alpha | 5.5 | 24.0 | 34.7 | 131.9 |
| Beta | 14.2 | 34.2 | 34.3 | 118.8 |
| Gamma | 9.6 | 29.2 | 9.9 | 56.1 |
| Delta | 8.0 | 34.1 | 54.4 | 114.5 |
| Delta⁺ | 3.6 | 14.9 | 20.6 | 89.0 |
| S-Fuse neutralization | | | | |
| Wuhan | 1.5 | 3.5 | 3.0 | 9.8 |
| D614G | 2.1 | 5.7 | 14.5 | 145.4 |
| Alpha | 1.6 | 4.0 | 4.3 | 5.4 |
| Beta | 2.3 | 7.2 | 5.2 | 20.0 |
| Gamma | 1.6 | 8.6 | 4.5 | 5.6 |
| Delta | 2.1 | 6.4 | 6.6 | 54.7 |
| Omicron BA.1 | 265.3 | 7592 | 24.2 | 709 |
| Omicron BA.2 | 331.5 | 9694 | 40.5 | 582 |

### 11. Fc-Dependent Effector Functions of Potent CoV-2 Neutralizers

FIG. 14A illustrates that potent SARS-Cov-2 neutralizers according to the present disclosure tend to induce Antibody-Dependent Cellular Cytotoxicity (ADCC) activity, although the induction is moderate compared to non-neutralizers (non-nAbs).

FIG. 14B illustrates that potent SARS-Cov-2 neutralizers according to the present disclosure tend to induce midly Complement-Dependent Cytotoxicity (CDC) activity, but the induction is moderate compared to non-neutralizers (non-nAbs).

FIG. 14C illustrates that potent SARS-Cov-2 neutralizer Cv2.1169 according to the present disclosure tends to induce significantly Antibody-Dependent Cellular Phagocytosis (ADCP) activity, both for recombinant IgG1, monomeric IgA1 and dimeric IgA1 antibodies.

### 12. Structural Characterization of the Epitopes (Data not Shown)

To precisely define the epitopes and neutralization mechanisms of the most potent mAbs, the corresponding Fab were produced to make co-crystallization trials in complex with the Wuhan RBD. Crystals of the Cv2.3235 Fab/RBD and the Cv2.6264 Fab/RBD complexes were obtained, and their X-ray structure to 2.3 Å and 2.8 Å resolution were determined, respectively. The Cv2.1169 Fab did not crystallize as a binary complex with the RBD, but crystallized as a Cv2.1169 IgA Fab/CR3022 IgG1 Fab/RBD ternary complex, which allowed to determine the X-ray structure to 2.9 Å. These crystals did not show interpretable density for the Cv2.1169 Fab constant domains, which were mobile, but the variable domains and the paratope/epitope region was clearly resolved. The crystal structure showed that Cv2.1169 binds the RBM and straddles the RBD ridge leaning toward the face that is occluded in the “down” conformation of the RBD on a “closed” spike. The binding mode of Cv2.1169 is similar to other VH1-58/VK3-20-derived neutralizing antibodies, as shown in the superposition model with A23-58.1, COVOX-253 and S2E12 mAbs. Superposing the structures of the RBD-Cv2.1169 and RBD-ACE2 complexes showed extensive clashes between the antibody and the receptor. The steric hindrance introduced by Cv2.1169 binding provides the structural basis for its neutralization mechanism, and is in agreement with its RBD-ACE2 blocking capacity (FIGS. 3C, 3F, 4F and 4I). The RBD-binding of Cv2.1169 differed from the one of two less potent VH3-53 and VH1-69 anti-RBD neutralizing antibodies (Cv2.3235 and Cv2.6264, respectively). Compared to Cv2.3235 and Cv2.6264 antibodies, Cv2.1169 bound with a modest total buried surface area (BSA) (˜1400 Å2, ˜2620 Å2 and ˜1820 Å2, for Cv2.1169, Cv2.3235 and Cv2.6264, respectively). In addition, all the CDRs of Cv2.1169, but not of Cv2.3235 and Cv2.6264, contacted the RBD with a major contribution of the heavy chain that provides most of the interaction surface (˜80% of the paratope's BSA), mainly in a CDRH3-dependent manner. The Cv2.1169 CDRH3 (14 amino acid length by Kabat definition) contains kinks at P99 and F110 that delimit a tongue-like loop, which is stabilized by a disulfide bond between C101CDRH3 and C106CDRH3. This particular shape allows the recognition of the RBD tip by several residues between G104 and F110 from only one side of the CDRH3 “tongue” and form hydrogen bonds with the RBD through their main-chain atoms. In addition, polar interactions at the interface involve the side chains of D108 in the CDRH3 and Y33 in the CDRL1.

Overall, Cv2.1169 interacts with the RBD segments 417-421, 455-458, 473-478 and 484-493. Apart from T478, all the mutated RBD residues present in the SARS-CoV-2 VOCs prior to Omicron are at the rim of the contact area (K417, E484) or outside (L452, N501). Conversely, the Cv2.3235 antibody heavy and light chains interact with several residues mutated in several VOCs, e.g., K417 and N501, explaining their reduced capacity to bind and to neutralize α, β, γ and δ+ variants (FIGS. 3E, 3F and 14A). The RBD residue T478 forms hydrogen bonds with both Cv2.1169 heavy and light chains, and is mutated in the δ and δ+ variants (T478K). However, despite this substitution of a threonine for a lysine, the Cv2.1169 antibody is still able to efficiently bind and neutralize both variants (FIGS. 3E, 3F, 14A and 14B). This indicates that the interface's integrity does not depend on the hydrogen bonds formed with the T478 side chain, and that there is enough space for the lysine residue to adopt a rotamer with an orientation that reduces the clashes with the antibody. Unlike the Cv2.6264 antibody, which also straddles the RBD ridge but lost reactivity against the δ and δ+ variants (FIG. 3E), Cv2.1169 buries the RBD F486 and N487 residues within a hydrophobic cavity. This pocket is formed by aromatic residues of the FWRH2 (W50), the CDRH3 (F110), the CDRL1 (Y33) and the CDRL3 (Y92 and W97), and mimics the environment encountered by these residues when interacting with ACE2. Thus, the F486 and N487 residues most likely act as an anchor for Cv2.1169, which strengthens its interaction with the RBM allowing to tolerate the T478K mutation in the δ and δ+ variants. Four of the Cv2.1169-RBD contact residues are mutated in BA.1 and BA.2 variants, including the substitution K417N already present in β and γ, and T478K in δ, as well as two Omicron-specific mutations S477N and Q493R. Although all located at the periphery of Cv2.1169 binding site, those combined mutations explain the decreased binding and neutralization of SARS-CoV-2 BA.1 and BA.2 as compared to the other VOCs (FIG. 17).

As afore-mentioned, the Cv2.1169 antibody leans towards the RBD's occluded face, making the epitope inaccessible on the ‘down’ RBD conformation due to the proximity with the neighboring protomer. This implies that the antibody would only bind to the RBD in its ‘up’ conformation. This is indeed the case by determining the Å cryo-EM reconstruction of the SARS-CoV-2 S_6P protein trimer in complex with Cv2.1169 IgA Fab to 3.Å resolution. This structure showed that each protomer in the 3-up RBD open spike is bound by a Cv2.1169 Fab fragment. Considering that Cv2.1169 blocked SARS-CoV-2 tri-S binding to soluble ACE2 receptor, and that its binding site is only accessible in the up-RBD conformation, our data suggest that the antibody belongs to the class 1 category (or Ia), with an epitope in the RBD-B group. Accordingly, Cv2.1169 cross-competed with class 1 benchmarked SARS-CoV-2 neutralizers (CT-P59, COV2-2196, REGN10933, and CB6), but also moderately with class 2 antibody LY-CoV555, for the binding to spike and RBD proteins.

### 13. In Vivo Therapeutic Activity of Potent SARS-CoV-2 Neutralizing Antibody Cv2.1169 Against the Beta Variant.

The therapeutic efficacy of potent SARS-CoV-2 neutralizer Cv2.1169 was further tested in SARS-CoV-2-infected K18-hACE2 mice following inoculation of the Beta variant, as shown in FIG. 15, and as previously illustrated for FIG. 6 with the Wuhan strain.

To determine whether Cv2.1169 is active in vivo against infection with SARS-CoV-2 VOCs, the prophylactic activity of Cv2.1169 IgA antibodies and the therapeutic activity of Cv2.1169 IgG antibodies against SARS-CoV-2 VOC β were tested in K18-hACE2 knock-in mice. A single administration of Cv2.1169 IgA antibodies at ˜10 mg/kg (0.25 mg i.p.) 6h prior to infection with 10⁴ PFU of SARS-CoV-2 variant R protected 87.5% of the animals from death (FIG. 6F). Despite the fact that human SARS-CoV-2 IgA antibodies did not persist in the mouse circulation (FIG. 15C), Cv2.1169 IgA-treated mice also recovered their initial body weight during the follow-up (FIG. 6F). Likewise, treating once SARS-CoV-2 β-infected mice with Cv2.1169 IgG antibodies (0.25 mg i.p., ˜10 mg/kg) 6h post-infection led to 100% survival, while all animals receiving the control antibodies succumbed at 7-8 dpi (FIG. 6F). Of note, human Cv2.1169 IgG antibodies were still detectable in mouse sera at the end of the follow-up (FIGS. 15B and 15C). In addition, mice pre-treated with Cv2.1169 IgAs developed higher anti-spike IgG antibody titers as compared to those treated with Cv2.1169 IgG antibodies, suggesting a weaker viral control in the former group (FIG. 15).

Overall, the data confirm that pre-treatment with IgA immunoglobulins comprising the variable region of Cv2.1169 or treatment with IgG immunoglobulins comprising the same variable region are potent neutralizers in vivo for a plurality of SARS-CoV2 variants of concern.

In particular, FIG. 15A confirms that the SARS-CoV2 RNA levels are significantly decreased four days post-infection (dpi) following an intraperitoneal injection of 5 mg/kg Cv2.1169 IgG or IgA when compared to control mice.

For activity against Wuhan SARS-CoV-2 see also Section 6. In vivo therapeutic activity of potent SARS-CoV-2 neutralizing antibody. For activity against VOCs see also Section 15. In vivo therapeutic and prophylactic activity of Cv2.1169 and benchmarked antibodies in hamsters infected with SARS-CoV-2 Delta and Omicron BA.1 variants.

14. Benchmarking with Other Therapeutic Antibodies Directed Against the RBD-Domain of SARS-CoV2.

FIGS. 16A and 16B provide a comparative study of Cv2.1169 and Cv2.3194 binding properties with a selection of other reference therapeutic antibodies having neutralizing properties against one or more variants of concerns. The tested reference therapeutic antibodies include Adintrevimab, Cilgavimab, Sotrovimab (also reported in the Art as Xevudy™), Imdevimab (also reported in the Art as Ronapreve™), Tixagevimab, Regdanvimab (also reported in the Art as Regkirona™), Casirivimab, Etesivimab, Bamlanivimab and combinations thereof, including: Casirivimab in combination with Imdevimab, Bamlanivimab in combination with Etesevimab, Cilgavimab in combination with Tixagevimab. Overall, the neutralizing antibodies according to the present disclosure are demonstrated as potent binders for the RBDs of all the tested variants.

FIGS. 16C and 16D and 16E further illustrate the binding competition for each couple of antibodies and for a plurality of RBD constructs. Overall, Cv2.1169 and Cv2.3194 are demonstrated as among the best class 1 competitors. It is further demonstrated herein with an ELISA assay that their binding interface is complementary to a selection of other therapeutic antibodies; in particular those selected from a group consisting of: Adintrevimab, Cilgavimab, Sotrovimab, Imdevimab.

FIGS. 17 and 18 further illustrate that, remarkably, Cv2.1169 also ranked among the strongest cross-neutralizers when compared to the parental versions of benchmarked antibodies used in clinics or in development. In particular, Cv2.1169 cross-competed with class 1 benchmarked SARS-CoV-2 neutralizers (CT-P59, COV2-2196, REGN10933, and CB6), but also moderately with class 2 antibody LY-CoV555, for the binding to Spike and RBD proteins.

15. In Vivo Therapeutic and Prophylactic Activity of Cv2.1169 and Benchmarked Antibodies in Hamsters Infected with SARS-CoV-2 Delta and Omicron BA.1 Variants.

The therapeutic efficacy of Cv2.1169 and selected benchmarked antibodies (Evusheld [Cilgavimab+Tixagevimab] and S309/Sotrovimab) was tested in golden Syrian hamsters infected with SARS-CoV-2 variants Delta and Omicron BA.1 (FIG. 19B).

Animals were infected intranasally (i.n.) with 10⁴ plaque forming units (PFU) of SARS-CoV-2 Delta (FIG. 19A) or Omicron BA.1 (FIG. 19C) and received two different treatment regimen:
24 h post-infection with SARS-CoV-2 Delta, an intraperitoneal (i.p.) injection of Cv2.1169 IgG or Evusheld at 2 mg/kg (and 6 mg/kg (FIG. 19A);
24 h post-infection with SARS-CoV-2 BA.1, an intraperitoneal (i.p.) injection of Cv2.1169, Sotrovimab, or Evusheld antibodies at a concentration of 3 mg/kg. Cv2.1169 IgG antibody was also administrated at 30 mg/kg (FIG. 19C).

The results show that a single injection of Cv2.1169 IgG and Evusheld in infected hamsters induced a significant decrease of intra-lung SARS-CoV-2 Delta viral infectivity and viremia at 2 mg/kg and 6 mg/kg (FIG. 19A). Similar data were obtained with hamsters infected with Omicron BA.1 receiving a single injection of Cv2.1169 IgG and Evusheld preparation at 6 mg/kg (FIG. 19C).

The prophylactic activity of Cv2.1169 was also tested against SARS-CoV-2 Omicron BA.1 in Syrian Hamsters. A single administration of Cv2.1169 IgG antibodies 24 h prior to infection with 10⁴ PFU of SARS-CoV-2 Omicron BA.1 variant significantly decreased intra-lung viral infectivity and viremia, as well as body weight loss 80 h post-infection at both 3 mg/kg and 30 mg/kg as compared to Evusheld preparation (FIG. 19C).

Overall, in vivo experiments in Syrian Hamsters show that Cv2.1169 has higher therapeutic & prophylactic potentials than Sotrovimab and Evusheld against SARS-CoV-2 VOCs Delta and Omicron BA.1.

For activity against Wuhan SARS-CoV-2 see also Section 6. In vivo therapeutic activity of potent SARS-CoV-2 neutralizing antibody. For activity against VOCs see also Section 13. In vivo therapeutic activity of potent SARS-CoV-2 neutralizing antibody Cv2.1169 against the Beta variant.

### CONCLUSIONS

To examine the protective humoral response against SARS-CoV2, the inventors have cloned and characterized 101 human antibodies specific to the SARS-CoV2-S from memory B cells of ten COVID-19 convalescents selected based on high neutralizing antibody titers. They found that although human SARS-CoV-2 antibodies are encoded by a diverse set of immunoglobulin genes, several B-cell clones were shared among different individuals. Antibodies recognized various conformational epitopes, with the vast majority targeting the S2 subunit. About 10% of all SARS-CoV2-S-specific antibodies recognized the receptor binding domain (RBD), and among those, 5 potently neutralized SARS-CoV2 in vitro. SARS-CoV-2 neutralizers did not react with other Coronaviruses and showed cross-competition for RBD binding. None of the anti-S2 were neutralizing however, many harbored Fc-dependent effector functions when tested in vitro for ADCC and CDC potential. Noteworthy, the most potent antibody, Cv2.1169 also neutralized D614G, B1.1.7, B.1.351 and P.1 viral variants in vitro comparably to the original Wuhan strain. Remarkably, monotherapy with the ultra-potent neutralizing antibody Cv2.1169 induced a viremia decline in vivo in mouse and Hamster SARS-CoV2 models and protected all infected mice from death.

The inventors further show herein that the SARS-CoV2-S-specific antibodies also efficiently neutralize a plurality of Variants of Concern, including the kappa (κ) delta (δ), delta+(δ+) and omicron (o) variants.

Among the 102 SARS-CoV-2 antibodies described in this study, Cv2.1169 and Cv2.3194 were the sole potent neutralizers with a sustained activity against all SARS-CoV-2 variants including Omicron BA.1 and BA.2 subtypes. Comparably to typical class 1 anti-RBD antibodies, Cv2.3194 uses VH3-53 variable genes and displays a short CDRH3, but differs from the others by its resistance to escape mutations in the VOCs. Indeed, VH3-53-encoded anti-RBD antibodies usually lose their capacity to neutralize SARS-CoV-2 viruses with mutations in position K417 and N501 including the α, β, γ, and o variants. A rare mutation in the CDRκ1 of Vκ3-20-expressing class 1 anti-RBD antibodies (P30S) has been proposed to rescue VOC neutralization, but is absent in Cv2.3194. As the Cv2.3194 Fab/RBD complex did not crystallize, the molecular basis for its unaltered potent cross-neutralizing capacity against all VOCs remain to be solved. Besides Cv2.3194, we isolated another potent SARS-CoV-2 cross-neutralizing antibody, Cv2.1169, a class 1 neutralizer binding to RBD with a modest total buried surface area. Excepted for Omicron BA.1 and BA.2, all mutated RBD residues in the SARS-CoV-2 VOCs had a negligeable impact on the SARS-CoV-2 binding and neutralizing capacity of Cv2.1169. Based on structural data analysis, inventors identified the RBM residues in position F486 and N487 as critical for Cv2.1169 binding, acting as anchors and contributing to tolerate the T478K mutation present in several VOCs. Importantly, as previously shown for VH1-58-class antibody S2E12, substitutions in position F486 and N487 are unlikely to occur in potential future VOCs because of their deleterious effects in reducing both, RBD-binding to ACE2 and viral replicative fitness. Hence, Cv2.1169 belongs to a class of broad SARS-CoV-2 neutralizer (i.e., S2E12, A23.58.1, AZD8895 [COV2-2196]) with a high barrier to viral escape and one of the lowest escapability. Also, the diminished potency of Cv2.1169 against SARS-CoV-2 Omicron appears moderate when compared to other neutralizing antibodies to the RBD “VH1-58 supersite” that drastically reduced or lost their activity against BA.1 and BA.2.

SARS-CoV-2 animal models using rodents and non-human primates have been pivotal in demonstrating the in vivo prophylactic and therapeutic capacity of human neutralizing anti-spike antibodies. The inventors show that Cv2.1169 IgG efficiently prevents and/or protects animals from infection with SARS-CoV-2 and its VOC β. Cv2.1169 was originally expressed by circulating blood IgA-expressing activated memory B cells likely developing in mucosal tissues, and we established that Cv2.1169 IgA antibodies can protect mice from SARS-CoV-2 VOC β. Hence, one can assume that such antibodies if locally present at mucosal surfaces, particularly as dimeric IgAs, could efficiently neutralize and/or eliminate virions and therefore, potentially diminish the risk of infection by SARS-CoV-2 variants. In this regard, longer hinge region and multivalency of IgA1 antibody dimers allow enhancing SARS-CoV-2 neutralization in vitro as compared to their IgG1 counterparts (20, 78). In line with this, inventors found that the loss of neutralization activity of Cv2.1169 against BA.1 and BA.2 was greatly rescued by avidity effects of the antibody produced in its dimeric IgA form.

Several escape mutations in the spike of SARS-CoV-2 variants caused resistance to antibody neutralization, compromising vaccine and therapeutic antibody efficacy. Remarkably, Cv2.1169 and Cv2.3194 demonstrated a broad activity, neutralizing not only VOCs α, β, γ, δ and δ+ but also BA.1 and BA.2, and ranked as the most potent cross-neutralizer when compared to benchmarked antibodies used in clinics. Adjunct to its neutralizing activity, the strong ADCP potential of Cv2.1169 IgG antibodies could contribute to eliminating cell-free and cell-associated virions and stimulating adaptive immunity via vaccinal effects. Taking into account healthcare benefits afforded by antibody therapies to fight COVID-19, and considering the excellent antiviral attributes of Cv2.1169 and Cv2.3194, they represent promising candidates for prophylactic and/or therapeutic strategies against COVID-19. Long-acting versions of these broadly SARS-CoV-2 neutralizing antibodies with extended half-life could be used to provide protective immunity in immunocompromised populations.

### REFERENCES

1. Buchrieser J et al. Syncytia formation by SARS-CoV-2-infected cells. EMBO J. 2020; 39(23):1-12.
2. Grzelak L et al. A comparison of four serological assays for detecting anti-SARS-CoV-2 antibodies in human serum samples from different populations. Sci. Transl. Med. 2020; 12(559). doi:10.1126/scitranslmed.abc3103
3. Wardemann H. Predominant Autoantibody Production by Early Human B Cell Precursors. Science (80-.). 2003; 301(5638):1374-1377.
4. Meffre E et al. Surrogate Light Chain Expressing Human Peripheral B Cells Produce Self-reactive Antibodies. J. Exp. Med. 2004; 199(1):145-150.
5. Lorin V, Mouquet H. Efficient generation of human IgA monoclonal antibodies. J. Immunol. Methods 2015; 422:102-110.
6. Tiller T et al. Efficient generation of monoclonal antibodies from single human B cells by single cell RT-PCR and expression vector cloning. J. Immunol. Methods 2008; 329(1-2):112-124.
7. Mouquet H et al. Memory B Cell Antibodies to HIV-1 gp140 Cloned from Individuals Infected with Clade A and B Viruses. PLoS One 2011; 6(9):e24078.
8. Mouquet H et al. Complex-type N-glycan recognition by potent broadly neutralizing HIV antibodies. Proc. Natl. Acad. Sci. 2012; 109(47):E3268-E3277.
9. Planchais C et al. HIV-1 Envelope Recognition by Polyreactive and Cross-Reactive Intestinal B Cells. Cell Rep. 2019; 27(2):572-585.e7.
10. Jérémy Dufloo, Luivine Grzelak, Isabelle Staropoli, Yoann Madec, Laura Tondeur, Frangois Anna, Stéphane Pelleau, Aurélie Wiedemann, CyrilPlanchais, Julian Buchrieser, Rémy Robinot, Marie-Noelle Ungeheuer, Hugo Mouquet, Pierre Charneau, Michael White, Yv TB. Asymptomatic and symptomatic SARS-CoV-2 infections elicit polyfunctional antibodies.2020;
11. Melo GD De et al. Anti-COVID-19 efficacy of ivermectin in the golden hamster The devastating coronavirus disease 2019 (COVID-19) pandemic, due to SARS-CoV-2, has caused more than 47 million confirmed cases and more than 1.2 million human deaths around the globe 1, and most of the severe cases of COVID-19 in humans are associated with neurological symptoms such as anosmia and ageusia, and uncontrolled inflammatory immune response 2-5. Among therapeutic options 6-8, the use of the anti-parasitic drug ivermectin (IVM), has been proposed, given its possible anti-SARS-CoV-2 activity 9. Ivermectin is a positive allosteric modulator of the α—7 nicotinic acetylcholine receptor 10, which has been suggested to represent a target for the control of Covid-19 infection 11, with a potential immunomodulatory activity 12. We assessed the effects of IVM in SARS-CoV-2-intranasally-inoculated golden Syrian hamsters. Even though ivermectin had no effect on viral load, SARS-Cov-2-associated pathology was greatly attenuated. IVM had a sex-dependent and compartmentalized immunomodulatory effect, preventing clinical deterioration and reducing olfactory deficit in infected animals. Importantly, ivermectin dramatically reduced the I1-6/I1-10 ratio in lung tissue, which likely accounts for the more favorable clinical presentation in treated animals. Our data support IVM as a promising anti-COVID-19 drug2020; 1-20.
12. Baer A, Kehn-Hall K. Viral concentration determination through plaque assays: Using traditional and novel overlay systems. J. Vis. Exp. 2014(93):1-10.
13. Prigent et al., Eur. J. Immunol., 2016, 46, 2340-2351.

1-85. (canceled)

86. A monoclonal antibody directed against the viral Spike protein receptor binding-domain (RBD) of Severe Acute Respiratory Syndrome-related Coronavirus 2 (SARS-CoV-2), or antigen-binding fragment thereof, characterized in that it comprises:
a heavy chain variable domain comprising all three of: a heavy chain CDR1 of SEQ ID NO: 23, a heavy chain CDR2 of SEQ ID NO: 24 and a heavy chain CDR3 of SEQ ID NO: 25, or variants thereof comprising up to 1 amino acid mutation in the sequence of 1, 2 or 3 CDRs; and a light chain variable domain comprising all three of: a light chain CDR1 of SEQ ID NO: 26, a light chain CDR2 of SEQ ID NO: 27 and a light chain CDR3 of SEQ ID NO: 28, or variants thereof comprising up to 1 amino acid mutation in the sequence of 1, 2 or 3 CDRs; or
a heavy chain variable domain comprising all three of: a heavy chain CDR1 of SEQ ID NO: 35, a heavy chain CDR2 of SEQ ID NO: 36 and a heavy chain CDR3 of SEQ ID NO: 37, or variants thereof comprising up to 2 amino acid mutations in the sequence of 1, 2 or 3 CDRs; and a light chain variable domain comprising all three of: a light chain CDR1 of SEQ ID NO: 38, a light chain CDR2 of SEQ ID NO: 39 and a light chain CDR3 of SEQ ID NO: 40, or variants thereof comprising up to 2 amino acid mutations in the sequence of 1, 2 or 3 CDRs.

87. A human neutralizing monoclonal antibody against Severe Acute Respiratory Syndrome-related Coronavirus 2 (SARS-CoV-2), or antigen-binding fragment thereof, which specifically binds to the viral Spike protein receptor-binding domain (RBD) and is a competitive inhibitor of binding to the RBD of at least one of the following reference antibodies:
the reference human antibody Cv2.1169 comprising (i) a heavy chain variable region comprising the amino acid sequence of SEQ ID NO: 3 and (ii) a light chain variable region comprising the amino acid sequence of SEQ ID NO: 4; and
the reference human antibody Cv2.3194 comprising (i) a heavy chain variable region comprising the amino acid sequence of SEQ ID NO: 7 and a light chain variable region comprising the amino acid sequence of SEQ ID NO: 8;
said human neutralizing antibody, or antigen-binding fragment thereof, comprising:
a) a heavy chain variable domain comprising: a heavy chain CDR1 of SEQ ID NO: 23, a heavy chain CDR2 of SEQ ID NO: 24, and a heavy chain CDR3 of SEQ ID NO: 25, and a light chain variable domain comprising: a light chain CDR1 of SEQ ID NO: 26, a light chain CDR2 of SEQ ID NO: 27 and a light chain CDR3 of SEQ ID NO: 2, or
b) a heavy chain variable domain comprising: a heavy chain CDR1 of SEQ ID NO: 35, a heavy chain CDR2 of SEQ ID NO: 36 and a heavy chain CDR3 of SEQ ID NO: 37, or variants thereof comprising up to 2 amino acid mutations in the sequence of 1, 2 or 3 CDRs; and a light chain variable domain comprising: a light chain CDR1 of SEQ ID NO: 38, a light chain CDR2 of SEQ ID NO: 39, and a light chain CDR3 of SEQ ID NO: 40, or variants thereof comprising up to 2 amino acid mutations in the sequence of 1, 2 or 3 CDRs.

88. The antibody according to claim 86, or the antigen-binding fragment thereof, which comprises: a heavy chain CDR1 of SEQ ID NO: 23, a heavy chain CDR2 of SEQ ID NO: 24, and a heavy chain CDR3 of SEQ ID NO: 25, and a light chain variable domain comprising: a light chain CDR1 of SEQ ID NO: 26, a light chain CDR2 of SEQ ID NO: 27 and a light chain CDR3 of SEQ ID NO: 28.

89. The antibody according to claim 86, or the antigen-binding fragment thereof, which comprises: a heavy chain CDR1 of SEQ ID NO: 35, a heavy chain CDR2 of SEQ ID NO: 36, and a heavy chain CDR3 of SEQ ID NO: 37, and a light chain variable domain comprising: a light chain CDR1 of SEQ ID NO: 38, a light chain CDR2 of SEQ ID NO: 39 and a light chain CDR3 of SEQ ID NO: 40.

90. The antibody according to claim 86, or the antigen-binding fragment thereof, which comprises a heavy chain amino acid sequence of SEQ ID NO: 13 or 133 and a light chain amino acid sequence of SEQ ID NO: 14; or a heavy chain amino acid sequence of SEQ ID NO: 17 or 135 and a light chain amino acid sequence of SEQ ID NO: 18.

91. The antibody according to claim 86, or the binding fragment thereof, wherein said heavy chain variable domains are associated with IgG or IgA constant region.

92. The antibody according to claim 91, or the antigen-binding fragment thereof wherein the constant region comprises mutation(s) and/or modifications that silence antibody effector functions and/or increase antibody half-life in vivo.

93. The antibody according to claim 86, or the antigen-binding fragment thereof, which neutralizes at least one SARS-CoV-2 chosen from isolate Wuhan-Hu-1, SARS-CoV-2 variant D614G, and SARS-CoV-2 variants comprising mutation(s) in the RBD domain; which neutralizes at least one SARS-CoV-2 variant chosen from the lineages B.1.1.7, P.1 and B.1.351 and B.1.617 and B.1.1.529 and BA.2; or which does not cross-react with at least one human coronavirus selected from SARS-CoV-1, MERS-CoV, NL63-CoV, OC43-CoV, HKU1-CoV and 229E-CoV.

94. The antibody according to claim 86, or the antigen-binding fragment thereof, which is produced recombinantly and comprises a non-native human glycosylation pattern and/or a non-human glycosylation pattern.

95. The antibody according to claim 86, or the antigen-binding fragment thereof, which further comprises a detectable label.

96. An isolated nucleic acid encoding an antibody or the antigen-binding fragment thereof, according to claim 86.

97. An expression vector for the recombinant production of an antibody or the antigen-binding fragment thereof according to claim 86 in a host cell, comprising at least one nucleic acid encoding said antibody or said antigen-binding fragment thereof.

98. A host cell comprising an expression vector according to claim 97.

99. A method of producing the antibody or antigen-binding fragment thereof according to claim 86, comprising: culturing a host cell comprising an expression vector comprising at least one nucleic acid encoding the antibody or the antigen-binding fragment thereof.

100. A pharmaceutical composition, comprising
the antibody according to claim 86 or the antigen-binding fragment thereof, or a nucleic acid encoding the antibody or the antigen-binding fragment thereof, or a vector comprising the nucleic acid, and
at least one of a pharmaceutically acceptable carrier, an adjuvant, and a preservative.

101. The antibody according claim 86, or antigen-binding fragment thereof, a nucleic acid or vector encoding the antibody or the antigen-binding fragment thereof, or a pharmaceutical composition comprising the antibody or the antigen-binding fragment thereof; for use as a medicament in combination with a second antibody which specifically neutralizes the Severe Acute Respiratory Syndrome-related Coronavirus 2 (SARS-CoV-2), said second antibody being not a competitive inhibitor of binding to the RBD with the first antibody.

102. A method for the manufacturing medicament comprising at least the step of combining an antibody according to claim 86 or the antigen-binding fragment thereof, or a pharmaceutical composition comprising the antibody or the antigen-binding fragment thereof, with at least one pharmaceutical carrier, diluent and/or adjuvant, wherein said medicament is for the prevention or treatment of SARS-CoV-2 infection and associated COVID-19 disease.

103. A kit comprising the antibody according to claim 86, or the antigen-binding fragment thereof, a nucleic acid encoding the antibody or the antigen-binding fragment, or a vector comprising the nucleic acid, or pharmaceutical composition comprising the antibody or the antigen-binding fragment thereof, or the nucleic acid or the vector.

104. A method for detecting a Coronaviridae in a sample comprising: contacting said sample with the antibody according to claim 86 or the antigen-binding fragment thereof, and detecting antigen-antibody complexes formed, thereby detecting a presence, absence or level of Coronaviridae in the sample.

105. A method for preventing and/or reducing the likelihood of occurrence of a Coronaviridae infection, or of an occurrence of a complication of a Coronaviridae infection; or improving an immune response against a Coronaviridae virus; or improving an immune response against a viral Spike protein receptor-binding domain (RBD) of a Coronaviridae virus or a fragment thereof, or treating a Coronaviridae-associated disease; comprising
administering, to a subject in need thereof, the antibody according to claim 86 or the antigen-binding fragment thereof, a nucleic acid encoding the antibody or the antigen-binding fragment thereof, a vector comprising the nucleic acid, or a pharmaceutical composition comprising the antibody or the antigen-binding fragment thereof or the nucleic acid or the vector.

106. A medical device, comprising an antibody according to claim 86, or the antigen-binding fragment thereof, a nucleic acid encoding the antibody or the antigen-binding fragment thereof, a vector comprising the nucleic acid, or a pharmaceutical composition comprising the antibody or the antigen-binding fragment thereof or the nucleic acid or the vector.

107. The antibody according to claim 86 or the antigen-binding fragment thereof, which comprises a heavy chain variable region comprising an amino acid sequence having at least 90% identity with SEQ ID NO: 7, and a light chain variable region comprising an amino acid sequence having at least 90% identity with SEQ ID NO: 8.

108. The antibody according to claim 86, or the antigen-binding fragment thereof, which comprises a heavy chain variable region comprising an amino acid sequence having at least 96% identity with SEQ ID NO: 7, and a light chain variable region comprising an amino acid sequence having at least 96% identity with SEQ ID NO: 8.

109. The antibody according to claim 86, or the antigen-binding fragment thereof, which comprises a heavy chain amino acid sequence having at least 90% identity with SEQ ID NO: 17 or 135 and a light chain amino acid sequence having at least 90% identity with SEQ ID NO: 18.

110. The antibody according to claim 86, or the antigen-binding fragment thereof, which comprises a heavy chain amino acid sequence having at least 96% identity with SEQ ID NO: 17 or 135 and a light chain amino acid sequence having at least 96% identity with SEQ ID NO: 18.
